(19) United States
(12) Patent Application Publication
(10) Pub. No.: US 2025/0026745 A1
(43) Pub. Date: Jan. 23, 2025

(54) 17-BETA-HYDROXYSTEROID DEHYDROGENASE TYPE 13 INHIBITORS AND METHODS OF USE THEREOF

(71) Applicant: Bluejay Therapeutics, Inc., San Mateo, CA (US)

(72) Inventors: Jeffery Zablocki, San Mateo, CA (US); Dmitry Koltun, San Mateo, CA (US); Hassan Javanbakht, San Mateo, CA (US); Keting Chu, San Mateo, CA (US); Jerome Deval, San Mateo, CA (US)

(21) Appl. No.: 18/747,436

(22) Filed: Jun. 18, 2024

Related U.S. Application Data
(60) Provisional application No. 63/509,027, filed on Jun. 19, 2023.
(60) Provisional application No. 63/568,312, filed on Mar. 21, 2024.

(51) Int. Cl. C07D 417/06; A61K 31/433; A61K 31/4439; A61K 31/506; A61K 45/06; C07D 417/14

(52) U.S. Cl. CPC C07D 417/06; A61K 31/433; A61K 31/4439; A61K 31/506; A61K 45/06; C07D 417/14

(57) ABSTRACT

17-Beta-hydroxysteroid dehydrogenase type 13 (17β-HSD13) plays an important role in the pathogenesis and progression of Non-alcoholic fatty liver disease (NAFLD), also known as Metabolic dysfunction-associated steatotic liver disease (MASLD), and Alcohol-related liver disease (ARLD). Loss-of-function variant of 17β-HSD13 has been associated with a significantly reduced risk of NAFLD (MASLD), cirrhosis associated with nonalcoholic steatohepatitis (NASH (MASH)), alcoholic-related liver disease, alcoholic cirrhosis, hepatocellular carcinoma (HCC), hepatitis C chronic liver disease, pediatric liver disease, NASH (MASH) disease severity, ballooning degeneration, lobular inflammation, and fibrosis. The present invention relates to hydantoin compounds useful as inhibitors of 17β-HSD13 and methods for their preparation and use for treatment and prevention of above mentioned liver diseases.

### TECHNICAL FIELD

The present invention relates generally to compounds and pharmaceutical compositions useful as inhibitors. Specifically, the present invention relates to compounds useful as inhibitors of 17β-HSD13 and methods for their preparation and use.

### BACKGROUND OF THE INVENTION

Non-alcoholic fatty liver disease (NAFLD) also known as Metabolic dysfunction-associated steatotic liver disease (MASLD), characterized by ≥5% hepatic fat accumulation, encompasses a heterogenous series of disorders ranging from liver steatosis to more severe non-alcoholic steatohepatitis (NASH) also known as Metabolic dysfunction-associated steatohepatitis (MASH), which may include inflammatory cell infiltration, hepatocyte ballooning, and fibrosis (Perumpail B J World J Gastroenterol. 2017, 23(47):8263-76; Anstee Q M, Nat Rev Gastroenterol Hepatol. 2013; 10(6):330-44).

Livers of NAFLD (MASLD) patients contain abnormally high levels of neutral lipids such as triglycerides and cholesterol ester stored in lipid droplets (LD), a subcellular organelle in hepatocytes. LDs are complex and metabolically active organelles. Alteration of LD biogenesis, growth, or degradation affects their sizes and numbers in liver cells. Excessive biogenesis and constant growth of LDs are the most distinctive characteristics of NAFLD (MASLD) and are closely associated with the progression of NAFLD towards NASH (MASH) and cirrhosis (Scorletti E., Carr R. M. 2021, J. Hepatol. 10.1016/j.jhep.2021.11.009).

In its most severe form, NASH can progress to liver cirrhosis and hepatocellular carcinoma. Although estimates vary among studies, the worldwide prevalence of NAFLD (MASLD) could be as high as 25% (Younossi Z M, Hepatology. 2016; 64(1):73-84). The American Liver Foundation estimated that NAFLD (MASLD) is the most common cause of chronic liver disease in the United States, affecting between 80 and 100 million individuals. Twenty percent of these patients develop NASH (MASH), representing approximately 5% of total adults.

Common NAFLD (MASLD) NASH (MASH) comorbidities include obesity, type II diabetes, hyperlipidemia, hypertension, and metabolic syndrome. In the absence of any approved treatment, the medical burden and healthcare costs associated with NASH (MASH) are immense.

Research on the medical treatment of NASH (MASH) consists of modulating either sugar or fat metabolism or targeting one of the downstream pathways associated with liver inflammation and fibrosis.

Alcohol-related liver disease (ARLD) is also prevalent worldwide and refers to a progressive liver disease brought on by excessive, prolonged alcohol use. Various diseases states of ARLD exist and include alcoholic fatty liver (alcoholic steatosis), alcoholic hepatitis, and cirrhosis.

Human genome-wide association studies (GWAS) have revealed that certain genes encoding LD-associated proteins such as fat storage inducing transmembrane protein 2 (FIT2), adipose triglyceride lipase (ATGL; PNPLA2), patatin-like phospholipase domain containing 3 (PNPLA3) and 17-Beta-hydroxysteroid dehydrogenase type 13 (17β-HSD13) play an important role in the pathogenesis and progression of NAFLD (MASLD).

17β-HSD13 belongs to the HSD17B family with NAD(P)H/NAD(P)+-dependent oxidoreductase activity that catalyzes the interconversion between 17-ketosteroids and 17-hydroxysteroids to maintain the balance between less potent (17-keto) and more potent (17β-hydroxy) forms of estrogens and androgens (Poutanen M., 2019, Mol. Cell Endocrinol. 489, 1-2. 10.1016/j.mce.2019.04.008). There are 15 members of this family identified. Most of them are related to the activation or inactivation of sex hormones (17β-HSD1, 17β-HSD2, 17β-HSD3, 17β-HSD5, 17β-HSD6), and the other members are involved in fatty acid metabolism, cholesterol biosynthesis, and bile acid production (Saloniemi T., 2012, J. Endocrinol. 212 (1), 27-40. 10.1530/JOE-11-0315).

The levels of 17β-HSD13 protein are up regulated in the livers of patients and mice with NAFLD (MASLD) (Su W, 2014, Proc. Natl. Acad. Sci. 111 (31), 11437-11442. 10.1073/pnas.1410741111). Overexpression results in an increase in the number and size of LDs, whereas gene silencing of 17β-HSD13 attenuates oleic acid-induced LD formation in cultured hepatocytes. Hepatic overexpression of 17β-HSD13 protein in C57BL/6 mice has been shown to significantly increase lipogenesis and triglyceride (TG) contents in the livers, leading to a fatty liver phenotype.

Loss-of-function variant of 17β-HSD13 has been associated with a significantly reduced risk of NAFLD (MASLD), cirrhosis associated with nonalcoholic steatohepatitis (NASH (MASH)), alcoholic-related liver disease, alcoholic cirrhosis, hepatocellular carcinoma (HCC), hepatitis C chronic liver disease, pediatric liver disease, NASH (MASH) disease severity, ballooning degeneration, lobular inflammation, and fibrosis (N. S. Abul-Husn, et al., N Engl. J Med. 2018, 378, 1096-1106; C. J. Pirola, et al., J Lipid Res. 2019, 60, 176-185).

Small molecules and oligonucleotides targeting 17β-HSD13 have been disclosed in WO 2021/003295A1, WO 2023/023310, WO 2022/020730, WO 2020/132564, WO 2020/061177, WO 2019/075181, WO 2019/183164, WO 2019/183329, and WO 2018/136758.

Early-stage clinical data from NASH (MASH) patients treated with oligonucleotides provide preliminary proof-of concept that targeting 17β-HSD13 is accompanied with reduction in alanine aminotransferase and improvement in non-alcoholic fatty liver disease activity score (Mak L-Y, J Hepatol 2023 April; 78(4):684-692. doi: 10.1016; Sanyal A, EASL Congress 2023, oral presentation, abstract #OS-062).

Small molecules and oligonucleotides targeting 17β-HSD13 have been disclosed in WO 2021/003295A1, WO 2023/023310, WO 2022/020730, WO 2020/132564, WO 2020/061177, WO 2019/075181, WO 2019/183164, WO 2019/183329, WO 2018/136758, BI patent WO 2023/237504, and Thamm et al. J. Med. Chem. 2022, 66, 2832-2850. In the latter publication, Boehringer Ingelheim (BI) describes the tool molecule BI-3231, a novel potent and selective 17β-HSD13 inhibitor shown below. While BI-3231 is useful as a tool molecule, it is prone to rapid glucuronidation on the phenol moiety leading to fast in vivo clearance.

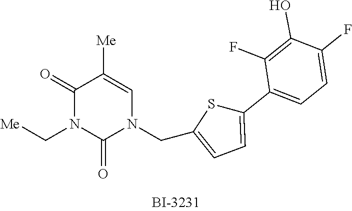 [SMILES: CCn1c(=O)c(C)cn(Cc2ccc(-c3ccc(F)c(O)c3F)s2)c1=O]

One potential way to address rapid glucuronidation of phenolic based 17β-HSD13 inhibitors is to sterically encumber the phenol to diminish the glucuronidation. This can be potentially accomplished using larger halogens like chloro groups for example. In addition, structural changes to the molecule that decrease the amount of glucuronidation can be accomplished by making a pyridone moiety that exists as a tautomeric hydroxy group with the keto form. Alternatively, functional groups can be added to the phenol ring or other portions of the molecule that shift the ratio of parent:glucuronide to increased amounts of parent compound in the liver for 17β-HSD13 target inhibition. Levels of parent:glucuronide in the liver across species can be measured by LC-MS assays of liver tissue at various time points following oral dosing to establish sufficient parent compound is present for 17β-HSD13 target inhibition. The analytical measurement of excess parent in the liver to cover the EC₉₀ for 17β-HSD13 target inhibition is strong support for the utility of the compounds of this invention. In addition, the presence of sufficient parent compound for target inhibition in the liver may potentially be further confirmed by changes in pharmacodynamic biomarkers of 17β-HSD13 target engagement (e.g. ceramide). Several pre-clinical studies with 17β-HSD13 inhibitors provide support for changes in pharmacodynamic biomarkers that support 17β-HSD13 for the treatment of liver diseases. For instance, reduction in certain liver lipids such as cholesteryl ester, ceramide, sphingosine 1-phosphate, and hexosylceramide has been reported in C57BL/6 mice fed with choline-deficient high fat diet and dosed for one week with an 17β-HSD13 inhibitor (Enanta EASL Poster 2022). In a separate study, knocking down 17β-HSD13 expression in C57BL/6 mice fed with choline-deficient high fat diet resulted in changes in several metabolites such as pyrimidines and phospholipids (doi.org/10.1073/pnas.2217543120). Direct evidence of target engagement reflected by changes in levels of certain plasma keto- and hydroxy-lipids has been obtained in Sprague-Dawley rats fed with choline-deficient high fat diet and dosed for two weeks with an 17β-HSD13 inhibitor (IniPharm EASL Poster 2023).

As used herein, “prodrugs” are molecules that are converted to active parent drug in vivo often addressing physical properties of the active parent that are unfavorable for high oral bioavailability. The “prodrugs” have a built-in structural lability, whether by chance or by design, that permits bioconversion in vivo to the active parent drug. This conversion can occur through a chemical or enzymatic process or a combination of the two. Conversion liberates the active drug from the masking “promoiety” or drug carrier in a way that the resulting molecule—the active metabolite—projects the full complement of the desired therapeutic effects. Examples of prodrugs are esters of carboxylic acids, boronic acids, and phosphonic acids. For more detailed review of prodrug strategy and examples see: Rautio, J., Meanwell, N., Di, L. et al. Nat Rev Drug Discov 2018, 17, 559-587; Erion, M. JPET 2005, Vol. 302, P. 554. The latter reference highlights the use of pro-drugs to enhance liver to plasma ratios of the parent drug by invoking Cyp 450 mediated cleavage of the pro-drug.

There is a need for the development of 17β-HSD13 inhibitors for the treatment and prevention of disease.

### SUMMARY OF THE INVENTION

As discussed above there is a strong need to develop effective treatments for liver diseases in general, and alcoholic or nonalcoholic liver disease, and cirrhosis. The present disclosure addresses these and other needs by providing new compounds, pharmaceutical compositions, and methods of treatment based on such compounds and pharmaceutical compositions.

The present invention relates to compounds which inhibit 17β-HSD13 as well as methods of using these compounds to treat diseases.

In one aspect, the disclosure provides compound, a pharmaceutically acceptable salt thereof, and a pharmaceutically acceptable prodrug thereof, of Formula I:

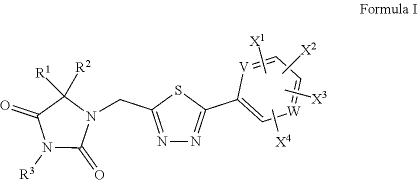

where X¹, X², X³, and X⁴ are each independently selected from the group consisting of H, F, Cl, Br, optionally substituted C₁-C₆ alkyl, C₁-C₄ haloalkyl, optionally substituted C₃-C₆ cycloalkyl, optionally substituted alkoxy, and hydroxy;
V is CH or N;
W is CH or N;
with a proviso that V and W are not simultaneously N and when either one or both of V and W are CH they can be independently substituted by X¹, X², X³, or X⁴ thereby becoming CX¹, CX², CX³, or CX⁴;
where R¹ and R² are each independently selected from hydrogen or optionally substituted C₁-C₈ linear or branched alkyl, with the proviso that R¹ and R² may be connected to one another to form a ring of 3 to 6 ring-carbons. Examples of the group

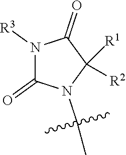

in Formula I can include:

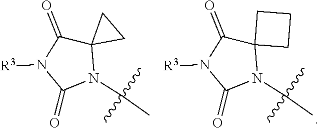

R³ in Formula I can be selected from hydrogen, optionally substituted C₁-C₈ linear or branched alkyl, optionally substituted C₁-C₈ cycloalkyl (including bicyclo- or tricycloalkyl), optionally substituted phenyl, optionally substituted aralkyl, optionally substituted heteroaralkyl, optionally substituted cyclopropylalkyl, optionally substituted C₁-C₈ alkynyl. In some embodiments, R³ is selected from hydrogen, optionally substituted C₁-C₈ linear or branched alkyl, optionally substituted C₁-C₈ cycloalkyl (including bicyclo- or tricycloalkyl), optionally substituted phenyl, optionally substituted benzyl, optionally substituted 2-phenethyl, optionally substituted cyclopropylalkyl, optionally substituted C₁-C₈ alkynyl.

In some embodiments of the compound of Formula I, R¹ and R² form a cyclopropyl ring as shown in Formula Ib:

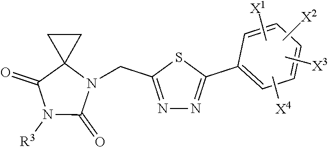

where X¹, X², X³, X⁴, and R³ are defined in the same way as in Formula I.

In some embodiments of the compound of Formula I, X⁴ is hydrogen as shown in Formula Ic:

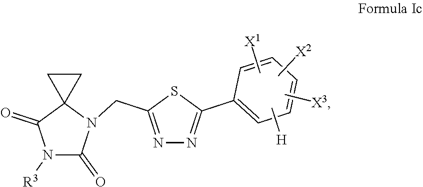

where X¹, X², X³, and R³ are defined in the same way as in Formula I.

In some embodiments of the compound of Formula I, R³ is CF₃—CH₂— as shown in Formula Id:

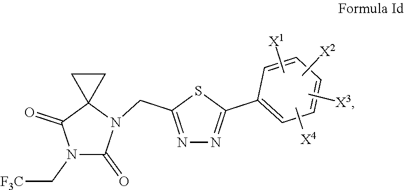

where X¹, X², X³, and X⁴ are defined in the same way as in Formula I.

In some embodiments of the compound of Formula I, R³ is Ph-CH₂— as shown in Formula Ie:

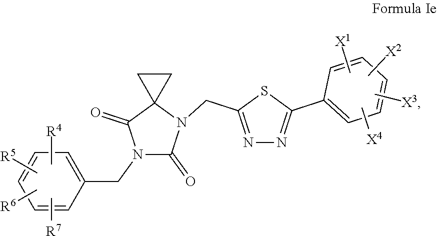

where X¹, X², X³, and X⁴ are defined in the same way as in Formula I; and where R⁴, R⁵, R⁶, and R⁷ are independently selected from halo, CN, amino, hydroxy, —C₁₋₃ alkyl, C₁-C₄ haloalkyl, optionally substituted phenyl, —OR*, —NR*₂, —SR*, —SO₂R*, —COOR*, or —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons.

In some embodiments of the compound of Formula I, R³ is as shown in Formula If:

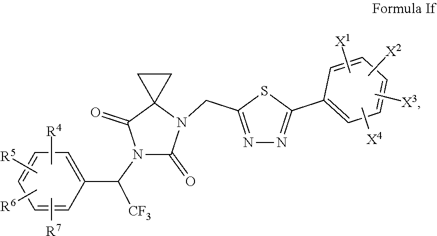

where X¹, X², X³, and X⁴ are defined in the same way as in Formula I; and where R⁴, R⁵, R⁶, and R⁷ are independently selected from halo, CN, amino, hydroxy, —C₁₋₃ alkyl, C₁-C₄ haloalkyl, optionally substituted phenyl, —OR*, —NR*₂, —SR*, —SO₂R*, —COOR*, or —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons.

In some embodiments of the compound of Formula I, a compound shown in Formula Ig is provided:

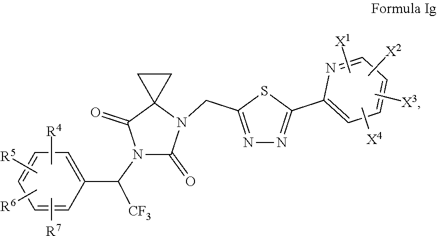

where X¹, X², X³, and X⁴ are defined in the same way as in Formula I; and where R⁴, R⁵, R⁶, and R⁷ are independently selected from halo, CN, amino, hydroxy, —C₁₋₃ alkyl, C₁-C₄ haloalkyl, optionally substituted phenyl, optionally substituted cyclopropyl, —OR*, —NR*₂, —SR*, —SO₂R*, —COOR*, or —CONR*₂, where each R* is independently H, C₁₋₄ alkyl, C₁-C₄ haloalkyl or C₃₋₈ cycloalkyl with the proviso that two R* on —CONR*₂ may form a ring of C₃-C₈ carbons.

### BRIEF DESCRIPTION OF THE DRAWINGS

FIGS. 1A-1C are graphs showing liver levels of certain compounds of the present invention following PO dosing in mice at 20 MPK.

### DETAILED DESCRIPTION OF THE INVENTION

The present invention provides novel compounds that inhibit 17β-HSD13 inhibitors for the treatment and prevention of diseases including non-alcoholic fatty liver disease (NAFLD (MASLD)), characterized by ≥5% hepatic fat accumulation, encompasses a heterogenous series of disorders ranging from liver steatosis to more severe non-alcoholic steatohepatitis (NASH (MASH)). In addition, the present invention provides novel compounds that inhibit 17β-HSD13 inhibitors for the treatment and prevention of diseases including various diseases states of ARLD exist and include alcoholic fatty liver (alcoholic steatosis), alcoholic hepatitis, and cirrhosis. Thus, the compounds of the invention are suitable for treatment of patients with liver disease including NAFLD (MASLD), NASH (MASH), alcoholic fatty liver (alcoholic steatosis), alcoholic hepatitis, and cirrhosis.

Each of the compounds of the Examples, including each compound listed in Tables, is a specific embodiment of the compounds of the invention.

The term “an optical isomer” or “a stereoisomer” refers to any of the various stereoisomeric configurations which may exist for a given compound of the present invention and includes geometric isomers. It is understood that a substituent may be attached at a chiral center of a carbon atom. The term “chiral” refers to molecules which have the property of non-superimposable on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold- Prelog R—S system. When a compound is a pure enantiomer the stereochemistry at each chiral carbon may be specified by either R or S.

Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

As used herein, “prodrugs” are molecules that are converted to active parent drug in vivo often addressing physical properties of the active parent that are unfavorable for high oral bioavailability. The “prodrugs” have a built-in structural lability, whether by chance or by design, that permits bioconversion in vivo to the active parent drug. This conversion can occur through a chemical or enzymatic process or a combination of the two. Conversion liberates the active drug from the masking “promoiety” or drug carrier in a way that the resulting molecule—the active metabolite—projects the full complement of the desired therapeutic effects. For more detailed review of prodrug strategy and examples see Rautio, J., Meanwell, N., Di, L. et al. Nat Rev Drug Discov 2018, 17, 559-587.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified. Unless otherwise stated, all crystalline forms of the compounds of the disclosure and salts thereof are also within the scope of the disclosure. The compounds of the disclosure can be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the disclosure are anhydrous and non-solvated. By “anhydrous” is meant that the crystalline form of the compound contains essentially no bound water in the crystal lattice structure, i.e., the compound does not form a crystalline hydrate. As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content. Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the disclosure are substantially isolated. By “substantially isolated” means that a particular compound is at least partially isolated from impurities. For example, in some embodiments a compound of the disclosure comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances. The present disclosure also includes salts of the compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid salts of basic residues such as amines); alkali (such as Li, Na, K, Mg, Ca) or organic (such as trialkylammonium) salts of acidic residues such can be synthesized from the parent compound which contains a basic or acidic as carboxylic acids; and the like. The salts of the present application moiety conventional chemical methods. Generally, such salts can be prepared by reacting the free acid or base forms of these compounds with a stoichiometric amount of the appropriate base or acid in water or in an organic solvent, or in a mixture of the two. In some embodiments, non-aqueous media like ether, ethyl acetate, ethanol, isopropanol, or acetonitrile (ACN) can be used.

The present application also includes pharmaceutically acceptable salts of the compounds described herein. The “pharmaceutically acceptable salts” include a subset of the “salts” described above which are conventional non-toxic salts of the parent compound formed, for example, from non-toxic inorganic or organic acids. Lists of suitable salts are found in Remington's Pharmaceutical Sciences, 17th ed., Mack Publishing Company, Easton, Pa., 1985, p. 1418 and Berge, S M et al, Journal of Pharmaceutical Science, 1977, 66, 1, 1-19. By way of an example, in an embodiment of the disclosure pharmaceutically acceptable salts can comprise a suitable anion selected from F—, Cl, Br—, I, OH—, —BF₄, CF₃SO₃—, monobasic sulfate, dibasic sulfate, monobasic phosphate, dibasic phosphate, or tribasic phosphate, NO₃—, PF₆—, NO₂—, carboxylate, aspartate, benzenesulfonate, benzoate, besylate, bicarbonate, bitartrate, camsylate, carbonate, citrate, decanoate, edetate, fumarate, gluceptate, gluconate, glutamate, glycolate, glycollyalarsanilate, hexanoate, hydrabamine, hydroxynaphthoate, isthionate, lactate, lactobionate, malate, maleate, mandelate, mesylate, methylbromide, methylnitrate, mucate, napsylate, octanoate, oleate, oxalate, palmitate, pamoate, pantothenate, polygalacturonate, propionate, salicylate, stearate, subacetate, succinate, tartrate, teoclate, tosylate, or triethiiodide. By way of another example, in an embodiment of the disclosure pharmaceutically acceptable salts can comprise a suitable cation selected from aluminum, arginine, benzathine, calcium, chloroprocaine, choline, diethanolamine, ethanolamine, ethylenediamine, lysine, magnesium, histidine, lithium, meglumine, potassium, procaine, sodium, triethylamine, or zinc. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Compounds as well as their pharmaceutically acceptable salts and prodrugs of the invention may be used in the treatment of states/conditions, disorders or diseases as described herein, or for the manufacture of pharmaceutical compositions for use in the treatment of these states/conditions, disorders or diseases. Further, use of compounds as well as their pharmaceutically acceptable salts and prodrugs of the invention for use in a therapy, e.g., treating a liver disease is provided. Use of the compounds as well as their pharmaceutically acceptable salts and prodrugs of the invention in manufacturing of medicament for treating states/conditions, disorders or diseases are provided. The invention provides methods of use of compounds of the present invention in the treatment of these diseases or for preparation of pharmaceutical compositions having compounds of the present invention for the treatment of these diseases. For example, the invention provides a method for treating a disease or condition in a subject in need thereof, comprising administering to the subject a therapeutically effective amount of a compound or a composition as described herein. The disease or condition can be a liver disease, and can be mediated by 17β-HSD13. For example, the disease or condition can be selected from the group consisting of nonalcoholic fatty liver disease (NAFLD), nonalcoholic steatohepatitis (NASH), alcoholic fatty liver (alcoholic steatosis), alcoholic hepatitis, liver cirrhosis, liver fibrosis, and hepatocellular carcinoma (HCC). The present invention also provides methods of use of compounds of the present invention for reducing the development of liver cirrhosis, cirrhotic decompensation, progression to model of end-stage liver disease (MELD), need for liver transplant, liver-related death, or hepatocellular carcinoma.

The term “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of at least one compound of Formula (I) or any subgenus thereof as active ingredient in combination with a pharmaceutically acceptable carrier, or optionally two or more pharmaceutically acceptable carriers. The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body.

Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; tale; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Typically, pharmaceutically acceptable carriers are sterilized and/or substantially pyrogen-free.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BIA), butylated hydroxytoluene (BHT), lecithin, propyl gallate, \hat{ }-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, inhalation, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored base, for example, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration may comprise one or more compounds of the invention in combination with one or more pharmaceutically acceptable carriers such as sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, glycol ethers, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Intravenous infusion is sometimes a preferred method of delivery for compounds of the invention. Infusion may be used to deliver a single daily dose or multiple doses. In some embodiments, a compound of the invention is administered by infusion over an interval between 15 minutes and 4 hours, typically between 0.5 and 3 hours. Such infusion may be used once per day, twice per day or up to three times per day.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration. These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated effects, will range from about 0.0001 to about 100 mg per kilogram of body weight per day, more preferably from about 0.01 to about 50 mg per kg per day, and still more preferably from about 0.11 to about 20 mg per kg per day. The daily dose of the compound may be administered once or twice or three times daily, to be determined.

The compound(s) (as well as their pharmaceutically acceptable salts and products) of the present invention may be administered alone or in combination (either sequentially or simultaneously) with other therapeutics including but not limited to immunomodulators, anti-viral and/or anti-inflammatory agents. Thus, methods of using the compounds of the invention include administering the compound as a pharmaceutical composition, wherein at least one compound of the invention is admixed with a pharmaceutically acceptable carrier prior to administration.

For example, pharmaceutical combination composition can include a first compound, a pharmaceutically acceptable salt or prodrug of the compound; a second compound being an anti-diabetic agent, a non-alcoholic steatohepatitis treatment agent, or a non-alcoholic fatty liver disease treatment agent; and a pharmaceutically acceptable carrier. The non-alcoholic steatohepatitis treatment agent or non-alcoholic fatty liver disease treatment agent can be an ACC inhibitor, a FASN inhibitor, a KHK inhibitor, a DGAT-2 inhibitor, an FXR agonist, metformin, incretin analogs, or an incretin receptor modulator. The anti-diabetic agent can be an SGLT-2 inhibitor, metformin, incretin analogs, an incretin receptor modulator, a DPP-4 inhibitor, or a PPAR agonist.

In general, it is expected that each of the therapeutic agents utilized in combination be utilized at levels that do not exceed the levels at which they are utilized individually. In some embodiments, the doses utilized in combination may be lower than those utilized individually.

The daily dose of the compound may be administered once or twice daily or three times daily, to be determined.

The compound(s) of the present invention may be administered alone or in combination (either sequentially or simultaneously) with other therapeutics. Thus, methods of using the compounds of the invention include administering the compound as a pharmaceutical composition, wherein at least one compound of the invention is admixed with a pharmaceutically acceptable carrier prior to administration.

By a “therapeutically effective amount” or “effective amount” of a compound or composition of the invention is meant an amount of the compound or composition that causes a therapeutic effect on the treated subject, at a reasonable benefit/risk ratio applicable to such treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene Protective Groups in Organic Synthesis, 4^{th} Ed., John Wiley & Sons: New York, 2006. In one non-limiting embodiment, protecting groups can include 1-chloroethyl carbonyl (ACE), acetoyl, benzyl (Bn), benzyloxy carbonyl (CBz), formyl, methyl carbonyl, trifluoroacetyl, t-butoxy carbonyl (Boc), and fluorenylmethyloxycarbonyl (Fmoc).

List of specific examples of Compounds of Formula I:

| Compound | Structure | Chemical name |
|---|---|---|
| I-1 | 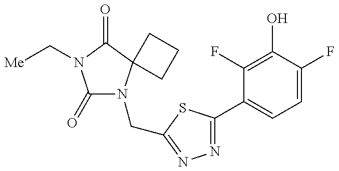 [SMILES: CCN1C(=O)N(Cc2nnc(-c3ccc(F)c(O)c3F)s2)C2(CCC2)C1=O] | 5-((5-(2,4-difluoro-3- hydroxyphenyl)-1,3,4-thiadiazol-2- yl)methyl)-7-ethyl-5,7- diazaspiro[3.4]octane-6,8-dione |
| I-2 | 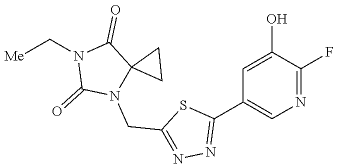 [SMILES: CCN1C(=O)N(Cc2nnc(-c3cnc(F)c(O)c3)s2)C2(CC2)C1=O] | 6-ethyl-4-((5-(6-fluoro-5- hydroxypyridin-3-yl)-1,3,4- thiadiazol-2-yl)methyl)-4,6- diazaspiro[2.4]heptane-5,7-dione |
| I-3 | 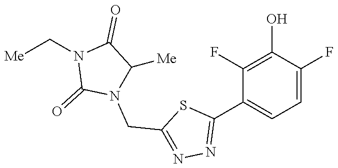 [SMILES: CCN1C(=O)C(C)N(Cc2nnc(-c3ccc(F)c(O)c3F)s2)C1=O] | 1-((5-(2,4-difluoro-3- hydroxyphenyl)-1,3,4-thiadiazol-2- yl)methyl)-3-ethyl-5- methylimidazolidine-2,4-dione |
| I-4 | 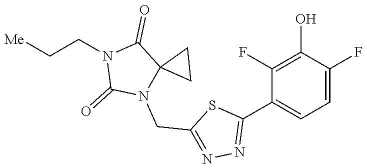 [SMILES: CCCN1C(=O)N(Cc2nnc(-c3ccc(F)c(O)c3F)s2)C2(CC2)C1=O] | 4-((5-(2,4-difluoro-3- hydroxyphenyl)-1,3,4-thiadiazol-2- yl)methyl)-6-propyl-4,6- diazaspiro[2.4]heptane-5,7-dione |
| I-5 | 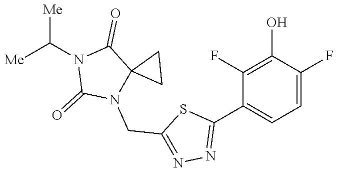 [SMILES: CC(C)N1C(=O)N(Cc2nnc(-c3ccc(F)c(O)c3F)s2)C2(CC2)C1=O] | 4-((5-(2,4-difluoro-3- hydroxyphenyl)-1,3,4-thiadiazol-2- yl)methyl)-6-isopropyl-4,6- diazaspiro[2.4]heptane-5,7-dione |
| I-6 | 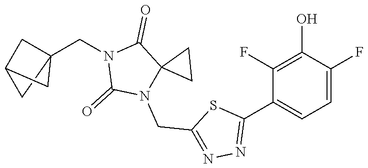 [SMILES: O=C1N(CC23CC(C2)C3)C(=O)C2(CC2)N1Cc1nnc(-c2ccc(F)c(O)c2F)s1] | 6-(bicyclo[1.1.1 ]pentan-1- ylmethyl)-4-((5-(2,4-difluoro-3- hydroxyphenyl)-1,3,4-thiadiazol-2- yl)methyl)-4,6- diazaspiro[2.4]heptane-5,7-dione |
| I-7 | 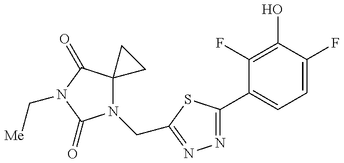 [SMILES: CCN1C(=O)N(Cc2nnc(-c3ccc(F)c(O)c3F)s2)C2(CC2)C1=O] | 4-((5-(2,4-difluoro-3- hydroxyphenyl)-1,3,4-thiadiazol-2- yl)methyl)-6-ethyl-4,6- diazaspiro[2.4]heptane-5,7-dione |
| I-8 | 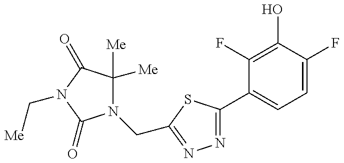 [SMILES: CCN1C(=O)N(Cc2nnc(-c3ccc(F)c(O)c3F)s2)C(C)(C)C1=O] | 1-((5-(2,4-difluoro-3- hydroxyphenyl)-1,3,4-thiadiazol-2- yl)methyl)-3-ethyl-5,5- dimethylimidazolidine-2,4-dione |
| I-9 | 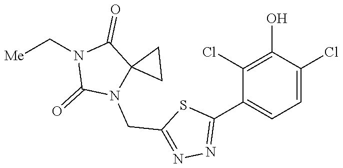 [SMILES: CCN1C(=O)N(Cc2nnc(-c3ccc(Cl)c(O)c3Cl)s2)C2(CC2)C1=O] | 4-((5-(2,4-dichloro-3- hydroxyphenyl)-1,3,4-thiadiazol-2- yl)methyl)-6-ethyl-4,6- diazaspiro[2.4]heptane-5,7-dione |
| I-10 | 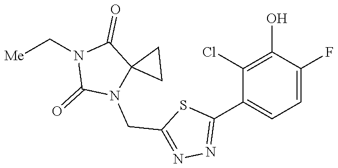 [SMILES: CCN1C(=O)N(Cc2nnc(-c3ccc(F)c(O)c3Cl)s2)C2(CC2)C1=O] | 4-((5-(2-chloro-4-fluoro-3- hydroxyphenyl)-1,3,4-thiadiazol-2- yl)methyl)-6-ethyl-4,6- diazaspiro[2.4]heptane-5,7-dione |
| I-11 | 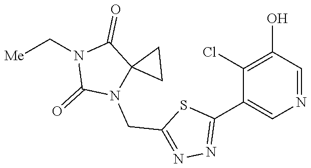 [SMILES: CCN1C(=O)N(Cc2nnc(-c3cncc(O)c3Cl)s2)C2(CC2)C1=O] | 4-((5-(4-chloro-5-hydroxypyridin-3- yl)-1,3,4-thiadiazol-2-yl)methyl)-6- ethyl-4,6-diazaspiro[2.4]heptane- 5,7-dione |
| I-12 | 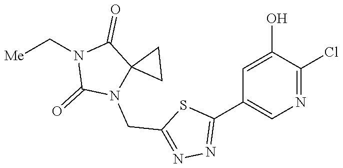 [SMILES: CCN1C(=O)N(Cc2nnc(-c3cnc(Cl)c(O)c3)s2)C2(CC2)C1=O] | 4-((5-(6-chloro-5-hydroxypyridin-3- yl)-1,3,4-thiadiazol-2-yl)methyl)-6- ethyl-4,6-diazaspiro[2.4]heptane- 5,7-dione |
| I-13 | 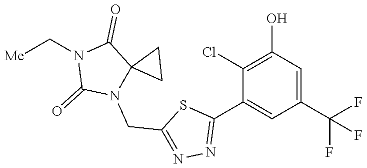 [SMILES: CCN1C(=O)N(Cc2nnc(-c3cc(C(F)(F)F)cc(O)c3Cl)s2)C2(CC2)C1=O] | 4-((5-(2-chloro-3-hydroxy-5- (trifluoromethyl)phenyl)-1,3,4- thiadiazol-2-yl)methyl)-6-ethyl-4,6- diazaspiro[2.4]heptane-5,7-dione |
| I-14 | 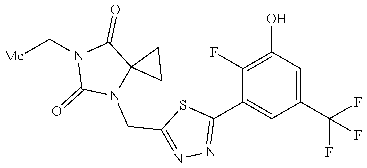 [SMILES: CCN1C(=O)N(Cc2nnc(-c3cc(C(F)(F)F)cc(O)c3F)s2)C2(CC2)C1=O] | 6-ethyl-4-((5-(2-fluoro-3-hydroxy- 5-(trifluoromethyl)phenyl)-1,3,4- thiadiazol-2-yl)methyl)-4,6- diazaspiro[2.4]heptane-5,7-dione |
| I-15 | 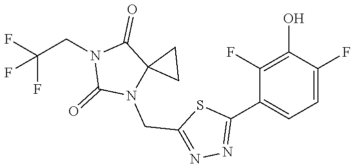 [SMILES: O=C1N(CC(F)(F)F)C(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-6-(2,2,2-trifluoroethyl)- 4,6-diazaspiro[2.4]heptane-5,7- dione |
| I-16 | 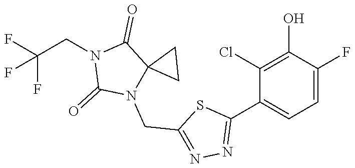 [SMILES: O=C1N(CC(F)(F)F)C(=O)C2(CC2)N1Cc1nnc(-c2ccc(F)c(O)c2Cl)s1] | 4-((5-(2-chloro-4-fluoro-3- hydroxyphenyl)-1,3,4-thiadiazol-2- yl)methyl)-6-(2,2,2-trifluoroethyl)- 4,6-diazaspiro[2.4]heptane-5,7- dione |
| I-17 | 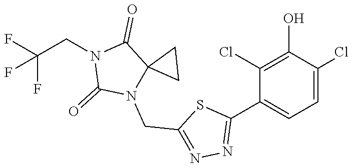 [SMILES: O=C1N(CC(F)(F)F)C(=O)C2(CC2)N1Cc1nnc(-c2ccc(Cl)c(O)c2Cl)s1] | 4-((5-(2,4-dichloro-3- hydroxyphenyl)-1,3,4-thiadiazol-2- yl)methyl)-6-(2,2,2-trifluoroethyl)- 4,6-diazaspiro[2.4]heptane-5,7- dione |
| I-18 | 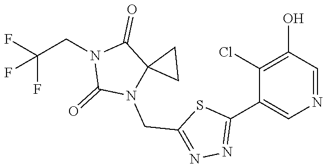 [SMILES: O=C1N(CC(F)(F)F)C(=O)C2(CC2)N1Cc1nnc(-c2cncc(O)c2Cl)s1] | 4-((5-(4-chloro-5-hydroxypyridin-3- yl)-1,3,4-thiadiazol-2-yl)methyl)-6- (2,2,2-trifluoroethyl)-4,6- diazaspiro[2.4]heptane-5,7-dione |
| I-19 | 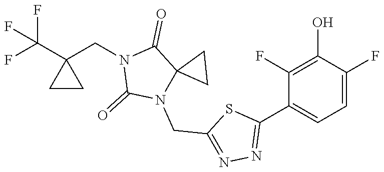 [SMILES: O=C1N(CC2(C(F)(F)F)CC2)C(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-6-((1- (trifluoromethyl)cyclopropyl)methyl)- 4,6-diazaspiro[2.4]heptane-5,7- dione |
| I-20 | 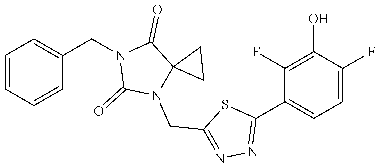 [SMILES: O=C1N(Cc2ccccc2)C(=O)C2(CC2)N1Cc1nnc(-c2ccc(F)c(O)c2F)s1] | 6-benzyl-4-((5-(2,4-difluoro-3- hydroxyphenyl)-1,3,4-thiadiazol-2- yl)methyl)-4,6- diazaspiro[2.4]heptane-5,7-dione |
| I-21 | 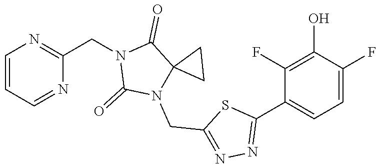 [SMILES: O=C1N(Cc2ncccn2)C(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-6-(pyrimidin-2- ylmethyl)-4,6- diazaspiro[2.4]heptane-5,7-dione |
| I-22 | 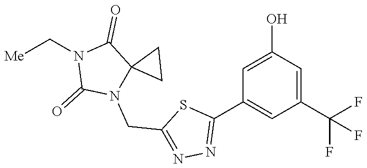 [SMILES: CCN1C(=O)N(Cc2nnc(-c3cc(O)cc(C(F)(F)F)c3)s2)C2(CC2)C1=O] | 6-ethyl-4-((5-(3-hydroxy-5- (trifluoromethyl)phenyl)-1,3,4- thiadiazol-2-yl)methyl)-4,6- diazaspiro[2.4]heptane-5,7-dione |
| I-23 | 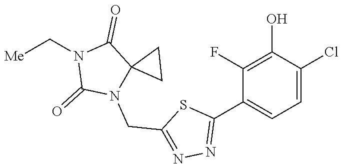 [SMILES: CCN1C(=O)N(Cc2nnc(-c3ccc(Cl)c(O)c3F)s2)C2(CC2)C1=O] | 4-((5-(4-chloro-2-fluoro-3- hydroxyphenyl)-1,3,4-thiadiazol-2- yl)methyl)-6-ethyl-4,6- diazaspiro[2.4]heptane-5,7-dione |
| I-24 | 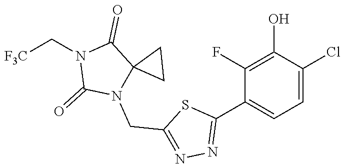 [SMILES: O=C1N(CC(F)(F)F)C(=O)C2(CC2)N1Cc1nnc(-c2ccc(Cl)c(O)c2F)s1] | 4-((5-(4-chloro-2-fluoro-3- hydroxyphenyl)-1,3,4-thiadiazol-2- yl)methyl)-6-(2,2,2-trifluoroethyl)- 4,6-diazaspiro[2.4]heptane-5,7- dione |
| I-25 | 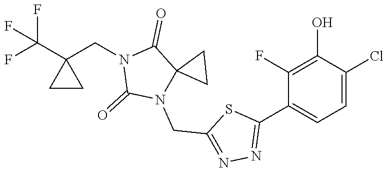 [SMILES: O=C1N(CC2(C(F)(F)F)CC2)C(=O)C2(CC2)N1Cc1nnc(-c2ccc(Cl)c(O)c2F)s1] | 4-((5-(4-chloro-2-fluoro-3- hydroxyphenyl)-1,3,4-thiadiazol-2- yl)methyl)-6-((1- (trifluoromethyl)cyclopropyl)methyl)- 4,6-diazaspiro[2.4]heptane-5,7- dione |
| I-26 | 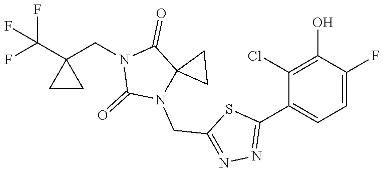 [SMILES: O=C1N(CC2(C(F)(F)F)CC2)C(=O)C2(CC2)N1Cc1nnc(-c2ccc(F)c(O)c2Cl)s1] | 4-((5-(2-chloro-4-fluoro-3- hydroxyphenyl)-1,3,4-thiadiazol-2- yl)methyl)-6-((1- (trifluoromethyl)cyclopropyl)methyl)- 4,6-diazaspiro[2.4]heptane-5,7- dione |
| I-27 | 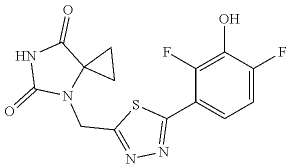 [SMILES: O=C1NC(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-4,6- diazaspiro[2.4]heptane-5,7-dione |
| I-28 | 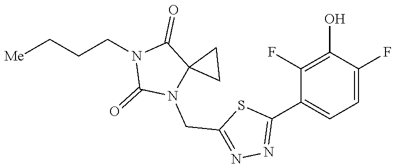 [SMILES: CCCCN1C(=O)N(Cc2nnc(-c3ccc(F)c(O)c3F)s2)C2(CC2)C1=O] | 6-butyl-4-((5-(2,4-difluoro-3- hydroxyphenyl)-1,3,4-thiadiazol-2- yl)methyl)-4,6- diazaspiro[2.4]heptane-5,7-dione |
| I-29 | 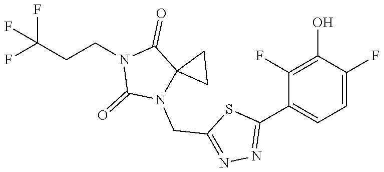 [SMILES: O=C1N(CCC(F)(F)F)C(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-6-(3,3,3-trifluoropropyl)- 4,6-diazaspiro[2.4]heptane-5,7- dione |
| I-30 | 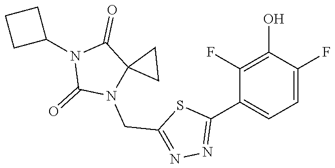 [SMILES: O=C1N(C2CCC2)C(=O)C2(CC2)N1Cc1nnc(-c2ccc(F)c(O)c2F)s1] | 6-cyclobutyl-4-((5-(2,4-difluoro-3- hydroxyphenyl)-1,3,4-thiadiazol-2- yl)methyl)-4,6- diazaspiro[2.4]heptane-5,7-dione |
| I-31 | 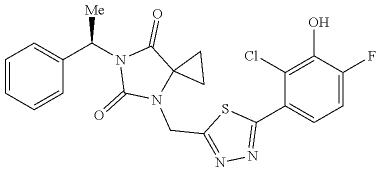 [SMILES: C[C@H](c1ccccc1)N1C(=O)N(Cc2nnc(-c3ccc(F)c(O)c3Cl)s2)C2(CC2)C1=O] | (R)-4-((5-(2-chloro-4-fluoro-3- hydroxyphenyl)-1,3,4-thiadiazol-2- yl)methyl)-6-(1-phenylethyl)-4,6- diazaspiro[2.4]heptane-5,7-dione |
| I-32 | 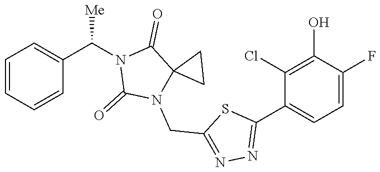 [SMILES: C[C@@H](c1ccccc1)N1C(=O)N(Cc2nnc(-c3ccc(F)c(O)c3Cl)s2)C2(CC2)C1=O] | (S)-4-((5-(2-chloro-4-fluoro-3- hydroxyphenyl)-1,3,4-thiadiazol-2- yl)methyl)-6-(1-phenylethyl)-4,6- diazaspiro[2.4]heptane-5,7-dione |
| I-33 | 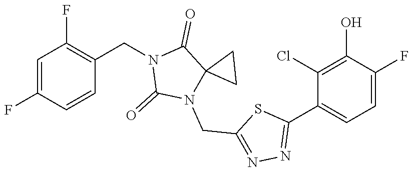 [SMILES: O=C1N(Cc2ccc(F)cc2F)C(=O)C2(CC2)N1Cc1nnc(-c2ccc(F)c(O)c2Cl)s1] | 4-((5-(2-chloro-4-fluoro-3- hydroxyphenyl)-1,3,4-thiadiazol-2- yl)methyl)-6-(2,4-difluorobenzyl)- 4,6-diazaspiro[2.4]heptane-5,7- dione |
| I-34 | 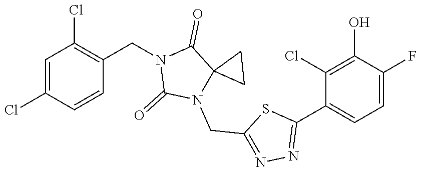 [SMILES: O=C1N(Cc2ccc(Cl)cc2Cl)C(=O)C2(CC2)N1Cc1nnc(-c2ccc(F)c(O)c2Cl)s1] | 4-((5-(2-chloro-4-fluoro-3- hydroxyphenyl)-1,3,4-thiadiazol-2- yl)methyl)-6-(2,4-dichlorobenzyl)- 4,6-diazaspiro[2.4]heptane-5,7- dione |
| I-35 | 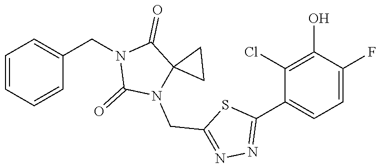 [SMILES: O=C1N(Cc2ccccc2)C(=O)C2(CC2)N1Cc1nnc(-c2ccc(F)c(O)c2Cl)s1] | 6-benzyl-4-((5-(2-chloro-4-fluoro-3- hydroxyphenyl)-1,3,4-thiadiazol-2- yl)methyl)-4,6- diazaspiro[2.4]heptane-5,7-dione |
| I-36 | 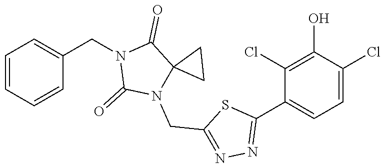 [SMILES: O=C1N(Cc2ccccc2)C(=O)C2(CC2)N1Cc1nnc(-c2ccc(Cl)c(O)c2Cl)s1] | 6-benzyl-4-((5-(2,4-dichloro-3- hydroxyphenyl)-1,3,4-thiadiazol-2- yl)methyl)-4,6- diazaspiro[2.4]heptane-5,7-dione |
| I-37 | 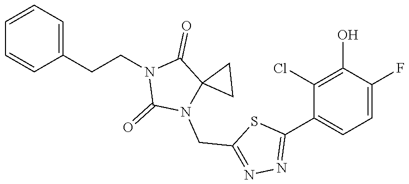 [SMILES: O=C1N(CCc2ccccc2)C(=O)C2(CC2)N1Cc1nnc(-c2ccc(F)c(O)c2Cl)s1] | 4-((5-(2-chloro-4-fluoro-3- hydroxyphenyl)-1,3,4-thiadiazol-2- yl)methyl)-6-phenethyl-4,6- diazaspiro[2.4]heptane-5,7-dione |
| I-38 | 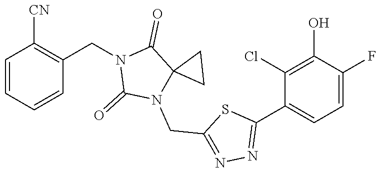 [SMILES: N#Cc1ccccc1CN1C(=O)N(Cc2nnc(-c3ccc(F)c(O)c3Cl)s2)C2(CC2)C1=O] | 2-((4-((5-(2-chloro-4-fluoro-3- hydroxyphenyl)-1,3,4-thiadiazol-2- yl)methyl)-5,7-dioxo-4,6- diazaspiro[2.4]heptan-6- yl)methyl)benzonitrile |
| I-39 | 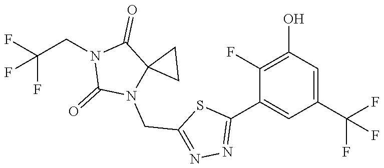 [SMILES: O=C1N(CC(F)(F)F)C(=O)C2(CC2)N1Cc1nnc(-c2cc(C(F)(F)F)cc(O)c2F)s1] | 4-((5-(2-fluoro-3-hydroxy-5- (trifluoromethyl)phenyl)-1,3,4- thiadiazol-2-yl)methyl)-6-(2,2,2- trifluoroethyl)-4,6- diazaspiro[2.4]heptane-5,7-dione |
| I-40 | 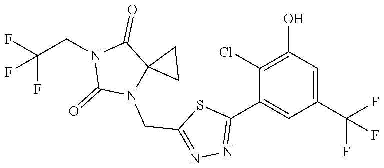 [SMILES: O=C1N(CC(F)(F)F)C(=O)C2(CC2)N1Cc1nnc(-c2cc(C(F)(F)F)cc(O)c2Cl)s1] | 4-((5-(2-chloro-3-hydroxy-5- (trifluoromethyl)phenyl)-1,3,4- thiadiazol-2-yl)methyl)-6-(2,2,2- trifluoroethyl)-4,6- diazaspiro[2.4]heptane-5,7-dione |
| I-41 | 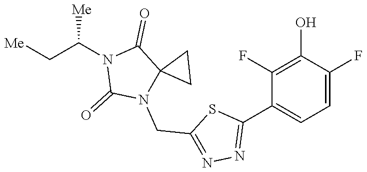 [SMILES: CC[C@H](C)N1C(=O)N(Cc2nnc(-c3ccc(F)c(O)c3F)s2)C2(CC2)C1=O] | (S)-6-(sec-butyl)-4-((5-(2,4- difluoro-3-hydroxyphenyl)-1,3,4- thiadiazol-2-yl)methyl)-4,6- diazaspiro[2.4]heptane-5,7-dione |
| I-42 | 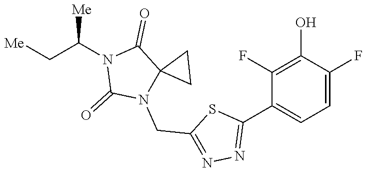 [SMILES: CC[C@@H](C)N1C(=O)N(Cc2nnc(-c3ccc(F)c(O)c3F)s2)C2(CC2)C1=O] | (R)-6-(sec-butyl)-4-((5-(2,4- difluoro-3-hydroxyphenyl)-1,3,4- thiadiazol-2-yl)methyl)-4,6- diazaspiro[2.4]heptane-5,7-dione |
| I-43 | 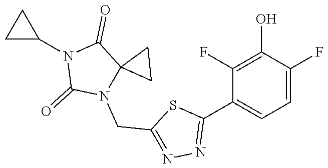 [SMILES: O=C1N(C2CC2)C(=O)C2(CC2)N1Cc1nnc(-c2ccc(F)c(O)c2F)s1] | 6-cyclopropyl-4-((5-(2,4-difluoro-3- hydroxyphenyl)-1,3,4-thiadiazol-2- yl)methyl)-4,6- diazaspiro[2.4]heptane-5,7-dione |
| I-44 | 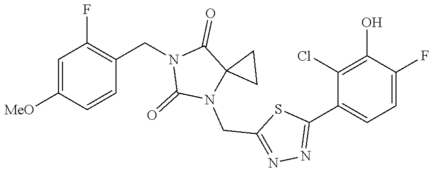 [SMILES: COc1ccc(CN2C(=O)N(Cc3nnc(-c4ccc(F)c(O)c4Cl)s3)C3(CC3)C2=O)c(F)c1] | 4-((5-(2-chloro-4-fluoro-3- hydroxyphenyl)-1,3,4-thiadiazol-2- yl)methyl)-6-(2-fluoro-4- methoxybenzyl)-4,6- diazaspiro[2.4]heptane-5,7-dione |
| I-45 | 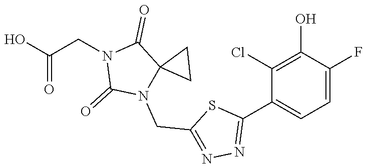 [SMILES: O=C(O)CN1C(=O)N(Cc2nnc(-c3ccc(F)c(O)c3Cl)s2)C2(CC2)C1=O] | 2-(4-((5-(2-chloro-4-fluoro-3- hydroxyphenyl)-1,3,4-thiadiazol-2- yl)methyl)-5,7-dioxo-4,6- diazaspiro[2.4]heptan-6-yl)acetic acid |
| I-46 | 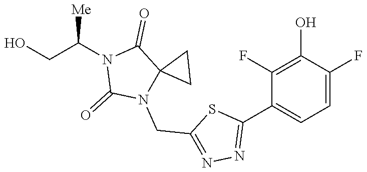 [SMILES: C[C@H](CO)N1C(=O)N(Cc2nnc(-c3ccc(F)c(O)c3F)s2)C2(CC2)C1=O] | (R)-4-((5-(2,4-difluoro-3- hydroxyphenyl)-1,3,4-thiadiazol-2- yl)methyl)-6-(1-hydroxypropan-2- yl)-4,6-diazaspiro[2.4]heptane-5,7- dione |
| I-47 | 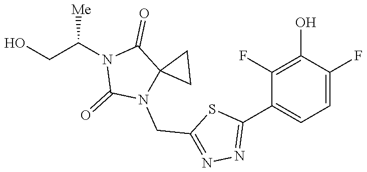 [SMILES: C[C@@H](CO)N1C(=O)N(Cc2nnc(-c3ccc(F)c(O)c3F)s2)C2(CC2)C1=O] | (S)-4-((5-(2,4-difluoro-3- hydroxyphenyl)-1,3,4-thiadiazol-2- yl)methyl)-6-(1-hydroxypropan-2- yl)-4,6-diazaspiro[2.4]heptane-5,7- dione |
| I-48 | 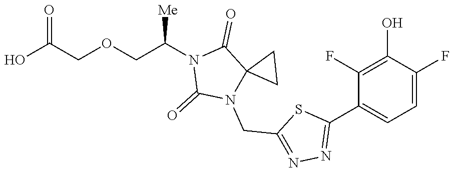 [SMILES: C[C@H](COCC(=O)O)N1C(=O)N(Cc2nnc(-c3ccc(F)c(O)c3F)s2)C2(CC2)C1=O] | (R)-2-(2-(4-((5-(2,4-difluoro-3- hydroxyphenyl)-1,3,4-thiadiazol-2- yl)methyl)-5,7-dioxo-4,6- diazaspiro[2.4]heptan-6- yl)propoxy)acetic acid |
| I-49 | 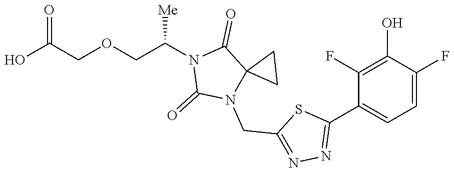 [SMILES: C[C@@H](COCC(=O)O)N1C(=O)N(Cc2nnc(-c3ccc(F)c(O)c3F)s2)C2(CC2)C1=O] | (S)-2-(2-(4-((5-(2,4-difluoro-3- hydroxyphenyl)-1,3,4-thiadiazol-2- yl)methyl)-5,7-dioxo-4,6- diazaspiro[2.4]heptan-6- yl)propoxy)acetic acid |
| I-50 | 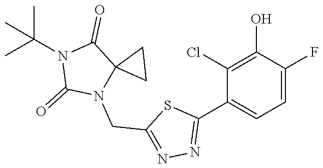 [SMILES: CC(C)(C)N1C(=O)N(Cc2nnc(-c3ccc(F)c(O)c3Cl)s2)C2(CC2)C1=O] | 6-(tert-butyl)-4-((5-(2-chloro-4- fluoro-3-hydroxyphenyl)-1,3,4- thiadiazol-2-yl)methyl)-4,6- diazaspiro[2.4]heptane-5,7-dione |
| I-51 Metabolite | 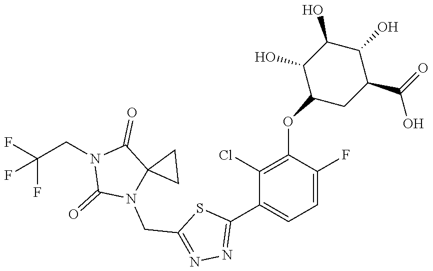 | (2S,3S,4S,5R,6S)-6-(2-chloro-3-(5- ((5,7-dioxo-6-(2,2,2-trifluoroethyl)- 4,6-diazaspiro[2.4]heptan-4- yl)methyl)-1,3,4-thiadiazol-2-yl)-6- fluorophenoxy)-3,4,5- trihydroxytetrahydro-2H-pyran-2- carboxylic acid |
| I-52 | 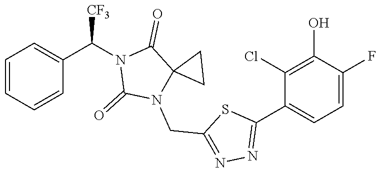 [SMILES: O=C1N([C@@H](c2ccccc2)C(F)(F)F)C(=O)C2(CC2)N1Cc1nnc(-c2ccc(F)c(O)c2Cl)s1] | (S)-4-((5-(2-chloro-4-fluoro-3- hydroxyphenyl)-1,3,4-thiadiazol-2- yl)methyl)-6-(2,2,2-trifluoro-1- phenylethyl)-4,6- diazaspiro[2.4]heptane-5,7-dione |
| I-53 | 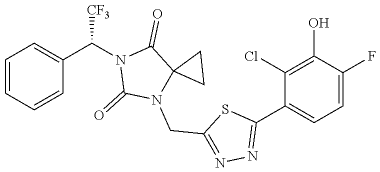 [SMILES: O=C1N([C@H](c2ccccc2)C(F)(F)F)C(=O)C2(CC2)N1Cc1nnc(-c2ccc(F)c(O)c2Cl)s1] | (R)-4-((5-(2-chloro-4-fluoro-3- hydroxyphenyl)-1,3,4-thiadiazol-2- yl)methyl)-6-(2,2,2-trifluoro-1- phenylethyl)-4,6- diazaspiro[2.4]heptane-5,7-dione |
| I-54 | 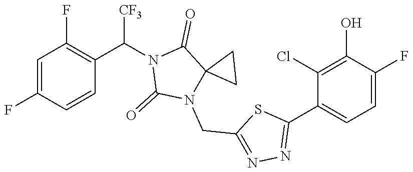 [SMILES: O=C1N(C(c2ccc(F)cc2F)C(F)(F)F)C(=O)C2(CC2)N1Cc1nnc(-c2ccc(F)c(O)c2Cl)s1] | 4-((5-(2-chloro-4-fluoro-3- hydroxyphenyl)-1,3,4-thiadiazol-2- yl)methyl)-6-(1-(2,4- difluorophenyl)-2,2,2- trifluoroethyl)-4,6- diazaspiro[2.4]heptane-5,7-dione |
| I-55 | 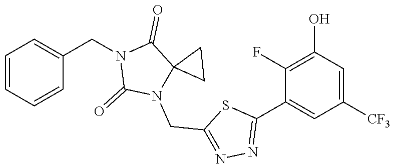 [SMILES: O=C1N(Cc2ccccc2)C(=O)C2(CC2)N1Cc1nnc(-c2cc(C(F)(F)F)cc(O)c2F)s1] | 6-benzyl-4-((5-(2-fluoro-3-hydroxy- 5-(trifluoromethyl)phenyl)-1,3,4- thiadiazol-2-yl)methyl)-4,6- diazaspiro[2.4]heptane-5,7-dione |
| I-56 | 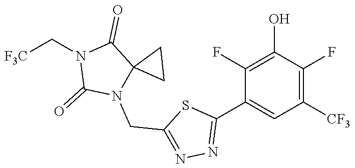 [SMILES: O=C1N(CC(F)(F)F)C(=O)C2(CC2)N1Cc1nnc(-c2cc(C(F)(F)F)c(F)c(O)c2F)s1] | 4-((5-(2,4-difluoro-3-hydroxy-5- (trifluoromethyl)phenyl)-1,3,4- thiadiazol-2-yl)methyl)-6-(2,2,2- trifluoroethyl)-4,6- diazaspiro[2.4]heptane-5,7-dione |
| I-57 | 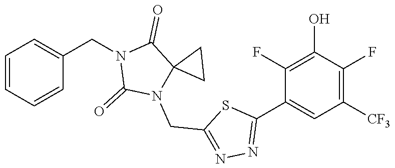 [SMILES: O=C1N(Cc2ccccc2)C(=O)C2(CC2)N1Cc1nnc(-c2cc(C(F)(F)F)c(F)c(O)c2F)s1] | 6-benzyl-4-((5-(2,4-difluoro-3- hydroxy-5-(trifluoromethyl)phenyl)- 1,3,4-thiadiazol-2-yl)methyl)-4,6- diazaspiro[2.4]heptane-5,7-dione |
| I-58 | 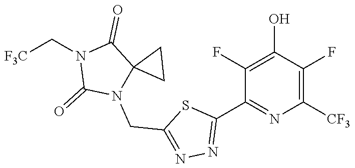 [SMILES: O=C1N(CC(F)(F)F)C(=O)C2(CC2)N1Cc1nnc(-c2nc(C(F)(F)F)c(F)c(O)c2F)s1] | 4-((5-(3,5-difluoro-4-hydroxy-6- (trifluoromethyl)pyridin-2-yl)-1,3,4- thiadiazol-2-yl)methyl)-6-(2,2,2- trifluoroethyl)-4,6- diazaspiro[2.4]heptane-5,7-dione |
| I-59 | 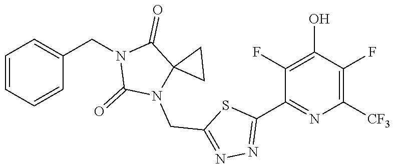 [SMILES: O=C1N(Cc2ccccc2)C(=O)C2(CC2)N1Cc1nnc(-c2nc(C(F)(F)F)c(F)c(O)c2F)s1] | 6-benzyl-4-((5-(3,5-difluoro-4- hydroxy-6-(trifluoromethyl)pyridin- 2-yl)-1,3,4-thiadiazol-2-yl)methyl)- 4,6-diazaspiro[2.4]heptane-5,7- dione |
| I-60 | 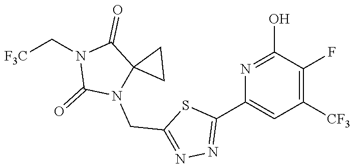 [SMILES: O=C1N(CC(F)(F)F)C(=O)C2(CC2)N1Cc1nnc(-c2cc(C(F)(F)F)c(F)c(O)n2)s1] | 4-((5-(5-fluoro-6-hydroxy-4- (trifluoromethyl)pyridin-2-yl)-1,3,4- thiadiazol-2-yl)methyl)-6-(2,2,2- trifluoroethyl)-4,6- diazaspiro[2.4]heptane-5,7-dione |
| I-61 | 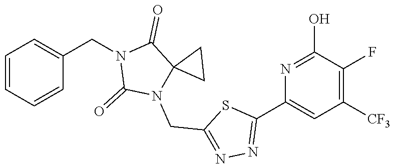 [SMILES: O=C1N(Cc2ccccc2)C(=O)C2(CC2)N1Cc1nnc(-c2cc(C(F)(F)F)c(F)c(O)n2)s1] | 6-benzyl-4-((5-(5-fluoro-6-hydroxy- 4-(trifluoromethyl)pyridin-2-yl)- 1,3,4-thiadiazol-2-yl)methyl)-4,6- diazaspiro[2.4]heptane-5,7-dione |
| I-62 | 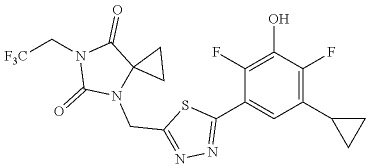 [SMILES: O=C1N(CC(F)(F)F)C(=O)C2(CC2)N1Cc1nnc(-c2cc(C3CC3)c(F)c(O)c2F)s1] | 4-((5-(5-cyclopropyl-2,4-difluoro-3- hydroxyphenyl)-1,3,4-thiadiazol-2- yl)methyl)-6-(2,2,2-trifluoroethyl)- 4,6-diazaspiro[2.4]heptane-5,7- dione |
| I-63 | 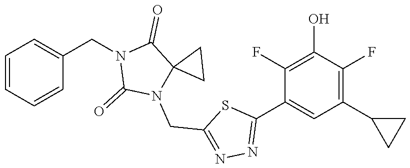 [SMILES: O=C1N(Cc2ccccc2)C(=O)C2(CC2)N1Cc1nnc(-c2cc(C3CC3)c(F)c(O)c2F)s1] | 6-benzyl-4-((5-(5-cyclopropyl-2,4- difluoro-3-hydroxyphenyl)-1,3,4- thiadiazol-2-yl)methyl)-4,6- diazaspiro[2.4]heptane-5,7-dione |
| I-64 | 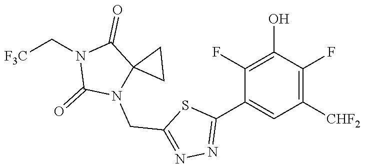 [SMILES: O=C1N(CC(F)(F)F)C(=O)C2(CC2)N1Cc1nnc(-c2cc(C(F)F)c(F)c(O)c2F)s1] | 4-((5-(5-(difluoromethyl)-2,4- difluoro-3-hydroxyphenyl)-1,3,4- thiadiazol-2-yl)methyl)-6-(2,2,2- trifluoroethyl)-4,6- diazaspiro[2.4]heptane-5,7-dione |
| I-65 | 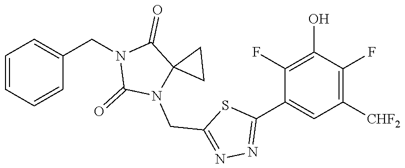 [SMILES: O=C1N(Cc2ccccc2)C(=O)C2(CC2)N1Cc1nnc(-c2cc(C(F)F)c(F)c(O)c2F)s1] | 6-benzyl-4-((5-(5-(difluoromethyl)- 2,4-difluoro-3-hydroxyphenyl)- 1,3,4-thiadiazol-2-yl)methyl)-4,6- diazaspiro[2.4]heptane-5,7-dione |
| I-68 | 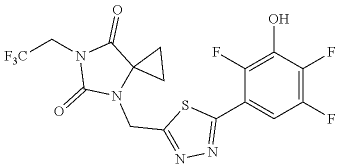 [SMILES: O=C1N(CC(F)(F)F)C(=O)C2(CC2)N1Cc1nnc(-c2cc(F)c(F)c(O)c2F)s1] | 4-((5-(2,4,5-trifluoro-3- hydroxyphenyl)-1,3,4-thiadiazol-2- yl)methyl)-6-(2,2,2-trifluoroethyl)- 4,6-diazaspiro[2.4]heptane-5,7- dione |
| I-69 | 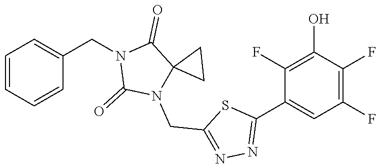 [SMILES: O=C1N(Cc2ccccc2)C(=O)C2(CC2)N1Cc1nnc(-c2cc(F)c(F)c(O)c2F)s1] | 6-benzyl-4-((5-(2,4,5-trifluoro-3- hydroxyphenyl)-1,3,4-thiadiazol-2- yl)methyl)-4,6- diazaspiro[2.4]heptane-5,7-dione |
| I-72 Metabolite | 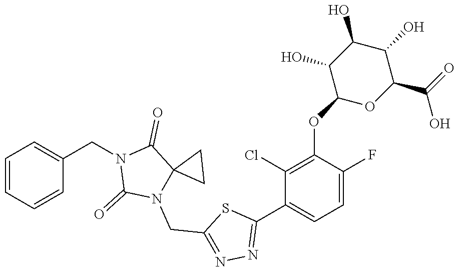 [SMILES: O=C(O)[C@H]1O[C@@H](Oc2c(F)ccc(-c3nnc(CN4C(=O)N(Cc5ccccc5)C(=O)C45CC5)s3)c2Cl)[C@H](O)[C@@H](O)[C@@H]1O] | (2S,3S,4S,5R,6S)-6-(3-(5-((6- benzyl-5,7-dioxo-4,6- diazaspiro[2.4]heptan-4-yl)methyl)- 1,3,4-thiadiazol-2-yl)-2-chloro-6- fluorophenoxy)-3,4,5- trihydroxytetrahydro-2H-pyran-2- carboxylic acid |
| I-73 | 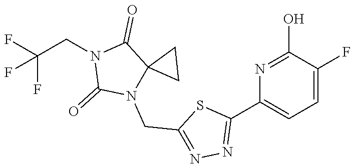 [SMILES: O=C1N(CC(F)(F)F)C(=O)C2(CC2)N1Cc1nnc(-c2ccc(F)c(O)n2)s1] | 4-((5-(5-fluoro-6-hydroxypyridin-2- yl)-1,3,4-thiadiazol-2-yl)methyl)-6- (2,2,2-trifluoroethyl)-4,6- diazaspiro[2.4]heptane-5,7-dione |
| I-74 | 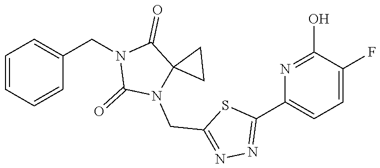 [SMILES: O=C1N(Cc2ccccc2)C(=O)C2(CC2)N1Cc1nnc(-c2ccc(F)c(O)n2)s1] | 6-benzyl-4-((5-(5-fluoro-6- hydroxypyridin-2-yl)-1,3,4- thiadiazol-2-yl)methyl)-4,6- diazaspiro[2.4]heptane-5,7-dione |
| I-75 | 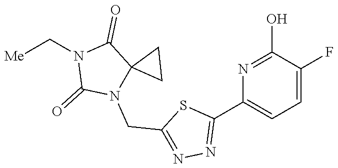 [SMILES: CCN1C(=O)N(Cc2nnc(-c3ccc(F)c(O)n3)s2)C2(CC2)C1=O] | 6-ethyl-4-((5-(5-fluoro-6- hydroxypyridin-2-yl)-1,3,4- thiadiazol-2-yl)methyl)-4,6- diazaspiro[2.4]heptane-5,7-dione |
| I-76 | 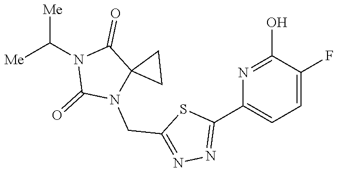 [SMILES: CC(C)N1C(=O)N(Cc2nnc(-c3ccc(F)c(O)n3)s2)C2(CC2)C1=O] | 4-((5-(5-fluoro-6-hydroxypyridin-2- yl)-1,3,4-thiadiazol-2-yl)methyl)-6- isopropyl-4,6- diazaspiro[2.4]heptane-5,7-dione |
| I-77 | 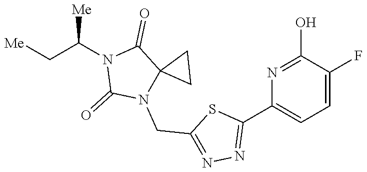 [SMILES: CC[C@@H](C)N1C(=O)N(Cc2nnc(-c3ccc(F)c(O)n3)s2)C2(CC2)C1=O] | (R)-6-(sec-butyl)-4-((5-(5-fluoro-6- hydroxypyridin-2-yl)-1,3,4- thiadiazol-2-yl)methyl)-4,6- diazaspiro[2.4]heptane-5,7-dione |
| I-78 | 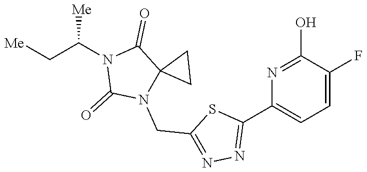 [SMILES: CC[C@H](C)N1C(=O)N(Cc2nnc(-c3ccc(F)c(O)n3)s2)C2(CC2)C1=O] | (S)-6-(sec-butyl)-4-((5-(5-fluoro-6- hydroxypyridin-2-yl)-1,3,4- thiadiazol-2-yl)methyl)-4,6- diazaspiro[2.4]heptane-5,7-dione |
| I-79 | 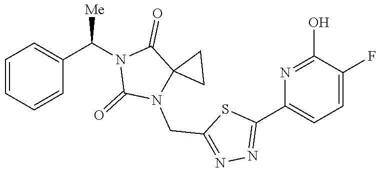 [SMILES: C[C@H](c1ccccc1)N1C(=O)N(Cc2nnc(-c3ccc(F)c(O)n3)s2)C2(CC2)C1=O] | (R)-4-((5-(5-fluoro-6- hydroxypyridin-2-yl)-1,3,4- thiadiazol-2-yl)methyl)-6-(1- phenylethyl)-4,6- diazaspiro[2.4]heptane-5,7-dione |
| I-80 | 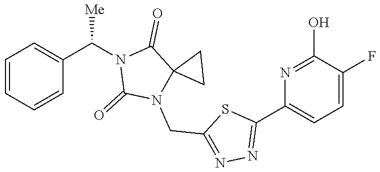 [SMILES: C[C@@H](c1ccccc1)N1C(=O)N(Cc2nnc(-c3ccc(F)c(O)n3)s2)C2(CC2)C1=O] | (S)-4-((5-(5-fluoro-6- hydroxypyridin-2-yl)-1,3,4- thiadiazol-2-yl)methyl)-6-(1- phenylethyl)-4,6- diazaspiro[2.4]heptane-5,7-dione |
| I-81 | 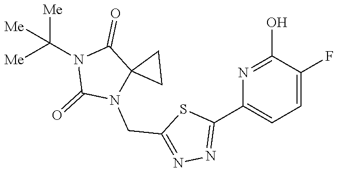 [SMILES: CC(C)(C)N1C(=O)N(Cc2nnc(-c3ccc(F)c(O)n3)s2)C2(CC2)C1=O] | 6-(tert-butyl)-4-((5-(5-fluoro-6- hydroxypyridin-2-yl)-1,3,4- thiadiazol-2-yl)methyl)-4,6- diazaspiro[2.4]heptane-5,7-dione |
| I-82 | 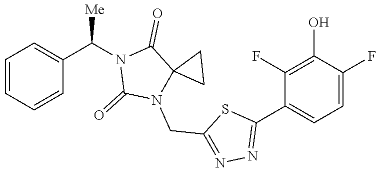 [SMILES: C[C@H](c1ccccc1)N1C(=O)N(Cc2nnc(-c3ccc(F)c(O)c3F)s2)C2(CC2)C1=O] | (R)-4-((5-(2,4-difluoro-3- hydroxyphenyl)-1,3,4-thiadiazol-2- yl)methyl)-6-(1-phenylethyl)-4,6- diazaspiro[2.4]heptane-5,7-dione |
| I-83 | 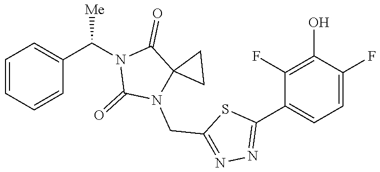 [SMILES: C[C@@H](c1ccccc1)N1C(=O)N(Cc2nnc(-c3ccc(F)c(O)c3F)s2)C2(CC2)C1=O] | (S)-4-((5-(2,4-difluoro-3- hydroxyphenyl)-1,3,4-thiadiazol-2- yl)methyl)-6-(1-phenylethyl)-4,6- diazaspiro[2.4]heptane-5,7-dione |
| I-84 | 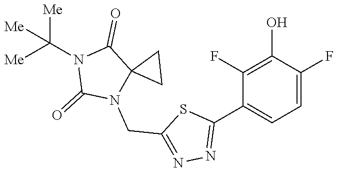 [SMILES: CC(C)(C)N1C(=O)N(Cc2nnc(-c3ccc(F)c(O)c3F)s2)C2(CC2)C1=O] | 6-(tert-butyl)-4-((5-(2,4-difluoro-3- hydroxyphenyl)-1,3,4-thiadiazol-2- yl)methyl)-4,6- diazaspiro[2.4]heptane-5,7-dione |
| I-85 | 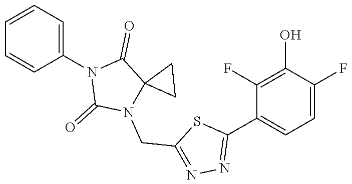 [SMILES: O=C1N(c2ccccc2)C(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-6-phenyl-4,6- diazaspiro[2.4]heptane-5,7-dione |
| I-86 | 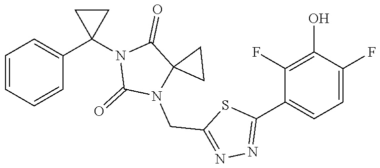 [SMILES: O=C1N(Cc2nnc(-c3ccc(F)c(O)c3F)s2)C2(CC2)C(=O)N1C1(c2ccccc2)CC1] | 4-((5-(2,4-difluoro-3- hydroxyphenyl)-1,3,4-thiadiazol-2- yl)methyl)-6-(1- phenylcyclopropyl)-4,6- diazaspiro[2.4]heptane-5,7-dione |
| I-87 | 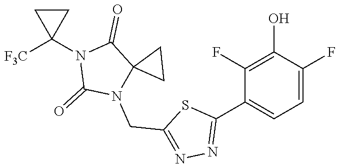 [SMILES: O=C1N(Cc2nnc(-c3ccc(F)c(O)c3F)s2)C2(CC2)C(=O)N1C1(C(F)(F)F)CC1] | 4-((5-(2,4-difluoro-3- hydroxyphenyl)-1,3,4-thiadiazol-2- yl)methyl)-6-(1- (trifluoromethyl)cyclopropyl)-4,6- diazaspiro[2.4]heptane-5,7-dione |
| I-88 | 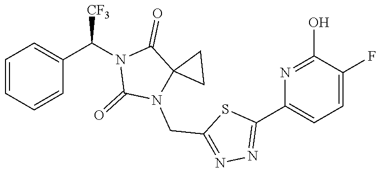 [SMILES: O=C1N([C@@H](c2ccccc2)C(F)(F)F)C(=O)C2(CC2)N1Cc1nnc(-c2ccc(F)c(O)n2)s1] | (S)-4-((5-(5-fluoro-6- hydroxypyridin-2-yl)-1,3,4- thiadiazol-2-yl)methyl)-6-(2,2,2- trifluoro-1-phenylethyl)-4,6- diazaspiro[2.4]heptane-5,7-dione |
| I-89 | 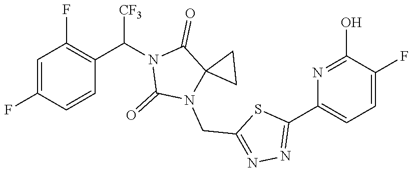 [SMILES: O=C1N(C(c2ccc(F)cc2F)C(F)(F)F)C(=O)C2(CC2)N1Cc1nnc(-c2ccc(F)c(O)n2)s1] | 6-(1-(2,4-difluorophenyl)-2,2,2- trifluoroethyl)-4-((5-(5-fluoro-6- hydroxypyridin-2-yl)-1,3,4- thiadiazol-2-yl)methyl)-4,6- diazaspiro[2.4]heptane-5,7-dione |
| I-90A | 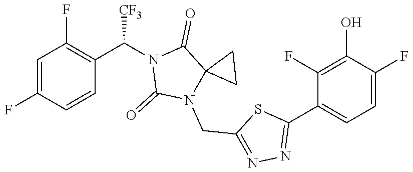 [SMILES: O=C1N([C@H](c2ccc(F)cc2F)C(F)(F)F)C(=O)C2(CC2)N1Cc1nnc(-c2ccc(F)c(O)c2F)s1] | (R)-4-((5-(2,4-difluoro-3- hydroxyphenyl)-1,3,4-thiadiazol-2- yl)methyl)-6-(1-(2,4- difluorophenyl)-2,2,2- trifluoroethyl)-4,6- diazaspiro[2.4]heptane-5,7-dione |
| I-90B | 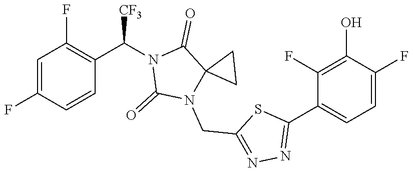 [SMILES: O=C1N([C@@H](c2ccc(F)cc2F)C(F)(F)F)C(=O)C2(CC2)N1Cc1nnc(-c2ccc(F)c(O)c2F)s1] | (S)-4-((5-(2,4-difluoro-3- hydroxyphenyl)-1,3,4-thiadiazol-2- yl)methyl)-6-(1-(2,4- difluorophenyl)-2,2,2- trifluoroethyl)-4,6- diazaspiro[2.4]heptane-5,7-dione |
| I-91 | 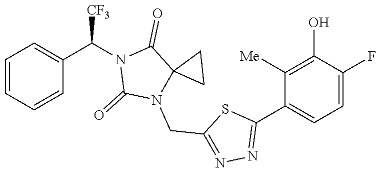 [SMILES: Cc1c(-c2nnc(CN3C(=O)N([C@@H](c4ccccc4)C(F)(F)F)C(=O)C34CC4)s2)ccc(F)c1O] | (S)-4-((5-(4-fluoro-3-hydroxy-2- methylphenyl)-1,3,4-thiadiazol-2- yl)methyl)-6-(2,2,2-trifluoro-1- phenylethyl)-4,6- diazaspiro[2.4]heptane-5,7-dione |
| I-92 | 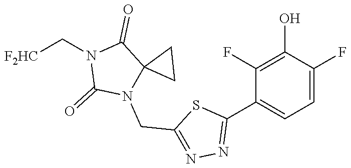 [SMILES: O=C1N(CC(F)F)C(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-6-(2,2-difluoroethyl)- 4,6-diazaspiro[2.4]heptane-5,7- dione |
| I-93 | 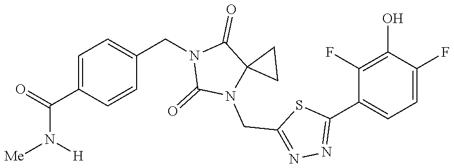 [SMILES: CNC(=O)c1ccc(CN2C(=O)N(Cc3nnc(-c4ccc(F)c(O)c4F)s3)C3(CC3)C2=O)cc1] | 4-((4-((5-(2,4-difluoro-3- hydroxyphenyl)-1,3,4-thiadiazol-2- yl)methyl)-5,7-dioxo-4,6- diazaspiro[2.4]heptan-6-yl)methyl)- N-methylbenzamide |
| I-94 | 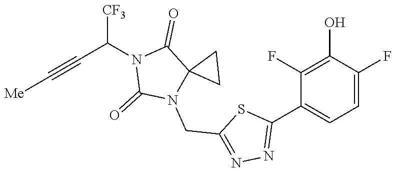 [SMILES: CC#CC(N1C(=O)N(Cc2nnc(-c3ccc(F)c(O)c3F)s2)C2(CC2)C1=O)C(F)(F)F] | 4-((5-(2,4-difluoro-3- hydroxyphenyl)-1,3,4-thiadiazol-2- yl)methyl)-6-(1,1,1-trifluoropent-3- yn-2-yl)-4,6-diazaspiro[2.4]heptane- 5,7-dione |
| I-95 | 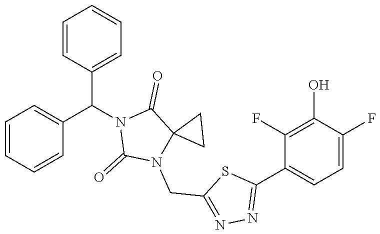 [SMILES: O=C1N(C(c2ccccc2)c2ccccc2)C(=O)C2(CC2)N1Cc1nnc(-c2ccc(F)c(O)c2F)s1] | 6-benzhydryl-4-((5-(2,4-difluoro-3- hydroxyphenyl)-1,3,4-thiadiazol-2- yl)methyl)-4,6- diazaspiro[2.4]heptane-5,7-dione |
| I-96 | 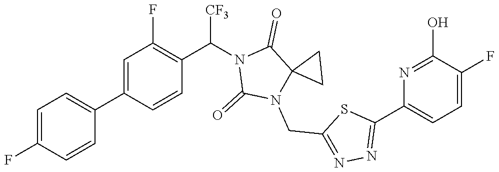 [SMILES: O=C1N(C(c2ccc(-c3ccc(F)cc3)cc2F)C(F)(F)F)C(=O)C2(CC2)N1Cc1nnc(-c2ccc(F)c(O)n2)s1] | 6-(1-(3,4′-difluoro-[1,1′-biphenyl]- 4-yl)-2,2,2-trifluoroethyl)-4-((5-(5- fluoro-6-hydroxypyridin-2-yl)- 1,3,4-thiadiazol-2-yl)methyl)-4,6- diazaspiro[2.4]heptane-5,7-dione |
| I-97 | 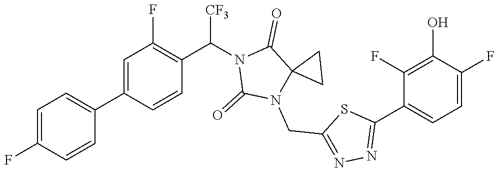 [SMILES: O=C1N(C(c2ccc(-c3ccc(F)cc3)cc2F)C(F)(F)F)C(=O)C2(CC2)N1Cc1nnc(-c2ccc(F)c(O)c2F)s1] | 4-((5-(2,4-difluoro-3- hydroxyphenyl)-1,3,4-thiadiazol-2- yl)methyl)-6-(1-(3,4′-difluoro-[1,1′- biphenyl]-4-yl)-2,2,2- trifluoroethyl)-4,6- diazaspiro[2.4]heptane-5,7-dione |
| I-99A | 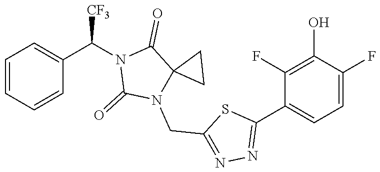 [SMILES: O=C1N([C@@H](c2ccccc2)C(F)(F)F)C(=O)C2(CC2)N1Cc1nnc(-c2ccc(F)c(O)c2F)s1] | (S)-4-((5-(2,4-difluoro-3- hydroxyphenyl)-1,3,4-thiadiazol-2- yl)methyl)-6-(2,2,2-trifluoro-1- phenylethyl)-4,6- diazaspiro[2.4]heptane-5,7-dione |
| I-100 Metabolite | 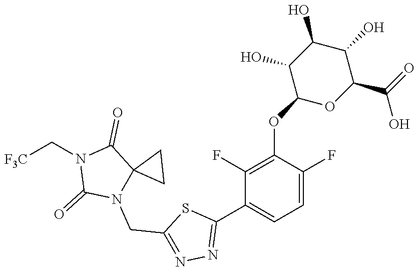 [SMILES: O=C(O)[C@H]1O[C@@H](Oc2c(F)ccc(-c3nnc(CN4C(=O)N(CC(F)(F)F)C(=O)C45CC5)s3)c2F)[C@H](O)[C@@H](O)[C@@H]1O] | (2S,3S,4S,5R,6S)-6-(3-(5-((5,7- dioxo-6-(2,2,2-trifluoroethyl)-4,6- diazaspiro[2.4]heptan-4-yl)methyl)- 1,3,4-thiadiazol-2-yl)-2,6- difluorophenoxy)-3,4,5- trihydroxytetrahydro-2H-pyran-2- carboxylic acid |
| I-101 metabolite | 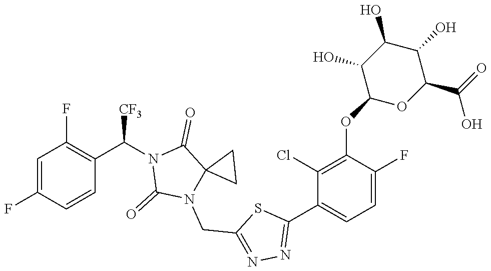 [SMILES: O=C(O)[C@H]1O[C@@H](Oc2c(F)ccc(-c3nnc(CN4C(=O)N([C@@H](c5ccc(F)cc5F)C(F)(F)F)C(=O)C45CC5)s3)c2Cl)[C@H](O)[C@@H](O)[C@@H]1O] | (2S,3S,4S,5R,6S)-6-(2-chloro-3-(5- ((6-((S)-1-(2,4-difluorophenyl)- 2,2,2-trifluoroethyl)-5,7-dioxo-4,6- diazaspiro[2.4]heptan-4-yl)methyl)- 1,3,4-thiadiazol-2-yl)-6- fluorophenoxy)-3,4,5- trihydroxytetrahydro-2H-pyran-2- carboxylic acid |
| I-102 metabolite | 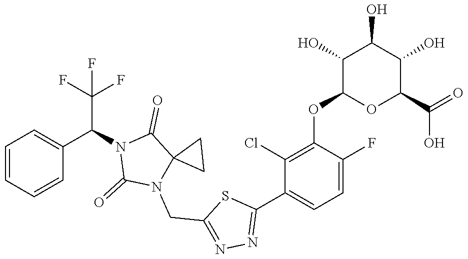 [SMILES: O=C(O)[C@H]1O[C@@H](Oc2c(F)ccc(-c3nnc(CN4C(=O)N([C@@H](c5ccccc5)C(F)(F)F)C(=O)C45CC5)s3)c2Cl)[C@H](O)[C@@H](O)[C@@H]1O] | (2S,3S,4S,5R,6S)-6-(2-chloro-3-(5- ((5,7-dioxo-6-((S)-2,2,2-trifluoro-1- phenylethyl)-4,6- diazaspiro[2.4]heptan-4-yl)methyl)- 1,3,4-thiadiazol-2-yl)-6- fluorophenoxy)-3,4,5- trihydroxytetrahydro-2H-pyran-2- carboxylic acid |
| I-103 | 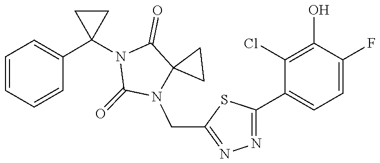 [SMILES: O=C1N(Cc2nnc(-c3ccc(F)c(O)c3Cl)s2)C2(CC2)C(=O)N1C1(c2ccccc2)CC1] | 4-((5-(2-chloro-4-fluoro-3- hydroxyphenyl)-1,3,4-thiadiazol-2- yl)methyl)-6-(1- phenylcyclopropyl)-4,6- diazaspiro[2.4]heptane-5,7-dione |
| I-104 | 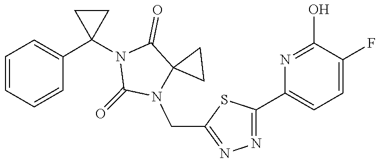 [SMILES: O=C1N(Cc2nnc(-c3ccc(F)c(O)n3)s2)C2(CC2)C(=O)N1C1(c2ccccc2)CC1] | 4-((5-(5-fluoro-6-hydroxypyridin-2- yl)-1,3,4-thiadiazol-2-yl)methyl)-6- (1-phenylcyclopropyl)-4,6- diazaspiro[2.4]heptane-5,7-dione |
| I-105 Metabolite | 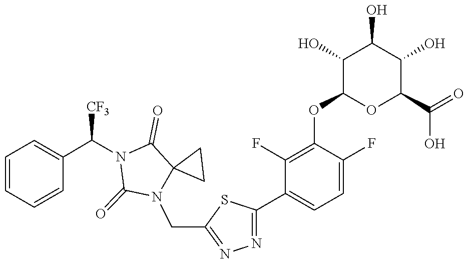 [SMILES: O=C(O)[C@H]1O[C@@H](Oc2c(F)ccc(-c3nnc(CN4C(=O)N([C@@H](c5ccccc5)C(F)(F)F)C(=O)C45CC5)s3)c2F)[C@H](O)[C@@H](O)[C@@H]1O] | (2S,3S,4S,5R,6S)-6-(3-(5-((5,7- dioxo-6-((S)-2,2,2-trifluoro-1- phenylethyl)-4,6- diazaspiro[2.4]heptan-4-yl)methyl)- 1,3,4-thiadiazol-2-yl)-2,6- difluorophenoxy)-3,4,5- trihydroxytetrahydro-2H-pyran-2- carboxylic acid |

### General Synthetic Methods for the Preparation of Compounds of Formula I

Examples of syntheses: the compounds of this invention are synthesized as outlined in General Scheme A. Commercially available thiadiazoles containing suitable halogens are modified to contain an electrophilic leaving group such that they can be reacted with hydantoins under basic conditions, e.g. potassium carbonate in dimethylformamide (DMF). The hydantoin can be alkylated by starting material A on an acidic nitrogen on the hydantoin as illustrated in General Scheme A. In some cases, stronger bases like lithium diisopropylamide (LDA) or lithium hexamethyldisilazide (LHMDS) can be used to selectively deprotonate the hydantoin or. In some cases, protecting groups can be used on the hydantoin or to ensure regioselectivity for the deprotonation. Intermediate B was purified using flash chromatography or preparative high pressure liquid chromatography (HPLC) using standard solvent systems like ethyl acetate/hexane, methylene chloride/methanol, or acetonitrile/water (reverse phase HPLC). Typically, a gradient of low polarity to higher polarity solvents can be used to separate byproducts. Following purification of Intermediate B, it can be coupled with an appropriately substituted phenyl ring where Z is a standard coupling functional group like boronic acids or esters (Suzuki reaction), or stannanes like trimethyl- or tributylstannane (Stille reaction) where the coupling reaction is mediated by a transition metal like palladium (e.g. Tetrakis(triphenylphosphine)palladium(O)), copper (e.g. Buchwald or Fu conditions), or zinc mediated coupling (e.g. Negishi conditions). This led to compounds of Formula I directly or after protecting group removal or further substitution reactions as exemplified in specific reaction schemes below. Typically, the final compounds of Formula I are purified by chromatography methods (flash or preparative HPLC) to greater than 95% purity prior to biological testing.

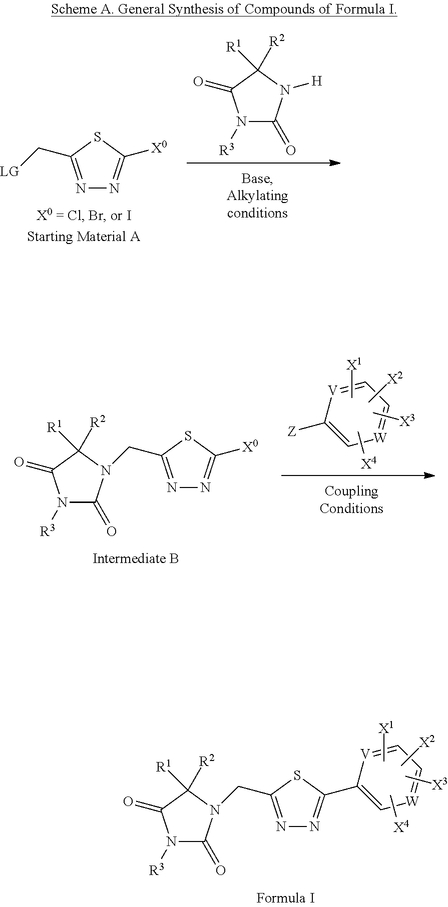

### General Scheme B. General Synthesis of Hydantoin Starting Material C

Hydantoin Starting Material C was prepared using general hydantoin synthetic routes well known to those skilled in the art. One general method is outlined in General Scheme B where amino ester Starting Material B is either commercially available or prepared using standard amino acid synthesis followed by ester formation (e.g. anhydrous HCl in ethanol). Starting Material B was reacted with 1.5 equivalents of P-nitrophenyl chloroformate 1B using 1.5 equivalents of organic base (e.g. diisopropylethylamine, DIEA) in a halogenated solvent (e.g. dichloromethane, DCM) starting at 0° C. and allowing to warm to 20° C. over 1-2 h (typically reaction complete in 1 h) to form Intermediate 2B. Typically, Intermediate 2B is taken directly into the next reaction without purification. Urea Intermediate 4B is formed by reacting amine 3B using 1.0 equivalents of organic base (e.g. diisopropylethylamine, DIEA) in a halogenated solvent (e.g. dichloromethane, DCM) at a temperature of 20° C. Amine 3B is typically commercially available but it can be prepared using standard amine synthesis well known to one skilled in the art. Urea Intermediate 4B is typically purified using flash chromatography or preparative high pressure liquid chromatography (HPLC) using standard solvent systems like ethyl acetate/hexane, methylene chloride/methanol, or acetonitrile/water (reverse phase HPLC). Typically, a gradient of low polarity to higher polarity solvents can be used to separate byproducts. Urea Intermediate 4B was typically converted to hydantoin Starting Material C by treating with 2 equivalents of an inorganic base (e.g. cesium carbonate, Cs₂CO₃) in a dipolar aprotic solvent (e.g. dimethylformamide, DMF) at 20° C. for a period of 2-4 h (e.g. 2 h). In some cases, the reaction may require warming to induce the hydantoin formation. The Hydantoin Starting Material C was typically purified using flash chromatography or preparative high pressure liquid chromatography (HPLC) using standard solvent systems like ethyl acetate/hexane, methylene chloride/methanol, or acetonitrile/water (reverse phase HPLC).

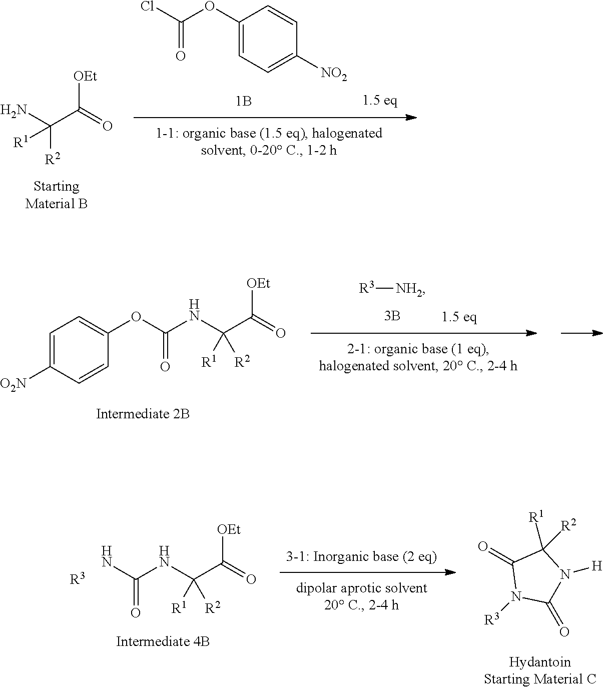

### Specific Examples of Preparation of Formula I Compounds Via Synthetic Route 1

Compound I-7 of Formula I was prepared as outlined in Scheme 1 for Synthetic route 1 (C1). Reduction of commercially available ester 1-C1 CAS [1030613-07-0] with 1.01 equivalents of sodium borohydride (NaBH₄) afforded primary alcohol 2-C1 that was used in the next step after quenching with water and standard extraction. Compound 2-C1 was oxidized to the aldehyde 3-C1 using 1.5 equivalents of Dess-Martin Periodinane CAS [87413-09-0] in DCM at 0° C. for 2 h. After quenching with aqueous saturated sodium thiosulfate and standard workup, the crude product was purified by flash column chromatography to afford compound 3-C1. Aldehyde 3-C1 was reacted with commercially available 1 equivalent of methyl 1-aminocyclopropane-1-carboxylate [CAS #72784-42-0] using 1.1 equivalents of titanium (IV) isopropylate to induce imine formation in tetrahydrofuran (THF) for 1 h at 25° C. under an inert atmosphere followed by reduction through the addition of 2 equivalents sodium borohydride (NaBH₄) in ethanol to the same reaction flask. After 6 h at 25° C., thin layer chromatography indicated that the reaction was complete and the reaction was quenched with saturated sodium bicarbonate. Following a standard aqueous extraction, drying, and concentration in vacuo the product was purified by flash chromatography to afford compound 4-C1. Amine 4-C1 was further reacted with 2 equivalents of commercially available ethyl isocyanate [Cas #109-90-0] in acetonitrile with 2 equivalents of DIEA at 50° C. under an inert nitrogen atmosphere. After 12 h at 50° C., the reaction was determined to be complete as determined by TLC analysis. The reaction was concentrated, standard workup, followed by purification by flash chromatography to afford compound 5-C1. Compound 5-C1 was cyclized to hydantoin 6-C1 by treatment with 2 equivalents of cesium carbonate in DMF at 25° C. for 1 h under an inert nitrogen atmosphere. After standard workup, the compound was purified by flash column chromatography to afford hydantoin 6-C1. Hydantoin 6-C1 containing the bromothiadiazole was further reacted with 1 equivalent of (2,4-difluoro-3-hydroxyphenyl)boronic acid [CAS #2894848-23-6] using 0.1 equivalents of palladium tetrakis [Pd(PPh₃)₄] as catalyst and 2 equivalents of sodium carbonate as base in a mixed solvent of toluene:ethanol:water (1:1:1) at 80° C. for 2 h under an argon atmosphere. Analysis by LC-MS indicated the reaction was complete. The reaction was diluted with water and pH adjusted to 6 with citric acid followed by standard extraction, drying, and purification by reverse phase preparative HPLC to afford compound I-7 as a white solid. The following compounds in Table 1 were made in a similar manner to compound I-7.

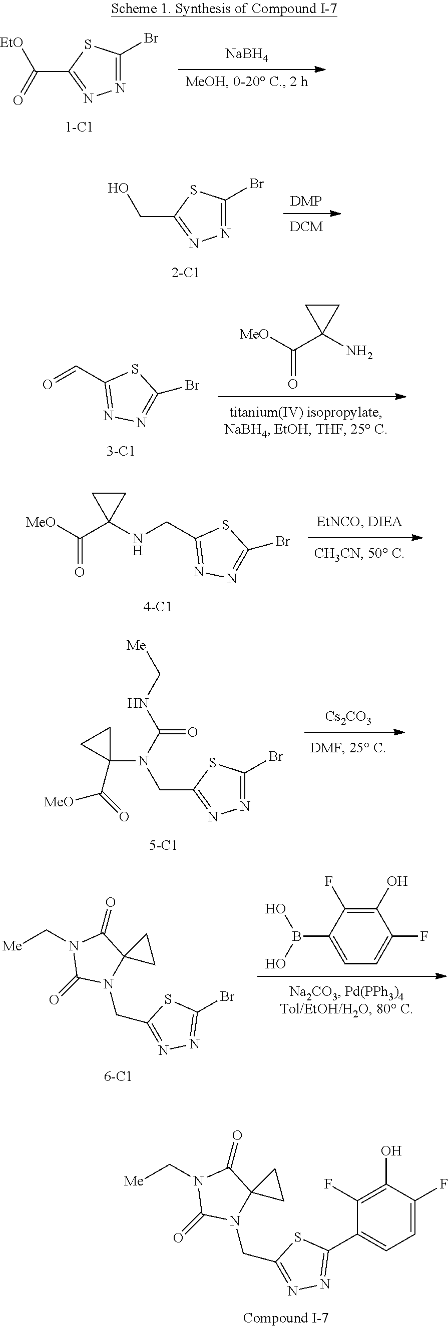

TABLE 1
Targets associated with synthetic route 1

| | Compound | | |
|---|---|---|---|
| Example | ID | Name | Structure |
| 1 | I-7 | 4-((5-(2,4-difluoro-3- hydroxyphenyl)-1,3,4- thiadiazol-2-yl)methyl)- 6-ethyl-4,6- diazaspiro[2.4]heptane- 5,7-dione | 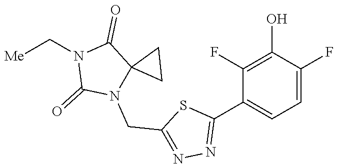 [SMILES: CCN1C(=O)N(Cc2nnc(-c3ccc(F)c(O)c3F)s2)C2(CC2)C1=O] |
| 2 | I-9 | 4-((5-(2,4-dichloro-3- hydroxyphenyl)-1,3,4- thiadiazol-2-yl)methyl)- 6-ethyl-4,6- diazaspiro[2.4]heptane- 5,7-dione | 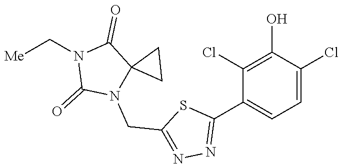 [SMILES: CCN1C(=O)N(Cc2nnc(-c3ccc(Cl)c(O)c3Cl)s2)C2(CC2)C1=O] |
| 3 | I-10 | 4-((5-(2-chloro-4-fluoro- 3-hydroxyphenyl)-1,3,4- thiadiazol-2-yl)methyl)- 6-ethyl-4,6- diazaspiro[2.4]heptane- 5,7-dione | 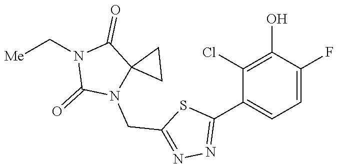 [SMILES: CCN1C(=O)N(Cc2nnc(-c3ccc(F)c(O)c3Cl)s2)C2(CC2)C1=O] |
| 4 | I-11 | 4-((5-(4-chloro-5- hydroxypyridin-3-yl)- 1,3,4-thiadiazol-2- yl)methyl)-6-ethyl-4,6- diazaspiro[2.4]heptane- 5,7-dione | 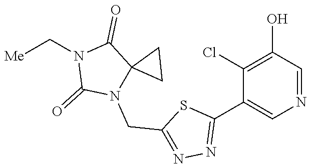 [SMILES: CCN1C(=O)N(Cc2nnc(-c3cncc(O)c3Cl)s2)C2(CC2)C1=O] |
| 5 | I-13 | 4-((5-(2-chloro-3- hydroxy-5- (trifluoromethyl)phenyl)- 1,3,4-thiadiazol-2- yl)methyl)-6-ethyl-4,6- diazaspiro[2.4]heptane- 5,7-dione | 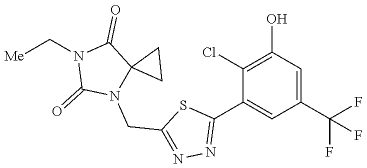 [SMILES: CCN1C(=O)N(Cc2nnc(-c3cc(C(F)(F)F)cc(O)c3Cl)s2)C2(CC2)C1=O] |
| 6 | I-14 | 6-ethyl-4-((5-(2-fluoro- 3-hydroxy-5- (trifluoromethyl)phenyl)- 1,3,4-thiadiazol-2- yl)methyl)-4,6- diazaspiro[2.4]heptane- 5,7-dione | 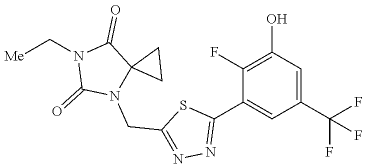 [SMILES: CCN1C(=O)N(Cc2nnc(-c3cc(C(F)(F)F)cc(O)c3F)s2)C2(CC2)C1=O] |
| 7 | I-23 | 4-((5-(4-chloro-2-fluoro- 3-hydroxyphenyl)-1,3,4- thiadiazol-2-yl)methyl)- 6-ethyl-4,6- diazaspiro[2.4]heptane- 5,7-dione | 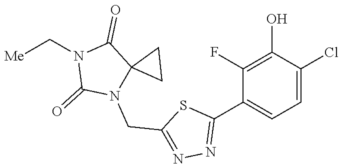 [SMILES: CCN1C(=O)N(Cc2nnc(-c3ccc(Cl)c(O)c3F)s2)C2(CC2)C1=O] |

### List of Abbreviations

| | |
|---|---|
| ACN | Acetonitrile |
| AR | Argon |
| BPD | Bis(pinacolato)diboron |
| DCE | Dichloroethane |
| DCM | Dichloromethane |
| DIEA | Diisopropylethylamine |
| DMAP | 4-Dimethylaminopyridine |
| DMF | Dimethylformamide |
| DMP | Dess-Martin Periodinane |
| DMSO | Dimethylsulfoxide |
| EtOH | Ethanol |
| eq | equivalents |
| EtOAc | Ethyl acetate |
| FA | Formic acid |
| h, hr, hrs | hour(s) |
| HPLC | High Pressure Liquid Chromatography or High |
| | Performance Liquid Chromatography |
| LC-MS, | Liquid Chromatography - Mass Spectrometry |
| LCMS | |
| LDA | Lithium Diisopropylamide |
| NCS | N-Chlorosuccinamide |
| RT | Retention Time |
| N2 | nitrogen |
| PE | Petroleum ether |
| SFC | Supercritical fluid chromatography |
| T4P | 1,3,5,2,4,6-Trioxatriphosphorinane, 2,4,6- |
| | tributyl, 2,4,6-trioxide CAS # 163755-62-2 |
| TBAA | Tetrabutylammonium acetate CAS# 10534-59-5 |
| TEA | Triethylamine |
| TFA | Trifluoroacetic acid |
| THF | Tetrahydrofuran |
| TLC | Thin Layer Chromatography |
| x, * | Means repeat - extracted 2x ethyl acetate or 2* |
| | ethyl acetate, or by for size of column - 20 * 100 |
| | mm, or 20 × 100 mm |
| um | micron |

### Example 1

### Preparation of (5-bromo-1,3,4-thiadiazol-2-yl)methanol Compound 2-C1

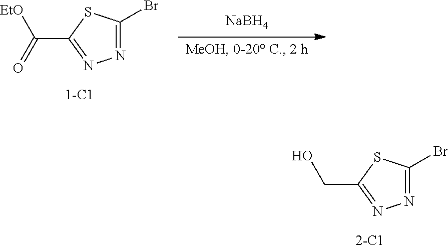 [SMILES: CCOC(=O)c1nnc(Br)s1 ; "MeOH, 0-20° C., 2 h" ; "NaBH4" ; OCc1nnc(Br)s1]

To a mixture of Compound 1-C1 (30 g, 126.54 mmol, 1 eq) in methanol (600 mL) was added NaBH₄ (4.84 g, 127.81 mmol, 1.01 eq) in several portions at 0° C. under N₂. The mixture was stirred at 20° C. for 2 hours. TLC (petroleum ether:ethyl acetate=3:1, R_{f}=0.43) showed the reaction was completed. The mixture was quenched with H₂O (250 mL), extracted with ethyl acetate (100 mL×3). The combined organic phases were washed with brine, dried with anhydrous Na₂SO₄, filtered and concentrated in vacuum to afford crude Compound 2-C1 (17 g, 87.16 mmol,) as a yellow solid, which was used in next step directly.

¹H NMR (400 MHz, DMSO-d₆) δ 6.36 (t, J=6.0 Hz, 1H), 4.85 (d, J=6.0 Hz, 2H).

### Preparation of 5-bromo-1,3,4-thiadiazole-2-carbaldehyde Compound 3-C1

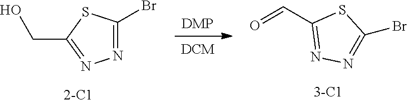

To a solution of compound 2-C1 (5 g, 25.6 mmol, 1 eq) in DCM (50 mL) was added Dess-Martin Periodinane (DMP, 16.3 g, 38.4 mmol, 11.9 mL, 1.5 eq) at 0° C. The mixture was stirred at 20° C. for 2 hr. TLC indicated one new spot formed. The mixture was quenched by aqueous saturated sodium thiosulfate (10 mL), diluted with H₂O (30 mL) and extracted with ethyl acetate (20 mL×5). The combined organic layers were washed with aqueous NaHCO₃ (5%), dried over Na₂SO₄, filtered and concentrated under reduced pressure. The residue was purified by column chromatography (SiO₂, petroleum ether:ethyl acetate=90:10) to give compound 3-C1 (2.7 g, 13.9 mmol,) as a yellow solid.

¹H NMR (400 MHz, DMSO-d₆) δ 10.10 (s, 1H).

### Preparation of methyl 1-[(5-bromo-1,3,4-thiadiazol-2-yl)methylamino]cyclopropanecarboxylate Compound 4-C1

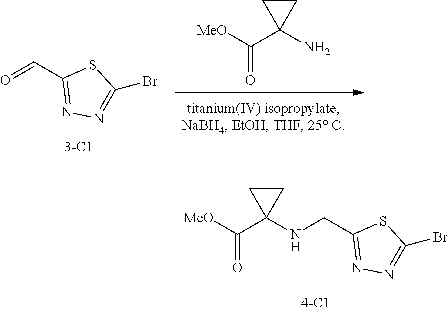 [SMILES: O=Cc1nnc(Br)s1 ; COC(=O)C1(N)CC1 ; COC(=O)C1(NCc2nnc(Br)s2)CC1]

A mixture of compound 3-C1 (400 mg, 2.07 mmol, 1 eq), methyl 1-aminocyclopropanecarboxylate (238.58 mg, 2.07 mmol, 1 eq), tetraisopropoxytitanium (647.88 mg, 2.28 mmol, 672.77 μL, 1.1 eq) in THF (8 mL) was degassed and purged with N₂ for 3 times. The mixture was stirred at 25° C. for 1 hour under N₂ atmosphere. Ethanol (1.91 g, 41.45 mmol, 20 eq) and NaBH₄ (156.80 mg, 4.14 mmol, 2 eq) was added. The mixture was stirred at 25° C. for 6 hours under N₂ atmosphere. TLC indicated the reactant was consumed completely. The mixture was quenched by saturated NaHCO₃ (10 mL), diluted with H₂O (5 mL) and extracted with ethyl acetate (20 mL*2). The combined organic layers were dried over Na₂SO₄, filtered and concentrated under reduced pressure. The residue was purified by column chromatography (SiO₂, petroleum ether:ethyl acetate=1:0 to 1:1) to give compound 4-C1 (180 mg, 616.12 μmol) as a yellow oil.

¹H NMR (400 MHz, DMSO-d₆) δ 4.23 (d, J=7.2 Hz, 2H), 4.17-4.05 (m, 1H), 3.62 (s, 3H), 1.21-1.12 (m, 2H), 1.04-0.90 (m, 2H).

### Preparation of methyl 1-[(5-bromo-1,3,4-thiadiazol-2-yl)methyl-(ethylcarbamoyl)amino]cyclopropanecarboxylate Compound 5-C1

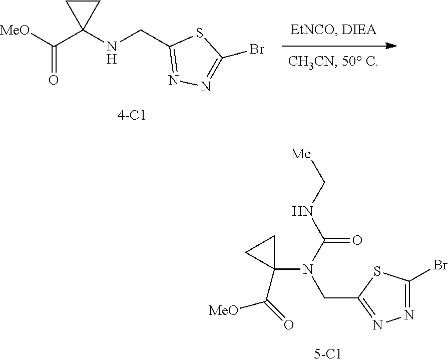 [SMILES: COC(=O)C1(NCc2nnc(Br)s2)CC1 ; CCNC(=O)N(Cc1nnc(Br)s1)C1(C(=O)OC)CC1]

A mixture of compound 4-C1 (360 mg, 1.23 mmol, 1 eq), isocyanatoethane (175.17 mg, 2.46 mmol, 195.07 μL, 2 eq), DIEA (318.52 mg, 2.46 mmol, 429.27 μL, 2 eq) in ACN (5 mL) was degassed and purged with N₂ for 3 times. The mixture was stirred at 50° C. for 12 hours under N₂ atmosphere. TLC indicated the reactant was consumed completely. The mixture was concentrated under reduced pressure. The residue was diluted with H₂O (10 mL), extracted with ethyl acetate (10 mL×2). The combined organic layers were dried over Na₂SO₄, filtered and concentrated in vacuum. The residue was purified by column chromatography (SiO₂, petroleum ether:ethyl acetate=1:0 to 1:1) to give compound 5-C1 (320 mg, 880.98 μmol) as a yellow oil.

¹H NMR (400 MHz, DMSO-d₆) δ 6.61 (t, J=6.0 Hz, 1H), 4.71 (s, 2H), 3.48 (s, 3H), 3.08 (quin, J=7.2 Hz, 2H), 1.82-1.63 (m, 1H), 1.60-1.40 (m, 1H), 1.39-1.25 (m, 2H), 0.99 (t, J=7.2 Hz, 3H).

LCMS: RT=0.371 min, MS cal.: 362.0, MS found: [M+H]⁺=363.0, 364.9.

### Preparation of 4-[(5-bromo-1,3,4-thiadiazol-2-yl)methyl]-6-ethyl-4,6-diazaspiro[2.4]heptane-5,7-dione Compound 6-C1

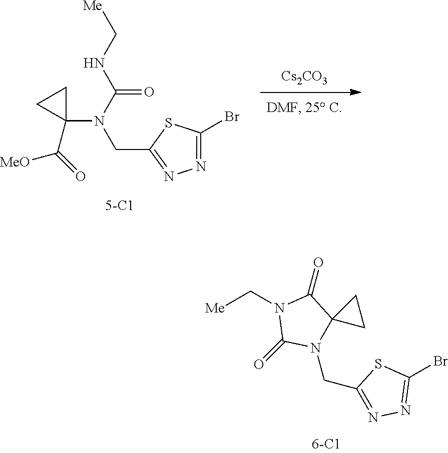 [SMILES: CCNC(=O)N(Cc1nnc(Br)s1)C1(C(=O)OC)CC1 ; "Cs2CO3" ; CCN1C(=O)N(Cc2nnc(Br)s2)C2(CC2)C1=O]

A mixture of compound 5-C1 (320 mg, 880.98 μmol, 1 eq), Cs₂CO₃ (574.08 mg, 1.76 mmol, 2 eq) in DMF (4 mL) was degassed and purged with N₂ for 3 times. The mixture was stirred at 25° C. for 1 hour under N₂ atmosphere. LC-MS showed the reactant was consumed completely. The reaction mixture was diluted with H₂O (20 mL), extracted with ethyl acetate (20 mL×2). The combined organic layers were dried over Na₂SO₄, filtered and concentrated in vacuum. The residue was purified by column chromatography (SiO₂, petroleum ether:ethyl acetate=1:0 to 1:1) to give compound 6-C1 (170 mg, 513.30 μmol) as a yellow oil.

¹H NMR (400 MHz, DMSO-d₆) δ 4.83 (s, 2H), 3.49 (q, J=7.2 Hz, 2H), 1.59-1.45 (m, 2H), 1.25-1.20 (m, 2H), 1.12 (t, J=7.2 Hz, 3H).

LCMS: RT=0.361 min, MS cal.: 330.0, MS found: [M+H]⁺=330.9, 333.0.

### Preparation of 4-[[5-(2,4-difluoro-3-hydroxy-phenyl)-1,3,4-thiadiazol-2-yl]methyl]-6-ethyl-4,6-diazaspiro[2.4]heptane-5,7-dione Compound I-7

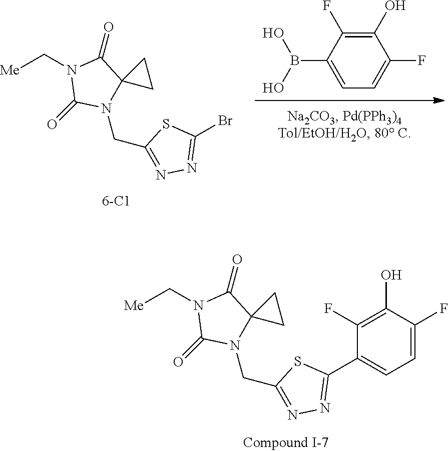 [SMILES: CCN1C(=O)N(Cc2nnc(Br)s2)C2(CC2)C1=O ; OB(O)c1ccc(F)c(O)c1F ; CCN1C(=O)N(Cc2nnc(-c3ccc(F)c(O)c3F)s2)C2(CC2)C1=O]

A mixture of compound 6-C1 (170 mg, 513.30 μmol, 1 eq), (2,4-difluoro-3-hydroxy-phenyl)boronic acid (89.27 mg, 513.30 μmol, 1 eq), Pd(PPh₃)₄ (59.32 mg, 51.33 μmol, 0.1 eq) and Na₂CO₃ (108.81 mg, 1.03 mmol, 2 eq) in toluene (0.8 mL), ethanol (0.8 mL) and H₂O (0.8 mL) was degassed and purged with Ar for 3 times. The mixture was stirred at 80° C. for 2 hours under Ar atmosphere. LC-MS showed the reactant was consumed completely. The reaction mixture was diluted with H₂O (20 mL), adjust pH=6 using citric acid and extracted with ethyl acetate (20 mL×2). The combined organic layers were dried over Na₂SO₄, filtered and concentrated in vacuum. The residue was purified by prep-HPLC (column: Phenomenex luna C18 100*40 mm*3 um; mobile phase: [H₂O (0.2% formic acid)-ACN]; gradient: 35%-65% B over 8.0 min) to give Compound I-7 (61.71 mg, 162.11 μmol, 99.92% purity) as a white solid.

¹H NMR (400 MHz, DMSO-d₆) δ 10.81 (s, 1H), 7.85-7.49 (m, 1H), 7.46-7.09 (m, 1H), 4.90 (s, 2H) 3.51 (q, J=7.2 Hz, 2H), 1.64-1.49 (m, 2H), 1.31-1.21 (m, 2H), 1.13 (t, J=7.2 Hz, 3H).

LCMS: RT=2.173 min, MS cal.: 308.08, MS found: [M+H]⁺=381.1.

### Example 2

### Route:

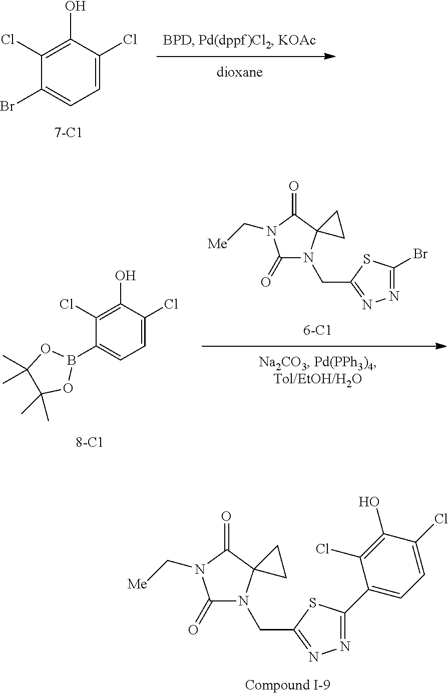 [SMILES: Oc1c(Cl)ccc(Br)c1Cl ; CC1(C)OB(c2ccc(Cl)c(O)c2Cl)OC1(C)C ; CCN1C(=O)N(Cc2nnc(Br)s2)C2(CC2)C1=O ; CCN1C(=O)N(Cc2nnc(-c3ccc(Cl)c(O)c3Cl)s2)C2(CC2)C1=O]

### Preparation of 2,6-dichloro-3-(4,4,5,5-tetramethyl-1,3,2-dioxaborolan-2-yl)phenol Compound 8-C1

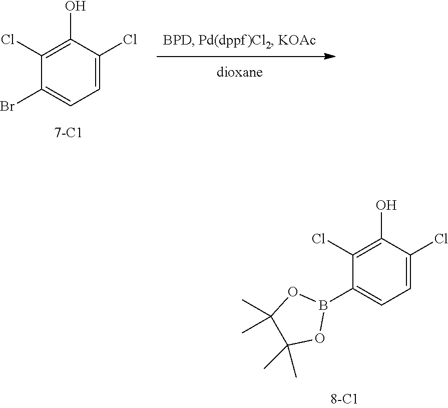 [SMILES: Oc1c(Cl)ccc(Br)c1Cl ; CC1(C)OB(c2ccc(Cl)c(O)c2Cl)OC1(C)C]

To a solution of Compound 7-C1 (0.2 g, 826.80 μmol, 1 eq) and 4,4,5,5-tetramethyl-2-(4,4,5,5-tetramethyl-1,3,2-dioxaborolan-2-yl)-1,3,2-dioxaborolane (230.95 mg, 909.48 μmol, 1.1 eq) in dioxane (2 mL) was added potassium acetate (202.86 mg, 2.07 mmol, 2.5 eq) and Pd(dppf)Cl₂ (60.50 mg, 82.68 μmol, 0.1 eq) at 20° C. under N₂. The mixture was stirred at 90° C. for 16 hours. The mixture was cooled to 20° C. and filtered and concentrated in vacuum. The residue was purified by silica gel chromatography (column height: 250 mm, diameter: 100 mm, 100-200 mesh silica gel, petroleum ether:ethyl acetate=10:1 to 3:1) to give Compound 8-C1 (100 mg, 346.07 mol) as a white solid, which was used directly for next step.

### Preparation of 4-((5-(2,4-dichloro-3-hydroxyphenyl)-1,3,4-thiadiazol-2-yl)methyl)-6-ethyl-4,6-diazaspiro[2.4]heptane-5,7-dione Compound I-9

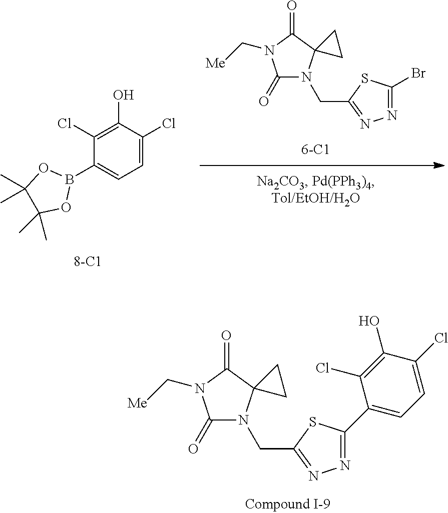 [SMILES: CC1(C)OB(c2ccc(Cl)c(O)c2Cl)OC1(C)C ; CCN1C(=O)N(Cc2nnc(Br)s2)C2(CC2)C1=O ; CCN1C(=O)N(Cc2nnc(-c3ccc(Cl)c(O)c3Cl)s2)C2(CC2)C1=O]

To a solution of 6-C1 (50 mg, 150.97 μmol, 1 eq) and Compound 8-C1 (52.35 mg, 181.17 μmol, 1.2 eq) in toluene (0.2 mL), Ethanol (0.2 mL) and H₂O (0.2 mL) was added Na₂CO₃ (32.00 mg, 301.94 μmol, 2 eq) under N₂. Pd(PPh₃)₄ (17.45 mg, 15.10 μmol, 0.1 eq) was added to the reaction solution at 20° C. under N₂. Then the mixture was stirred at 80° C. for 16 h. The mixture was cooled to 20° C., filtered and concentrated in vacuum. The residue was purified by prep-HPLC (column: Waters xbridge 150*25 mm*10 um; mobile phase: [H₂O (10 mM NH₄HCO₃)-ACN]; gradient: 5%-48% B over 8.0 min) to get Compound I-9 (54 mg, 130.39 μmol 99.79% purity) as white solid.

¹H NMR (400 MHz, DMSO-d₆) δ 7.28 (d, J=8.4 Hz, 1H), 7.06 (d, J=8.4 Hz, 1H), 4.88 (s, 2H), 3.51 (q, J=7.2 Hz, 2H), 1.61-1.54 (m, 2H), 1.29-1.22 (m, 2H), 1.13 (t, J=7.2 Hz, 3H) LCMS: MS found: [M+H]⁺=413.0.

### Example 3

### Route:

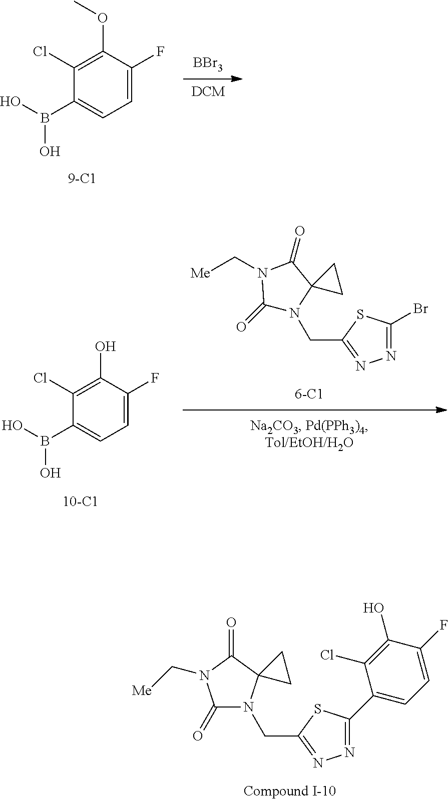

### Preparation of (2-chloro-4-fluoro-3-hydroxyphenyl)boronic acid Compound 10-C1

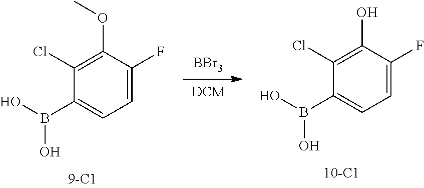

To a solution of Compound 9-C1 (0.55 g, 2.69 mmol, 1 eq) in DCM (12 mL) was added tribromoborane (2 M, 4.04 mL, 3 eq). The mixture was stirred at 25° C. for 1 hour under N₂ atmosphere. The mixture was poured into water (10 mL), extracted with ethyl acetate (15 mL×3). The combined organic layers were dried over Na₂SO₄, filtered and concentrated under reduced pressure. The residue was triturated with ethyl acetate (10 mL), filtered and the organic layers was concentrated under reduced pressure to afford compound 10-C1 (460 mg, 2.42 mmol) as a yellow solid.

¹H NMR (400 MHz, DMSO-d₆) δ 10.02 (s, 1H), 8.23 (s, 2H), 7.09 (dd, J=8.0, 10.4 Hz, 1H), 6.83 (dd, J=6.0, 8.0 Hz, 1H).

### Preparation of 4-((5-(2-chloro-4-fluoro-3-hydroxyphenyl)-1,3,4-thiadiazol-2-yl)methyl)-6-ethyl-4,6-diazaspiro[2.4]heptane-5,7-dione Compound I-10

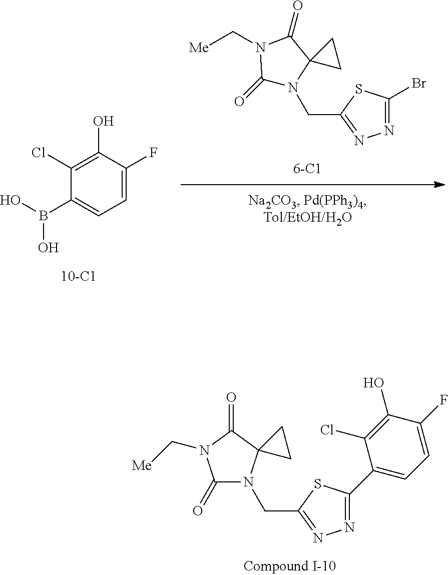 [SMILES: OB(O)c1ccc(F)c(O)c1Cl ; CCN1C(=O)N(Cc2nnc(Br)s2)C2(CC2)C1=O ; CCN1C(=O)N(Cc2nnc(-c3ccc(F)c(O)c3Cl)s2)C2(CC2)C1=O]

To a solution of compound 10-C1 (68.97 mg, 362.33 μmol, 1.2 eq) and compound 6-C1 (100 mg, 301.94 μmol, 1 eq) in Tol. (0.33 mL), Ethanol (0.33 mL) and H₂O (0.33 mL) was added Pd(PPh₃)₄ (34.89 mg, 30.19 μmol, 0.1 eq) and Na₂CO₃ (64.01 mg, 603.89 μmol, 2 eq). The mixture was stirred at 80° C. for 16 hours under Argon atmosphere. The solution was adjusted to pH ˜6 with citric acid and extracted with Ethyl acetate (10 mL×3). The combined organic layers were dried over Na₂SO₄, filtered, and concentrated under reduced pressure. The residue was purified by prep-HPLC (column: 3_Phenomenex Luna C18 75×30 mm×3 um; mobile phase: [H₂O (0.2% formic acid)-ACN]; gradient: 25%-55% B over 8.0 min) to afford Compound I-10 (53 mg, 131.52 μmol 98.473% purity) as white solid.

¹H NMR (400 MHz, DMSO-d₆) δ 11.15-10.88 (m, 1H), 7.60 (dd, J=5.6, 8.8 Hz, 1H), 7.40 (dd, J=8.8, 10.0 Hz, 1H), 4.92 (s, 2H), 3.52 (q, J=7.2 Hz, 2H), 1.63-1.53 (m, 2H), 1.30-1.23 (m, 2H), 1.14 (t, J=7.2 Hz, 3H).

LCMS: MS found: [M+H]⁺=397.1.

### Example 4

### Route:

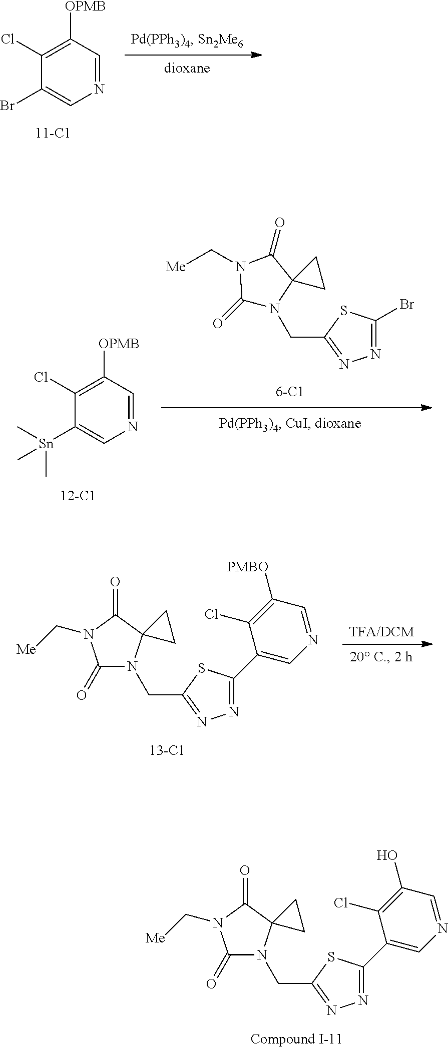

### Preparation of [4-chloro-5-[(4-methoxyphenyl)methoxy]-3-pyridyl]-trimethyl-stannane Compound 12-C1

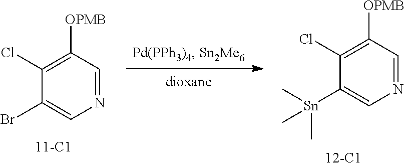

A mixture of Compound 11-C1 (500 mg, 1.52 mmol, 1 eq), trimethyl(trimethylstannyl)stannane (747.80 mg, 2.28 mmol, 473.29 μL, 1.5 eq), Pd(PPh₃)₄ (175.84 mg, 152.17 μmol, 0.1 eq) in dioxane (10 mL) was degassed and purged with N₂ atmosphere for 3 times. The mixture was stirred at 100° C. for 6 hours under N₂ atmosphere. LC-MS showed the reactant was consumed completely. The reaction mixture was quenched by aqueous KF (1M, 20 mL) at 20° C., diluted with H₂O (20 mL) and extracted with ethyl acetate (40 mL×2). The combined organic layers were dried over Na₂SO₄, filtered and concentrated under reduced pressure. The residue was purified by column chromatography (SiO₂, petroleum ether:ethyl acetate=1:0 to 2:1) to give Compound 12-C1 (500 mg, 1.21 mmol,) as a yellow solid.

LCMS: RT=0.484 min, MS cal.: 413.02, MS found: [M+H]⁺=414.1.

¹H NMR (400 MHz, DMSO-d₆) δ ppm 8.44 (s, 1H), 8.05 (s, 1H), 7.40 (d, J=8.4 Hz, 2H), 6.96 (d, J=8.4 Hz, 2H), 5.22 (s, 2H), 3.76 (s, 3H), 0.29-0.47 (m, 9H).

### Preparation of 4-[[5-[4-chloro-5-[(4-methoxyphenyl)methoxy]-3-pyridyl]-1,3,4-thiadiazol-2-yl]methyl]-6-ethyl-4,6-diazaspiro[2.4]heptane-5,7-dione Compound 13-C1

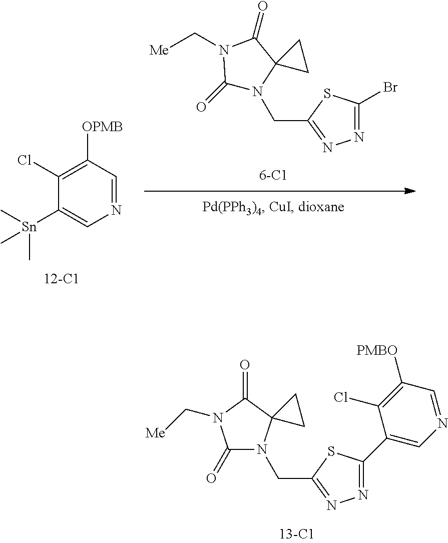

A mixture of Compound 12-C1 (200 mg, 484.85 μmol, 1 eq), 6-C1 (160.58 mg, 484.85 μmol, 1 eq), Pd(PPh₃)₄ (56.03 mg, 48.49 μmol, 0.1 eq), CuI (9.23 mg, 48.49 μmol, 0.1 eq) in dioxane (4 mL) was degassed and purged with N₂ for 3 times. The mixture was stirred at 90° C. for 12 hours under N₂ atmosphere. LC-MS showed the reactant was consumed completely. The reaction mixture was concentrated under reduced pressure. The residue was diluted with H₂O (30 mL), extracted with ethyl acetate (30 mL*2). The combined organic layers were dried over Na₂SO₄, filtered and concentrated under reduced pressure. The residue was purified by column chromatography (SiO₂, petroleum ether:ethyl acetate=1:0 to 0:1) to give Compound 13-C1 (200 mg, 400.02 μmol) as yellow solid.

¹H NMR (400 MHz, DMSO-d₆) δ 8.83 (s, 1H), 8.73 (s, 1H), 7.45 (d, J=8.4 Hz, 2H), 6.98 (d, J=8.4 Hz, 2H), 5.35 (s, 2H), 4.94 (s, 2H), 3.77 (s, 3H), 3.51 (q, J=7.2 Hz, 2H), 1.55-1.62 (m, 2H), 1.23-1.29 (m, 2H), 1.13 (t, J=7.2 Hz, 3H).

### Preparation of 4-[[5-(4-chloro-5-hydroxy-3-pyridyl)-1,3,4-thiadiazol-2-yl]methyl]-6-ethyl-4,6-diazaspiro[2.4]heptane-5,7-dione—Compound I-11

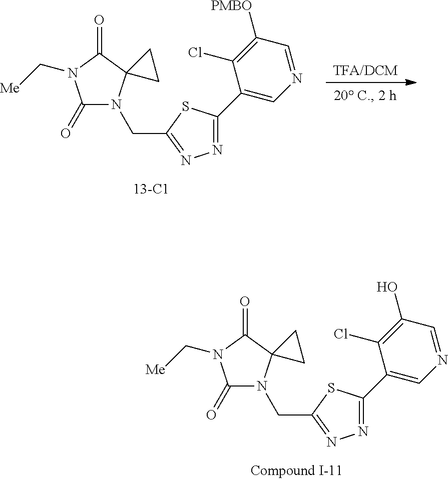

To a solution of Compound 13-C1 (250 mg, 500.03 μmol, 1 eq) in TFA (1 mL) and DCM (3 mL). The mixture was stirred at 20° C. for 2 hours. LC-MS showed the reactant was consumed completely. The reaction mixture was diluted with H₂O (20 mL), extracted with solvent ethyl acetate (20 mL×2). Dried over Na₂SO₄, filtered and concentrated under reduced pressure. The residue was purified by prep-HPLC (column: Phenomenex Luna C18 100*30 mm*3 um; mobile phase: [H₂O (0.2% formic acid)-ACN]; gradient: 10%-40% B over 8.0 min) to give Compound I-11 (163.37 mg, 425.31 μmol, 98.88% purity) as a white solid.

¹H NMR (400 MHz, DMSO-d₆) δ ppm 11.41 (d, J=7.2 Hz, 1H), 8.65 (s, 1H), 8.39 (s, 1H), 4.94 (s, 2H), 3.52 (q, J=7.2 Hz, 2H), 1.55-1.64 (m, 2H), 1.24-1.31 (m, 2H), 1.13 (t, J=7.2 Hz, 3H).

LCMS: MS found: [M+H]⁺=380.0.

### Example 5

### Route:

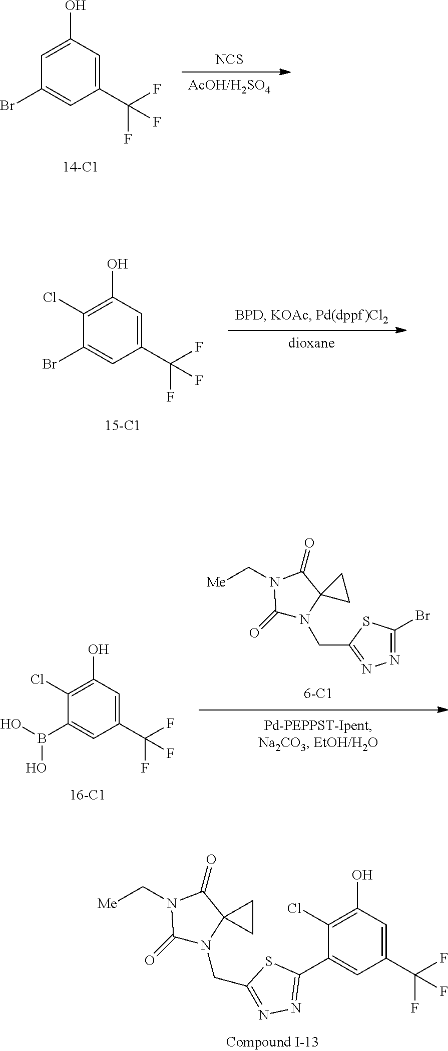

### Preparation of 3-bromo-2-chloro-5-(trifluoromethyl)phenol Compound 15-C1

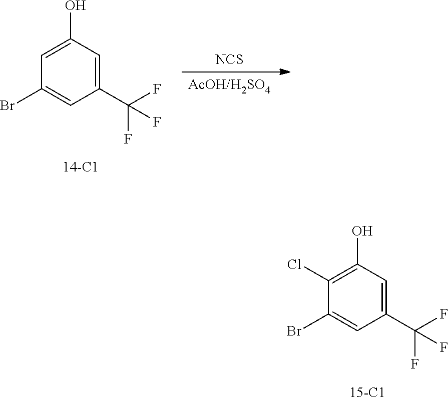 [SMILES: Oc1cc(Br)cc(C(F)(F)F)c1 ; "AcOH/H2SO4" ; "NCS" ; Oc1cc(C(F)(F)F)cc(Br)c1Cl]

To a solution of Compound 14-C1 (4 g, 16.60 mmol, 1 eq) in AcOH (40 mL) and H₂SO₄ (4 mL) was added N-Chlorosuccinamide (NCS, 2.22 g, 16.60 mmol, 1 eq) at 15° C. under N₂ atmosphere. The mixture was stirred at 60° C. for 2 hours. The residue was poured into water (50 mL), extracted with DCM (100 mL×3). The combined organic phases were washed with brine, dried with anhydrous Na₂SO₄, filtered and concentrated in vacuum. The residue was purified by column chromatography (SiO₂, petroleum ether:ethyl acetate=10:1 to 5:1) to get Compound 15-C1 (4 g, 7.84 mmol, 54% purity) as a colorless oil.

### Preparation of (2-chloro-3-hydroxy-5-(trifluoromethyl)phenyl)boronic acid Compound 16-C1

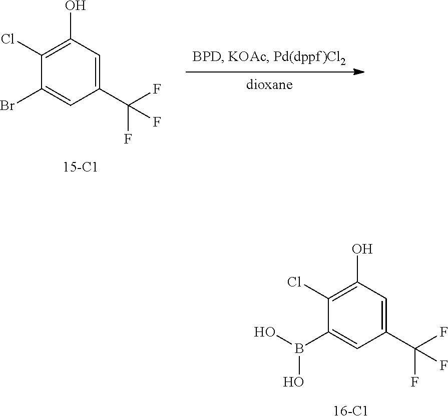 [SMILES: Oc1cc(C(F)(F)F)cc(Br)c1Cl ; "BPD, KOAc, Pd(dppf)Cl2" ; "dioxane" ; OB(O)c1cc(C(F)(F)F)cc(O)c1Cl]

To a solution of Compound 15-C1 (5 g, 18.15 mmol, 1 eq) and Pin₂B₂ (5.53 g, 21.78 mmol, 1.2 eq) in dioxane (50 mL) was added potassium acetate (5.34 g, 54.46 mmol, 3 eq) and Pd(dppf)Cl₂ (1.33 g, 1.82 mmol, 0.1 eq) at 15° C. under N₂ atmosphere. Then mixture was stirred at 90° C. for 3 h. The residue was poured into ice-water (100 mL), extracted with ethyl acetate (50 mL×3). The combined organic phases were washed with brine, dried with anhydrous Na₂SO₄, filtered and concentrated in vacuum. The residue was purified by prep-HPLC (column: Phenomenex luna C18 (250*70 mm, 15 um); mobile phase: [H₂O (0.04% HCl)-ACN]; gradient: 30%-60% B over 18.0 min) to get Compound 16-C1 (100 mg, 416.02 μmol) as a white solid.

### Preparation of 4-((5-(2-chloro-3-hydroxy-5-(trifluoromethyl)phenyl)-1,3,4-thiadiazol-2-yl)methyl)-6-ethyl-4,6-diazaspiro[2.4]heptane-5,7-dione Compound I-13

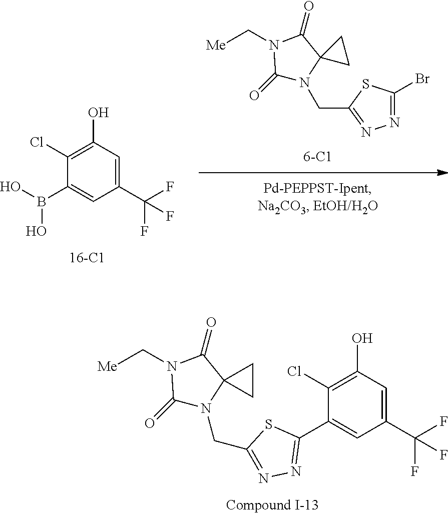 [SMILES: OB(O)c1cc(C(F)(F)F)cc(O)c1Cl ; CCN1C(=O)N(Cc2nnc(Br)s2)C2(CC2)C1=O ; CCN1C(=O)N(Cc2nnc(-c3cc(C(F)(F)F)cc(O)c3Cl)s2)C2(CC2)C1=O]

To a solution of 6-C1 (25 mg, 75.49 μmol, 1 eq) and Compound 16-C1 (43.55 mg, 90.58 μmol, 1.2 eq) in ethanol (0.4 mL) and H₂O (0.1 mL) was added Na₂CO₃ (12.00 mg, 113.23 μmol, 1.5 eq) and [1,3-Bis(2,6-Diisopropylphenyl)imidazol-2-ylidene](3-chloropyridyl)palladium(II) dichloride (6.50 mg, 7.55 μmol, 0.1 eq) at 15° C. under N₂ atmosphere. The mixture was stirred at 80° C. for 1 hour. The mixture was cooled to 15° C. TFA (0.05 mL) was poured into the reaction, extracted with ethyl acetate (1 mL×3). The combined organic phases were washed with brine, dried with anhydrous Na₂SO₄, filtered and concentrated in vacuum. The residue was purified by prep-HPLC (column: Phenomenex Luna C18 100*30 mm*3 um; mobile phase: [H₂O (0.2% formic acid)-ACN]; gradient: 30%-60% B over 8.0 min) to get Compound I-13 (2.05 mg, 4.51 mol, 98.266% purity) as a white solid.

¹H NMR (400 MHz, DMSO-d₆) δ 11.83-11.36 (m, 1H), 7.86 (d, J=1.2 Hz, 1H), 7.40 (d, J=2.0 Hz, 1H), 4.94 (s, 2H), 3.52 (q, J=7.2 Hz, 2H), 1.62-1.55 (m, 2H), 1.29-1.22 (m, 2H), 1.13 (t, J=7.2 Hz, 3H).

LCMS: MS found: [M+H]⁺=447.0.

### Example 6

### Route:

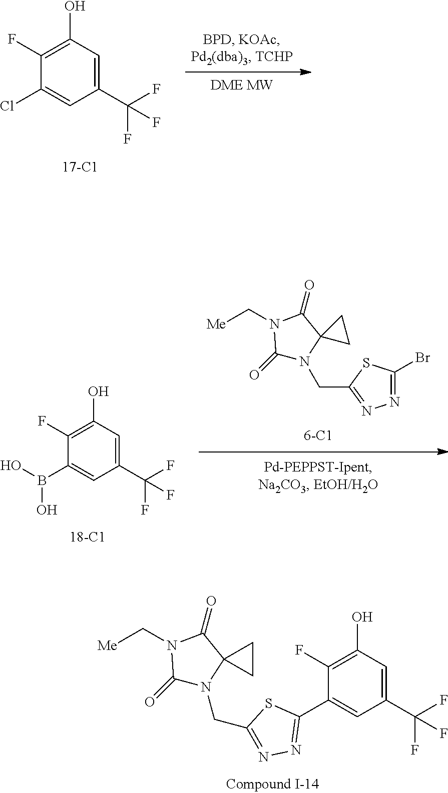 [SMILES: Oc1cc(C(F)(F)F)cc(Cl)c1F ; "BPD, KOAc,Pd2(dba)3, TCHP" ; "DME MW" ; OB(O)c1cc(C(F)(F)F)cc(O)c1F ; CCN1C(=O)N(Cc2nnc(Br)s2)C2(CC2)C1=O ; CCN1C(=O)N(Cc2nnc(-c3cc(C(F)(F)F)cc(O)c3F)s2)C2(CC2)C1=O]

### Preparation of [2-fluoro-3-hydroxy-5-(trifluoromethyl) phenyl] boronic acid Compound 18-C1

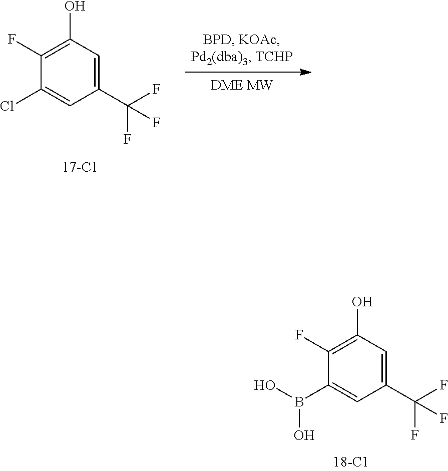 [SMILES: Oc1cc(C(F)(F)F)cc(Cl)c1F ; "BPD, KOAc,Pd2(dba)3, TCHP" ; "DME MW" ; OB(O)c1cc(C(F)(F)F)cc(O)c1F]

To a solution of Compound 17-C1 (500 mg, 2.33 mmol, 1 eq) in 1,2-dimethoxyethane (5 mL) was added bis(pinacolato)diboron (BPD, 1.18 g, 4.66 mmol, 2 eq) and (1E,4E)-1,5-Diphenyl-1,4-pentadien-3-one-palladium (1:1) (213.41 mg, 233.05 μmol, 0.1 eq) and tricyclohexylphosphane (261.42 mg, 932.21 μmol, 302.22 μL, 0.4 eq) and potassium acetate (343.08 mg, 3.50 mmol, 1.5 eq) at 25° C. The reaction mixture was stirred at 140° C. for 6 hours under N₂ atmosphere in microwave. The reaction mixture was quenched by addition water (10 mL), extracted with ethyl acetate (10 mL×3). The combined organic layers were washed with brine, dried over Na₂SO₄, filtered and concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 5%-55% B over 8.0 min) to afford Compound 18-C1 (600 mg, 2.28 mmol, 85% purity) as a white solid.

### Preparation of 6-ethyl-4-[[5-[2-fluoro-3-hydroxy-5-(trifluoromethyl) phenyl]-1,3,4-thiadiazol-2-yl]methyl]-4,6-diazaspiro[2.4]heptane-5,7-dione Compound I-14

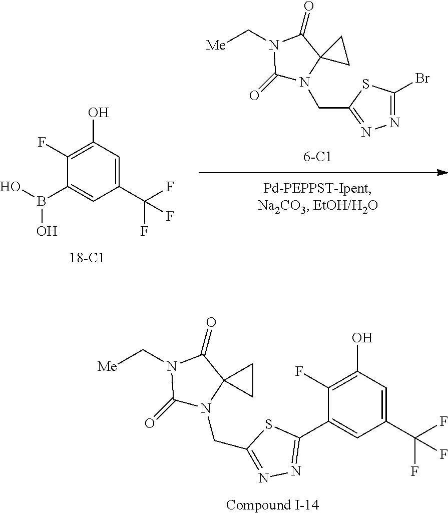 [SMILES: OB(O)c1cc(C(F)(F)F)cc(O)c1F ; CCN1C(=O)N(Cc2nnc(Br)s2)C2(CC2)C1=O ; CCN1C(=O)N(Cc2nnc(-c3cc(C(F)(F)F)cc(O)c3F)s2)C2(CC2)C1=O]

To a solution of compound 18-C1 (65 mg, 290.29 μmol, 1 eq) and 6-C1 (96.14 mg, 290.29 μmol, 1 eq) in ethanol (0.6 mL) and H₂O (0.15 mL) was added Na₂CO₃ (46.15 mg, 435.43 μmol, 1.5 eq) and 1,3-bis[2,6-bis(1-ethylpropyl)phenyl]-4,5-dichloro-2H-imidazol-1-ium-2-ide dichloro(3-chloropyridine-κN)palladium (12.49 mg, 14.51 μmol, 0.05 eq) at 25° C., then the reaction mixture was stirred at 80° C. for 2 hours under N₂ atmosphere. The reaction mixture was quenched by addition water (10 mL), extracted with ethyl acetate (10 mL×3). The combined organic layers were washed with brine, dried over Na₂SO₄, filtered and concentrated under reduced pressure. The residue was purified by prep-HPLC (column: Phenomenex Luna C18 75*30 mm*3 um; mobile phase: [H₂O (0.1% TFA)-ACN]; gradient: 30%-60% B over 8.0 min) to afford Compound I-14 (19 mg, 44.15 μmol, 100% purity) as a white solid.

¹H NMR (400 MHz, DMSO-d₆) δ 7.70 (d, J=3.2 Hz, 1H), 7.27 (d, J=6.8 Hz, 1H), 4.93 (s, 2H), 3.56-3.49 (m, 2H), 1.63-1.51 (m, 2H), 1.29-1.22 (m, 2H), 1.13 (t, J=7.2 Hz, 3H).

LCMS: MS found: [M+H]⁺=431.1.

### Example 7

### Route:

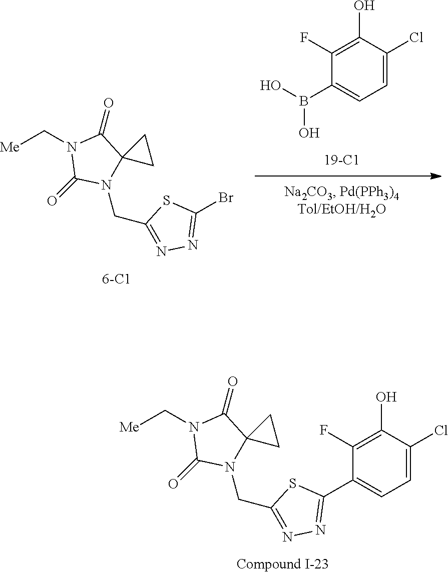 [SMILES: CCN1C(=O)N(Cc2nnc(Br)s2)C2(CC2)C1=O ; OB(O)c1ccc(Cl)c(O)c1F ; CCN1C(=O)N(Cc2nnc(-c3ccc(Cl)c(O)c3F)s2)C2(CC2)C1=O]

### Preparation of 4-[[5-(4-chloro-2-fluoro-3-hydroxy-phenyl)-1,3,4-thiadiazol-2-yl]methyl]-6-ethyl-4,6-diazaspiro[2.4]heptane-5,7-dione Compound I-23

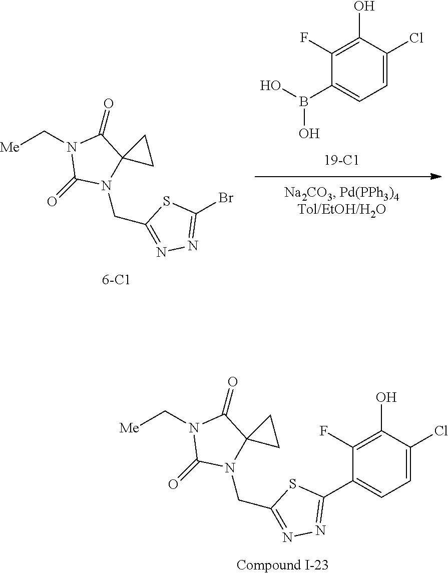 [SMILES: CCN1C(=O)N(Cc2nnc(Br)s2)C2(CC2)C1=O ; OB(O)c1ccc(Cl)c(O)c1F ; CCN1C(=O)N(Cc2nnc(-c3ccc(Cl)c(O)c3F)s2)C2(CC2)C1=O]

A mixture of 6-C1 (150 mg, 452.91 μmol, 1 eq), compound 19-C1 (86.22 mg, 452.91 μmol, 1 eq), Pd(PPh₃)₄ (52.34 mg, 45.29 μmol, 0.1 eq), Na₂CO₃ (96.01 mg, 905.83 μmol, 2 eq) in toluene (1 mL), ethanol (1 mL) and H₂O (1 mL) was degassed and purged with N₂ for 3 times. The mixture was stirred at 80° C. for 2 hours under N₂ atmosphere. LC-MS showed the reactant was consumed completely. The reaction mixture was diluted with H₂O (20 mL), adjust pH=5-6 with citric acid and extracted with Ethyl acetate (20 mL×2). The combined organic layers were dried over Na₂SO₄, filtered and concentrated under reduced pressure. The residue was purified by prep-HPLC (column: Phenomenex luna C18 100*40 mm*5 um; mobile phase: [H₂O (0.2% formic acid)-ACN]; gradient: 25%-65% B over 8.0 min) to give Compound I-23 (67.67 mg, 169.68 μmol, 99.5% purity) as a white solid.

¹H NMR (400 MHz, DMSO-d₆) δ 11.21-10.84 (m, 1H) 7.71-7.59 (m, 1H) 7.41 (dd, J=7.2, 1.2 Hz, 1H) 4.91 (s, 2H) 3.51 (q, J=7.2 Hz, 2H) 1.53-1.61 (m, 2H) 1.22-1.29 (m, 2H) 1.13 (t, J=7.2 Hz, 3H).

LCMS: MS found: [M+H]⁺=397.1.

### Specific Examples of Preparation of Formula I Compounds Via Synthetic Route 2

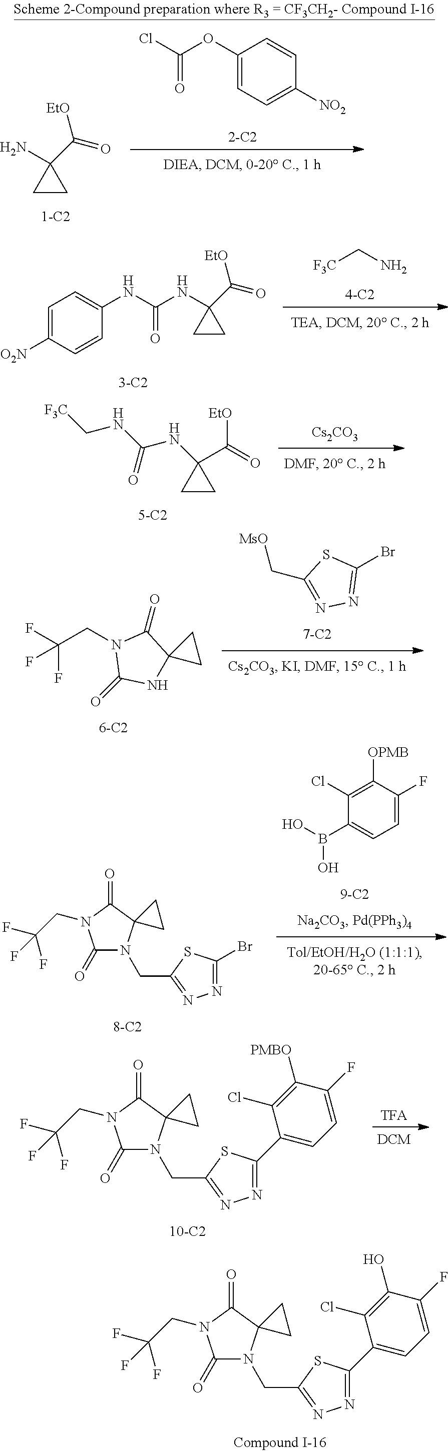

Compounds where R³=CF₃CH₂— can be prepared following Scheme 2 illustrated above with the preparation of Compound I-16. Commercially available 1-Aminocyclopropyl carboxylic acid ethyl ester hydrochloride 1-C2 [CAS #42303-42-4] was reacted with 1.5 equivalents of P-nitrophenyl chloroformate 2-C2 using 1.5 equivalents of diisopropylethylamine (DIEA) in dichloromethane (DCM) starting at 0° C. and allowing to warm to 20° C. over 1 h to form Intermediate 3-C2. Intermediate 3-C2 was taken directly into the next reaction without purification. Urea Intermediate 5-C2 is formed by reacting trifluoroethylamine 4-C2 using 1.0 equivalents of DIEA in DCM at a temperature of 20° C. After 2 h, TLC analysis indicated the reaction was complete, so the reaction was concentrated under reduced pressure and the residue was purified by flash column chromatography. The isolated urea 5-C2 was further purified by trituration with petroleum ether:ethyl acetate (1:1) to afford urea 5-C2 as a white solid. Urea Intermediate 5-C2 was converted to hydantoin Starting Material 6-C2 by treating with 2 equivalents of cesium carbonate (Cs₂CO₃) in dimethylformamide (DMF) at 20° C. for a period of 2 h. After TLC analysis confirmed product was formed, the reaction was diluted with water, standard extraction and workup. The Hydantoin Starting Material 6-C2 was purified using flash chromatography (ethyl acetate/petroleum ether) to afford hydantoin 6-C2 as a yellow oil. Hydantoin 6-C2 was reacted with mesylate 7-C2 (1 Equivalent) with one equivalent of potassium iodide in DMF at 15° C. for 1 h at which time TLC analysis indicated the reaction was complete. After standard workup, the bromothiadiazole 8-C2 was purified by flash column chromatography (petroleum ether:ethyl acetate, 9:1) to afford 8-C2 as a yellow solid. Bromide 8-C2 was reacted with 1.3 equivalents of (2-chloro-4-fluoro-3-((4-methoxybenzyl)oxy)phenyl)boronic acid 9-C2, 2 equivalents of sodium carbonate (Na₂CO₃), and 0.1 equivalents of Pd(PPh₃)₄ in water:ethanol:toluene (1:1:1) with a 3 cycle degassing (vacuum, nitrogen purging) at 80° C. for 2 h. After TLC analysis confirmed product was formed, the reaction was diluted with water, standard extraction and workup. The residue was purified by preparative TLC (silica gel, petroleum ether:ethyl acetate, 1:1) to afford intermediate 10-C2 as a yellow oil. The 4-methoxybenzyl protecting group was removed from 10-C2 by treatment with TFA in DCM at 30° C. for 8 h. After concentration in vacuo, the residue was purified by reverse phase preparative HPLC to afford Compound I-16.

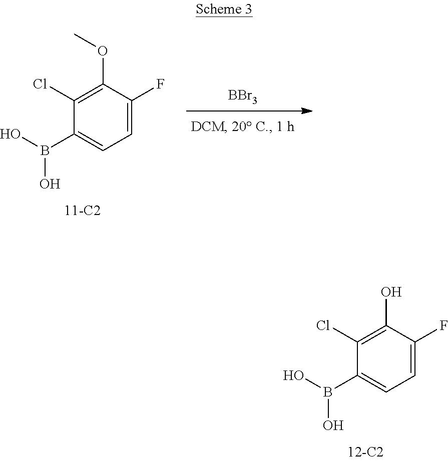

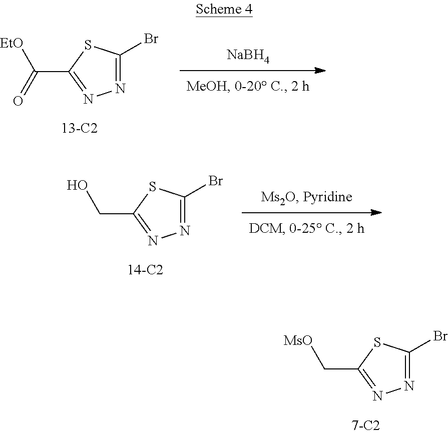 [SMILES: CCOC(=O)c1nnc(Br)s1 ; "NaBH4" ; OCc1nnc(Br)s1 ; "Ms2O, Pyridine" ; CS(=O)(=O)OCc1nnc(Br)s1]

Commercially available (2-chloro-4-fluoro-3-methoxyphenyl)boronic acid 11-C2 [943831-11-6] was converted to the free phenol by treatment with 3 equivalents of boron tribromide in DCM at 20° C. for 1 h (Scheme 3). After quenching with water and a standard workup, the residue was triturated with ethyl acetate, filtered, and the filtrate was concentrated to afford phenol 12-C2 as a yellow solid. Phenol 12-C2 was converted to (2-chloro-4-fluoro-3-((4-methoxybenzyl)oxy)phenyl)boronic acid 9-C2 using standard conditions. Compound 13-C2 was converted to mesylate 7-C2 by treatment with several portions of 1.25 equivalents of methanesulfonic anhydride (Ms₂O), pyridine (2 equivalents) in DCM at 0° C. The reaction was allowed to warm to 20° C. for 2 h (Scheme 4). The reaction was quenched into ice water followed by a standard workup to afford mesylate 7-C2 as a yellow solid that can be used directly in the next step.

TABLE 2
Synthetic route 2 Examples

| | Compound | | |
|---|---|---|---|
| Example | ID | Name | Structure |
| 8 | I-16 | 4-((5-(2-chloro-4- fluoro-3- hydroxyphenyl)-1,3,4- thiadiazol-2- yl)methyl)-6-(2,2,2- trifluoroethyl)-4,6- diazaspiro[2.4]heptane- 5,7-dione | 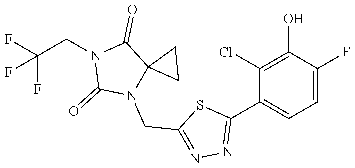 [SMILES: O=C1N(CC(F)(F)F)C(=O)C2(CC2)N1Cc1nnc(-c2ccc(F)c(O)c2Cl)s1] |
| 9 | I-15 | 4-((5-(2,4-difluoro-3- hydroxyphenyl)-1,3,4- thiadiazol-2- yl)methyl)-6-(2,2,2- trifluoroethyl)-4,6- diazaspiro[2.4]heptane- 5,7-dione | 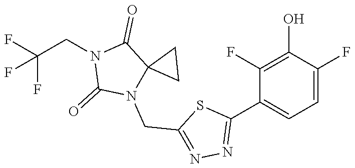 [SMILES: O=C1N(CC(F)(F)F)C(=O)C2(CC2)N1Cc1nnc(-c2ccc(F)c(O)c2F)s1] |
| 10 | I-17 | 4-((5-(2,4-dichloro-3- hydroxyphenyl)-1,3,4- thiadiazol-2- yl)methyl)-6-(2,2,2- trifluoroethyl)-4,6- diazaspiro[2.4]heptane- 5,7-dione | 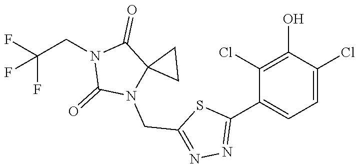 [SMILES: O=C1N(CC(F)(F)F)C(=O)C2(CC2)N1Cc1nnc(-c2ccc(Cl)c(O)c2Cl)s1] |
| 11 | I-24 | 4-((5-(4-chloro-2- fluoro-3- hydroxyphenyl)-1,3,4- thiadiazol-2- yl)methyl)-6-(2,2,2- trifluoroethyl)-4,6- diazaspiro[2.4]heptane- 5,7-dione | 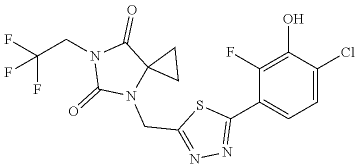 [SMILES: O=C1N(CC(F)(F)F)C(=O)C2(CC2)N1Cc1nnc(-c2ccc(Cl)c(O)c2F)s1] |

### Preparation of ethyl 1-(3-(2,2,2-trifluoroethyl)ureido)cyclopropane-1-carboxylate Compound 5-C2

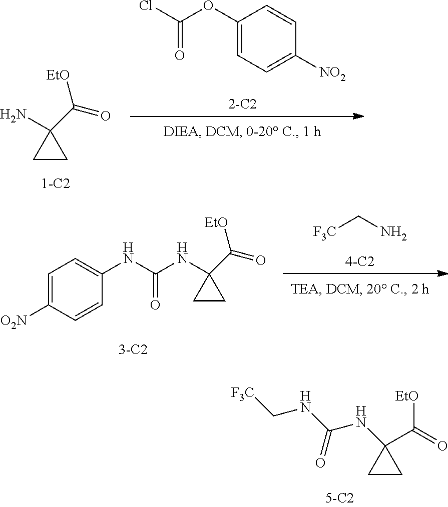

A mixture of Compound 1-C2 (11 g, 85.17 mmol, 1 eq), DIEA (16.51 g, 127.75 mmol, 22.25 mL, 1.5 eq) in DCM (100 mL) was degassed and purged with N₂ for 3 times. Then (4-nitrophenyl) carbonochloridate 2-C2 (25.75 g, 127.75 mmol, 1.5 eq) was added to the reaction solution at 0° C. The mixture was stirred at 20° C. for 1 hour under N₂ atmosphere. TLC (petroleum ether:ethyl acetate=3:1, R_{f}=0.4) indicated one new spot formed. The reaction solution was used in the next step without further work-up and purification.

To a solution of Compound 3-C2 (11.50 g, 116.12 mmol, 9.11 mL, 1.5 eq) and DIEA (10.01 g, 77.41 mmol, 13.48 mL, 1 eq) in DCM (100 mL) was added Compound 4-C2 (22.78 g, 77.41 mmol, 1 eq). The mixture was stirred at 20° C. for 2 hours. TLC (petroleum ether:ethyl acetate=3:1, R_{f}=0.18) indicated one new spot formed. The reaction mixture was concentrated under reduced pressure. The residue was purified by column chromatography (SiO₂, petroleum ether:ethyl acetate=0:100). The crude product was triturated with the mixture solvent (petroleum ether:ethyl acetate=1:1) (3 mL) at 15° C. for 3 times to give Compound 5-C2 (9.0 g, 35.4 mmol) as a white solid.

¹H NMR (400 MHz, DMSO-d₆) δ 6.89 (s, 1H), 6.58 (t, J=6.0 Hz, 1H), 4.02 (q, J=7.2 Hz, 2H), 3.85-3.73 (m, 2H), 1.36-1.28 (m, 2H), 1.13 (t, J=7.2 Hz, 3H), 1.05-0.98 (m, 2H).

### Preparation of 6-(2,2,2-trifluoroethyl)-4,6-diazaspiro[2.4]heptane-5,7-dione Compound 6-C2

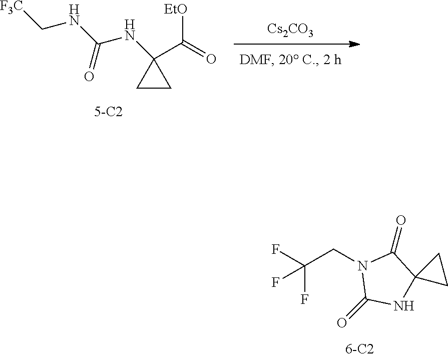

To a solution of Compound 5-C2 (1.5 g, 5.90 mmol, 1 eq) in DMF (30 mL) was added Cs₂CO₃ (3.85 g, 11.80 mmol, 2 eq). The mixture was stirred at 20° C. for 2 hours. TLC (petroleum ether:ethyl acetate=3:1, R_{f}=0.17) indicated one new spot formed. The reaction mixture diluted with H₂O (50 mL), extracted with ethyl acetate (20 mL×3). The combined organic layers were washed with brine, dried over Na₂SO₄, filtered and concentrated under reduced pressure. The residue was purified by column chromatography (SiO₂, Petroleum ether:ethyl acetate=85:15) to give Compound 6-C2 (0.5 g, 2.40 mmol) as a yellow oil.

¹H NMR (400 MHz, CDCl₃) δ 6.19 (s, 1H), 4.23-4.10 (m, 2H), 1.62-1.58 (m, 2H), 1.41-1.34 (m, 2H).

### Preparation of 4-((5-bromo-1,3,4-thiadiazol-2-yl)methyl)-6-(2,2,2-trifluoroethyl)-4,6-diazaspiro[2.4]heptane-5,7-dione Compound 8-C2

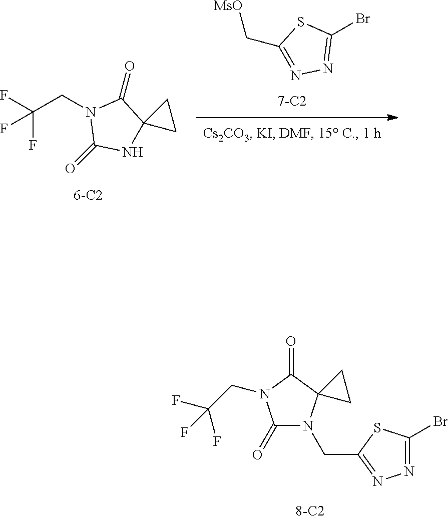 [SMILES: O=C1NC2(CC2)C(=O)N1CC(F)(F)F ; CS(=O)(=O)OCc1nnc(Br)s1 ; O=C1N(CC(F)(F)F)C(=O)C2(CC2)N1Cc1nnc(Br)s1]

To a solution of Compound 6-C2 (0.5 g, 2.01 mmol, 1 eq) and Compound 7-C2 (502 mg, 2.42 mmol, 1.2 eq) in DMF (5 mL) was added Cs₂CO₃ (1.31 g, 4.03 mmol, 2 eq) and KI (334 mg, 2.01 mmol, 1 eq). The mixture was stirred at 15° C. for 1 hour. TLC (Petroleum ether:ethyl acetate=1:1, R_{f}=0.31) indicated one new spot formed. The reaction mixture diluted with H₂O (40 mL), extracted with ethyl acetate (20 mL×3). The combined organic layers were washed with brine, dried over Na₂SO₄, filtered and concentrated under reduced pressure. The residue was purified by column chromatography (SiO₂, petroleum ether:ethyl acetate=9:1) to give Compound 8-C2 (0.6 g, 1.56 mmol) as a yellow solid.

¹H NMR (400 MHz, CDCl₃) δ 4.72 (s, 2H), 4.21 (q, J=8.4 Hz, 2H), 1.62-1.58 (m, 2H), 1.55-1.51 (m, 2H).

### Preparation of (5-bromo-1,3,4-thiadiazol-2-yl)methanol Compound 14-C2

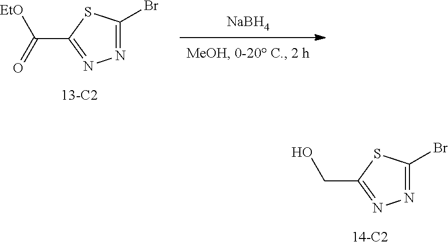 [SMILES: CCOC(=O)c1nnc(Br)s1 ; "NaBH4" ; OCc1nnc(Br)s1]

To a mixture of Compound 13-C2 (30 g, 126.54 mmol, 1 eq) in Methanol (600 mL) was added NaBH₄ (4.84 g, 127.81 mmol, 1.01 eq) in several portions at 0° C. under N₂ atmosphere. The mixture was stirred at 20° C. for 2 hours. TLC (petroleum ether:ethyl acetate=3:1, R_{f}=0.43) showed the reaction was completed. The mixture was quenched with H₂O (250 mL). The aqueous phase was extracted with ethyl acetate (100 mL×3). The combined organic phases were washed with brine, dried with anhydrous Na₂SO₄, filtered and concentrated in vacuum to afford crude Compound 14-C2 (17 g, 87.16 mmol) as yellow solid, which was used to next step directly.

¹H NMR (400 MHz, DMSO-d₆) δ 6.36 (t, J=6.0 Hz, 1H), 4.85 (d, J=6.0 Hz, 2H).

### Preparation of (5-bromo-1,3,4-thiadiazol-2-yl)methyl methanesulfonate compound 7-C2

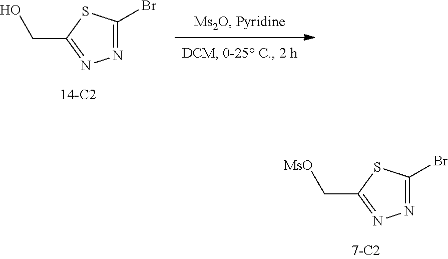 [SMILES: OCc1nnc(Br)s1 ; "Ms2O, Pyridine" ; CS(=O)(=O)OCc1nnc(Br)s1]

To a mixture of Compound 14-C2 (16.7 g, 85.62 mmol, 1 eq) and pyridine (13.55 g, 171.25 mmol, 13.82 mL, 2 eq) in DCM (145 mL) was added methanesulfonic anhydride (18.64 g, 107.03 mmol, 1.25 eq) in several portions at 0° C. under N₂ atmosphere. The mixture was stirred at 25° C. for 2 hours. The mixture was poured into ice-water (50 mL), extracted with DCM (30 mL×3). The combined organic phases were washed with 1M HCl solution (20 mL), brine (30 mL), dried with anhydrous Na₂SO₄, filtered and concentrated in vacuum to afford crude Compound 7-C2 (16.2 g, 59.31 mmol) as a yellow solid, which was used to next step directly.

¹H NMR (400 MHz, DMSO-d₆) δ 5.73 (s, 2H), 3.34 (s, 3H).

### Preparation of (2-chloro-4-fluoro-3-hydroxyphenyl)boronic acid

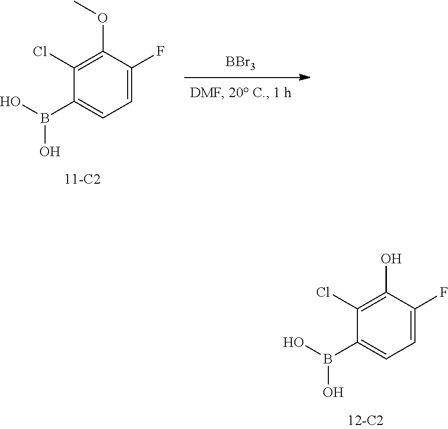

To a solution of Compound 11-C2 (0.55 g, 2.69 mmol, 1 eq) in DCM (12 mL) was added tribromoborane (2 M, 4.04 mL, 3 eq). The mixture was stirred at 20° C. for 1 h under N₂ atmosphere. The mixture was poured into water (10 mL), extracted with ethyl acetate (15 mL×3). The combined organic layers were dried over Na₂SO₄, filtered and concentrated under reduced pressure. The residue was triturated with ethyl acetate (10 mL), filtered and the organic layer was concentrated under reduced pressure to afford Compound 12-C2 (460 mg, 2.42 mmol) as a yellow solid.

¹H NMR (400 MHz, DMSO-d₆) δ 10.02 (s, 1H), 8.23 (s, 2H), 7.09 (dd, J=8.0, 10.4 Hz, 1H), 6.83 (dd, J=6.0, 8.0 Hz, 1H).

### Preparation of 4-((5-(2-chloro-4-fluoro-3-hydroxyphenyl)-1,3,4-thiadiazol-2-yl)methyl)-6-(2,2,2-trifluoroethyl)-4,6-diazaspiro[2.4]heptane-5,7-dione Compound I-16

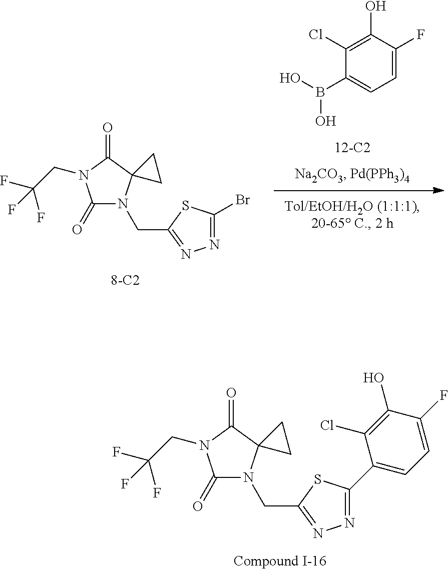 [SMILES: OB(O)c1ccc(F)c(O)c1Cl ; O=C1N(CC(F)(F)F)C(=O)C2(CC2)N1Cc1nnc(Br)s1 ; O=C1N(CC(F)(F)F)C(=O)C2(CC2)N1Cc1nnc(-c2ccc(F)c(O)c2Cl)s1]

To a solution of Compound 8-C2 (120 mg, 311.56 μmol, 1 eq) and Compound 12-C2 (71.17 mg, 373.87 μmol, 1.2 eq) in toluene (0.5 mL), ethanol (0.5 mL) and H₂O (0.5 mL) was added Na₂CO₃ (66.04 mg, 623.12 μmol, 2 eq) and Pd(PPh₃)₄ (36.00 mg, 31.16 μmol, 0.1 eq) at 20° C. under N₂ atmosphere. The mixture was stirred at 65° C. for 2 hour. LC-MS showed the reaction was completed. The mixture was cooled to 20° C. and filtered and concentrated in vacuum. The residue was purified by prep-HPLC (column: Phenomenex Luna C18 100*30 mm*3 um; mobile phase: [H₂O (0.2% FORMIC ACID)-ACN]; gradient: 30%-60% B over 8.0 min) to get Compound I-16 (12 mg, 26.35 μmol, 99% purity) as a white solid.

¹H NMR (400 MHz, DMSO-d₆) δ 11.25-10.76 (s, 1H), 7.63-7.51 (m, 1H), 7.44-7.30 (m, 1H), 4.96 (s, 2H), 4.35 (q, J=9.6 Hz, 2H), 1.72-1.65 (m, 2H), 1.38-1.31 (m, 2H).

LCMS: MS found: [M+H]⁺=451.0.

### Example 9

### Route:

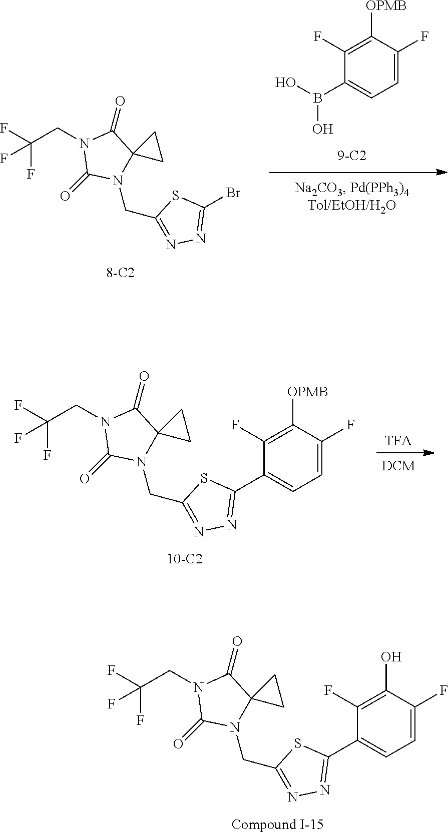

### Preparation of 4-[[5-[2,4-difluoro-3-[(4-methoxyphenyl)methoxy]phenyl]-1,3,4-thiadiazol-2-yl]methyl]-6-(2,2,2-trifluoroethyl)-4,6-diazaspiro[2.4]heptane-5,7-dione Compound 10-C2

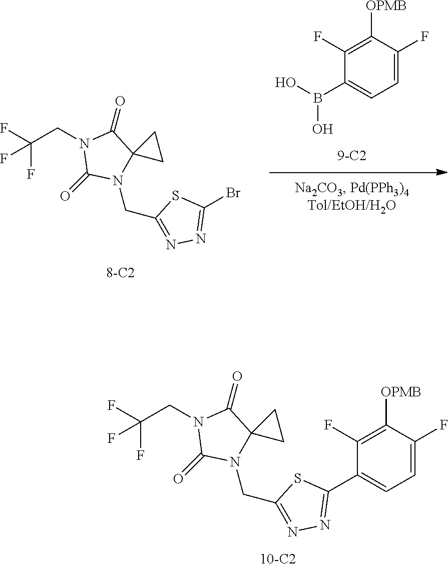

A mixture of 8-C2 (0.1 g, 259 μmol, 1 eq), Compound 9-C2 (99.2 mg, 337 μmol, 1.3 eq), Na₂CO₃ (55.0 mg, 519 μmol, 2 eq), Pd(PPh₃)₄ (30.0 mg, 25.9 μmol, 0.1 eq) in H₂O (0.1 mL), ethanol (0.1 mL) and toluene (0.1 mL) was degassed and purged with N₂ for 3 times. The mixture was stirred at 80° C. for 2 hours under N₂ atmosphere. TLC (petroleum ether:ethyl acetate=1:1, R_{f}=0.47) indicated one new spot formed. The reaction mixture diluted with H₂O (30 mL), extracted with ethyl acetate (20 mL×2). The combined organic layers were washed with brine, dried over Na₂SO₄, filtered and concentrated under reduced pressure. The residue was purified by prep-TLC (SiO₂, petroleum ether:ethyl acetate=1:1) to give compound 10-C2 (0.1 g, 180 μmol) as a yellow oil.

¹H NMR (400 MHz, CDCl₃-d) δ 8.01 (ddd, J=5.6, 7.2, 9.2 Hz, 1H), 7.41-7.33 (m, 2H), 7.04 (dt, J=1.6, 9.2 Hz, 1H), 6.93-6.83 (m, 2H), 5.17 (s, 2H), 4.83 (s, 2H), 4.23 (q, J=9.6 Hz, 2H), 3.81 (s, 3H), 1.66-1.60 (m, 2H), 1.55-1.49 (m, 2H).

### Preparation of 4-[[5-(2,4-difluoro-3-hydroxy-phenyl)-1,3,4-thiadiazol-2-yl]methyl]-6-(2,2,2-trifluoroethyl)-4,6-diazaspiro[2.4]heptane-5,7-dione Compound I-15

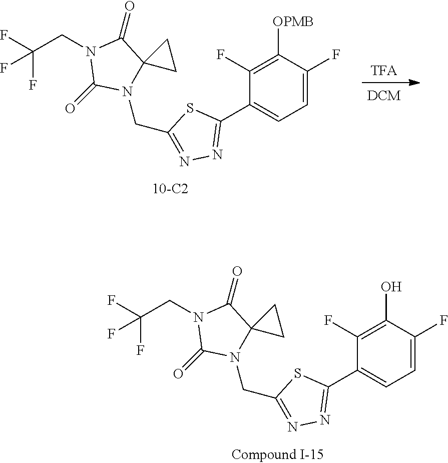

To a solution of compound 10-C2 (0.1 g, 180 μmol, 1 eq) in DCM (1 mL) was added TFA (61.6 mg, 541 μmol, 40.1 μL, 3 eq). The mixture was stirred at 30° C. for 8 hours. The reaction mixture was concentrated under reduced pressure. The residue was purified by prep-HPLC (column: Waters Xbridge BEH C18 100*30 mm*10 um; mobile phase: [H₂O (10 mM NH₄HCO₃)-ACN]; gradient: 5%-35% B over 8.0 min) to give Compound I-15 (50 mg, 113 μmol, 99% purity) as a white solid.

¹H NMR (400 MHz, DMSO-d₆) δ 7.30-7.13 (m, 1H), 7.01 (t, J=9.6 Hz, 1H), 4.93 (s, 2H), 4.34 (q, J=9.6 Hz, 2H), 1.72-1.62 (m, 2H), 1.44-1.20 (m, 2H).

LCMS: MS found: [M+H]⁺=435.1.

### Example 10

### Route:

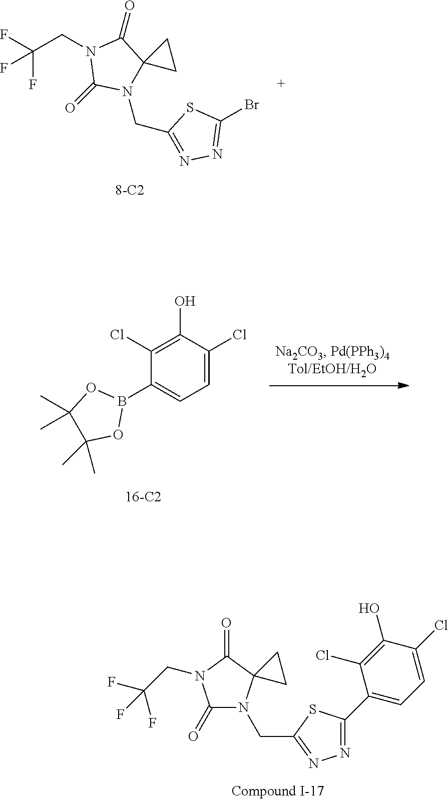 [SMILES: O=C1N(CC(F)(F)F)C(=O)C2(CC2)N1Cc1nnc(Br)s1 ; CC1(C)OB(c2ccc(Cl)c(O)c2Cl)OC1(C)C ; O=C1N(CC(F)(F)F)C(=O)C2(CC2)N1Cc1nnc(-c2ccc(Cl)c(O)c2Cl)s1]

### Preparation of 4-[[5-(2,4-dichloro-3-hydroxy-phenyl)-1,3,4-thiadiazol-2-yl]methyl]-6-(2,2,2-trifluoroethyl)-4,6-diazaspiro[2.4]heptane-5,7-dione Compound I-17

A mixture of 8-C2 (120 mg, 311.56 μmol, 1 eq), compound 16-C2 (135.04 mg, 467.34 μmol, 1.5 eq), Na₂CO₃ (66.04 mg, 623.12 μmol, 2 eq), Pd(PPh₃)₄ (36.00 mg, 31.16 μmol, 0.1 eq) in Tol. (0.5 mL), ethanol (0.5 mL), H₂O (0.5 mL) was degassed and purged with N₂ for 3 times. The mixture was stirred at 65° C. for 6 hours under N₂ atmosphere. LCMS showed the reaction was completed. Silica gel was added. The mixture was stirred at 20° C. for 0.5 hour and filtered. The filtrate was concentrated under reduced pressure. The residue was purified by prep-HPLC (column: Phenomenex luna C18 100*40 mm*3 um; mobile phase: [H₂O (0.2% formic acid)-ACN]; gradient: 35%-65% B over 8.0 min) to afford Compound I-17 (60.28 mg, 126.12 μmol, 97.763% purity) as a white solid.

¹H NMR (400 MHz, DMSO-d₆) δ 11.00-10.41 (m, 1H), 7.66-7.47 (m, 2H), 4.97 (s, 2H), 4.35 (q, J=9.6 Hz, 2H), 1.71-1.65 (m, 2H), 1.38-1.32 (m, 2H).

LCMS: MS found: [M+H]⁺=467.0.

### Example 11

### Route:

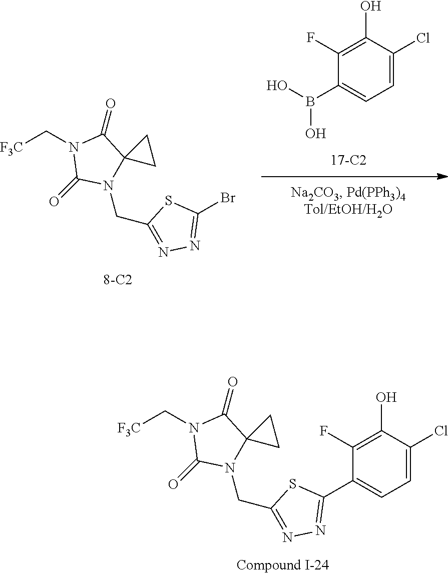 [SMILES: O=C1N(CC(F)(F)F)C(=O)C2(CC2)N1Cc1nnc(Br)s1 ; OB(O)c1ccc(Cl)c(O)c1F ; O=C1N(CC(F)(F)F)C(=O)C2(CC2)N1Cc1nnc(-c2ccc(Cl)c(O)c2F)s1]

### Preparation of 4-[[5-(4-chloro-2-fluoro-3-hydroxy-phenyl)-1,3,4-thiadiazol-2-yl]methyl]-6-(2,2,2-trifluoroethyl)-4,6-diazaspiro[2.4]heptane-5,7-dione—Compound I-24

A mixture of 8-C2 (200 mg, 519.26 μmol, 1 eq), Compound 17-C2 (98.85 mg, 519.26 μmol, 1 eq), Pd(PPh₃)₄ (60.00 mg, 51.93 μmol, 0.1 eq), Na₂CO₃ (110.07 mg, 1.04 mmol, 2 eq) in toluene (1 mL), ethanol (1 mL) and H₂O (1 mL) was degassed and purged with Argon for 3 times. The mixture was stirred at 70° C. for 2 h under Argon atmosphere. LC-MS showed the reactant was consumed completely. The reaction mixture was diluted with H₂O (20 mL), adjust pH=5-6 with citric acid and extracted with ethyl acetate (20 mL*2). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% formic acid)-ACN]; gradient: 35%-65% B over 8.0 min) to give Compound I-24 (61.16 mg, 135.67 mol, 100% purity) as a white solid.

¹H NMR (400 MHz, DMSO-d₆) δ ppm 11.03 (d, J=3.6 Hz, 1H), 7.70-7.56 (m, 1H), 7.41 (dd, J=8.8, 1.2 Hz, 1H), 4.97 (s, 2H), 4.35 (q, J=9.6 Hz, 2H), 1.72-1.57 (m, 2H), 1.40-1.29 (m, 2H).

LCMS: MS found: [M+H]⁺=451.0.

### Specific Examples of Preparation of Formula I Compounds Via Synthetic Route 3

### Scheme 5. Preparation of Compound I-27 Via Synthetic Route 3

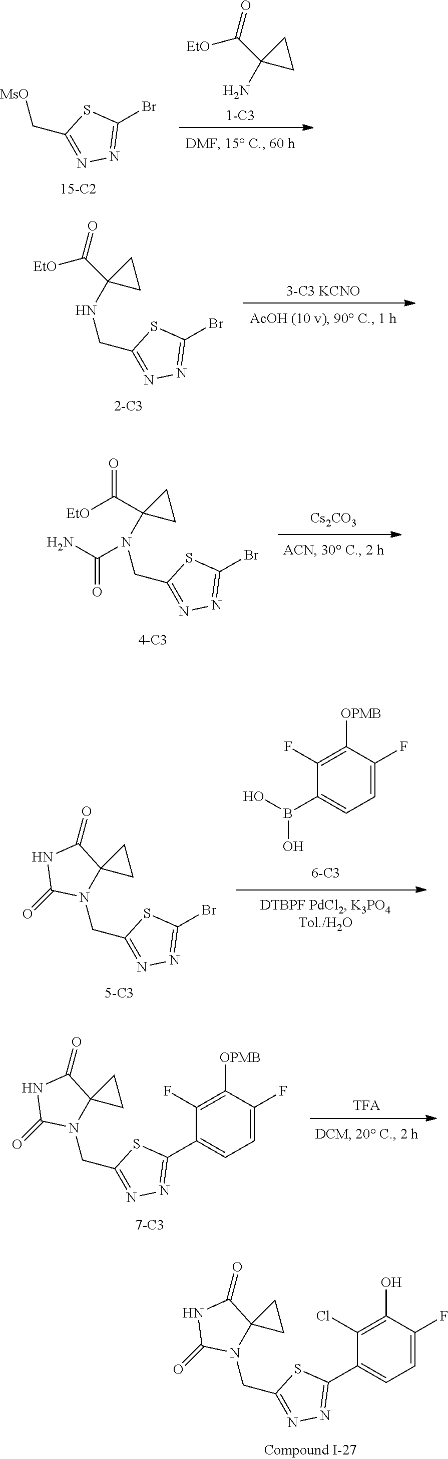

Compound 15-C2 was prepared as described above. Mesylate 15-C2 was reacted with 5 equivalents of commercially available ethyl 1-aminocyclopropane-1-carboxylate 1-C3 [42303-42-4] at 15° C. for 60 h under a nitrogen atmosphere at which time LC-MS indicated the reaction was complete. After concentration in vacuo, the reaction was diluted with DCM, washed with 0.5M HCl followed by a brine wash and drying (sodium sulfate). Concentration in vacuo afforded compound 2-C3 as a yellow solid that was used directly in the next step. Amine 2-C3 was reacted with 10 equivalents of potassium isocyanate 3-C3 in acetic acid at 90° C. for 1 h at which time TLC analysis indicated the reaction was complete. After concentration in vacuo, the residue was adjusted to pH 7 with sodium bicarbonate, extracted with DCM, and standard workup. The residue was purified by flash chromatography to afford compound 4-C3. Compound 4-C3 was cyclized to hydantoin 5-C3 by treatment with 2 equivalents of Cs₂CO₃ in acetonitrile with warming (2 h at 30° C. then 2 h at 60° C.) whereby TLC analysis indicated the reaction was complete. The reaction mixture was filtered to isolate product that was dissolved in water followed by adjustment of pH to 3 using 1 M HCl. This mixture was extracted with DCM followed by standard workup that afforded hydantoin 5-C3 after concentration in vacuo as a yellow solid. The bromide of compound 5-C3 was reacted with 1 equivalent of (2,4-difluoro-3-((4-methoxybenzyl)oxy)phenyl)boronic acid 6-C3, 1 equivalent K₃PO₄, and 0.1 equivalent of commercially available catalyst [1,1′-Bis(di-tert-butylphosphino)ferrocene]dichloropalladium(II) (CAS No. 95408-45-0) in toluene:water (4:1) at 60° C. for 2 h after degassing 3 times (in vacuo then nitrogen atmosphere). The reaction was complete as determined by LC-MS analysis. After standard workup, the residue obtained was triturated with acetonitrile to afford compound 7-C3 as a white solid. The 4-methoxybenzyl protecting group was removed from 7-C3 by treatment with TFA in DCM at 20° C. for 2 h. After concentration in vacuo, the residue was purified by reverse phase preparative HPLC to afford Compound 1-27. Compounds in Table 3 were prepared in a similar manner to Scheme 5 where changes are noted in procedures below.

TABLE 3

| | Compound | | |
|---|---|---|---|
| Example | ID | Name | Structure |
| 12 | I-27 | 4-((5-(2,4-difluoro-3- hydroxyphenyl)-1,3,4- thiadiazol-2- yl)methyl)-4,6- diazaspiro[2.4]heptane- 5,7-dione | 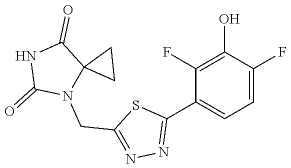 [SMILES: O=C1NC(=O)C2(CC2)N1Cc1nnc(-c2ccc(F)c(O)c2F)s1] |
| 13 | I-4 | 4-((5-(2,4-difluoro-3- hydroxyphenyl)-1,3,4- thiadiazol-2- yl)methyl)-6-propyl- 4,6- diazaspiro[2.4]heptane- 5,7-dione | 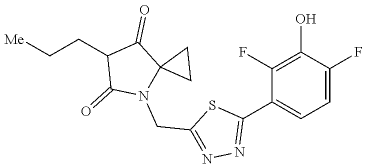 [SMILES: CCCC1C(=O)N(Cc2nnc(-c3ccc(F)c(O)c3F)s2)C2(CC2)C1=O] |
| 14 | I-5 | 4-((5-(2,4-difluoro-3- hydroxyphenyl)-1,3,4- thiadiazol-2- yl)methyl)-6- isopropyl-4,6- diazaspiro[2.4]heptane- 5,7-dione | 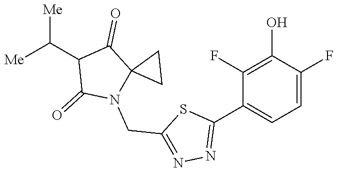 [SMILES: CC(C)C1C(=O)N(Cc2nnc(-c3ccc(F)c(O)c3F)s2)C2(CC2)C1=O] |
| 15 | I-6 | 6- (bicyclo[1.1.1]pentan- 1-ylmethyl)-4-((5-(2,4- difluoro-3- hydroxyphenyl)-1,3,4- thiadiazol-2- yl)methyl)-4,6- diazaspiro[2.4]heptane- 5,7-dione | 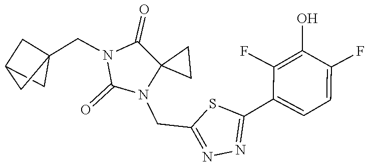 [SMILES: O=C1N(CC23CC(C2)C3)C(=O)C2(CC2)N1Cc1nnc(-c2ccc(F)c(O)c2F)s1] |
| 16 | I-21 | 4-((5-(2,4-difluoro-3- hydroxyphenyl)-1,3,4- thiadiazol-2- yl)methyl)-6- (pyrimidin-2- ylmethyl)-4,6- diazaspiro[2.4]heptane- 5,7-dione | 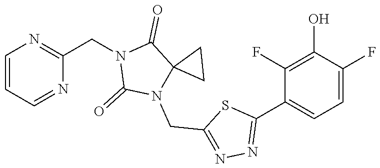 [SMILES: O=C1N(Cc2ncccn2)C(=O)C2(CC2)N1Cc1nnc(-c2ccc(F)c(O)c2F)s1] |

### Example 12

### Preparation of ethyl 1-[(5-bromo-1,3,4-thiadiazol-2-yl)methylamino]cyclopropanecarboxylate Compound 2-C3

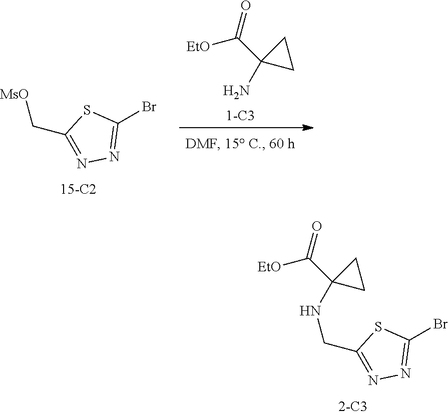

To a solution of compound 15-C2 (48.25 g, 176.66 mmol, 1 eq) in DMF (490 mL) was added compound 1-C3 (114.08 g, 883.29 mmol, 5 eq) under N₂. The mixture was stirred at 15° C. for 60 hr. LC-MS showed the reaction was completed. The mixture was concentrated on vacuum to remove most of DMF. The residue was poured into DCM (200 mL) and washed with 0.5 M HCl solution (100 mL×2). The organic phase was washed with brine (100 mL), dried over Na₂SO₄, filtered and concentrated under reduced pressure to give compound 2-C3 (53.8 g, 175.71 mmol) as yellow solid.

¹H NMR (400 MHz, MeOD) δ 4.33 (s, 2H), 4.15 (q, J=7.2 Hz, 2H), 1.31-1.23 (m, 5H), 1.07-1.00 (m, 2H).

### Preparation of ethyl 1-[(5-bromo-1,3,4-thiadiazol-2-yl)methyl-carbamoyl-amino]cyclopropanecarboxylate Compound 4-C3

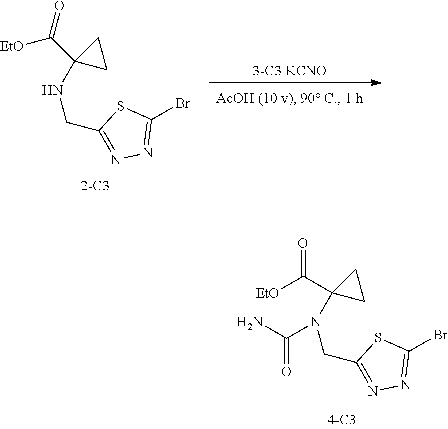 [SMILES: CCOC(=O)C1(NCc2nnc(Br)s2)CC1 ; "3-C3 KCNO" ; CCOC(=O)C1(N(Cc2nnc(Br)s2)C(N)=O)CC1]

To a mixture of compound 2-C3 (49.4 g, 161.34 mmol, 1 eq) in AcOH (495 mL) was added KCNO 3-C3 (137.38 g, 1.61 mol, 10 eq) in one portion at 25° C. The mixture was stirred at 90° C. for 1 hour. TLC (petroleum ether:ethyl acetate=0:1, Rt=0.3) showed the starting material was consumed completely. The mixture was concentrated in reduced pressure at 40° C. The residue was adjusted to pH=7 with NaHCO₃ solution. The aqueous phase was extracted with DCM (1000 mL×3). The combined organic phase was washed with brine (1000 mL), dried over Na₂SO₄, filtered and concentrated under reduced pressure to give a residue. The residue was purified by column chromatography (SiO₂, petroleum ether:ethyl acetate=70:30 to 55:45) to give compound 4-C3 (42.18 g, 120.79 mmol) as yellow solid.

¹H NMR (400 MHz, MeOD) δ 4.83 (d, J=7.2 Hz, 2H), 4.14-4.05 (m, 2H), 1.80 (dd, J=4.0, 10.0 Hz, 1H), 1.64-1.56 (m, 1H), 1.45 (d, J=4.0 Hz, 2H), 1.14 (t, J=7.2 Hz, 3H).

### Preparation of 4-[(5-bromo-1,3,4-thiadiazol-2-yl)methyl]-4,6-diazaspiro[2.4]heptane-5,7-dione Compound 5-C3

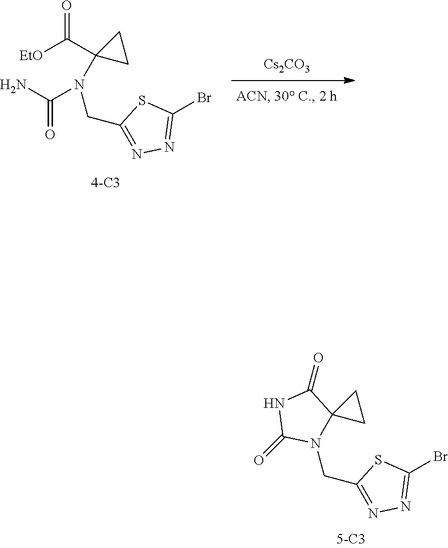 [SMILES: CCOC(=O)C1(N(Cc2nnc(Br)s2)C(N)=O)CC1 ; "Cs2CO3" ; O=C1NC(=O)C2(CC2)N1Cc1nnc(Br)s1]

To a solution of compound 4-C3 (42.18 g, 120.79 mmol, 1 eq) in ACN (430 mL) was added Cs₂CO₃ (78.71 g, 241.58 mmol, 2 eq). The mixture was stirred at 30° C. for 2 hrs. The mixture was stirred at 60° C. for 2 hrs. TLC (petroleum ether:ethyl acetate=0:1, R_{f}=0.7) showed the starting material was consumed completely. The mixture was filtered. The filter cake was poured into water (300 mL) and the residue was adjusted to pH=3 with 1 M HCl solution. The mixture was filtered. The aqueous phase was extracted with DCM (800 mL×3). The combined organic phase was washed with brine (200 mL), dried over Na₂SO₄, filtered and concentrated under reduced pressure to give compound 5-C3 (26.71 g, 87.90 mmol, 99.76% purity) as yellow solid.

¹H NMR (400 MHz, methanol-d₆) δ 4.76 (s, 2H), 1.55-1.50 (m, 2H), 1.35-1.29 (m, 2H).

### Preparation of 4-((5-(2,4-difluoro-3-((4-methoxybenzyl)oxy)phenyl)-1,3,4-thiadiazol-2-yl)methyl)-4,6-diazaspiro[2.4]heptane-5,7-dione Compound 7-C3

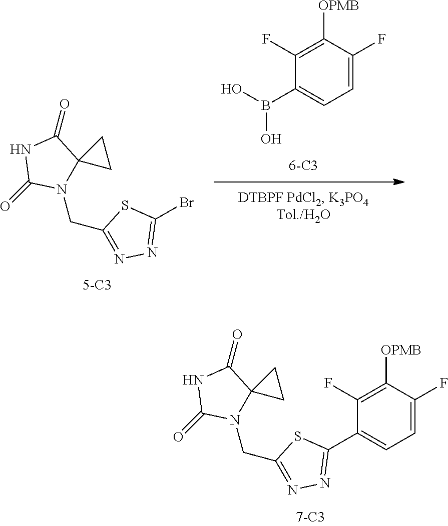

A mixture of compound 5-C3 (5 g, 16.49 mmol, 1 eq), compound 6-C3 (4.85 g, 16.49 mmol, 1 eq), catalyst [1,1′-Bis(di-tert-butylphosphino)ferrocene]dichloropalladium(II) (CAS No. 95408-45-0) (1.08 g, 1.65 mmol, 0.1 eq), K₃PO₄ (3.50 g, 16.49 mmol, 1 eq) in toluene (200 mL) and H₂O (50 mL) was degassed and purged with N₂ for 3 times, and then the mixture was stirred at 60° C. for 2 h under N₂ atmosphere. LCMS showed starting material was consumed completely and one main peak with desired mass was detected. The reaction mixture was filtered and concentrated under reduced pressure to remove toluene. The residue was diluted with H₂O 100 mL and extracted with ethyl acetate (100 mL×2). Dried over Na₂SO₄, filtered and concentrated under reduced pressure to give a residue. The crude product was triturated with ACN 40 ml at 20° C. for 0.5 h, the mixture was filtered and the filter cake was concentrated under reduced pressure to give compound 7-C3 (4 g, 8.47 mmol) as white solid.

¹H NMR (400 MHz, DMSO-d₆) δ 11.30 (br s, 1H), 7.84-8.00 (m, 1H), 7.24-7.44 (m, 3H), 6.93 (d, J=8.4 Hz, 2H), 5.16 (s, 2H), 4.84 (s, 2H), 3.74 (s, 3H), 1.47-1.55 (m, 2H), 1.16-1.24 (m, 2H).

### Preparation of 4-((5-(2,4-difluoro-3-hydroxyphenyl)-1,3,4-thiadiazol-2-yl)methyl)-4,6-diazaspiro[2.4]heptane-5,7-dione Compound I-27

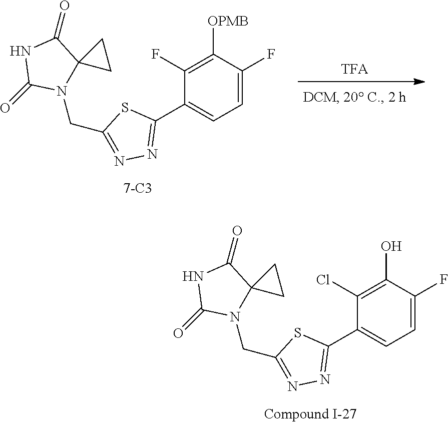

To a solution of compound 7-C3 (100 mg, 211.66 μmol, 1 eq) in DCM (1.5 mL) was added TFA (0.5 mL). The mixture was stirred at 20° C. for 2 hrs. LCMS showed compound 7-C3 was consumed completely and one main peak with desired mass was detected. The reaction mixture was diluted with H₂O 10 mL and extracted with ethyl acetate (10 mL×2). Dried over Na₂SO₄, filtered and concentrated under reduced pressure to give a residue. The residue was purified by prep-HPLC (column: Phenomenex luna C18 100*40 mm*5 um; mobile phase: [H₂O (0.2% formic acid)-ACN]; gradient: 1%-40% B over 8.0 min) to give Compound I-27 (26.06 mg, 73.52 μmol, 99.39% purity) as white solid.

¹H NMR (400 MHz, DMSO-d₆) δ ppm 11.29 (br s, 1H), 10.88 (br s, 1H), 7.53-7.72 (m, 1H), 7.17-7.31 (m, 1H), 4.84 (s, 2H), 1.46-1.55 (m, 2H), 1.15-1.23 (m, 2H).

LCMS: MS found: [M+H]⁺=353.0.

### Example 13

### Route:

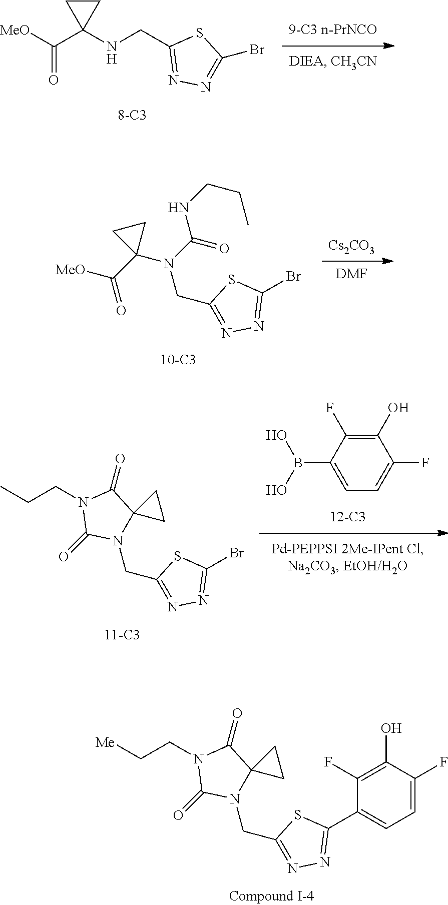

### Preparation of methyl 1-(1-((5-bromo-1,3,4-thiadiazol-2-yl)methyl)-3-propylureido)cyclopropane-1-carboxylate Compound 10-C3

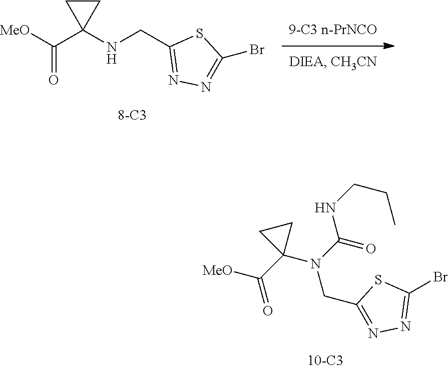 [SMILES: COC(=O)C1(NCc2nnc(Br)s2)CC1 ; "9-C3 n-PrNCO" ; CCCNC(=O)N(Cc1nnc(Br)s1)C1(C(=O)OC)CC1]

To a solution of compound 8-C3 (1 g, 3.42 mmol, 1 eq) in ACN (16 mL) was added 1-isocyanatopropane 9-C3 (1.17 g, 13.69 mmol, 1.29 mL, 4 eq) and DIEA (1.77 g, 13.69 mmol, 2.38 mL, 4 eq). The mixture was stirred at 25° C. for 12 h under N₂ atmosphere. TLC showed the reaction was completed. The reaction mixture was quenched by addition water (10 mL), and then extracted with ethyl acetate (10 mL×3). The combined organic layers were washed with brine (30 mL), dried over Na₂SO₄, filtered and concentrated under reduced pressure to give a residue. The residue was purified by column chromatography (silica gel, petroleum ether:ethyl acetate=10:1 to 0:1) to give compound 5 (872 mg, 2.17 mmol, 94% purity) as light yellow oil.

¹H NMR (400 MHz, DMSO-d₆) δ 6.59 (br t, J=5.6 Hz, 1H), 4.71 (s, 2H), 3.48 (s, 3H), 3.02 (q, J=7.2 Hz, 2H), 1.67-1.80 (m, 1H), 1.45-1.52 (m, 1H), 1.38-1.44 (m, 2H), 1.36-1.32 (m, 2H), 0.80 (t, J=7.2 Hz, 3H)

### Preparation of 4-((5-bromo-1,3,4-thiadiazol-2-yl)methyl)-6-propyl-4,6-diazaspiro[2.4]heptane-5,7-dione Compound 11-C3

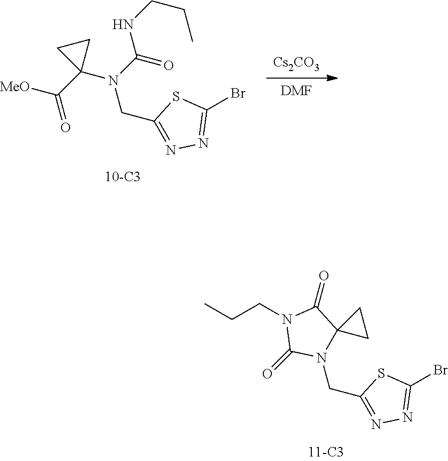

To a solution of compound 10-C3 (0.3 g, 795.21 μmol, 1 eq) in DMF (4 mL) was added Cs₂CO₃ (259.10 mg, 795.21 μmol, 1 eq) at 20° C., then the reaction mixture was stirred at 25° C. for 0.5 h. LC-MS showed the reaction was completed. The reaction mixture was poured into water (20 mL), and then extracted with ethyl acetate 30 mL (10 mL×3). The combined organic layers were washed with brine (15 mL), dried over anhydrous Na₂SO₄, filtered and concentrated under reduced pressure to give a residue. The residue was purified by prep-TLC (SiO₂, petroleum ether:ethyl acetate=2:1, R_{f}=0.3) to give compound 11-C3 (150 mg, 434.51 μmol) as white solid.

¹H NMR (400 MHz, DMSO-d₆) δ 4.83 (s, 2H), 3.42 (t, J=7.2 Hz, 2H), 1.63-1.47 (m, 4H), 1.28-1.21 (m, 2H), 0.84 (t, J=7.2 Hz, 3H).

LCMS: RT=0.399 min, MS cal.: 344.0, MS found: [M+H]⁺=345.0.

### Preparation of 4-((5-(2,4-difluoro-3-hydroxyphenyl)-1,3,4-thiadiazol-2-yl)methyl)-6-propyl-4,6-diazaspiro[2.4]heptane-5,7-dione Compound 1-4

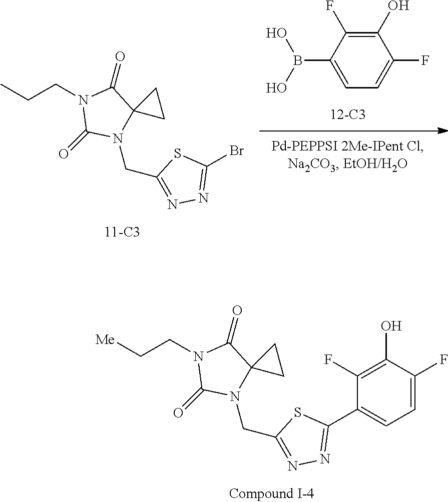 [SMILES: CCCN1C(=O)N(Cc2nnc(Br)s2)C2(CC2)C1=O ; OB(O)c1ccc(F)c(O)c1F ; CCCN1C(=O)N(Cc2nnc(-c3ccc(F)c(O)c3F)s2)C2(CC2)C1=O]

To a solution of compound 11-C3 (0.2 g, 579.35 μmol, 1 eq), compound 12-C3 (120.91 mg, 695.22 mol, 1.2 eq) in ethanol (4 mL) and H₂O (0.8 mL) was added Na₂CO₃ (153.51 mg, 1.45 mmol, 2.5 eq) and 1,3-bis[2,6-bis(1-ethylpropyl)phenyl]-2H-imidazole; 3-chloropyridine; dichloropalladium (45.98 mg, 57.93 μmol, 0.1 eq). The reaction mixture was degassed and purged with N₂ for 3 times, and then the mixture was stirred at 80° C. for 1.5 h under sealed tube. LC-MS showed the reaction was completed. After filtration, pH of the filtrate was adjusted to 5 by aqueous citric acid solution. Then the filtrate was extracted with ethyl acetate (10 mL×2). The organic layer was dried, filtered and concentrated to give a residue. The residue was purified by prep-HPLC (column: Phenomenex Luna C18 100*30 mm*3 um; mobile phase: [H₂O (0.2% formic acid)-ACN]; gradient: 25%-55% B over 8.0 min) to give Compound I-4 (50 mg, 126.78 μmol) was obtained as a white solid.

¹H NMR (400 MHz, DMSO-d₆) δ7.60-7.48 (m, 1H), 7.25-7.13 (m, 1H), 4.83 (s, 2H), 3.42 (t, J=7.2 Hz, 2H), 1.59 (m, 4H), 1.46-1.32 (s, 1H), 1.30-1.19 (m, 2H), 0.81 (t, J=7.2 Hz, 3H) LCMS: RT=2.270 min, MS cal.: 394.1, MS found: [M+H]⁺=394.9.

HPLC: RT=2.412 min, purity: 98.96%.

### Example 14

### Route:

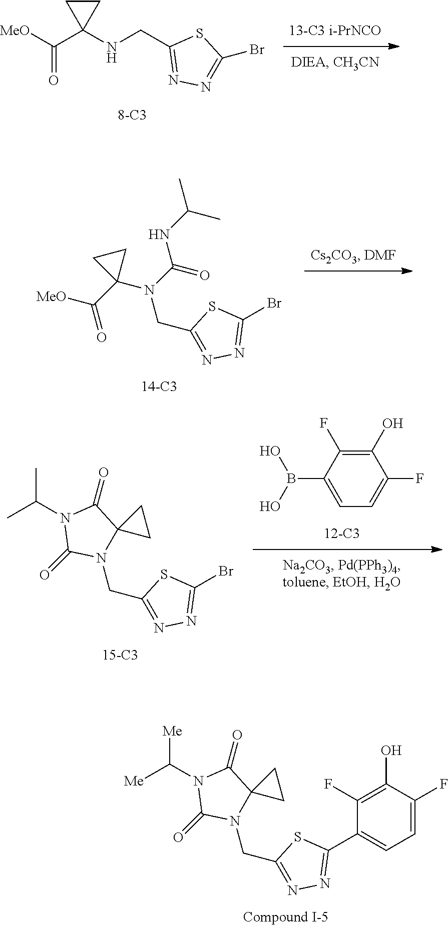 [SMILES: COC(=O)C1(NCc2nnc(Br)s2)CC1 ; COC(=O)C1(N(Cc2nnc(Br)s2)C(=O)NC(C)C)CC1 ; CC(C)N1C(=O)N(Cc2nnc(Br)s2)C2(CC2)C1=O ; OB(O)c1ccc(F)c(O)c1F ; CC(C)N1C(=O)N(Cc2nnc(-c3ccc(F)c(O)c3F)s2)C2(CC2)C1=O]

### Preparation of methyl 1-[(5-bromo-1,3,4-thiadiazol-2-yl)methyl-(isopropylcarbamoyl)amino]cyclopropanecarboxylate Compound 14-C3

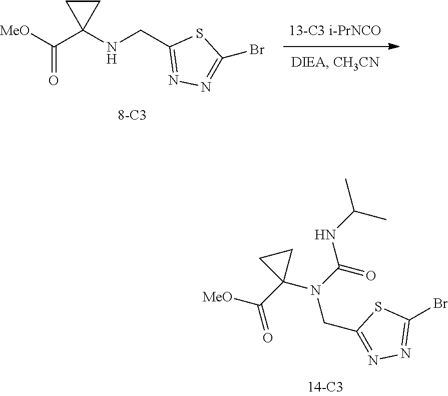 [SMILES: COC(=O)C1(NCc2nnc(Br)s2)CC1 ; COC(=O)C1(N(Cc2nnc(Br)s2)C(=O)NC(C)C)CC1]

A mixture of compound 8-C3 (550 mg, 1.88 mmol, 1 eq), 2-isocyanatopropane 13-C3 (1.28 g, 15.06 mmol, 1.48 mL, 8 eq), DIEA (1.95 g, 15.06 mmol, 2.62 mL, 8 eq) in ACN (5 mL) was degassed and purged with N₂ for 3 times. The mixture was stirred at 70° C. for 12 h under N₂ atmosphere. TLC indicated reactant was consumed completely. The reaction mixture was concentrated under reduced pressure. The residue was diluted with H₂O (20 mL), extracted with Ethyl acetate (20 mL×2). The combined organic layers were washed with citric acid (10 mL×2), dried over Na₂SO₄, filtered and concentrated under reduced pressure. The residue was purified by column chromatography (SiO₂, petroleum ether:ethyl acetate=1:0 to 1:1) to give compound 14-C3 (420 mg, 1.11 mmol) as yellow oil.

¹H NMR (400 MHz, chloroform-d) δ 5.01-4.89 (m, 1H), 4.77-4.66 (m, 2H), 4.07-3.92 (m, 1H), 3.64 (s, 3H), 1.82-1.74 (m, 1H), 1.67-1.61 (m, 1H), 1.56-1.49 (m, 1H), 1.33-1.27 (m, 1H), 1.18-1.15 (m, 6H).

LCMS: RT=0.423 min, MS cal.: 376.02, MS found: [M+H]⁺=379.1.

### Preparation of 4-[(5-bromo-1,3,4-thiadiazol-2-yl)methyl]-6-isopropyl-4,6-diazaspiro[2.4]heptane-5,7-dione Compound 15-C3

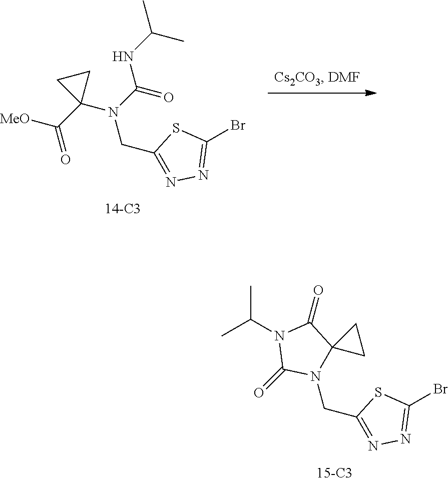 [SMILES: COC(=O)C1(N(Cc2nnc(Br)s2)C(=O)NC(C)C)CC1 ; CC(C)N1C(=O)N(Cc2nnc(Br)s2)C2(CC2)C1=O]

To a solution of compound 14-C3 (420 mg, 1.11 mmol, 1 eq) in DMF (8 mL) was added Cs₂CO₃ (362.73 mg, 1.11 mmol, 1 eq). The mixture was stirred at 25° C. for 2 h. TLC indicated reactant was consumed completely. The reaction mixture was diluted with H₂O (20 mL), extracted with ethyl acetate (20 mL×2). The combined organic layers were dried over Na₂SO₄, filtered and concentrated under reduced pressure. The residue was purified by column chromatography (SiO₂, petroleum ether:ethyl acetate=1:0 to 1:1) to give compound 15-C3 (250 mg, 724.19 μmol) as white solid.

¹H NMR (400 MHz, DMSO-d₆) δ 4.80 (s, 2H), 4.35-4.15 (m, 1H), 1.55-1.47 (m, 2H), 1.34 (d, J=6.8 Hz, 6H), 1.24-1.19 (m, 2H).

LCMS: RT=0.658 min, MS cal.: 343.99, MS found: [M+H]⁺=344.9.

### Preparation of 4-[[5-(2,4-difluoro-3-hydroxy-phenyl)-1,3,4-thiadiazol-2-yl]methyl]-6-isopropyl-4,6-diazaspiro[2.4]heptane-5,7-dione Compound I-5

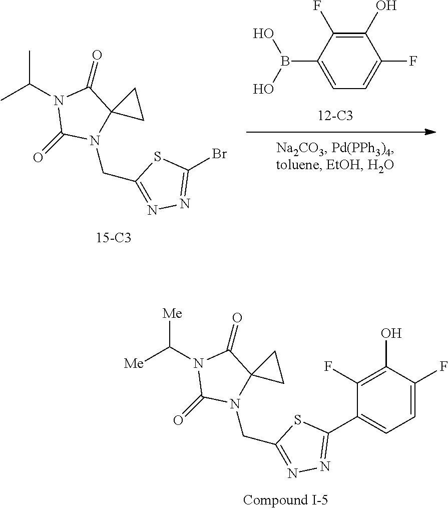 [SMILES: CC(C)N1C(=O)N(Cc2nnc(Br)s2)C2(CC2)C1=O ; OB(O)c1ccc(F)c(O)c1F ; CC(C)N1C(=O)N(Cc2nnc(-c3ccc(F)c(O)c3F)s2)C2(CC2)C1=O]

A mixture of compound 15-C3 (200 mg, 579.35 μmol, 1 eq), (2,4-difluoro-3-hydroxy-phenyl)boronic acid 12-C3 (100.75 mg, 579.35 μmol, 1 eq), Pd(PPh₃)₄ (66.95 mg, 57.93 μmol, 0.1 eq), Na₂CO₃ (122.81 mg, 1.16 mmol, 2 eq) in toluene (0.8 mL), ethanol (0.8 mL) and H₂O (0.8 mL) was degassed and purged with argon for 3 times. The mixture was stirred at 80° C. for 2 hrs under argon atmosphere. LCMS showed reactant was consumed completely. The reaction mixture was diluted with H₂O (10 mL), adjusted pH=5 with citric acid and extracted with ethyl acetate (10 mL*2). The combined organic layers were dried over Na₂SO₄, filtered and concentrated under reduced pressure. The residue was purified by prep-HPLC (column: Phenomenex Luna C18 100*40 mm*5 um; mobile phase: [H₂O (0.2% formic acid)-ACN]; gradient: 30%-60% B over 8.0 min) to give Compound I-5 (90.24 mg, 225.70 μmol, 98.64% purity) as a white solid.

¹H NMR (400 MHz, DMSO-d₆) δ10.84 (br s, 1H), 7.76-7.51 (m, 1H), 7.25 (t, J=7.2 Hz, 1H), 4.88 (s, 2H), 4.31-4.24 (m, 1H), 1.59-1.50 (m, 2H), 1.35 (d, J=7.2 Hz, 6H), 1.26-1.20 (m, 2H).

LCMS: RT=2.302 min, MS cal.: 394.09, MS found: [M+H]⁺=395.1.

HPLC: RT=2.949 min, purity: 98.641%.

### Example 15

### Route 1:

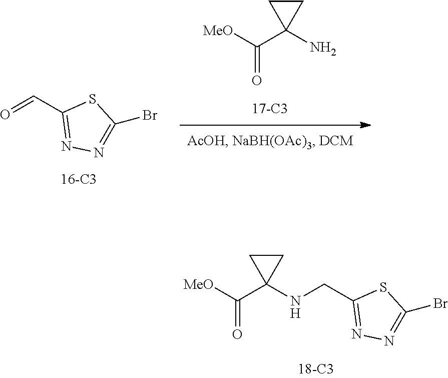 [SMILES: COC(=O)C1(N)CC1 ; O=Cc1nnc(Br)s1 ; COC(=O)C1(NCc2nnc(Br)s2)CC1]

### Route 2:

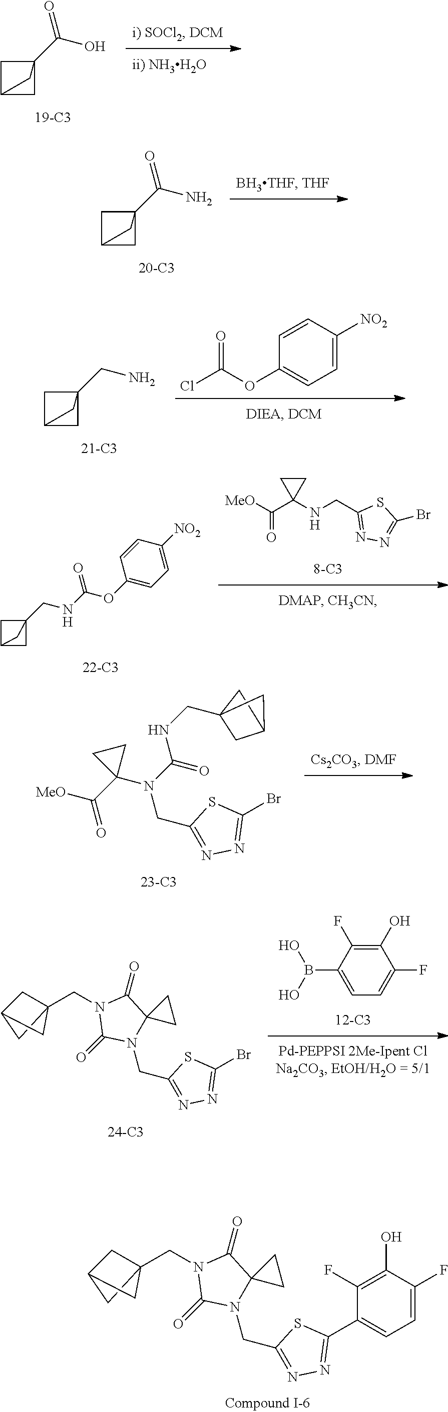

### Preparation of methyl 1-(((5-bromo-1,3,4-thiadiazol-2-yl)methyl)amino)cyclopropane-1-carboxylate Compound 8-C3

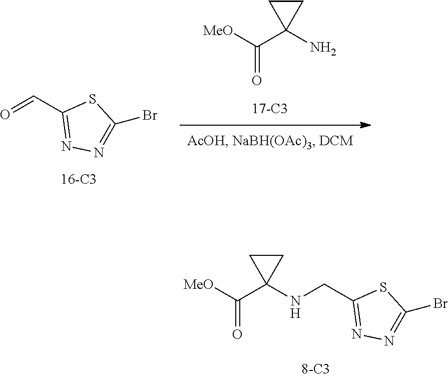 [SMILES: COC(=O)C1(N)CC1 ; O=Cc1nnc(Br)s1 ; COC(=O)C1(NCc2nnc(Br)s2)CC1]

To a solution of compound 16-C3 (3.18 g, 16.47 mmol, 1 eq) in DCM (70 mL) was added methyl 1-aminocyclopropanecarboxylate 17-C3 (1.90 g, 16.47 mmol, 1 eq) and acetic acid (1.48 g, 24.71 mmol, 1.41 mL, 1.5 eq). The reaction mixture was stirred at 0° C. for 1 h, then NaBH(OAc)₃ (6.98 g, 32.95 mmol, 2 eq) was added at 0° C. under N₂. The reaction mixture was stirred at 20° C. for 12 h. TLC showed the reaction was completed. The reaction mixture was quenched by saturated NaHCO₃ (10 mL), diluted with H₂O (10 mL) and extracted with DCM (10 mL×3). The combined organic layers were washed with brine (10 mL), dried over Na₂SO₄, filtered and concentrated under reduced pressure to give a residue. The residue was purified by column chromatography (SiO₂, petroleum ether:ethyl acetate=10:1 to 1:1) to give compound 8-C3 (3.28 g, 11.11 mmol) as colorless oil.

¹H NMR (400 MHz, DMSO-d₆) δ 4.23 (d, J=7.2 Hz, 2H), 4.08-4.14 (m, 1H), 3.62 (s, 3H), 1.15-1.20 (m, 2H), 0.95-1.00 (m, 2H)

### Preparation of bicyclo[1.1.1]pentane-1-carboxamide 20-C3

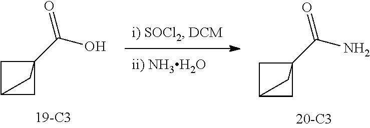 [SMILES: O=C(O)C12CC(C1)C2 ; NC(=O)C12CC(C1)C2]

To a solution of compound 19-C3 (2 g, 17.84 mmol, 1 eq) in DCM (20 mL) was added SOCl₂ (3.82 g, 32.11 mmol, 2.33 mL, 1.8 eq) under N₂ and stirred at 45° C. for 12 hrs. NH₃·H₂O (25.00 g, 178.37 mmol, 27.48 mL, 25% purity, 10 eq) was added to the mixture at 0° C., and then the mixture was stirred at 0° C. for 1 hr. TLC showed the reactant was consumed. The mixture was filtered to collect the solid. The filter cake was dried under reduced pressure. The solid was triturated with THF (10 mL) for 1 h at 25° C. After filtration, the filtrate was concentrated under reduced pressure to give compound 20-C3 (1.36 g, 12.24 mmol) as white solid.

¹H NMR (400 MHz, DMSO-d₆) δ 7.17 (s, 1H), 6.85 (s, 1H), 2.37 (s, 1H), 1.91 (s, 6H)

### Preparation of bicyclo[1.1.1]pentan-1-ylmethanamine Compound 21-C3

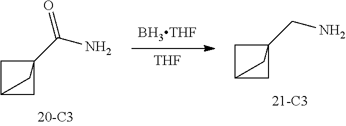 [SMILES: NC(=O)C12CC(C1)C2 ; NCC12CC(C1)C2 ; "BH3·THF" ; "THF"]

To a solution of compound 20-C3 (1.36 g, 12.24 mmol, 1 eq) in THF (15 mL) was added BH₃·THF (1 M, 36.71 mL, 3 eq) dropwise at 0° C. under N₂. After addition, the mixture was stirred at 25° C. for 12 h. TLC showed the reactant was consumed. After completion, the mixture was quenched slowly by methanol (10 mL) and stirred at 0° C. for 30 min. Aqueous HCl (1 M, 3 eq) was added and stirred for 30 min. The mixture was concentrated under reduced pressure to give compound 21-C3 (1.3 g, 9.73 mmol, HCl salt) as white solid.

¹H NMR (400 MHz, DMSO-d₆) δ 8.04 (br s, 3H), 2.82 (q, J=6.0 Hz, 2H), 2.48 (s, 1H), 1.77 (s, 6H)

### Preparation of 4-nitrophenyl (bicyclo[1.1.1]pentan-1-ylmethyl)carbamate—Compound 22-C3

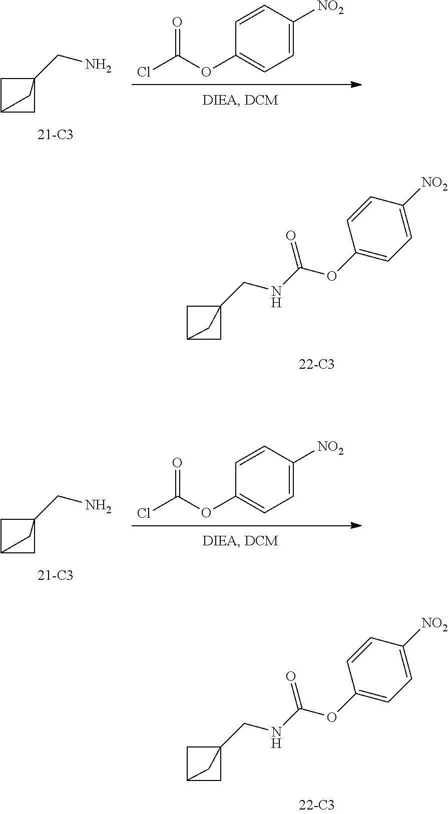 [SMILES: NCC12CC(C1)C2 ; O=C(Cl)Oc1ccc([N+](=O)[O-])cc1 ; O=C(NCC12CC(C1)C2)Oc1ccc([N+](=O)[O-])cc1 ; NCC12CC(C1)C2 ; O=C(Cl)Oc1ccc([N+](=O)[O-])cc1 ; O=C(NCC12CC(C1)C2)Oc1ccc([N+](=O)[O-])cc1]

To a solution of compound 21-C3 (2.6 g, 16.06 mmol, 1 eq) and compound 4-nitrophenylchloroformate (8.09 g, 40.14 mmol, 2.5 eq) in DCM (30 mL) was added DIEA (10.38 g, 80.28 mmol, 13.98 mL, 5 eq). The mixture was stirred at 25° C. for 1 h. TLC indicated reactant was consumed completely. The reaction mixture was quenched by addition water (10 mL) at 20° C., and extracted with ethyl acetate (10 mL×3). The combined organic layers were washed with brine (30 mL), dried over Na₂SO₄, filtered and concentrated under reduced pressure to give a residue. The residue was purified by column chromatography (SiO₂, petroleum ether:ethyl acetate=10:1 to 5:1) to compound 22-C3 (0.53 g, 2.02 mmol) as a white solid.

¹H NMR (400 MHz, DMSO-d₆) δ ppm 8.22-8.30 (m, 2H), 8.05-8.13 (br m, 1H), 7.34-7.45 (m, 2H), 3.11 (d, J=6.0 Hz, 2H), 2.49 (s, 1H), 1.71 (s, 6H)

### Preparation of methyl 1-(3-(bicyclo[1.1.1]pentan-1-ylmethyl)-1-((5-bromo-1,3,4-thiadiazol-2-yl)methyl)ureido)cyclopropane-1-carboxylate Compound 23-C3

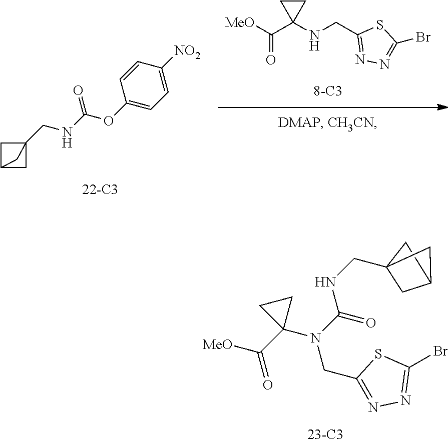 [SMILES: O=C(NCC12CC(C1)C2)Oc1ccc([N+](=O)[O-])cc1 ; COC(=O)C1(NCc2nnc(Br)s2)CC1 ; COC(=O)C1(N(Cc2nnc(Br)s2)C(=O)NCC23CC(C2)C3)CC1]

To a solution of compound 22-C3 (0.43 g, 1.64 mmol, 1 eq), compound 8-C3 (239.51 mg, 819.79 mol, 0.5 eq) in CH₃CN (8 mL) was added 4-Dimethylaminopyridine (DMAP, 200.30 mg, 1.64 mmol, 1 eq). The mixture was stirred at 60° C. for 12 hrs. LC-MS showed reactant was consumed completely. The reaction mixture was quenched by water (20 mL) and extracted with ethyl acetate (10 mL×3). The combined organic layers were washed with brine (15 mL), dried over, filtered and concentrated under reduced pressure to give a residue. The residue was purified by column chromatography (SiO₂, petroleum ether:ethyl acetate=100:1 to 1:1) to give compound 23-C3 (370 mg, 890.91 μmol) as white solid.

¹H NMR (400 MHz, DMSO-d₆) δ ppm 6.62 (t, J=6.0 Hz, 1H), 4.72 (s, 2H), 3.49 (s, 3H), 3.09 (d, J=6.0 Hz, 2H), 2.42 (s, 1H), 1.68-1.82 (m, 1H), 1.57-1.63 (m, 6H), 1.46 (s, 1H), 1.34 (d, J=6.8 Hz, 2H)

LCMS: RT=0.515 min, MS calc.: 414.0, MS found: [M+H]⁺=415.1, 417.1.

### Preparation of 6-(bicyclo[1.1.1]pentan-1-ylmethyl)-4-((5-bromo-1,3,4-thiadiazol-2-yl)methyl)-4,6-diazaspiro[2.4]heptane-5,7-dione Compound 24-C3

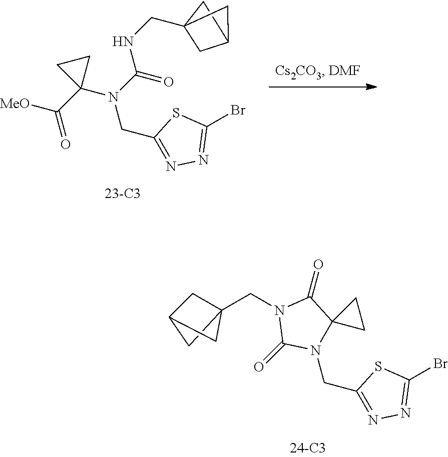 [SMILES: COC(=O)C1(N(Cc2nnc(Br)s2)C(=O)NCC23CC(C2)C3)CC1 ; O=C1N(CC23CC(C2)C3)C(=O)C2(CC2)N1Cc1nnc(Br)s1]

To a solution of compound 23-C3 (300 mg, 722.36 μmol, 1 eq) in DMF (3 mL) was added Cs₂CO₃ (470.72 mg, 1.44 mmol, 2 eq). The mixture was stirred at 25° C. for 1 h. LCMS showed reactant consumed. The mixture was poured into water (10 mL), and then extracted with ethyl acetate (10 mL×3). The combined organic layers were washed with brine (15 mL), dried over, filtered and concentrated under reduced pressure to give a residue. The residue was purified by column chromatography (SiO₂, petroleum ether:ethyl acetate=50:1 to 10:1) to compound 24-C3 (200 mg, 521.84 μmol) as white solid.

¹H NMR (400 MHz, DMSO-d₆) δ 4.84 (s, 2H), 3.51 (s, 2H), 2.43 (s, 1H), 1.65 (s, 6H), 1.49-1.56 (m, 2H), 1.21-1.28 (m, 2H)

LCMS: RT=0.478 min, MS cal.: 382.0, MS found: [M+H]⁺=383.0, 385.0.

### Preparation of 6-(bicyclo[1.1.1]pentan-1-ylmethyl)-4-((5-(2,4-difluoro-3-hydroxyphenyl)-1,3,4-thiadiazol-2-yl)methyl)-4,6-diazaspiro[2.4]heptane-5,7-dione Compound I-6

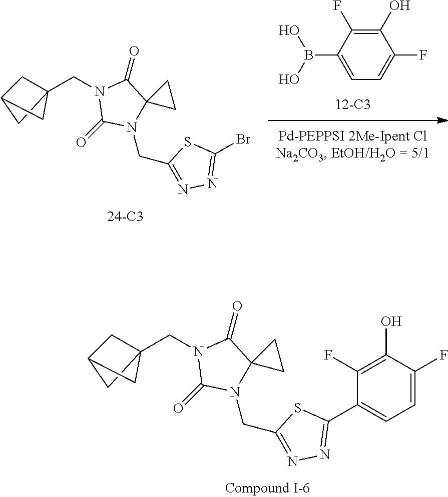 [SMILES: O=C1N(CC23CC(C2)C3)C(=O)C2(CC2)N1Cc1nnc(Br)s1 ; OB(O)c1ccc(F)c(O)c1F ; O=C1N(CC23CC(C2)C3)C(=O)C2(CC2)N1Cc1nnc(-c2ccc(F)c(O)c2F)s1]

To a solution of compound 24-C3 (200 mg, 521.84 μmol, 1 eq) in ethanol (5 mL) and H₂O (1 mL) was added compound 12-C3 (136.13 mg, 782.75 μmol, 1.5 eq) and Na₂CO₃ (110.62 mg, 1.04 mmol, 2 eq). After purged with N₂ for three times, [2-(2-aminophenyl)phenyl]-methylsulfonyloxy-palladium; ditert-butyl-[2-(2,4,6-triisopropylphenyl)phenyl]phosphane (41.45 mg, 52.18 μmol, 0.1 eq) was added. The mixture was stirred at 80° C. for 2 h under N₂ atmosphere. LCMS showed reactant consumed and desired MS detected. The mixture was concentrated under reduced pressure to give a residue. The residue was dissolved into water (5 mL), and then extracted with ethyl acetate (5 mL×2). The organic layer was dried, filtered and concentrated. The residue was purified by prep-HPLC (column: Phenomenex Luna C18 100*30 mm*3 um; mobile phase: [H₂O (0.2% formic acid)-ACN]; gradient: 35%-65% B over 8.0 min) to give Compound I-6 (52 mg, 120.25 μmol) as white solid.

¹H NMR (400 MHz, DMSO-d₆) δ (ppm) 10.85 (br s, 1H), 7.64 (q, J=7.2 Hz, 1H), 7.25 (t, J=10.4 Hz, 1H), 4.92 (s, 2H), 3.53 (s, 2H), 2.44 (s, 1H), 1.66 (s, 6H), 1.53-1.61 (m, 2H), 1.22-1.30 (m, 2H)

LCMS: RT=2.465 min, MS cal.: 432.1, MS found: [M+H]⁺=433.1.

HPLC: RT=2.763 min, purity: 100%.

### Example 16

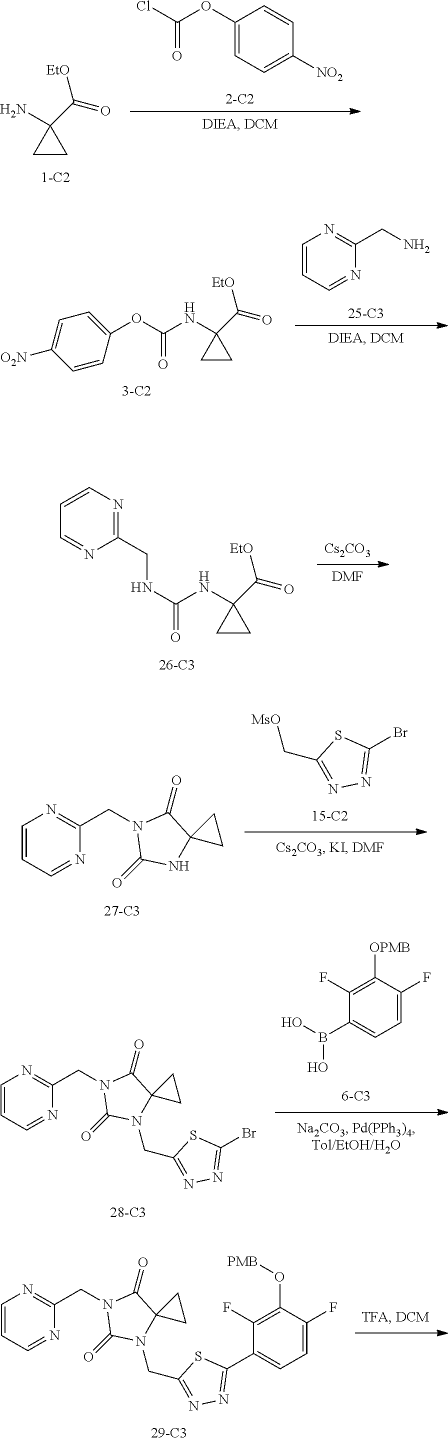

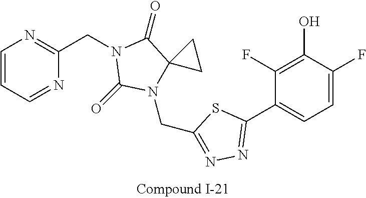 [SMILES: O=C1N(Cc2ncccn2)C(=O)C2(CC2)N1Cc1nnc(-c2ccc(F)c(O)c2F)s1]

### Preparation of ethyl 1-[(4-nitrophenoxy)carbonylamino]cyclopropanecarboxylate Compound 3-C2

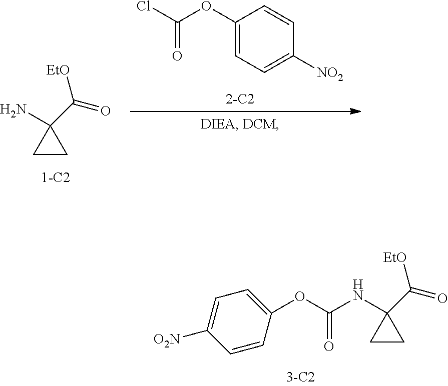

To a solution of compound 1-C2 (1 g, 7.74 mmol, 1 eq) in DCM (15 mL) was added DIEA (1.50 g, 11.61 mmol, 2.02 mL, 1.5 eq) and compound 2-C2 (2.34 g, 11.61 mmol, 1.5 eq) under N₂. Then the mixture was stirred at 15° C. for 0.5 h. LC-MS showed the reaction was completed. Compound 3-C2 (2.2 g, 7.48 mmol) was obtained as a yellow oil, which was used to next step directly.

### Preparation of ethyl 1-(pyrimidin-2-ylmethylcarbamoylamino)cyclopropanecarboxylate Compound 26-C3

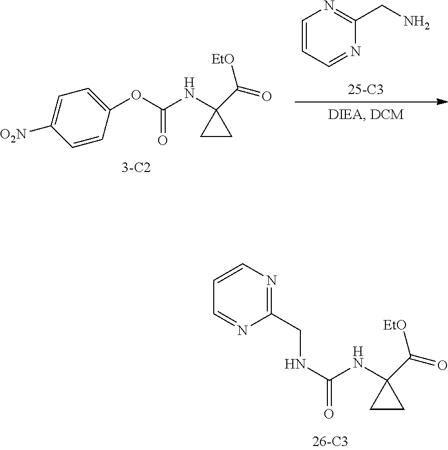

To a solution of compound 3-C2 (1.6 g, 5.44 mmol, 1 eq) in DCM (16 mL) was added DIEA (702.74 mg, 5.44 mmol, 947.09 μL, 1 eq) and compound 25-C3 (890.06 mg, 8.16 mmol, 1.5 eq) under N₂. Then the mixture was stirred at 15° C. for 1 h. LC-MS showed the reaction was completed. The reaction mixture was quenched by addition H₂O (100 mL) at 20° C., and extracted with DCM (50 mL*2). The combined organic layers were washed with brine (50 mL*2), dried over Na₂SO₄, filtered and concentrated under reduced pressure to give a residue. The residue was purified by column chromatography (SiO₂, ethyl acetate:methanol=95:5 to 90:10) to give compound 26-C3 (1.17 g, 4.37 mmol, 98.69% purity) was obtained as a yellow solid.

¹H NMR (400 MHz, CDCl₃) δ 8.70 (d, J=4.8 Hz, 2H), 7.20 (t, J=4.8 Hz, 1H), 6.14 (br s, 1H), 5.51 (br s, 1H), 4.70 (s, 2H), 4.15 (q, J=7.2 Hz, 2H), 1.66-1.53 (m, 2H), 1.29-1.24 (m, 2H), 1.21 (t, J=7.2 Hz, 3H).

### Preparation of 6-(pyrimidin-2-ylmethyl)-4,6-diazaspiro[2.4]heptane-5,7-dione Compound 27-C3

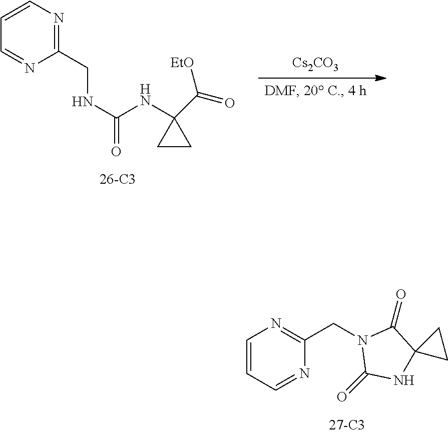 [SMILES: CCOC(=O)C1(NC(=O)NCc2ncccn2)CC1 ; "Cs2CO3" ; "DMF, 20° C., 4 h" ; O=C1NC2(CC2)C(=O)N1Cc1ncccn1]

A mixture of compound 26-C3 (810 mg, 3.06 mmol, 1 eq), Cs₂CO₃ (2.00 g, 6.13 mmol, 2 eq) in DMF (8 mL) was degassed and purged with N₂ for 3 times, and then the mixture was stirred at 20° C. for 4 hr under N₂ atmosphere. TLC (ethyl acetate:methanol=10:1, Rt=0.43) showed the starting material was consumed completely, which was used in the next step directly.

### Preparation of 4-[(5-bromo-1,3,4-thiadiazol-2-yl)methyl]-6-(pyrimidin-2-ylmethyl)-4,6-diazaspiro[2.4]heptane-5,7-dione Compound 28-C3

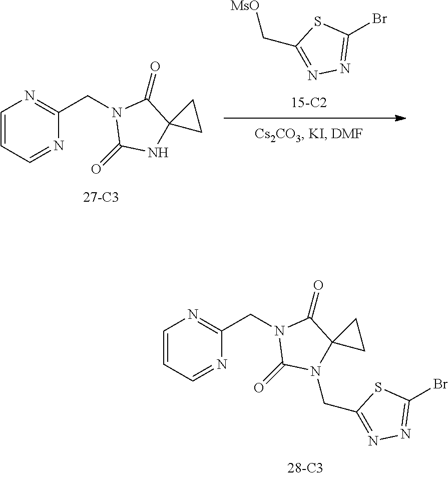 [SMILES: O=C1NC2(CC2)C(=O)N1Cc1ncccn1 ; CS(=O)(=O)OCc1nnc(Br)s1 ; O=C1N(Cc2ncccn2)C(=O)C2(CC2)N1Cc1nnc(Br)s1]

A mixture of compound 27-C3 (668.98 mg, 3.07 mmol, 1.2 eq), compound 15-C2 (697.78 mg, 2.55 mmol, 1 eq), Cs₂CO₃ (832.40 mg, 2.55 mmol, 1 eq), KI (424.10 mg, 2.55 mmol, 1 eq) in DMF (7 mL) was degassed and purged with N₂ for 3 times, and then the mixture was stirred at 20° C. for 3 hrs under N₂ atmosphere. TLC showed the starting material was consumed completely. The reaction mixture was quenched by addition H₂O (40 mL) at 20° C., and extracted with ethyl acetate (40 mL×2). The combined organic layers were washed with brine (40 mL×2), dried over Na₂SO₄, filtered and concentrated under reduced pressure to give a residue. The residue was purified by column chromatography (SiO₂, ethyl acetate:methanol=99:1) to give compound 28-C3 (460 mg, 1.16 mmol) as a yellow solid.

¹H NMR (400 MHz, CDCl₃) δ 8.68 (d, J=4.8 Hz, 2H), 7.22 (t, J=4.8 Hz, 1H), 5.05 (s, 2H), 4.76 (s, 2H), 1.56-1.53 (m, 2H), 1.52 (t, J=2.2 Hz, 2H).

### Preparation of 4-[[5-[2,4-difluoro-3-[(4-methoxyphenyl)methoxy]phenyl]-1,3,4-thiadiazol-2-yl]methyl]-6-(pyrimidin-2-ylmethyl)-4,6-diazaspiro[2.4]heptane-5,7-dione Compound 29-C3

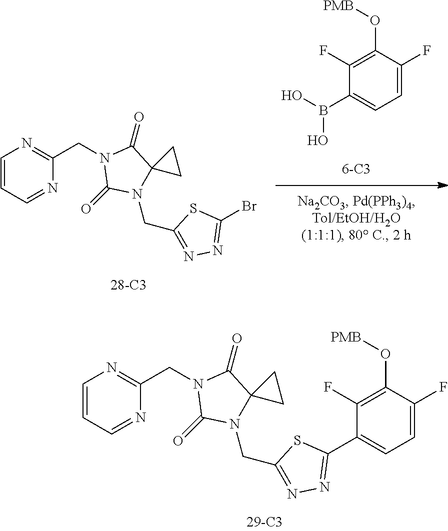

A mixture of compound 28-C3 (190 mg, 480.73 μmol, 1 eq), compound 6-C3 (183.77 mg, 624.95 mol, 1.3 eq), Na₂CO₃ (101.90 mg, 961.46 μmol, 2 eq), Pd(PPh₃)₄ (55.55 mg, 48.07 μmol, 0.1 eq) in toluene (0.7 mL), ethanol (0.7 mL), H₂O (0.7 mL) was degassed and purged with N₂ for 3 times, and then the mixture was stirred at 80° C. for 2 h under N₂ atmosphere. TLC showed the starting material was consumed completely. The reaction mixture was quenched by addition H₂O (20 mL) at 20° C., and extracted with ethyl acetate (10 mL*2). The combined organic layers were washed with brine (10 mL*2), dried over Na₂SO₄, filtered and concentrated under reduced pressure to give a residue. The residue was purified by prep-TLC (SiO₂, petroleum ether:ethyl acetate=0:1) to give compound 29-C3 (120 mg, 212.55 μmol) as a yellow oil.

¹H NMR (400 MHz, CDCl₃) δ 8.69 (d, J=4.8 Hz, 2H), 8.04 (ddd, J=5.6, 7.6, 9.2 Hz, 1H), 7.37 (d, J=8.8 Hz, 2H), 7.21 (t, J=4.8 Hz, 1H), 7.05 (dt, J=2.0, 9.2 Hz, 1H), 6.91-6.88 (m, 2H), 5.17 (s, 2H), 5.08 (s, 2H), 4.88 (s, 2H), 3.81 (s, 3H), 1.57 (d, J=5.2 Hz, 2H), 1.53-1.48 (m, 2H).

### Preparation of 4-[[5-(2,4-difluoro-3-hydroxy-phenyl)-1,3,4-thiadiazol-2-yl]methyl]-6-(pyrimidin-2-ylmethyl)-4,6-diazaspiro[2.4]heptane-5,7-dione Compound I-21

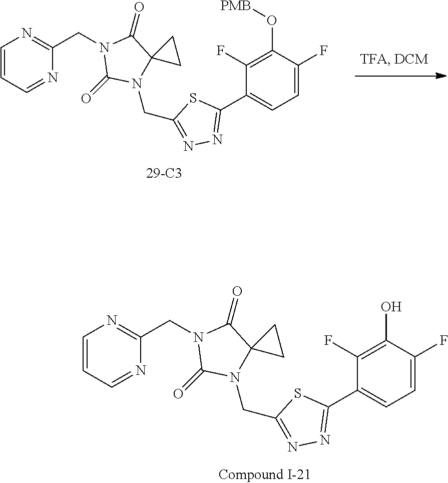

To a solution of compound 29-C3 (120 mg, 212.55 μmol, 1 eq) in DCM (3 mL) was added TFA (96.94 mg, 850.22 μmol, 63.16 μL, 4 eq). The mixture was stirred at 20° C. for 16 hrs. LC-MS showed the reaction was completed. The reaction mixture was concentrated under reduced pressure to give a residue. The residue was purified by prep-HPLC (column: Waters Xbridge BEH C18 100*30 mm*10 um; mobile phase: [H₂O (10 mM NH₄HCO₃)-ACN]; gradient: 1%-30% B over 8.0 min) to give Compound I-21 (19.45 mg, 42.77 μmol, 97.7% purity) as a white solid. ¹H NMR (400 MHz, DMSO-d₆) δ 8.77 (d, J=4.8 Hz, 2H), 7.47 (d, J=6.4 Hz, 1H), 7.45-7.41 (m, 1H), 7.15 (t, J=9.2 Hz, 1H), 4.95 (s, 2H), 4.89 (s, 2H), 1.70-1.65 (m, 2H), 1.34-1.28 (m, 2H).

LCMS: MS found: [M+H]⁺=445.1.

### Specific Examples of Preparation of Formula I Compounds Via Synthetic Route 4

Synthesis of compound I-20 has been outlined in Scheme 4. Oxidation of previously described compound 1-C4 with Dess-Martin Periodinane (DMP) (1.5 eq.) in first at 0° C. and then room temperature, following aqueous workup with thiosulfate, afforded aldehyde 2. A reductive amination was then performed by subjecting a mixture of compound 2-C4 (1 eq.), and compound 3-C4 (1 eq) to treatment with acetic acid (1.5 eq.) in dichloromethane at 0° C. Compound 4-C4 was obtained following aqueous workup and chromatographic purification. Compound 5-C4 was obtained by reaction of compound 4-C4 (1 eq.), isocyanatomethylbenzene (2 eq.), and triethylamine (2 eq.) in dichloromethane at elevated temperature for 2 h under N₂ atmosphere followed by aqueous workup and chromatographic purification. Cyclization is performed by reaction of compound 5-C4 (1 eq.) with Cs₂CO₃ (2 eq). followed by atmosphere followed by aqueous workup and chromatographic purification to yield compound 6-C4. Suzuki coupling is performed by reacting mixture of compound 6-C4 (1 eq), previously synthesized compound 7-C4 (1.3 eq), Na₂CO₃ (2 eq), Pd(PPh₃)₄ (0.1 eq) in a mixture of toluene, water and ethanol (1:1:1) which was degassed and purged with N₂ for 3 times, and then the mixture was stirred at 80° C. for 2 h under N₂ atmosphere. The reaction mixture was diluted with H₂O and extracted with ethyl acetate. The combined organic layers were washed with brine, dried over Na₂SO₄, filtered and concentrated under reduced pressure to give a residue. The residue was purified by prep-TLC to give compound 8-C4. Finally, to a solution of compound 8-C4 in dichloromethane was added trifluoroacetic acid and the mixture was stirred for 2 h. The reaction mixture was concentrated, and the residue was purified by prep-HPLC to give compound I-20 as white solid.

### Scheme 4. Synthesis of Compound I-20

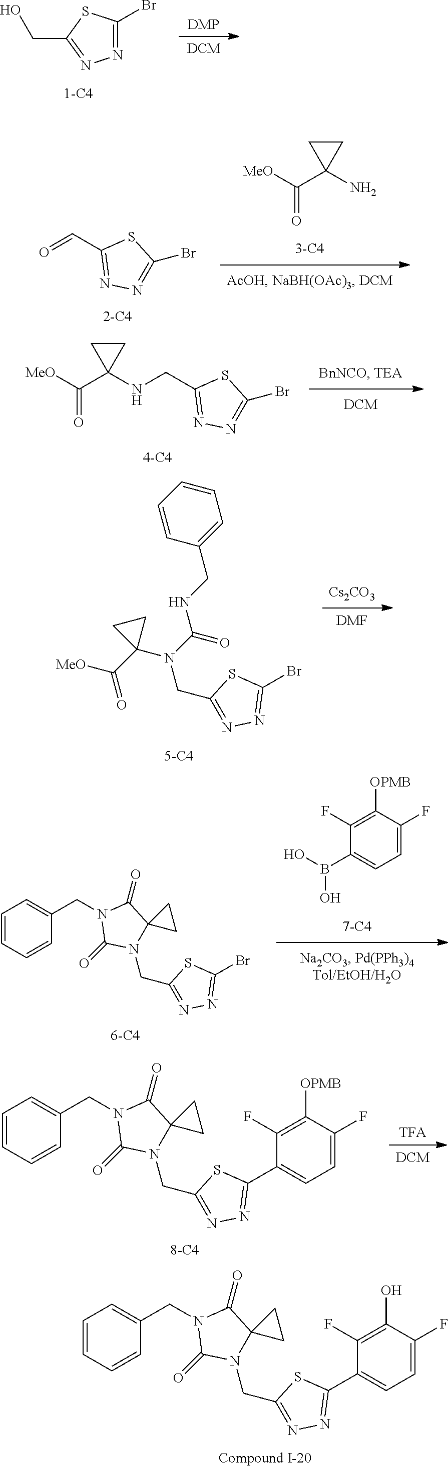

TABLE 4
Targets Associated with Synthetic Route 4

| Example | Compound ID | Name | Structure |
|---|---|---|---|
| 17 | I-20 | 6-benzyl-4-((5-(2,4- difluoro-3- hydroxyphenyl)-1,3,4- thiadiazol-2- yl)methyl)-4,6- diazaspiro[2.4]heptane- 5,7-dione | 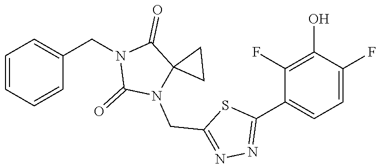 [SMILES: O=C1N(Cc2ccccc2)C(=O)C2(CC2)N1Cc1nnc(-c2ccc(F)c(O)c2F)s1] |
| 18 | I-35 | 6-benzyl-4-((5-(2- chloro-4-fluoro-3- hydroxyphenyl)-1,3,4- thiadiazol-2- yl)methyl)-4,6- diazaspiro[2.4]heptane- 5,7-dione | 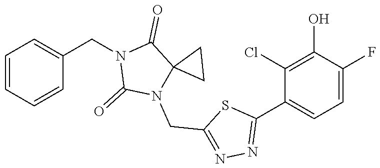 [SMILES: O=C1N(Cc2ccccc2)C(=O)C2(CC2)N1Cc1nnc(-c2ccc(F)c(O)c2Cl)s1] |
| 19 | I-36 | 6-benzyl-4-((5-(2,4- dichloro-3- hydroxyphenyl)-1,3,4- thiadiazol-2- yl)methyl)-4,6- diazaspiro[2.4]heptane- 5,7-dione | 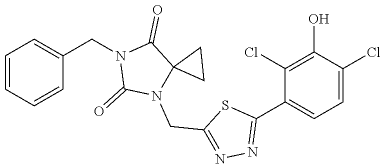 [SMILES: O=C1N(Cc2ccccc2)C(=O)C2(CC2)N1Cc1nnc(-c2ccc(Cl)c(O)c2Cl)s1] |
| 20 | I-34 | 4-((5-(2-chloro-4- fluoro-3- hydroxyphenyl)-1,3,4- thiadiazol-2- yl)methyl)-6-(2,4- dichlorobenzyl)-4,6- diazaspiro[2.4]heptane- 5,7-dione | 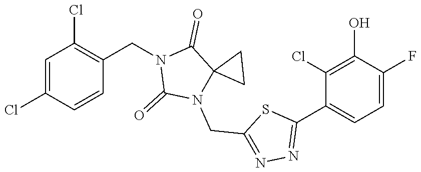 [SMILES: O=C1N(Cc2ccc(Cl)cc2Cl)C(=O)C2(CC2)N1Cc1nnc(-c2ccc(F)c(O)c2Cl)s1] |
| 21 | I-38 | 2-((4-((5-(2-chloro-4- fluoro-3- hydroxyphenyl)-1,3,4- thiadiazol-2- yl)methyl)-5,7-dioxo- 4,6- diazaspiro[2.4]heptan- 6- yl)methyl)benzonitrile | 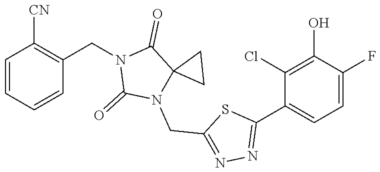 [SMILES: N#Cc1ccccc1CN1C(=O)N(Cc2nnc(-c3ccc(F)c(O)c3Cl)s2)C2(CC2)C1=O] |

### Preparation of 5-bromo-1,3,4-thiadiazole-2-carbaldehyde Compound 2-C4

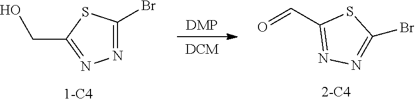

To a solution of compound 1-C4 (5 g, 25.6 mmol, 1 eq) in DCM (50 mL) was added Dess-Martin Periodinane (DMP) (16.3 g, 38.4 mmol, 11.9 mL, 1.5 eq) at 0° C. The mixture was stirred at 20° C. for 2 hr. The mixture was quenched by aqueous saturated sodium thiosulfate (10 mL), diluted with H₂O (30 mL) and extracted with ethyl acetate (5×20 mL). The combined organic layers were washed with aqueous NaHCO₃ (5%), dried over Na₂SO₄, filtered and concentrated under reduced pressure. The residue was purified by prep-TLC (SiO₂, petroleum ether:ethyl acetate=90:10) to give compound 2-C4 (2.7 g, 13.9 mmol) as a yellow solid.

¹H NMR (400 MHz, DMSO-d₆) δ 10.10 (s, 1H)

### Preparation of methyl 1-[(5-bromo-1,3,4-thiadiazol-2-yl)methylamino]cyclopropanecarboxylate Compound 4-C4

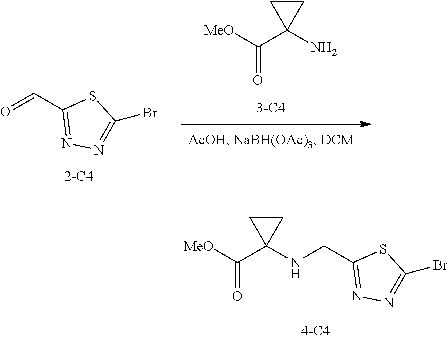 [SMILES: COC(=O)C1(N)CC1 ; O=Cc1nnc(Br)s1 ; COC(=O)C1(NCc2nnc(Br)s2)CC1]

A mixture of compound 2-C4 (2.7 g, 13.9 mmol, 1 eq), compound 3-C4 (1.61 g, 13.9 mmol, 1 eq) and acetic acid (1.26 g, 20.9 mmol, 1.20 mL, 1.5 eq) in DCM (30 mL) was degassed and purged with N₂ for 3 times, and NaBH(OAc)₃ (5.93 g, 27.9 mmol, 2 eq) was added at 0° C. The mixture was stirred at 0° C. for 1 hr, then the mixture was stirred at 15° C. for 12 hr under N₂ atmosphere. The reaction mixture was quenched by addition H₂O (30 mL) at 0° C., and then diluted with H₂O (10 mL) and extracted with DCM (15 mL×3). The combined organic layers were washed with brine 10 mL (5 mL×2), dried over Na₂SO₄, filtered and concentrated under reduced pressure to give a residue. The residue was purified by column chromatography (SiO₂, Petroleum ether:Ethyl acetate=93:7) to give compound 4-C4 (3.3 g, 11.3 mmol,) as a yellow oil.

¹H NMR (400 MHz, DMSO-d₆) δ 4.28-4.20 (m, 2H), 4.15-4.09 (m, 1H), 3.62 (s, 3H), 1.21-1.16 (m, 2H), 1.01-0.95 (m, 2H).

### Preparation of methyl 1-[benzylcarbamoyl-[(5-bromo-1,3,4-thiadiazol-2-yl)methyl]amino]cyclopropanecarboxylate—Compound 5-C4

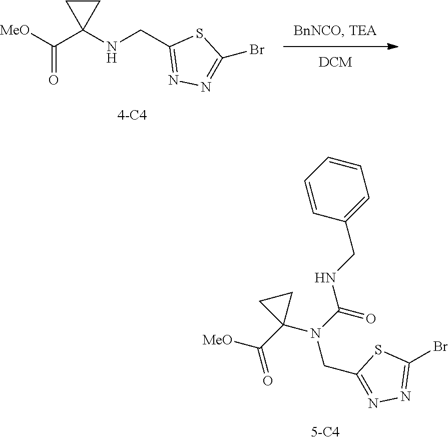

A mixture of compound 4-C4 (0.5 g, 1.71 mmol, 1 eq), isocyanatomethylbenzene (455 mg, 3.42 mmol, 419 μL, 2 eq), triethylamine (TEA, 346 mg, 3.42 mmol, 476 μL, 2 eq) in DCM (5 mL) was degassed and purged with N₂ for 3 times, and then the mixture was stirred at 50° C. for 2 hrs under N₂ atmosphere. TLC indicated one new spot formed. The reaction mixture diluted with H₂O (30 mL) and extracted with DCM (20 mL×3). The combined organic layers were washed with brine (10 mL), dried over Na₂SO₄, filtered and concentrated under reduced pressure to give a residue. The residue was purified by column chromatography (SiO₂, petroleum ether:ethyl acetate=85:15) to give compound 5-C4 (0.5 g, 1.18 mmol,) as a yellow oil.

¹H NMR (400 MHz, CDCl₃-d) δ 7.33-7.26 (m, 5H), 5.38-5.25 (m, 1H), 5.01 (d, J=15.2 Hz, 1H), 4.76 (d, J=15.2 Hz, 1H), 4.40 (s, 2H), 3.61 (s, 3H), 1.81-1.72 (m, 1H), 1.68-1.53 (m, 2H), 1.33 (ddd, J=3.6, 7.2, 10.4 Hz, 1H).

### Preparation of 6-benzyl-4-[(5-bromo-1,3,4-thiadiazol-2-yl)methyl]-4,6-diazaspiro[2.4]heptane-5,7-dione Compound 6-C4

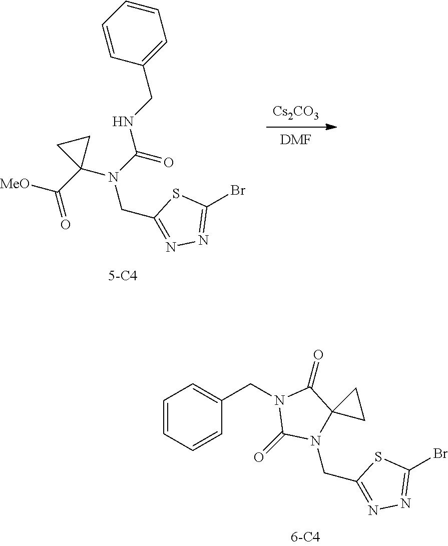

To a solution of compound 5-C4 (0.5 g, 1.18 mmol, 1 eq) in DMF (5 mL) was added Cs₂CO₃ (766 mg, 2.35 mmol, 2 eq). The mixture was stirred at 15° C. for 1 hr. TLC indicated one new spot formed. The reaction mixture was diluted with H₂O (50 mL) and extracted with ethyl acetate (20 mL×3). The combined organic layers were washed with brine (20 mL), dried over Na₂SO₄, filtered and concentrated under reduced pressure to give a residue. The residue was purified by column chromatography (SiO₂, petroleum ether:ethyl acetate=85:15) to give compound 6-C4 (0.2 g, 508 μmol,) as a yellow oil.

¹H NMR (400 MHz, CDCl₃-d) δ=7.45-7.41 (m, 2H), 7.36-7.32 (m, 3H), 4.75-4.72 (m, 2H), 4.68 (s, 2H), 1.46 (td, J=2.4, 7.6 Hz, 4H)

### Preparation of 6-benzyl-4-[[5-[2,4-difluoro-3-[(4-methoxyphenyl)methoxy]phenyl]-1,3,4-thiadiazol-2-yl]methyl]-4,6-diazaspiro[2.4]heptane-5,7-dione—Compound 8-C4

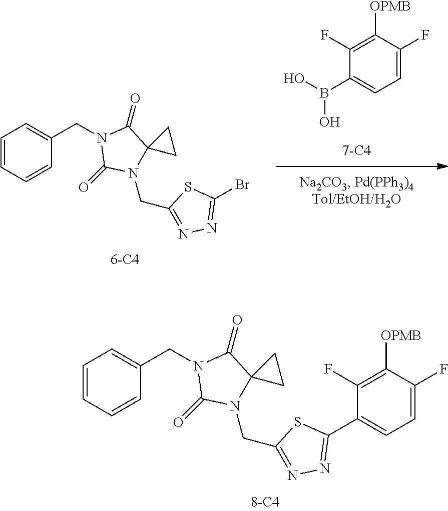

A mixture of compound 6-C4 (0.17 g, 432 μmol, 1 eq), compound 7-C4 (165 mg, 561 μmol, 1.3 eq), Na₂CO₃ (91.6 mg, 864 μmol, 2 eq), Pd(PPh₃)₄ (49.9 mg, 43.2 μmol, 0.1 eq) in Tol. (1 mL), H₂O (1 mL), Ethanol (1 mL) was degassed and purged with N₂ for 3 times, and then the mixture was stirred at 80° C. for 2 hr under N₂ atmosphere. The reaction mixture diluted with H₂O (30 mL) and extracted with ethyl acetate (30 mL×2). The combined organic layers were washed with brine 10 mL, dried over Na₂SO₄, filtered and concentrated under reduced pressure to give a residue. The residue was purified by prep-TLC (SiO₂, petroleum ether:ethyl acetate=33:67) to give compound 8-C4 (0.16 g, 284 μmol,) as yellow oil.

¹H NMR (400 MHz, CDCl₃-d) δ 8.06-7.95 (m, 1H), 7.46 (d, J=6.8 Hz, 2H), 7.41-7.30 (m, 5H), 7.04 (dt, J=2.0, 9.2 Hz, 1H), 6.90 (d, J=8.8 Hz, 2H), 5.17 (s, 2H), 4.78 (d, J=9.2 Hz, 4H), 3.82 (s, 3H), 1.54-1.41 (m, 4H)

### Preparation of 6-benzyl-4-[[5-(2,4-difluoro-3-hydroxy-phenyl)-1,3,4-thiadiazol-2-yl]methyl]-4,6-diazaspiro[2.4]heptane-5,7-dione Compound 1-20

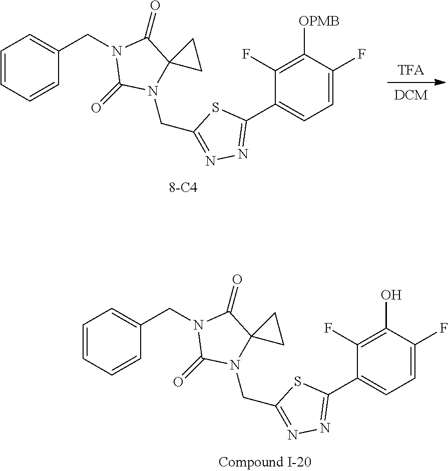

To a solution of compound 8-C4 (0.16 g, 284 μmol, 1 eq) in DCM (1.6 mL) was added TFA (97.2 mg, 853 μmol, 63.3 μL, 3 eq). The mixture was stirred at 15° C. for 2 hrs. The reaction pressure concentration of reaction liquid. The residue was purified by prep-HPLC (column: Waters Xbridge BEH C18 100*30 mm*10 um; mobile phase: [H₂O (10 mM NH₄HCO₃)-ACN]; gradient: 20%-50% B over 8.0 min) to give Compound I-20 (53 mg, 115 μmol, 96% purity) as white solid.

¹H NMR (400 MHz, DMSO-d₆) δ 7.47-7.24 (m, 6H), 7.10 (t, J=9.6 Hz, 1H), 4.92 (s, 2H), 4.67 (s, 2H), 1.69-1.53 (m, 2H), 1.36-1.21 (m, 2H).

LCMS: MS found: [M+H]⁺=443.1.

### Example 18

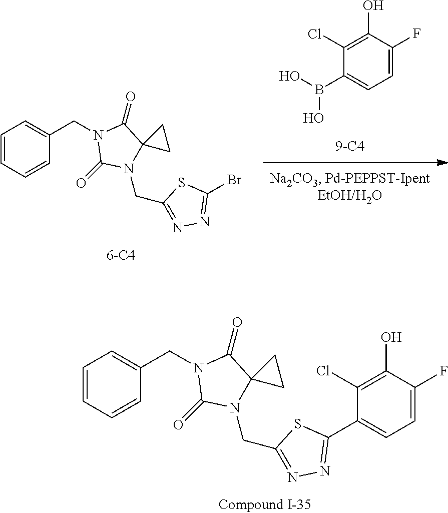 [SMILES: O=C1N(Cc2ccccc2)C(=O)C2(CC2)N1Cc1nnc(Br)s1 ; OB(O)c1ccc(F)c(O)c1Cl ; O=C1N(Cc2ccccc2)C(=O)C2(CC2)N1Cc1nnc(-c2ccc(F)c(O)c2Cl)s1]

### Preparation of 6-benzyl-4-((5-(2-chloro-4-fluoro-3-hydroxyphenyl)-1,3,4-thiadiazol-2-yl)methyl)-4,6-diazaspiro[2.4]heptane-5,7-dione Compound I-35

To a solution of Compound 6-C4 (150 mg, 381 μmol, 1 eq) in ethanol (1.2 mL), H₂O (0.3 mL) was added Compound 9-C4 (72.6 mg, 381 μmol, 1 eq), Na₂CO₃ (60.6 mg, 572 μmol, 1.5 eq) and 1,3-bis[2,6-bis(1-ethylpropyl)phenyl]-4,5-dichloro-2H-imidazol-1-ium-2-ide; 3-chloropyridine; dichloropalladium [Pd-PEPPST-Ipent] (32.8 mg, 38.1 μmol, 0.1 eq). The mixture was stirred at 80° C. for 1 h under N₂. The mixture was poured into water (5 mL) and extracted with ethyl acetate (5 mL×3). The combined organic layers were dried over Na₂SO₄, filtered and concentrated under reduced pressure. The residue was purified by prep-HPLC (column: Phenomenex Luna C18 75*30 mm*3 um; mobile phase: [H₂O (0.1% TFA)-ACN]; gradient: 30%-60% B over 8.0 min) to get Compound I-35 (78.02 mg, 168 μmol, 99.3% purity) as white solid.

¹H NMR (400 MHz, DMSO-d₆) δ 10.97 (br s, 1H), 7.61 (dd, J=5.6, 8.8 Hz, 1H), 7.43-7.27 (m, 6H), 4.94 (s, 2H), 4.68 (s, 2H), 1.64-1.58 (m, 2H), 1.33-1.27 (m, 2H).

LCMS: MS found: [M+H]⁺=459.0.

### Example 19

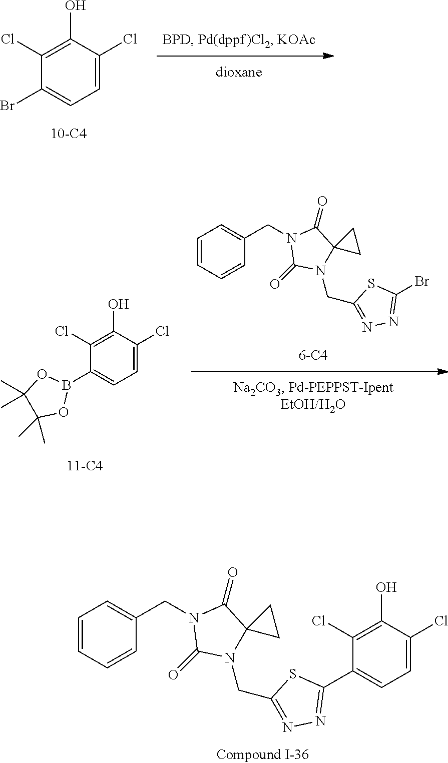 [SMILES: Oc1c(Cl)ccc(Br)c1Cl ; CC1(C)OB(c2ccc(Cl)c(O)c2Cl)OC1(C)C ; O=C1N(Cc2ccccc2)C(=O)C2(CC2)N1Cc1nnc(Br)s1 ; O=C1N(Cc2ccccc2)C(=O)C2(CC2)N1Cc1nnc(-c2ccc(Cl)c(O)c2Cl)s1]

### Preparation of 2,6-dichloro-3-(4,4,5,5-tetramethyl-1,3,2-dioxaborolan-2-yl)phenol Compound 11-C4

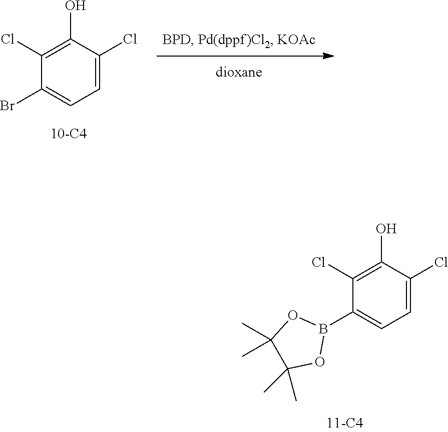 [SMILES: Oc1c(Cl)ccc(Br)c1Cl ; CC1(C)OB(c2ccc(Cl)c(O)c2Cl)OC1(C)C]

To a solution of Compound 10-C4 (0.2 g, 826.80 μmol, 1 eq) and 4,4,5,5-tetramethyl-2-(4,4,5,5-tetramethyl-1,3,2-dioxaborolan-2-yl)-1,3,2-dioxaborolane (230.95 mg, 909.48 μmol, 1.1 eq) in dioxane (2 mL) was added potassium acetate (202.86 mg, 2.07 mmol, 2.5 eq) and Pd(dppf)Cl₂ (60.50 mg, 82.68 μmol, 0.1 eq) at 20° C. under N₂. Then the mixture was stirred at 90° C. for 16 h. The mixture was cooled to 20° C. and filtered and concentrated in vacuum. The residue was purified by silica gel chromatography (column height: 250 mm, diameter: 100 mm, 100-200 mesh silica gel, petroleum ether:ethyl acetate=10:1 to 3:1) to get Compound 11-C4 (100 mg, 346.07 mol,) as white solid.

### Preparation of 6-benzyl-4-((5-(2,4-dichloro-3-hydroxyphenyl)-1,3,4-thiadiazol-2-yl)methyl)-4,6-diazaspiro[2.4]heptane-5,7-dione Compound I-36

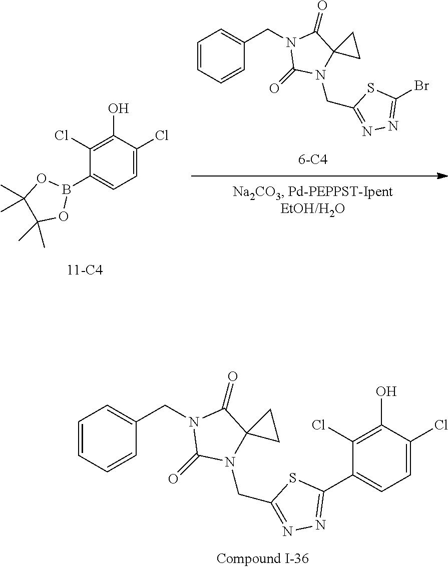 [SMILES: CC1(C)OB(c2ccc(Cl)c(O)c2Cl)OC1(C)C ; O=C1N(Cc2ccccc2)C(=O)C2(CC2)N1Cc1nnc(Br)s1 ; O=C1N(Cc2ccccc2)C(=O)C2(CC2)N1Cc1nnc(-c2ccc(Cl)c(O)c2Cl)s1]

To a solution of Compound 11-C4 (183.70 mg, 635.72 μmol, 1 eq) in ethanol (2 mL), H₂O (0.5 mL) was added previously synthesized compound 6-C4 (250 mg, 635.72 μmol, 1 eq), Na₂CO₃ (101.07 mg, 953.57 μmol, 1.5 eq) and 1,3-bis[2,6-bis(1-ethylpropyl)phenyl]-4,5-dichloro-2H-imidazol-1-ium-2-ide; 3-chloropyridine; dichloropalladium [Pd-PEPPST-Ipent] (27.35 mg, 31.79 mol, 0.05 eq). The mixture was stirred at 80° C. for 1 hr under N₂. The mixture was poured into water (5 mL) and extracted with ethyl acetate (5 mL×3). The combined organic layers were dried over Na₂SO₄, filtered and concentrated under reduced pressure. The residue was purified by prep-HPLC (column: Phenomenex Luna C18 75*30 mm*3 um; mobile phase: [H₂O (0.1% TFA)-ACN]; gradient: 40%-60% B over 8.0 min) to afford Compound I-36 (100 mg, 202.90 μmol, 96.45% purity) as white solid.

¹H NMR (400 MHz, DMSO-d₆) δ 10.68 (s, 1H), 7.66-7.54 (m, 2H), 7.38-7.25 (m, 5H), 4.94 (s, 2H), 4.68 (s, 2H), 1.65-1.59 (m, 2H), 1.34-1.27 (m, 2H).

LCMS: MS found: [M+H]⁺=475.0.

### Example 20

### Route:

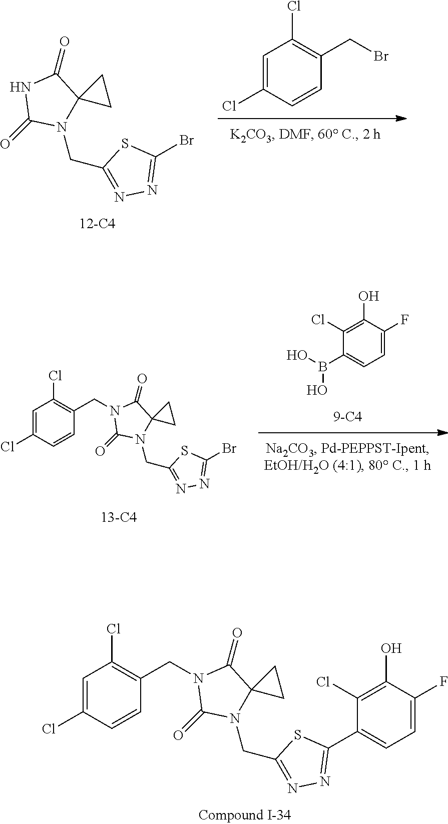 [SMILES: O=C1NC(=O)C2(CC2)N1Cc1nnc(Br)s1 ; Clc1ccc(CBr)c(Cl)c1 ; O=C1N(Cc2ccc(Cl)cc2Cl)C(=O)C2(CC2)N1Cc1nnc(Br)s1 ; OB(O)c1ccc(F)c(O)c1Cl ; O=C1N(Cc2ccc(Cl)cc2Cl)C(=O)C2(CC2)N1Cc1nnc(-c2ccc(F)c(O)c2Cl)s1]

### Preparation of 4-[(5-bromo-1,3,4-thiadiazol-2-yl)methyl]-6-[(2,4-dichlorophenyl)methyl]-4,6-diazaspiro[2.4]heptane-5,7-dione Compound 13-C4

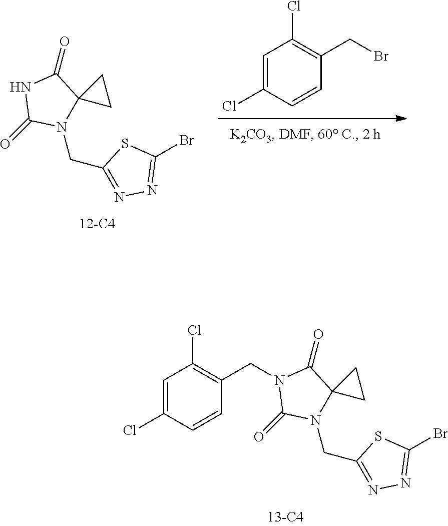 [SMILES: O=C1NC(=O)C2(CC2)N1Cc1nnc(Br)s1 ; Clc1ccc(CBr)c(Cl)c1 ; O=C1N(Cc2ccc(Cl)cc2Cl)C(=O)C2(CC2)N1Cc1nnc(Br)s1]

A mixture of compound 12-C4 (300 mg, 989.66 μmol, 1 eq), 2,4-dichlorobenzyl bromide (284.93 mg, 1.19 mmol, 1.2 eq), K₂CO₃ (410.33 mg, 2.97 mmol, 3 eq) in DMF (4.5 mL) was degassed and purged with N₂ for 3 times, and then the mixture was stirred at 60° C. for 2 hrs under N₂ atmosphere. showed the starting material was consumed completely. The reaction mixture was quenched by addition H₂O (40 mL) at 20° C., and extracted with ethyl acetate (20 mL*2). The combined organic layers were washed with brine (20 mL*2), dried over Na₂SO₄, filtered and concentrated under reduced pressure to give a residue. The residue was purified by column chromatography (SiO₂, petroleum ether:ethyl acetate=58:42) to give compound 13-C4 (330 mg, 714.06 μmol,) as yellow solid.

¹H NMR (400 MHz, CDCl₃) δ 7.12 (s, 1H), 7.09 (d, J=1.2 Hz, 2H), 4.71 (s, 2H), 4.56 (s, 2H), 1.43-1.37 (m, 2H), 1.36-1.31 (m, 2H).

### Preparation of 4-[[5-(2-chloro-4-fluoro-3-hydroxy-phenyl)-1,3,4-thiadiazol-2-yl]methyl]-6-[(2,4-dichlorophenyl)methyl]-4,6-diazaspiro[2.4]heptane-5,7-dione Compound I-34

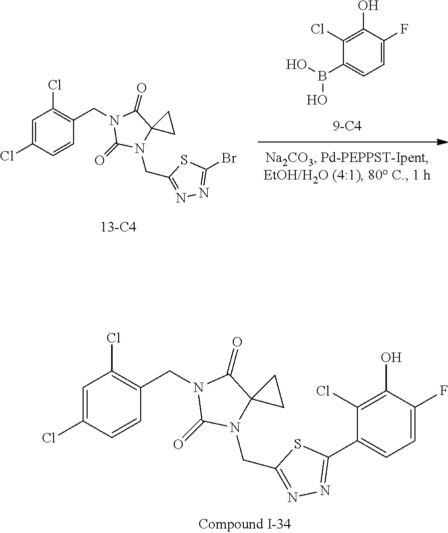 [SMILES: O=C1N(Cc2ccc(Cl)cc2Cl)C(=O)C2(CC2)N1Cc1nnc(Br)s1 ; OB(O)c1ccc(F)c(O)c1Cl ; O=C1N(Cc2ccc(Cl)cc2Cl)C(=O)C2(CC2)N1Cc1nnc(-c2ccc(F)c(O)c2Cl)s1]

A mixture of compound 13-C4 (150 mg, 324.57 μmol, 1 eq), previously synthesized compound 9-C4 (61.79 mg, 324.57 μmol, 1 eq), Na₂CO₃ (51.60 mg, 486.86 μmol, 1.5 eq), Pd-PEPPST-Ipent [1435347-24-2] (13.97 mg, 16.23 μmol, 0.05 eq) in ethanol (1.2 mL) and H₂O (0.3 mL) was degassed and purged with N₂ for 3 times, and then the mixture was stirred at 80° C. for 1 hr under N₂ atmosphere. The mixture was adjusted to pH=3 with 1M HCl solution. The aqueous phase was extracted with ethyl acetate (10 mL*3). The combined organic phase was washed with brine (10 mL), dried with anhydrous Na₂SO₄, filtered and concentrated in vacuum. The residue was purified by prep-HPLC (column: 3_Phenomenex Luna C18 75*30 mm*3 um; mobile phase: [H₂O (0.1% TFA)-ACN]; gradient: 40%-70% B over 8.0 min) to give Compound I-34 (79.6 mg, 150.72 μmol, 99.93% purity) was obtained as a white solid.

¹H NMR (400 MHz, DMSO-d₆) δ 10.97 (br d, J=1.0 Hz, 1H), 7.67-7.59 (m, 2H), 7.45-7.37 (m, 2H), 7.31 (d, J=8.4 Hz, 1H), 4.95 (s, 2H), 4.74 (s, 2H), 1.68-1.61 (m, 2H), 1.35-1.29 (m, 2H).

LCMS: MS found: [M+H]⁺=526.9/529.0.

### Example 21

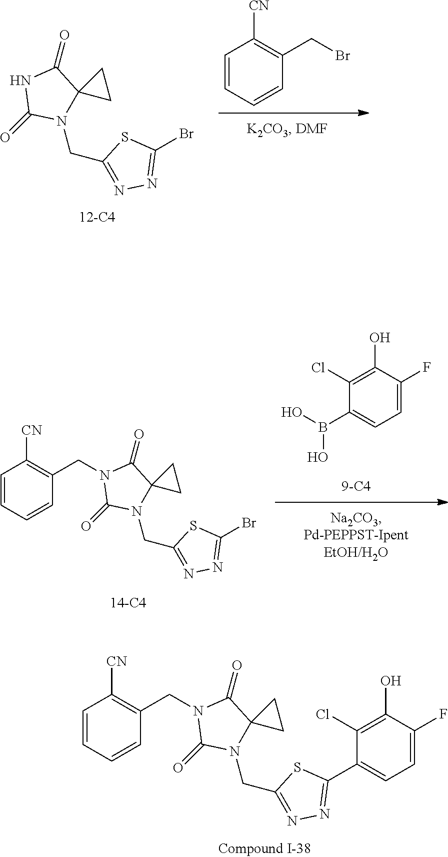 [SMILES: O=C1NC(=O)C2(CC2)N1Cc1nnc(Br)s1 ; N#Cc1ccccc1CBr ; N#Cc1ccccc1CN1C(=O)N(Cc2nnc(Br)s2)C2(CC2)C1=O ; OB(O)c1ccc(F)c(O)c1Cl ; N#Cc1ccccc1CN1C(=O)N(Cc2nnc(-c3ccc(F)c(O)c3Cl)s2)C2(CC2)C1=O]

### Preparation of 2-((4-((5-bromo-1,3,4-thiadiazol-2-yl)methyl)-5,7-dioxo-4,6-diazaspiro[2.4]heptan-6-yl)methyl)benzonitrile Compound 14-C4

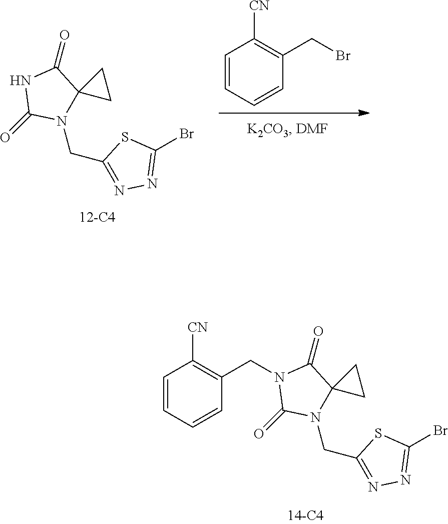 [SMILES: O=C1NC(=O)C2(CC2)N1Cc1nnc(Br)s1 ; N#Cc1ccccc1CBr ; N#Cc1ccccc1CN1C(=O)N(Cc2nnc(Br)s2)C2(CC2)C1=O]

To a solution of compound 12-C4 (0.3 g, 989.66 μmol, 1 eq) in DMF (4.5 mL) was added K₂CO₃ (410.33 mg, 2.97 mmol, 3 eq) and 2-cyanobenzyl bromide (232.82 mg, 1.19 mmol, 1.2 eq) at 15° C. under N₂. The mixture was stirred at 60° C. for 2 h. The mixture was cooled to 15° C. The residue was poured into ice-water (20 mL). The aqueous phase was extracted with ethyl acetate (5 mL×4). The combined organic phase was washed with brine (5 mL×3), dried with anhydrous Na₂SO₄, filtered and concentrated in vacuum to get compound 14-C4 (300 mg, 717.24 μmol,) as a yellow oil.

¹H NMR (400 MHz, CDCl₃) δ 7.72-7.67 (m, 1H), 7.63-7.56 (m, 1H), 7.46-7.39 (m, 2H), 5.00 (s, 2H), 4.72 (s, 2H), 1.56-1.52 (m, 2H), 1.52-1.48 (m, 2H)

### Preparation of 2-((4-((5-(2-chloro-4-fluoro-3-hydroxyphenyl)-1,3,4-thiadiazol-2-yl)methyl)-5,7-dioxo-4,6-diazaspiro[2.4]heptan-6-yl)methyl)benzonitrile Compound I-38

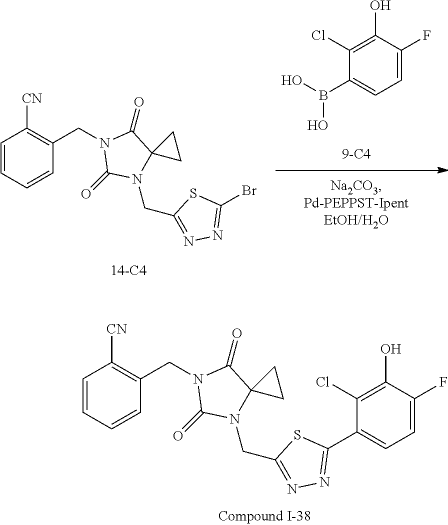 [SMILES: N#Cc1ccccc1CN1C(=O)N(Cc2nnc(Br)s2)C2(CC2)C1=O ; OB(O)c1ccc(F)c(O)c1Cl ; N#Cc1ccccc1CN1C(=O)N(Cc2nnc(-c3ccc(F)c(O)c3Cl)s2)C2(CC2)C1=O]

To a solution of compound 14-C4 (0.2 g, 478.16 μmol, 1 eq) and compound 9-C4 (109.23 mg, 573.80 μmol, 1.2 eq) in ethanol (1.6 mL) and H₂O (0.4 mL) was added Na₂CO₃ (76.02 mg, 717.24 mol, 1.5 eq) and 1,3-bis[2,6-bis(1-ethylpropyl)phenyl]-4,5-dichloro-2H-imidazol-1-ium-2-ide; 3-chloropyridine; dichloropalladium [1435347-24-2] Pd-PEPPST-Ipent (41.15 mg, 47.82 μmol, 0.1 eq) under N₂. Then the mixture was stirred at 80° C. for 1 h. The mixture was cooled to 15° C. The TFA (0.05 mL) was poured into reaction solution and stirred for 1 min. The aqueous phase was extracted with ethyl acetate (5 mL×3). The combined organic phase was washed with brine (5 mL×2), dried with anhydrous Na₂SO₄, filtered and concentrated in vacuum. The residue was purified by prep-HPLC (column: 3_Phenomenex Luna C18 75×30 mm×3 um; mobile phase: [H₂O (0.1% TFA)-ACN]; gradient: 25%-55% B over 8.0 min) to afford Compound I-38 (113 mg, 233.52 μmol, 100% purity) as a white solid.

¹H NMR (400 MHz, DMSO-d₆) δ 11.19-10.83 (br m, 1H), 7.89-7.82 (m, 1H), 7.70 (dt, J=1.2, 7.6 Hz, 1H), 7.61 (dd, J=5.6, 8.8 Hz, 1H), 7.54-7.48 (m, 1H), 7.46-7.36 (m, 2H), 4.95 (s, 2H), 4.87 (s, 2H), 1.72-1.60 (m, 2H), 1.39-1.29 (m, 2H)

LCMS: MS found: [M+H]⁺=484.0.

### Specific Examples of Preparation of Formula I Compounds Via Synthetic Route 5

A mixture of 4-nitrophenyl chloroformate [7693-46-1] (1.5 eq.), (1-(trifluoromethyl) cyclopropyl)methanamine hydrochloride [847926-83-4] (1 eq.) in dichloromethane was added diisopropylethylamine (DIEA, 2 eq.) under N₂. The mixture was stirred at for 16 h and quenched by addition H₂O. After the aqueous workup the residue was purified by column chromatography to give Compound 1-C5. To a solution of Compound 1-C5 (2.5 eq.), previously made compound 2-C5 (1 eq.) in acetonitrile was added dimethylaminopyridine (2.5 eq.). The mixture was stirred at 60° C. for 16 h. The reaction mixture underwent aqueous workup and the residue was purified by column chromatography to give Compound 3-C5. A mixture of Compound 3-C5 (1 eq.), Cs₂CO₃ (2 eq.) in DMF was degassed and then the mixture was stirred for 2 h under N₂ atmosphere. Following the aqueous workup the residue was purified by column chromatography to give Compound 4-C5. To a mixture of Compound 4-C5 (1 eq.) and previously synthesized compound 5-C5 (1.3 eq.) in Ethanol and H₂O (1 mL) was added Na₂CO₃ (1.5 eq), Pd-PEPPST-Ipent [CAS #1435347-24-2] (0.1 eq.) in one portion at 25° C. under N₂. The mixture was stirred at 80° C. for 1 h. The mixture was adjusted to pH=3 with TFA. The aqueous phase was extracted with ethyl acetate. The combined organic phase was washed with brine, dried with anhydrous Na₂SO₄, filtered and concentrated in vacuo. The residue was purified by prep-HPLC to afford Compound I-19.

### Scheme 5. Specific Examples of Preparation of Formula I Compounds Via Synthetic Route 5

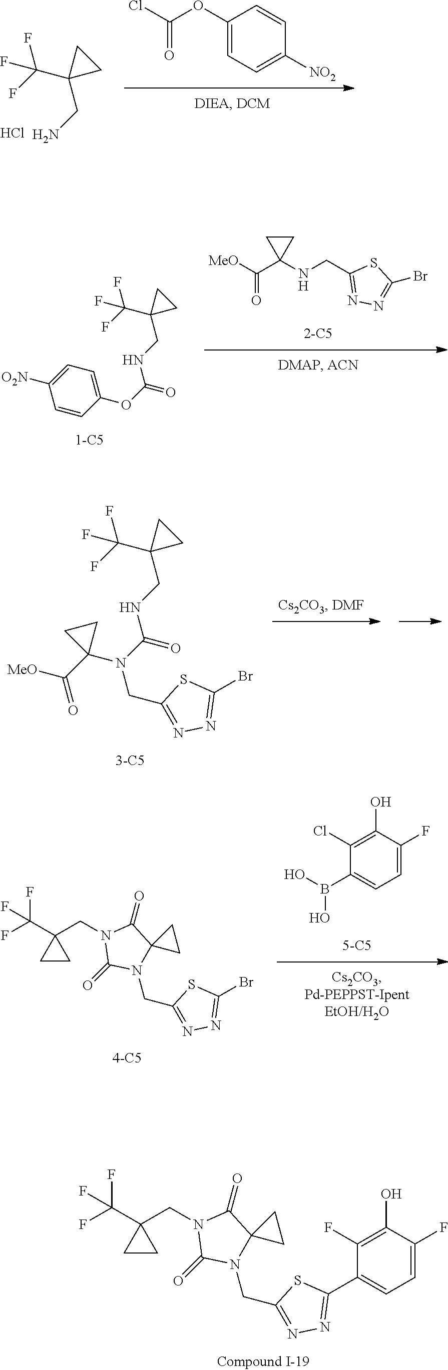

TABLE 5
Targets associated with Synthetic Route 5

| | Compound | | |
|---|---|---|---|
| Example | ID | Name | Structure |
| 22 | I-19 | 4-((5-(2,4-difluoro-3- hydroxyphenyl)-1,3,4-thiadiazol-2- yl)methyl)-6-((1- (trifluoromethyl)cyclopropyl)methyl)- 4,6-diazaspiro[2.4]heptane-5,7-dione | 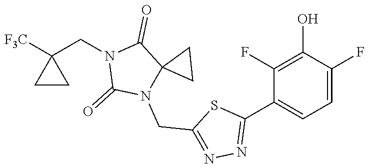 [SMILES: O=C1N(CC2(C(F)(F)F)CC2)C(=O)C2(CC2)N1Cc1nnc(-c2ccc(F)c(O)c2F)s1] |
| 23 | I-25 | 4-((5-(4-chloro-2-fluoro-3- hydroxyphenyl)-1,3,4-thiadiazol-2- yl)methyl)-6-((1- (trifluoromethyl)cyclopropyl)methyl)- 4,6-diazaspiro[2.4]heptane-5,7-dione | 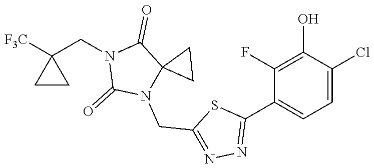 [SMILES: O=C1N(CC2(C(F)(F)F)CC2)C(=O)C2(CC2)N1Cc1nnc(-c2ccc(Cl)c(O)c2F)s1] |
| 24 | I-26 | 4-((5-(2-chloro-4-fluoro-3- hydroxyphenyl)-1,3,4-thiadiazol-2- yl)methyl)-6-((1- (trifluoromethyl)cyclopropyl)methyl)- 4,6-diazaspiro[2.4]heptane-5,7-dione | 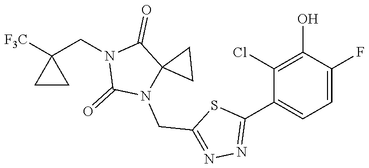 [SMILES: O=C1N(CC2(C(F)(F)F)CC2)C(=O)C2(CC2)N1Cc1nnc(-c2ccc(F)c(O)c2Cl)s1] |

### Preparation of (4-nitrophenyl) N-[[1-(trifluoromethyl)cyclopropyl]methyl]carbamate Compound I-C5

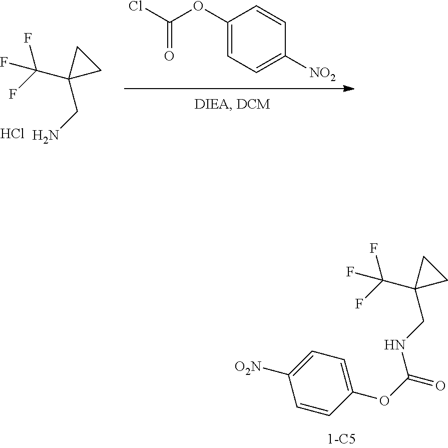 [SMILES: NCC1(C(F)(F)F)CC1 ; Cl ; O=C(Cl)Oc1ccc([N+](=O)[O-])cc1 ; O=C(NCC1(C(F)(F)F)CC1)Oc1ccc([N+](=O)[O-])cc1]

A mixture of 4-nitrophenyl chloroformate (998.75 mg, 4.96 mmol, 1.5 eq), (1-(trifluoromethyl)cyclopropyl)methanamine hydrochloride (580 mg, 3.30 mmol, 1 eq) in DCM (6 mL) was added DIEA (853.86 mg, 6.61 mmol, 1.15 mL, 2 eq) under N₂. The mixture was stirred at 25° C. for 16 h under N₂. TLC showed the starting material was consumed completely. The reaction mixture was quenched by addition H₂O (10 mL×2) at 20° C., and extracted with DCM (10 mL×2). The combined organic layers were washed with brine (10 mL×2), dried over Na₂SO₄, filtered and concentrated under reduced pressure to give a residue. The residue was purified by column chromatography (SiO₂, Petroleum ether:ethyl acetate=89:11 to 86:14) to give Compound 1-C5 (860 mg, 2.83 mmol) as a white solid. Two batches in parallel.

¹H NMR (400 MHz, CDCl₃) δ 8.30-8.23 (m, 2H), 7.37-7.30 (m, 2H), 5.44 (br s, 1H), 3.52 (d, J=6.0 Hz, 2H), 1.15-1.07 (m, 2H), 0.94-0.87 (m, 2H).

### Preparation of methyl 1-[(5-bromo-1,3,4-thiadiazol-2-yl)methyl-[[1-(trifluoromethyl)cyclopropyl]methylcarbamoyl]amino]cyclopropanecarboxylate Compound 3-C5

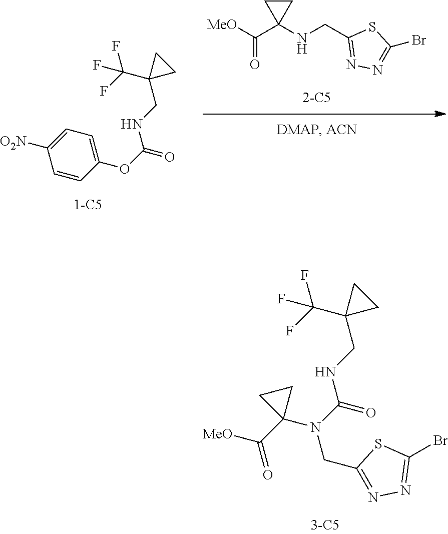 [SMILES: O=C(NCC1(C(F)(F)F)CC1)Oc1ccc([N+](=O)[O-])cc1 ; COC(=O)C1(NCc2nnc(Br)s2)CC1 ; COC(=O)C1(N(Cc2nnc(Br)s2)C(=O)NCC2(C(F)(F)F)CC2)CC1]

To a solution of Compound 1-C5 (1.14 g, 3.75 mmol, 2.5 eq), previously synthesized compound 2-C5 (437.91 mg, 1.50 mmol, 1 eq) in ACN (12 mL) was added DMAP (457.79 mg, 3.75 mmol, 2.5 eq). The mixture was stirred at 60° C. for 16 h. TLC showed the starting material was consumed completely. The reaction mixture was quenched by addition H₂O (40 mL×2) at 20° C., and extracted with EA (40 mL×2). The combined organic layers were washed with brine (40 mL×2), dried over Na₂SO₄, filtered and concentrated under reduced pressure to give a residue. The residue was purified by column chromatography (SiO₂, Petroleum ether:Ethyl acetate=76:24 to 70:30) to give Compound 3-C5 (635 mg, 1.39 mmol) as a yellow oil.

¹H NMR (400 MHz, CDCl₃) δ 5.36 (br t, J=5.8 Hz, 1H), 4.97 (d, J=15.2 Hz, 1H), 4.74 (d, J=15.2 Hz, 1H), 3.64 (s, 3H), 3.52 (d, J=6.0 Hz, 2H), 1.86-1.77 (m, 1H), 1.65 (ddd, J=4.8, 7.6, 10.4 Hz, 1H), 1.54 (ddd, J=4.8, 7.6, 10.4 Hz, 1H), 1.38-1.30 (m, 1H), 1.04-0.98 (m, 2H), 0.91-0.83 (m, 2H).

### Preparation of 4-[(5-bromo-1,3,4-thiadiazol-2-yl)methyl]-6-[[1-(trifluoromethyl)cyclopropyl]methyl]-4,6-diazaspiro[2.4]heptane-5,7-dione Compound 4-C5

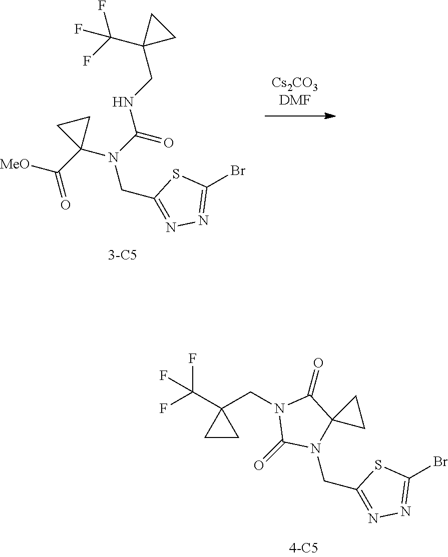 [SMILES: COC(=O)C1(N(Cc2nnc(Br)s2)C(=O)NCC2(C(F)(F)F)CC2)CC1 ; O=C1N(CC2(C(F)(F)F)CC2)C(=O)C2(CC2)N1Cc1nnc(Br)s1]

A mixture of Compound 3-C5 (660 mg, 1.44 mmol, 1 eq), Cs₂CO₃ (940.55 mg, 2.89 mmol, 2 eq) in DMF (7 mL) was degassed and purged with N₂ for 3 times, and then the mixture was stirred at 20° C. for 2 h under N₂ atmosphere. TLC showed the starting material was consumed completely. The reaction mixture was quenched by addition H₂O (20 mL×2) at 20° C., and extracted with ethyl acetate (20 mL×2). The combined organic layers were washed with brine (20 mL×2), dried over Na₂SO₄, filtered and concentrated under reduced pressure to give a residue. The residue was purified by column chromatography (SiO₂, petroleum ether:ethyl acetate=70:30) to give compound 4-C5 (635 mg, 1.39 mmol) as a yellow oil.

¹H NMR (400 MHz, CDCl₃) δ 4.70 (s, 2H), 3.81 (s, 2H), 1.54-1.39 (m, 4H), 1.13-0.98 (m, 4H).

### Preparation of 4-((5-(2,4-difluoro-3-hydroxyphenyl)-1,3,4-thiadiazol-2-yl)methyl)-6-((1-(trifluoromethyl)cyclopropyl)methyl)-4,6-diazaspiro[2.4]heptane-5,7-dione Compound I-19

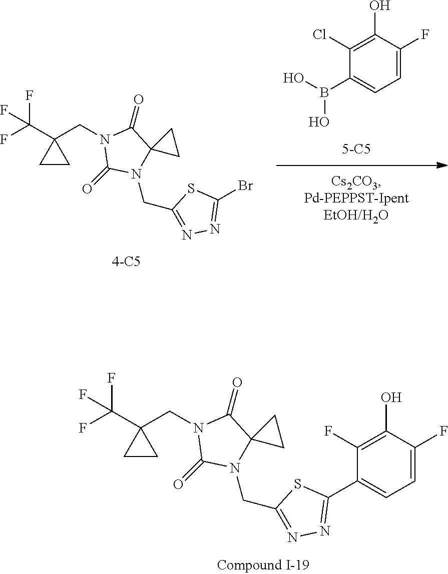 [SMILES: O=C1N(CC2(C(F)(F)F)CC2)C(=O)C2(CC2)N1Cc1nnc(Br)s1 ; OB(O)c1ccc(F)c(O)c1Cl ; O=C1N(CC2(C(F)(F)F)CC2)C(=O)C2(CC2)N1Cc1nnc(-c2ccc(F)c(O)c2F)s1]

To a mixture of Compound 4-C5 (0.1 g, 235.17 μmol, 1 eq) and Compound 5-C5 (53.17 mg, 305.72 μmol, 1.3 eq) in ethanol (4 mL) and H₂O (1 mL) was added Na₂CO₃ (37.39 mg, 352.76 mol, 1.5 eq), Pd-PEPPST-IPent [1435347-24-2] (20.24 mg, 23.52 μmol, 0.1 eq) in one portion at 25° C. under N₂. The mixture was stirred at 80° C. for 1 hour. The mixture was adjusted to pH=3 with TFA. The aqueous phase was extracted with ethyl acetate (5 mL×3). The combined organic phase was washed with brine (10 mL), dried with anhydrous Na₂SO₄, filtered and concentrated in vacuum. The residue was purified by prep-HPLC (column: Phenomenex Luna C18 75×30 mm×3 um; mobile phase: [H₂O (0.1% TFA)-ACN]; gradient: 35%-60% B over 8.0 min) to afford Compound I-19 (84.81 mg, 178.77 μmol) as white solid.

¹H NMR (400 MHz, DMSO-d₆) δ 10.79 (s, 1H), 7.70-7.64 (m, 1H), 7.29-7.24 (m, 1H), 4.93 (s, 2H), 3.76 (s, 2H), 1.63-1.60 (m, 2H), 1.29-1.28 (m, 2H), 1.27-1.26 (m, 2H), 1.09-0.99 (m, 2H).

LCMS: MS found: [M+H]⁺=475.1.

### Example 23

### Route:

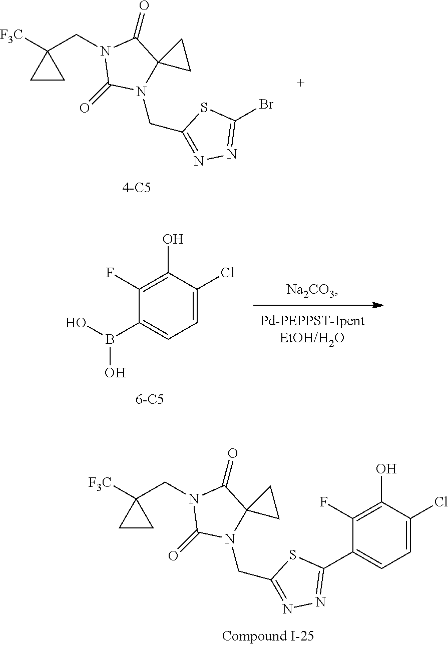 [SMILES: O=C1N(CC2(C(F)(F)F)CC2)C(=O)C2(CC2)N1Cc1nnc(Br)s1 ; OB(O)c1ccc(Cl)c(O)c1F ; O=C1N(CC2(C(F)(F)F)CC2)C(=O)C2(CC2)N1Cc1nnc(-c2ccc(Cl)c(O)c2F)s1]

### Preparation of 4-((5-(4-chloro-2-fluoro-3-hydroxyphenyl)-1,3,4-thiadiazol-2-yl)methyl)-6-((1-(trifluoromethyl)cyclopropyl)methyl)-4,6-diazaspiro[2.4]heptane-5,7-dione Compound I-25

To a solution of Compound 4-C5 (100 mg, 235.17 μmol, 1 eq) and Compound 6-C5 (53.72 mg, 282.20 μmol, 1.2 eq) in ethanol (1.6 mL) and H₂O (0.4 mL) was added Na₂CO₃ (37.39 mg, 352.76 mol, 1.5 eq) and 1,3-bis[2,6-bis(1-ethylpropyl)phenyl]-4,5-dichloro-2H-imidazol-1-ium-2-ide; 3-chloropyridine; dichloropalladium Pd-PEPPST-IPent [1435347-24-2] (10.12 mg, 11.76 μmol, 0.05 eq). The mixture was stirred at 80° C. for 1 h under N₂. The solution was adjusted to pH ˜4 with citric acid and extracted with ethyl acetate (10 mL×2). The combined organic layers were dried over Na₂SO₄, filtered and concentrated under reduced pressure. The residue was purified by prep-HPLC (column: 3_Phenomenex Luna C18 75×30 mm×3 um; mobile phase: [H₂O (0.1% TFA)-ACN]; gradient: 30%-60% B over 8.0 min) to afford Compound I-25 (89.6 mg, 181.55 mol, 99.46% purity) as white solid.

¹H NMR (400 MHz, DMSO-d₆) δ 10.98 (br s, 1H), 7.66 (dd, J=7.2, 8.8 Hz, 1H), 7.41 (dd, J=1.6, 8.8 Hz, 1H), 4.93 (s, 2H), 3.75 (s, 2H), 1.66-1.56 (m, 2H), 1.32-1.23 (m, 2H), 1.08 (s, 2H), 1.03-0.95 (m, 2H)

LCMS: MS found: [M+H]⁺=491.0.

### Example 24

### Route:

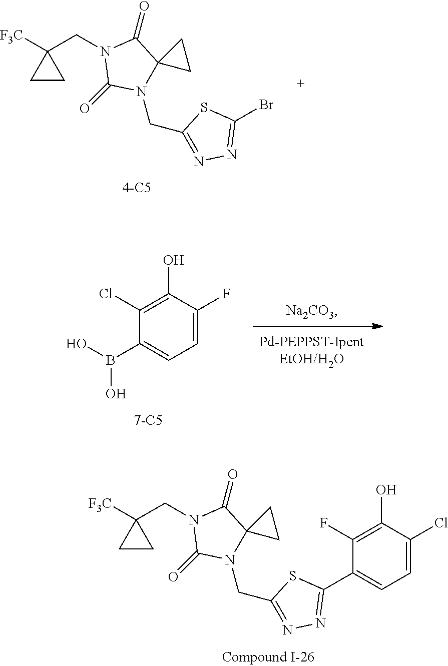 [SMILES: O=C1N(CC2(C(F)(F)F)CC2)C(=O)C2(CC2)N1Cc1nnc(Br)s1 ; OB(O)c1ccc(F)c(O)c1Cl ; O=C1N(CC2(C(F)(F)F)CC2)C(=O)C2(CC2)N1Cc1nnc(-c2ccc(Cl)c(O)c2F)s1]

### Preparation of 4-((5-(2-chloro-4-fluoro-3-hydroxyphenyl)-1,3,4-thiadiazol-2-yl)methyl)-6-((1-(trifluoromethyl)cyclopropyl)methyl)-4,6-diazaspiro[2.4]heptane-5,7-dione Compound I-26

To a solution of Compound 4-C5 (60.0 mg, 141 μmol, 1 eq), Compound 7-C5 (26.8 mg, 141 μmol, 1 eq) in ethanol (0.8 mL), H₂O (0.2 mL) was added Na₂CO₃ (22.4 mg, 211 μmol, 1.5 eq) and 1,3-bis[2,6-bis(1-ethylpropyl)phenyl]-4,5-dichloro-2H-imidazol-1-ium-2-ide; 3-chloropyridine; dichloropalladium [Pd-PEPPST-Ipent] (12.1 mg, 14.1 μmol, 0.1 eq). The mixture was stirred at 80° C. for 1 hr. The reaction mixture diluted with H₂O 2 mL and extracted with ethyl acetate (2 mL×2). The combined organic layers were washed with brine 2 mL, dried over Na₂SO₄, filtered and concentrated under reduced pressure to give a residue. The residue was purified by prep-HPLC (column: Waters Xbridge BEH C18 100×30 mm×10 um; mobile phase: [H₂O (10 mM NH₄HCO₃)-ACN]; gradient: 15%-50% B over 8.0 min) to afford Compound I-26 (30 mg, 60.5 μmol, 99% purity) as a white solid.

¹H NMR (400 MHz, DMSO-d₆) δ 7.56-7.49 (m, 1H), 7.35 (t, J=9.6 Hz, 1H), 4.92 (s, 2H), 3.75 (s, 2H), 1.65-1.59 (m, 2H), 1.31-1.25 (m, 2H), 1.08 (s, 2H), 1.01-0.96 (m, 2H).

LCMS: MS found: [M+H]⁺=491.0.

### Specific Examples of Preparation of Formula I Compounds Via Synthetic Route 6

A mixture of previously described compound 1-C6 in dichloromethane was degassed and purged with N₂ for 3 times, and added Dess-Martin Periodinane [87413-09-0] (DMP, 1.5 eq.) in first at 0° C. and then room temperature. Following aqueous workup with thiosulfate and chromatographic purification, aldehyde 2-C6 was obtained. A mixture of (+/−) alanine ethyl ester HCl salt [617-27-6] (1 eq.), diisopropylethylamine (DIEA, 1 eq.) in dichloromethane was degassed and purged with N₂, then stirred for 30 min, then previously described 5-bromo-1,3,4-thiadiazole-2-carbaldehyde (1 eq.), AcOH (1.5 eq.) and NaBH(OAc)₃ (2 eq.) were added at 0° C. The mixture was stirred at 0-25° C. for 12 h under N₂ atmosphere. After the aqueous workup and chromatography compound 3-C6 was obtained. A mixture of compound 3-C6 (1 eq.), isocyanatoethane (2 eq.), diisopropylethylaime (DIEA, 2 eq.) in ACN was stirred at 25° C. for 6 h under N₂ atmosphere. The mixture was concentrated under reduced pressure, and, after the aqueous workup purified by chromatography to yield compound 4-C6. A mixture of compound 4-C6 (1 eq.), previously used (2,4-difluoro-3-hydroxy-phenyl)boronic acid (5-C6, 1 eq.), Pd(PPh₃)₄ (0.1 eq.), Na₂CO₃ (2 eq) in a mixture of H₂, Ethanol, and toluene was stirred at 80° C. for 2 h under argon atmosphere. After the aqueous workup the mixture was purified by preparative reverse-phase HPLC to give Compound I-3 as a white solid.

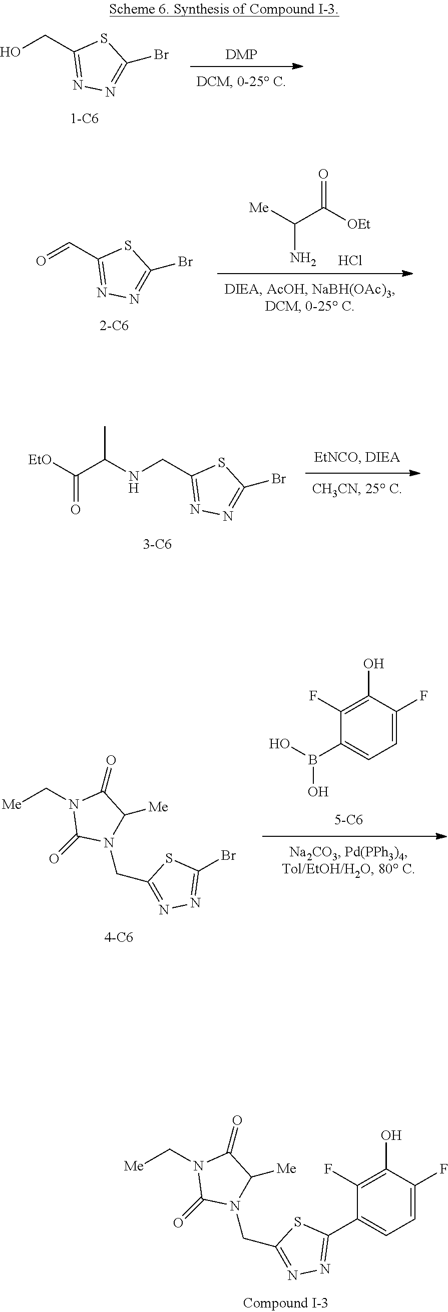

TABLE 6
Targets associated with Synthetic Route 6

| | Compound | | |
|---|---|---|---|
| Example | ID | Name | Structure |
| 25 | I-3 | 1-((5-(2,4-difluoro-3- hydroxyphenyl)-1,3,4- thiadiazol-2- yl)methyl)-3-ethyl-5- methylimidazolidine- 2,4-dione | 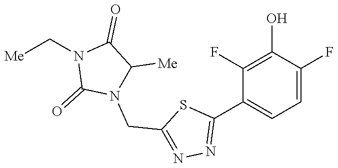 [SMILES: CCN1C(=O)C(C)N(Cc2nnc(-c3ccc(F)c(O)c3F)s2)C1=O] |
| 27 | I-8 | 1-((5-(2,4-difluoro-3- hydroxyphenyl)-1,3,4- thiadiazol-2- yl)methyl)-3-ethyl-5,5- dimethylimidazolidine- 2,4-dione | 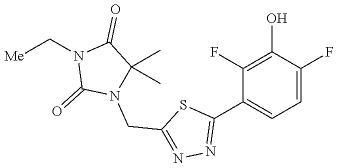 [SMILES: CCN1C(=O)N(Cc2nnc(-c3ccc(F)c(O)c3F)s2)C(C)(C)C1=O] |
| 28 | I-1 | 5-((5-(2,4-difluoro-3- hydroxyphenyl)-1,3,4- thiadiazol-2- yl)methyl)-7-ethyl-5,7- diazaspiro[3.4]octane- 6,8-dione | 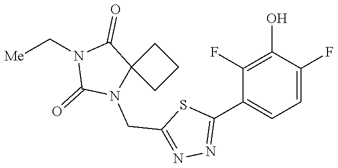 [SMILES: CCN1C(=O)N(Cc2nnc(-c3ccc(F)c(O)c3F)s2)C2(CCC2)C1=O] |

### Preparation of 5-bromo-1,3,4-thiadiazole-2-carbaldehyde Compound 2-C6

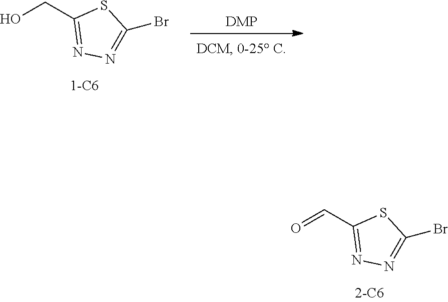

A mixture of previously synthesized compound 1-C6 (600 mg, 3.08 mmol, 1 eq) in dichloromethane (10 mL) was degassed and purged with N₂ for 3 times, and added Dess-Martin Periodinane [87413-09-0] (DMP, 1.96 g, 4.61 mmol, 1.43 mL, 1.5 eq) at 0° C. The mixture was stirred at 0-25° C. for 6 hrs under N₂ atmosphere. TLC (petroleum ether:ethyl acetate=2:1, R_{f}=0.5) indicated the reactant was consumed completely. The mixture was quenched by saturated Na₂S₂O₃ (20 mL), diluted with H₂O (10 mL) and extracted with dichloromethane:isopropanol=5:1 (30 mL*2). The combined organic layers were washed with saturated NaHCO₃ (20 mL*2), dried over Na₂SO₄, filtered and concentrated under reduced pressure. The residue was purified by column chromatography (SiO₂, petroleum ether:ethyl acetate=1:0 to 1:1) to give compound 2-C6 (480 mg, 2.49 mmol, -) as a yellow oil.

¹H NMR (400 MHz, DMSO-d₆) δ ppm 10.10 (s, 1H).

### Preparation of ethyl 2-[(5-bromo-1,3,4-thiadiazol-2-yl)methylamino]propanoate Compound 3-C6

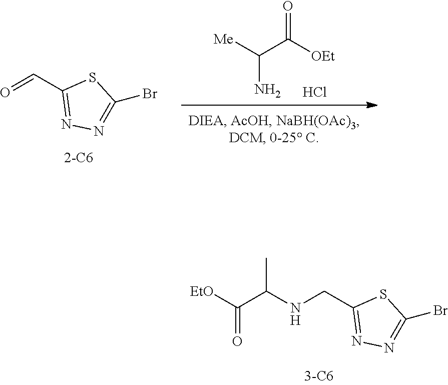

A mixture of (+/−) alanine ethyl ester HCl salt [617-27-6] (238.74 mg, 1.55 mmol, 1 eq.), DIEA (200.87 mg, 1.55 mmol, 270.72 μL, 1 eq) in DCM (10 mL) was degassed and purged with N₂ for 3 times. After stirred for 30 min, 5-bromo-1,3,4-thiadiazole-2-carbaldehyde (2-C6, 300 mg, 1.55 mmol, 1 eq), acetic acid (140.00 mg, 2.33 mmol, 133.46 μL, 1.5 eq) and NaBH(OAc)₃ (658.81 mg, 3.11 mmol, 2 eq) was added at 0° C. The mixture was stirred at 0-25° C. for 12 hrs under N₂ atmosphere. The reaction mixture was quenched by saturated NaHCO₃ (5 mL), diluted with H₂O (5 mL) and extracted with DCM (15 mL*2). The combined organic layers were dried over Na₂SO₄, filtered and concentrated under reduced pressure. The residue was purified by column chromatography (SiO₂, petroleum ether:ethyl acetate=10:1 to 1:1) to give compound 3-C6 (250 mg, 849.85 μmol,) as a yellow oil.

¹H NMR (400 MHz, DMSO-d₆) δ ppm 4.27-3.89 (m, 4H), 3.50-3.37 (m, 2H), 1.23-1.15 (m, 6H).

### Preparation of 1-[(5-bromo-1,3,4-thiadiazol-2-yl)methyl]-3-ethyl-5-methyl-imidazolidine-2,4-dione Compound 4-C6

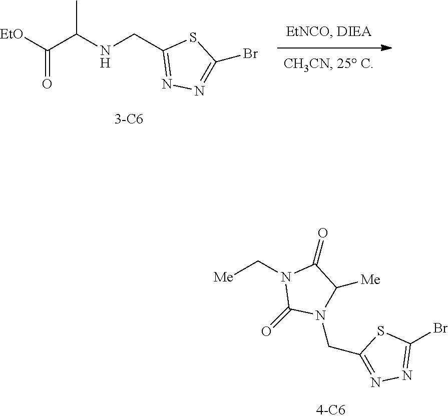 [SMILES: CCOC(=O)C(C)NCc1nnc(Br)s1 ; CCN1C(=O)C(C)N(Cc2nnc(Br)s2)C1=O]

A mixture of compound 3-C6 (250 mg, 849.85 μmol, 1 eq), isocyanatoethane (120.81 mg, 1.70 mmol, 134.53 μL, 2 eq), DIEA (219.68 mg, 1.70 mmol, 296.06 μL, 2 eq) in ACN (4 mL) was degassed and purged with N₂ for 3 times. The mixture was stirred at 25° C. for 6 hrs under N₂ atmosphere. TLC indicated the reactant was consumed completely. The mixture was concentrated under reduced pressure. The residue was diluted with H₂O (20 mL), extracted with ethyl acetate (20 mL×2). The combined organic layers were dried over Na₂SO₄, filtered and concentrated under reduced pressure. The residue was purified by column chromatography (SiO₂, petroleum ether:ethyl acetate=1:0 to 1:1) to give compound 4-C6 (200 mg, 626.61 μmol,) as a yellow oil.

¹H NMR (400 MHz, DMSO-d₆) δ ppm 5.11-5.01 (m, 1H), 4.93-4.80 (m, 1H), 4.19 (q, J=6.8 Hz, 1H), 3.72-3.58 (m, 1H), 3.42 (q, J=7.2 Hz, 2H), 1.34-1.27 (m, 3H), 1.09 (t, J=7.2 Hz, 3H).

LCMS: RT=0.580 min, MS cal.: 318.0, MS found: [M+H]⁺=319.0, 321.0.

### Preparation of 1-[[5-(2,4-difluoro-3-hydroxy-phenyl)-1,3,4-thiadiazol-2-yl]methyl]-3-ethyl-5-methyl-imidazolidine-2,4-dione Compound 1-3

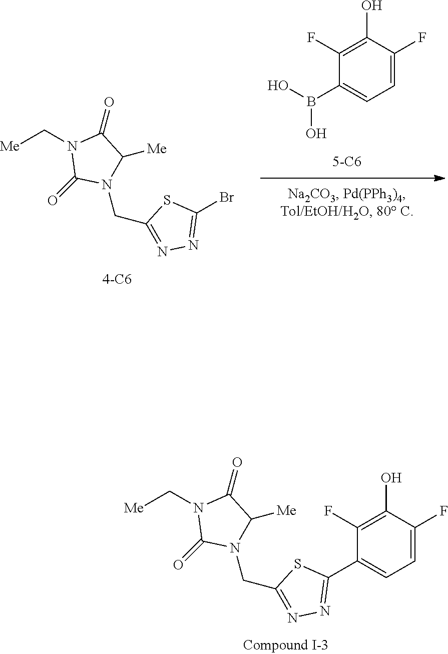 [SMILES: CCN1C(=O)C(C)N(Cc2nnc(Br)s2)C1=O ; OB(O)c1ccc(F)c(O)c1F ; CCN1C(=O)C(C)N(Cc2nnc(-c3ccc(F)c(O)c3F)s2)C1=O]

A mixture of compound 4-C6 (180 mg, 563.95 μmol, 1 eq), previously synthesized (2,4-difluoro-3-hydroxy-phenyl)boronic acid (5-C6, 98.08 mg, 563.95 μmol, 1 eq), Pd(PPh₃)₄ (65.17 mg, 56.39 mol, 0.1 eq), Na₂CO₃ (119.54 mg, 1.13 mmol, 2 eq) in H₂O (0.8 mL), ethanol (0.8 mL) and toluene (0.8 mL) was degassed and purged with Ar for 3 times. The mixture was stirred at 80° C. for 2 hrs under Ar atmosphere. LC-MS showed the reactant was consumed completely. The mixture was diluted with H₂O (10 mL), extracted with ethyl acetate (10 mL*2). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% formic acid)-ACN]; gradient: 30%-60% B over 8.0 min) to give Compound I-3 (101.64 mg, 274.48 μmol, 99.47% purity) as a white solid.

¹H NMR (400 MHz, DMSO-d₆) δ ppm 7.71-7.59 (m, 1H), 7.26 (t, J=9.6 Hz, 1H), 5.17-5.09 (m, 1H), 5.01-4.92 (m, 1H), 4.23 (q, J=6.8 Hz, 1H), 3.44 (d, J=7.2 Hz, 2H), 1.33 (d, J=6.8 Hz, 3H), 1.10 (t, J=7.2 Hz, 3H).

LCMS: RT=2.128 min, MS cal.: 368.08, MS found: [M+H]⁺=369.1.

HPLC: RT 2.105 min, purity: 99.474%.

### Example 27

### Route:

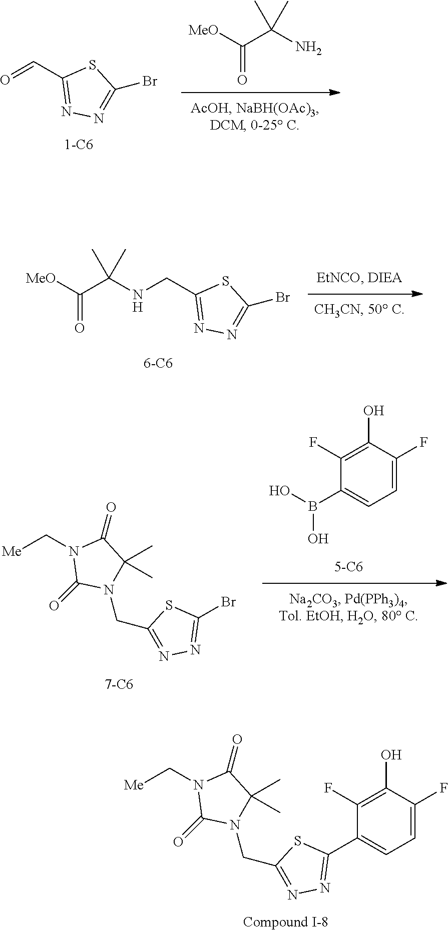

### Preparation of methyl 2-[(5-bromo-1,3,4-thiadiazol-2-yl)methylamino]-2-methyl-propanoate Compound 6-C6

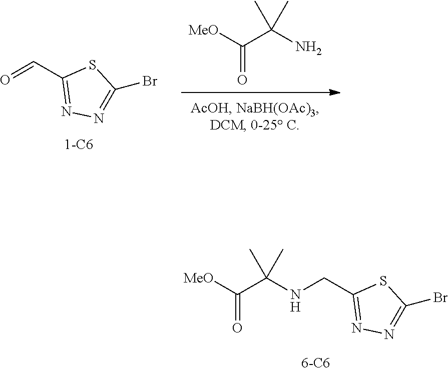

A mixture of previously synthesized compound 1-C6 (500 mg, 2.59 mmol, 1 eq), methyl 2-amino-2-methyl-propanoate (303.45 mg, 2.59 mmol, 1 eq), acetic acid (233.34 mg, 3.89 mmol, 222.44 L, 1.5 eq) in dichloromethane (10 mL) was degassed and purged with N₂ for 3 times. The mixture was stirred at 0-25° C. for 0.5 hr under N₂ atmosphere. NaBH(OAc)₃ (1.10 g, 5.18 mmol, 2 eq) was added at 0° C. The mixture was stirred at 0-25° C. for 5 h under N₂ atmosphere. The mixture was quenched by saturated NaHCO₃ (10 mL), diluted with H₂O (10 mL) and extracted with dichloromethane (25 mL×2). The combined organic layers were dried over Na₂SO₄, filtered and concentrated under reduced pressure. The residue was purified by column chromatography (SiO₂, petroleum ether:ethyl acetate=1:0 to 1:1) to give compound 6-C6 (420 mg, 1.43 mmol,) as a yellow oil.

¹H NMR (400 MHz, DMSO-d₆) δ ppm 4.04 (d, J=7.6 Hz, 2H), 3.73-3.56 (m, 4H), 1.29-1.22 (s, 6H).

### Preparation of 1-[(5-bromo-1,3,4-thiadiazol-2-yl)methyl]-3-ethyl-5,5-dimethyl-imidazolidine-2,4-dione Compound 7-C6

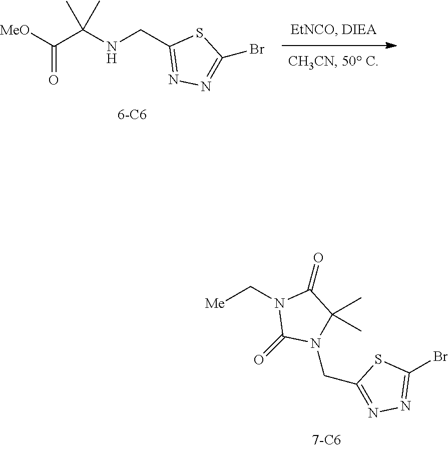 [SMILES: COC(=O)C(C)(C)NCc1nnc(Br)s1 ; CCN1C(=O)N(Cc2nnc(Br)s2)C(C)(C)C1=O]

A mixture of compound 5-C6 (420 mg, 1.43 mmol, 1 eq), isocyanatoethane (202.96 mg, 2.86 mmol, 226.02 μL, 2 eq), DIEA (369.05 mg, 2.86 mmol, 497.38 μL, 2 eq) in ACN (5 mL) was degassed and purged with N₂ for 3 times. The mixture was stirred at 50° C. for 12 h under N₂ atmosphere. The reaction mixture was concentrated under reduced pressure. The residue was diluted with H₂O (20 mL), extracted with ethyl acetate (20 mL×2). The combined organic layers were dried over Na₂SO₄, filtered and concentrated under reduced pressure. The residue was purified by column chromatography (SiO₂, petroleum ether:ethyl acetate=1:0 to 1:1) to give compound 7-C6 (420 mg, 1.26 mmol,) as a yellow solid.

¹H NMR (400 MHz, DMSO-d₆) δ 4.98 (s, 2H), 3.78-3.60 (m, 2H), 1.35 (s, 6H), 1.10 (t, J=7.2 Hz, 3H).

LCMS: RT=0.623 min, MS cal.: 331.99, MS found: [M+H]⁺=335.0.

### Preparation of 1-[[5-(2,4-difluoro-3-hydroxy-phenyl)-1,3,4-thiadiazol-2-yl]methyl]-3-ethyl-5,5-dimethyl-imidazolidine-2,4-dione Compound I-8

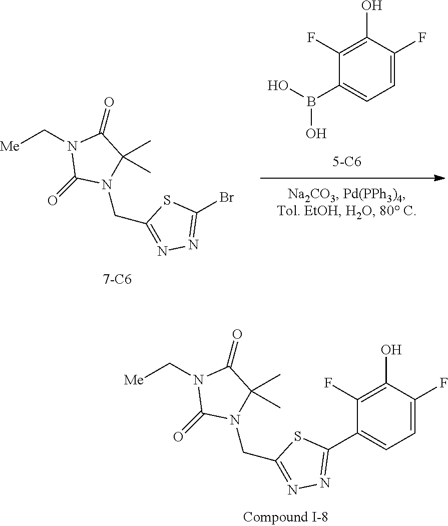 [SMILES: CCN1C(=O)N(Cc2nnc(Br)s2)C(C)(C)C1=O ; OB(O)c1ccc(F)c(O)c1F ; CCN1C(=O)N(Cc2nnc(-c3ccc(F)c(O)c3F)s2)C(C)(C)C1=O]

A mixture of compound 7-C6 (420 mg, 1.26 mmol, 1 eq), (2,4-difluoro-3-hydroxy-phenyl)boronic acid (5-C6, 219.21 mg, 1.26 mmol, 1 eq), Pd(PPh₃)₄ (145.66 mg, 126.05 μmol, 0.1 eq), Na₂CO₃ (267.20 mg, 2.52 mmol, 2 eq) in toluene (1.6 mL), ethanol (1.6 mL) and H₂O (1.6 mL) was degassed and purged with Ar for 3 times. The mixture was stirred at 80° C. for 2 h under Ar atmosphere. The mixture was diluted with H₂O (20 mL), extracted with ethyl acetate (20 mL*2). The combined organic layers were dried over Na₂SO₄, filtered and concentrated under reduced pressure. The residue was purified by prep-HPLC (column: Phenomenex luna C18 100*40 mm*3 um; mobile phase: [H₂O (0.2% formic acid)-ACN]; gradient: 35%-65% B over 8.0 min) to give Compound I-8 (208.36 mg, 541.49 μmol, 99.37% purity) as white solid.

¹H NMR (400 MHz, DMSO-d₆) δ 10.82 (brs, 1H), 7.68-7.62 (m, 1H), 7.28-7.20 (m, 1H), 5.04 (s, 2H), 3.45 (q, J=7.2 Hz, 2H), 1.34 (s, 6H), 1.11 (t, J=7.2 Hz, 3H).

LCMS: RT=2.207 min, MS cal.: 382.1, MS found: [M+H]⁺=383.1.

HPLC: RT=2.776 min, purity: 99.374%.

### Example 28

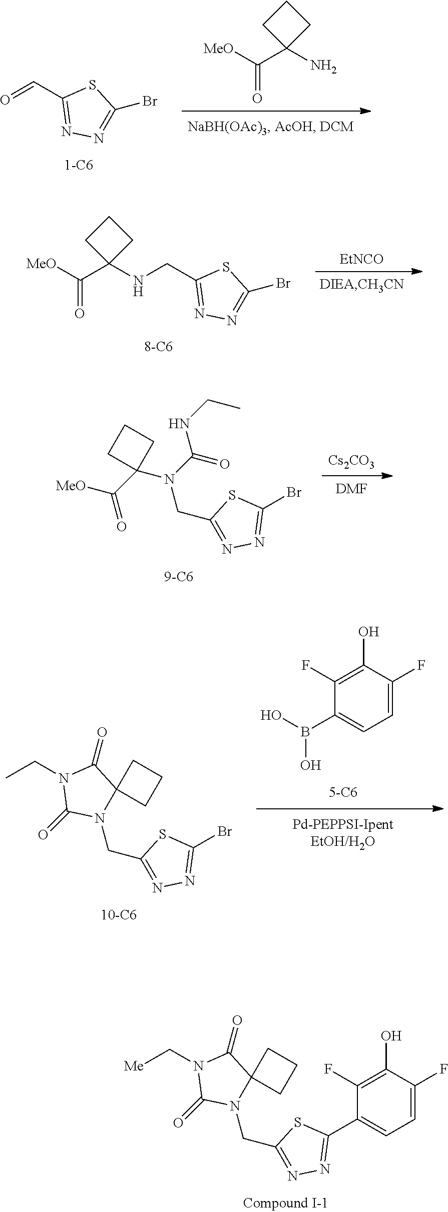

### Preparation of methyl 1-(((5-bromo-1,3,4-thiadiazol-2-yl)methyl)amino)cyclobutane-1-carboxylate Compound 8-C6

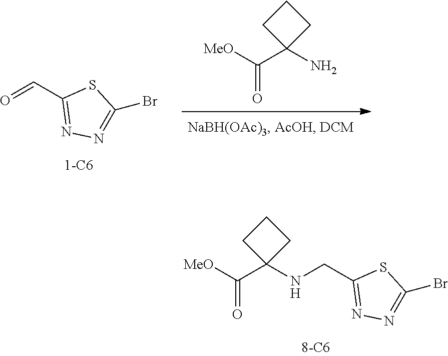 [SMILES: O=Cc1nnc(Br)s1 ; COC(=O)C1(N)CCC1 ; COC(=O)C1(NCc2nnc(Br)s2)CCC1]

To a solution of previously synthesized compound 1-C6 (1 g, 5.18 mmol, 1 eq) and 1-Aminocyclobutanecarboxylic acid [22264-50-2] (669.13 mg, 5.18 mmol, 1 eq) in DCM (15 mL) was added HOAc (466.67 mg, 7.77 mmol, 444.87 μL, 1.5 eq). After stirred at 0° C. for 1 h, NaBH(OAc)₃ (2.20 g, 10.36 mmol, 2 eq) was added to the mixture. The mixture was stirred at 25° C. for 12 h. The mixture was quenched with NaHCO₃ aqueous, and then extracted with DCM (20 mL×2). The organic layer was dried, filtered and concentrated. The residue was purified by column chromatography (SiO₂, petroleum ether:ethyl acetate=10:1 to 5:1) to give compound 8-C6 (610 mg, 1.99 mmol,) as a colorless oil.

¹H NMR (400 MHz, DMSO-d₆) δ 3.99 (d, J=7.6 Hz, 2H), 3.82-3.90 (m, 1H), 3.64 (s, 3H), 2.28-2.38 (m, 2H), 1.88-2.01 (m, 3H), 1.76-1.87 (m, 1H)

LCMS: RT=0.325 min, MS cal.: 305.0, MS found: [M+H]⁺=306.0, 308.0.

### Preparation of methyl 1-(1-((5-bromo-1,3,4-thiadiazol-2-yl)methyl)-3-ethylureido)cyclobutane-1-carboxylate Compound 9-C6

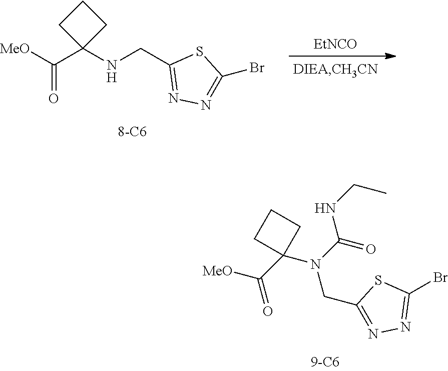 [SMILES: "EtNCO" ; COC(=O)C1(NCc2nnc(Br)s2)CCC1 ; "DIEA,CH3CN" ; CCNC(=O)N(Cc1nnc(Br)s1)C1(C(=O)OC)CCC1]

To a solution of compound 8-C6 (610 mg, 1.99 mmol, 1 eq) and isocyanatoethane (566.43 mg, 7.97 mmol, 630.77 μL, 4 eq) in CH₃CN (10 mL) was added DIEA (1.03 g, 7.97 mmol, 1.39 mL, 4 eq). The mixture was stirred at 60° C. for 12 h under sealed tube. After removed the solvent, the mixture was then extracted with DCM (20 mL×2). The organic layer was dried, filtered and concentrated. The residue was purified by column chromatography (SiO₂, petroleum ether:ethyl acetate=10:1 to 1:1) to give compound 9-C6 (310 mg, 821.72 μmol,) as a white solid.

¹H NMR (400 MHz, DMSO-d₆) δ 6.20 (t, J=5.2 Hz, 1H), 4.70 (s, 2H), 3.61 (s, 3H), 2.98-3.07 (m, 2H), 2.51-2.55 (m, 1H), 2.46-2.49 (m, 1H), 2.21-2.34 (m, 2H), 1.87-1.98 (m, 1H), 1.67-1.81 (m, 1H), 0.97 (t, J=7.2 Hz, 3H) LCMS: RT=0.393 min, MS cal.: 376.0, MS found: [M+H]⁺=376.9, 309.0.

### Preparation of 5-((5-bromo-1,3,4-thiadiazol-2-yl)methyl)-7-ethyl-5,7-diazaspiro[3.4]octane-6,8-dione Compound 10-C6

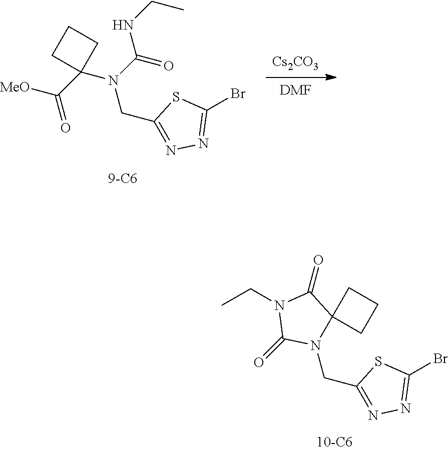

To a solution of compound 9-C6 (310 mg, 821.72 μmol, 1 eq) in DMF (5 mL) was added Cs₂CO₃ (535.46 mg, 1.64 mmol, 2 eq). The mixture was stirred at 25° C. for 1 h. The mixture was poured into water (10 mL), and then extracted with DCM (20 mL×2). The organic layer was dried, filtered and concentrated. The residue was purified by column chromatography (SiO₂, petroleum ether:ethyl acetate=10:1 to 1:1) to give compound 10-C6 (152 mg, 440.31 μmol,) as a colorless oil.

¹H NMR (400 MHz, DMSO-d₆) δ 5.11-5.18 (m, 2H), 3.37-3.47 (m, 2H), 2.51-2.59 (m, 2H), 2.17-2.29 (m, 2H), 1.90-2.05 (m, 1H), 1.70-1.84 (m, 1H), 1.04-1.15 (m, 3H)

LCMS: RT=0.395 min, MS cal.: 344.0, MS found: [M+H]⁺=345.0, 347.0.

### Preparation of 5-((5-(2,4-difluoro-3-hydroxyphenyl)-1,3,4-thiadiazol-2-yl)methyl)-7-ethyl-5,7-diazaspiro[3.4]octane-6,8-dione Compound I-1

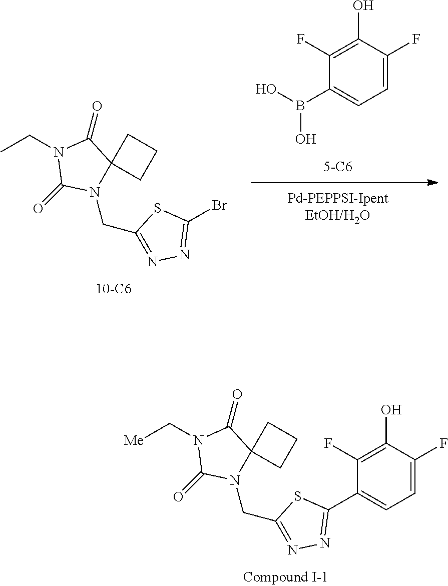 [SMILES: CCN1C(=O)N(Cc2nnc(Br)s2)C2(CCC2)C1=O ; OB(O)c1ccc(F)c(O)c1F ; CCN1C(=O)N(Cc2nnc(-c3ccc(F)c(O)c3F)s2)C2(CCC2)C1=O]

To a solution of compound 10-C6 (300 mg, 869.02 μmol, 1 eq) in ethanol (5 mL) and H₂O (1 mL) was added previously synthesized compound 5-C6 (226.70 mg, 1.30 mmol, 1.5 eq) and Na₂CO₃ (184.21 mg, 1.74 mmol, 2 eq). After purged N₂ for three times, 1,3-bis[2,6-bis(1-ethylpropyl)phenyl]-2H-imidazole; 3-chloropyridine; dichloropalladium (68.97 mg, 86.90 μmol, 0.1 eq) was added. The mixture was stirred at 80° C. for 2 hr under N₂ atmosphere. The mixture was poured into water (10 mL), and then extracted with DCM (20 mL×2). The organic layer was dried, filtered and concentrated. The residue was purified by prep-HPLC (column: Phenomenex Luna C18 100*30 mm*3 um; mobile phase: [H₂O (0.2% formic acid)-ACN]; gradient: 20%-50% B over 8.0 min) to give Compound I-1 (60 mg, 152.13 μmol,) as white solid.

¹H NMR (400 MHz, DMSO-d₆) δ (ppm) 8.40 (s, 1H), 7.22 (s, 1H), 7.01 (t, J=9.6 Hz, 1H), 5.18 (s, 2H), 3.48-3.42 (m, 2H), 2.63-2.53 (m, 2H), 2.30-2.19 (m, 2H), 2.06-1.91 (m, 1H), 1.85-1.73 (m, 1H), 1.10 (t, J=7.2 Hz, 3H)

LCMS: RT=2.261 min, MS calc.: 394.1, MS found: [M+H]⁺=395.1.

HPLC: RT=2.475 min, purity: 96.528%.

### Route to Metabolite:

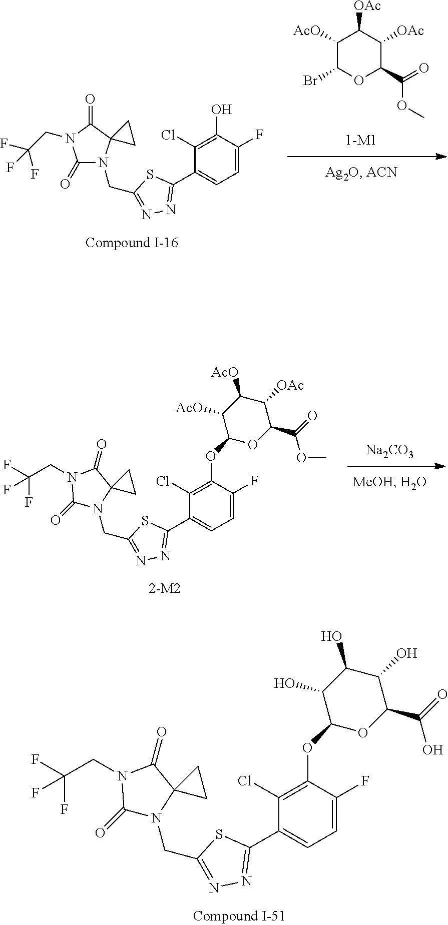

### Preparation of (2S,3R,4S,5S,6S)-2-(2-chloro-3-(5-((5,7-dioxo-6-(2,2,2-trifluoroethyl)-4,6-diazaspiro[2.4]heptan-4-yl)methyl)-1,3,4-thiadiazol-2-yl)-6-fluorophenoxy)-6-(methoxycarbonyl)tetrahydro-2H-pyran-3,4,5-triyl triacetate Compound I-M2

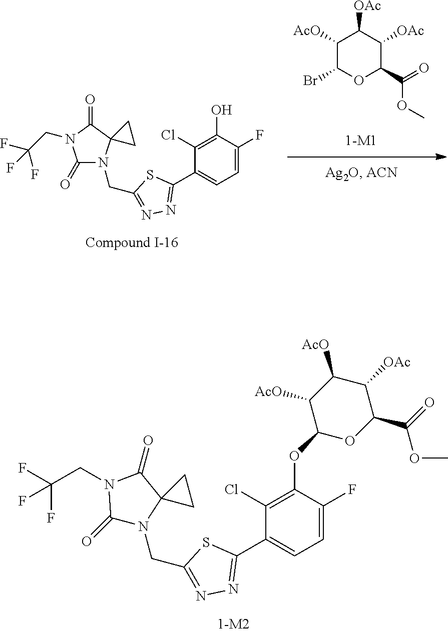 [SMILES: O=C1N(CC(F)(F)F)C(=O)C2(CC2)N1Cc1nnc(-c2ccc(F)c(O)c2Cl)s1 ; COC(=O)[C@H]1O[C@H](Br)[C@H](OC(C)=O)[C@@H](OC(C)=O)[C@@H]1OC(C)=O ; COC(=O)[C@H]1O[C@@H](Oc2c(F)ccc(-c3nnc(CN4C(=O)N(CC(F)(F)F)C(=O)C45CC5)s3)c2Cl)[C@H](OC(C)=O)[C@@H](OC(C)=O)[C@@H]1OC(C)=O]

To a solution of Compound I-16 (200 mg, 443.66 μmol, 1 eq) and compound 1-M1 (704.84 mg, 1.77 mmol, 4 eq) in ACN (4 mL) was added Ag₂O (257.03 mg, 1.11 mmol, 2.5 eq) under N₂. Then the mixture was stirred at 15° C. for 1 h. LCMS showed desired mass was detected. The reaction was quenched by addition of ethyl acetate (10 mL) and water (10 mL). The organic layer was separated, washed with brine (5 mL), dried over anhydrous Na₂SO₄, filtered and concentrated in vacuum. The residue was purified by prep-TLC to get compound 1-M2 (320 mg, 417.18 μmol, crude) as a yellow solid.

LCMS: RT=0.522 min, MS cal.: 766.1, MS found: [M+H]⁺=767.2.

### Preparation of (2S,3S,4S,5R,6S)-6-(2-chloro-3-(5-((5,7-dioxo-6-(2,2,2-trifluoroethyl)-4,6-diazaspiro[2.4]heptan-4-yl)methyl)-1,3,4-thiadiazol-2-yl)-6-fluorophenoxy)-3,4,5-trihydroxytetrahydro-2H-pyran-2-carboxylic acid Compound I-51

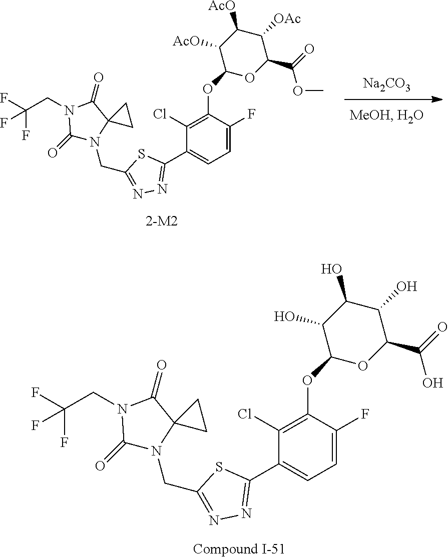

To a solution of compound 2-M2 (320 mg, 417.18 μmol, 1 eq) in methanol (10 mL) and H₂O (2 mL) was added Na₂CO₃ (88.43 mg, 834.36 μmol, 2 eq) at 15° C. under N₂. Then the mixture was stirred at 15° C. for 72 h. The mixture was concentrated in vacuum to remove most of methanol. The residue was dissolved in water and purified by prep-HPLC (column: Phenomenex luna C18 100*40 mm*5 um; mobile phase: [H₂O (0.2% formic acid)-ACN]; gradient: 10%-45% B over 8.0 min) to get Compound I-51 (70.34 mg, 112.20 μmol) as a white solid.

LCMS: RT=1.967 min, MS calc.: 626.0, MS found: [M+H]⁺=627.0.

¹H NMR (400 MHz, DMSO-d₆) δ 12.70 (br s, 1H), 7.94 (dd, J=5.6, 8.8 Hz, 1H), 7.52 (t, J=9.6 Hz, 1H), 5.58 (br d, J=5.2 Hz, 1H), 5.25 (br d, J=5.2 Hz, 1H), 5.04 (d, J=7.6 Hz, 1H), 4.97 (s, 2H), 4.35 (q, J=9.2 Hz, 2H), 3.68 (d, J=10.0 Hz, 1H), 3.45-3.38 (m, 1H), 3.37-3.34 (m, 1H), 3.30-3.25 (m, 1H), 1.73-1.64 (m, 2H), 1.39-1.30 (m, 2H)

The following compounds in Table 7 were made in a similar manner to the procedures described above. In general, the final compounds were purified prior to biological testing to achieve a purity >95% as determined by HPLC.

TABLE 7
List of specific examples of Compounds of Formula I:

| Compound | Structure | NMR (LC-MS MH⁺) |
|---|---|---|
| I-2 | 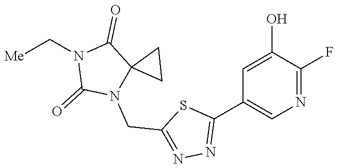 [SMILES: CCN1C(=O)N(Cc2nnc(-c3cnc(F)c(O)c3)s2)C2(CC2)C1=O] | 1H NMR (400 MHz, DMSO-d6) δ 11.24-10.93 (m, 1H), 8.20 (t, J = 2.0 Hz, 1H), 7.92 (dd, J = 2.0, 9.6 Hz, 1H), 4.89 (s, 2H), 3.56-3.47 (m, 2H), 1.61-1.54 (m, 2H), 1.28-1.23 (m, 2H), 1.14 (t, J = 7.2 Hz, 3H). (MH⁺ 364.1) |
| I-12 | 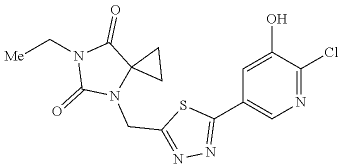 [SMILES: CCN1C(=O)N(Cc2nnc(-c3cnc(Cl)c(O)c3)s2)C2(CC2)C1=O] | 1H NMR (400 MHz, DMSO-d6) δ 11.51-11.23 (br m, 1H), 8.42 (d, J = 2.0 Hz, 1H), 7.86 (d, J = 2.0 Hz, 1H), 4.90 (s, 2H), 3.51 (q, J = 7.2 Hz, 2H), 1.61-1.55 (m, 2H), 1.30-1.22 (m, 2H), 1.14 (t, J = 7.2 Hz, 3H). (MH⁺ 380.1) |
| I-18 | 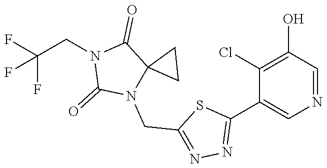 [SMILES: O=C1N(CC(F)(F)F)C(=O)C2(CC2)N1Cc1nnc(-c2cncc(O)c2Cl)s1] | 1H NMR (400 MHz, DMSO-d6) δppm 11.35 (br s, 1H) 8.67 (s, 1H) 8.39 (s, 1H) 4.99 (s, 2H) 4.35 (q, J = 9.2 Hz, 2H) 1.74-1.62 (m, 2H) 1.32-1.39 (m, 2H). (MH⁺ 434.0) |
| I-22 | 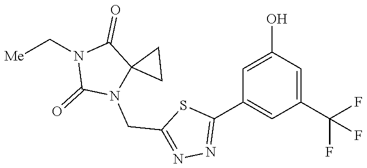 [SMILES: CCN1C(=O)N(Cc2nnc(-c3cc(O)cc(C(F)(F)F)c3)s2)C2(CC2)C1=O] | 1H NMR (400 MHz, DMSO-d6) δ 10.84-10.53 (br m, 1H), 7.66 (s, 1H), 7.62 (s, 1H), 7.23 (s, 1H), 4.89 (s, 2H), 3.52 (q, J = 7.2 Hz, 2H), 1.61-1.55 (m, 2H), 1.31-1.22 (m, 2H), 1.14 (t, J = 7.2 Hz, 3H). (MH⁺ 413.0) |
| I-28 | 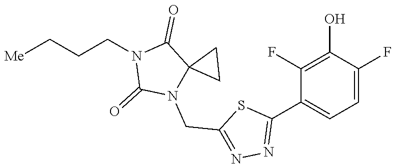 [SMILES: CCCCN1C(=O)N(Cc2nnc(-c3ccc(F)c(O)c3F)s2)C2(CC2)C1=O] | 1H NMR (400 MHz, DMSO-d6) δ 10.91-10.65 (br m, 1H), 7.73-7.53 (m, 1H), 7.33-7.17 (m, 1H), 4.90 (s, 2H), 3.48 (t, J = 7.2 Hz, 2H), 1.59-1.49 (m, 4H), 1.32-1.21 (m, 4H), 0.88 (t, J = 7.2 Hz, 3H). (MH⁺ 409.1) |
| I-29 | 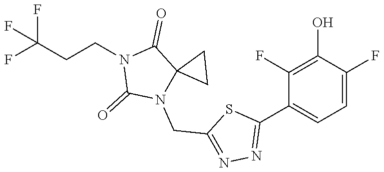 [SMILES: O=C1N(CCC(F)(F)F)C(=O)C2(CC2)N1Cc1nnc(-c2ccc(F)c(O)c2F)s1] | 1H NMR (400 MHz, DMSO-d6) δ 7.32-7.23 (m, 1H), 7.08-7.01 (m, 1H), 4.90 (s, 2H), 3.74 (t, J = 6.8 Hz, 2H), 2.72-2.58 (m, 2H), 1.64-1.57 (m, 2H), 1.29-1.23 (m, 2H). (MH⁺ 449.0) |
| I-30 | 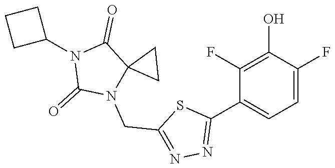 [SMILES: O=C1N(C2CCC2)C(=O)C2(CC2)N1Cc1nnc(-c2ccc(F)c(O)c2F)s1] | 1H NMR (400 MHz, DMSO-d6) δ 10.81 (br s, 1H), 7.65 (ddd, J = 6.0, 7.6, 8.8 Hz, 1H), 7.38-7.17 (m, 1H), 4.88 (s, 2H), 4.53 (quin, J = 8.8 Hz, 1H), 2.82-2.69 (m, 2H), 2.18-2.07 (m, 2H), 1.78-1.65 (m, 2H), 1.60-1.53 (m, 2H), 1.28-1.20 (m, 2H). (MH⁺ 407.0) |
| I-31 | 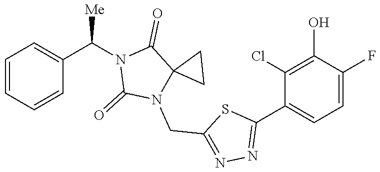 [SMILES: C[C@H](c1ccccc1)N1C(=O)N(Cc2nnc(-c3ccc(F)c(O)c3Cl)s2)C2(CC2)C1=O] | 1H NMR (400 MHz, DMSO-d6) δ 11.32-10.81 (br m, 1H), 7.59 (dd, J = 5.6, 8.8 Hz, 1H), 7.42-7.26 (m, 6H), 5.34 (q, J = 7.2 Hz, 1H), 4.91 (s, 2H), 1.78 (d, J = 7.2 Hz, 3H), 1.61-1.55 (m, 2H), 1.31-1.21 (m, 2H). (MH⁺ 473.0) |
| I-32 | 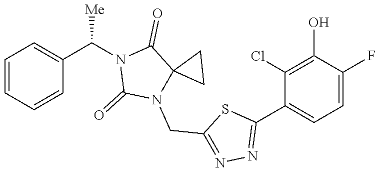 [SMILES: C[C@@H](c1ccccc1)N1C(=O)N(Cc2nnc(-c3ccc(F)c(O)c3Cl)s2)C2(CC2)C1=O] | 1H NMR (400 MHz, DMSO-d6) δ 10.97 (br s, 1H), 7.60 (dd, J = 5.6, 8.8 Hz, 1H), 7.46-7.24 (m, 6H), 5.34 (q, J = 7.2 Hz, 1H), 4.91 (s, 2H), 1.78 (d, J = 7.6 Hz, 3H), 1.64-1.54 (m, 2H), 1.32-1.21 (m, 2H). (MH⁺ 473.0) |
| I-33 | 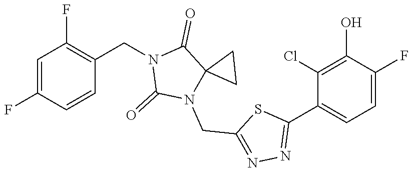 [SMILES: O=C1N(Cc2ccc(F)cc2F)C(=O)C2(CC2)N1Cc1nnc(-c2ccc(F)c(O)c2Cl)s1] | 1H NMR (400 MHz, DMSO-d6) δ 10.96 (br s, 1H), 7.61 (dd, J = 5.2, 8.8 Hz, 1H), 7.46-7.34 (m, 2H), 7.30-7.20 (m, 1H), 7.07 (dt, J = 1.6, 8.4 Hz, 1H), 4.93 (s, 2H), 4.70 (s, 2H), 1.66-1.57 (m, 2H), 1.34-1.26 (m, 2H). (MH⁺ 495.0) |
| I-29 | 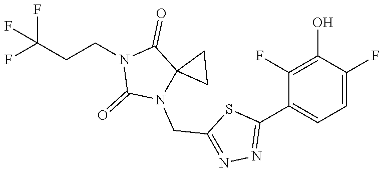 [SMILES: O=C1N(CCC(F)(F)F)C(=O)C2(CC2)N1Cc1nnc(-c2ccc(F)c(O)c2F)s1] | 1H NMR (400 MHz, DMSO-d6) δ 7.32-7.23 (m, 1H), 7.08-7.01 (m, 1H), 4.90 (s, 2H), 3.74 (t, J = 6.8 Hz, 2H), 2.72-2.58 (m, 2H), 1.64-1.57 (m, 2H), 1.29-1.23 (m, 2H). (MH⁺ 449.0) |
| I-30 | 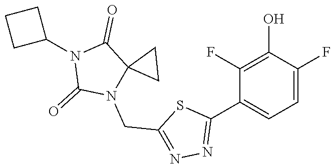 [SMILES: O=C1N(C2CCC2)C(=O)C2(CC2)N1Cc1nnc(-c2ccc(F)c(O)c2F)s1] | 1H NMR (400 MHz, DMSO-d6) δ 10.81 (br s, 1H), 7.65 (ddd, J = 6.0, 7.6, 8.8 Hz, 1H), 7.38-7.17 (m, 1H), 4.88 (s, 2H), 4.53 (quin, J = 8.8 Hz, 1H), 2.82-2.69 (m, 2H), 2.18-2.07 (m, 2H), 1.78-1.65 (m, 2H), 1.60-1.53 (m, 2H), 1.28-1.20 (m, 2H). (MH⁺ 407.0) |
| I-31 | 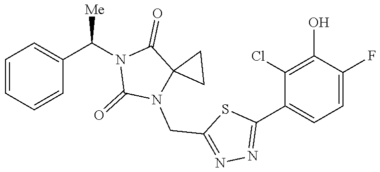 [SMILES: C[C@H](c1ccccc1)N1C(=O)N(Cc2nnc(-c3ccc(F)c(O)c3Cl)s2)C2(CC2)C1=O] | 1H NMR (400 MHz, DMSO-d6) δ 11.32-10.81 (br m, 1H), 7.59 (dd, J = 5.6, 8.8 Hz, 1H), 7.42-7.26 (m, 6H), 5.34 (q, J = 7.2 Hz, 1H), 4.91 (s, 2H), 1.78 (d, J = 7.2 Hz, 3H), 1.61-1.55 (m, 2H), 1.31-1.21 (m, 2H). (MH⁺ 473.0) |
| I-32 | 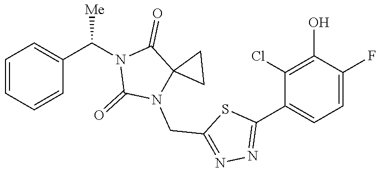 [SMILES: C[C@@H](c1ccccc1)N1C(=O)N(Cc2nnc(-c3ccc(F)c(O)c3Cl)s2)C2(CC2)C1=O] | 1H NMR (400 MHz, DMSO-d6) δ 10.97 (br s, 1H), 7.60 (dd, J = 5.6, 8.8 Hz, 1H), 7.46-7.24 (m, 6H), 5.34 (q, J = 7.2 Hz, 1H), 4.91 (s, 2H), 1.78 (d, J = 7.6 Hz, 3H), 1.64-1.54 (m, 2H), 1.32-1.21 (m, 2H). (MH⁺ 473.0) |
| I-33 | 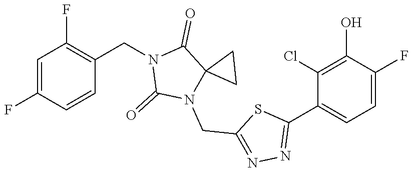 [SMILES: O=C1N(Cc2ccc(F)cc2F)C(=O)C2(CC2)N1Cc1nnc(-c2ccc(F)c(O)c2Cl)s1] | 1H NMR (400 MHz, DMSO-d6) δ 10.96 (br s, 1H), 7.61 (dd, J = 5.2, 8.8 Hz, 1H), 7.46-7.34 (m, 2H), 7.30-7.20 (m, 1H), 7.07 (dt, J = 1.6, 8.4 Hz, 1H), 4.93 (s, 2H), 4.70 (s, 2H), 1.66-1.57 (m, 2H), 1.34-1.26 (m, 2H). (MH⁺ 495.0) |
| I-36 | 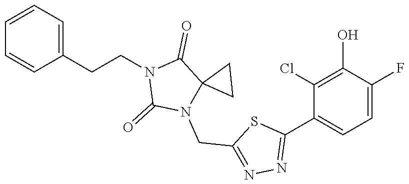 [SMILES: O=C1N(CCc2ccccc2)C(=O)C2(CC2)N1Cc1nnc(-c2ccc(F)c(O)c2Cl)s1] | 1H NMR (400 MHz, DMSO-d6) δ 10.99 (br s, 1H), 7.63-7.53 (m, 1H), 7.41 (d, J = 10.0 Hz, 1H), 7.28-7.18 (m, 2H), 7.18-7.08 (m, 3H), 4.87 (s, 2H), 3.71 (t, J = 7.2 Hz, 2H), 2.88 (t, J = 7.2 Hz, 2H), 1.63-1.51 (m, 2H), 1.25-1.12 (m, 2H). (MH⁺ 473.0) |
| I-38 | 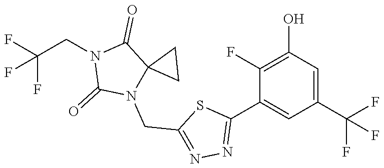 [SMILES: O=C1N(CC(F)(F)F)C(=O)C2(CC2)N1Cc1nnc(-c2cc(C(F)(F)F)cc(O)c2F)s1] | 1H NMR (400 MHz, DMSO-d6) δ 11.60-11.21 (br m, 1H), 7.90 (d, J = 3.2 Hz, 1H), 7.42 (dd, J = 1.6, 7.6 Hz, 1H), 4.99 (s, 2H), 4.35 (q, J = 9.2 Hz, 2H), 1.72-1.63 (m, 2H), 1.40-1.28 (m, 2H). (MH⁺ 485.0) |
| I-39 | 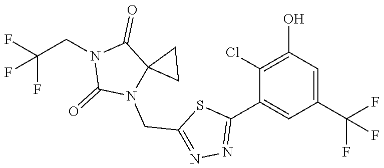 [SMILES: O=C1N(CC(F)(F)F)C(=O)C2(CC2)N1Cc1nnc(-c2cc(C(F)(F)F)cc(O)c2Cl)s1] | 1H NMR (400 MHz, DMSO-d6) δ 11.57 (br s, 1H), 7.88 (s, 1H), 7.41 (s, 1H), 4.99 (s, 2H), 4.35 (q, J = 9.2 Hz, 2H), 1.73-1.64 (m, 2H), 1.39-1.29 (m, 2H). (MH⁺ 501.0) |
| I-40 | 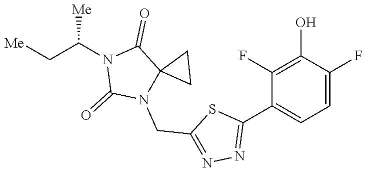 [SMILES: CC[C@H](C)N1C(=O)N(Cc2nnc(-c3ccc(F)c(O)c3F)s2)C2(CC2)C1=O] | 1H NMR (400 MHz, DMSO-d6) δ 10.78 (br s, 1H), 7.66 (ddd, J = 6.0, 7.6, 8.8 Hz, 1H), 7.31-7.22 (m, 1H), 4.90 (s, 2H), 4.02 (td, J = 6.0, 9.6 Hz, 1H), 1.89 (ddd, J = 7.6, 9.6, 13.6 Hz, 1H), 1.71-1.61 (m, 1H), 1.59-1.52 (m, 2H), 1.34 (d, J = 7.2 Hz, 3H), 1.27-1.21 (m, 2H), 0.80 (t, J = 7.6 Hz, 3H). (MH⁺ 409.1) |
| I-41 | 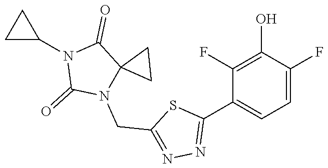 [SMILES: O=C1N(C2CC2)C(=O)C2(CC2)N1Cc1nnc(-c2ccc(F)c(O)c2F)s1] | 1H NMR (400 MHz, DMSO-d6) δ 10.90-10.68 (br m, 1H), 7.65 (ddd, J = 6.0, 7.6, 9.6 Hz, 1H), 7.26 (dt, J = 2.0, 9.6 Hz, 1H), 4.86 (s, 2H), 2.69-2.60 (m, 1H), 1.58-1.52 (m, 2H), 1.23-1.18 (m, 2H), 0.88-0.82 (m, 4H). (MH⁺ 393.0) |
| I-42 | 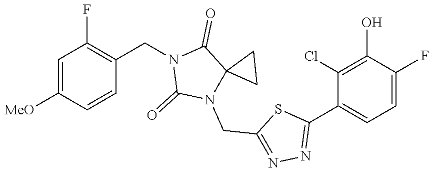 [SMILES: COc1ccc(CN2C(=O)N(Cc3nnc(-c4ccc(F)c(O)c4Cl)s3)C3(CC3)C2=O)c(F)c1] | 1H NMR (400 MHz, DMSO-d6) δ 10.96 (br s, 1H), 7.60 (dd, J = 5.6, 9.2 Hz, 1H), 7.45-7.36 (m, 1H), 7.24 (t, J = 8.8 Hz, 1H), 6.85-6.72 (m, 2H), 4.92 (s, 2H), 4.65 (s, 2H), 3.75 (s, 3H), 1.64-1.56 (m, 2H), 1.32-1.24 (m, 2H). (MH⁺ 507.0) |
| I-43 | 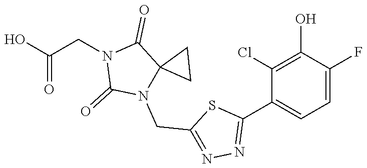 [SMILES: O=C(O)CN1C(=O)N(Cc2nnc(-c3ccc(F)c(O)c3Cl)s2)C2(CC2)C1=O] | 1H NMR (400 MHz, DMSO-d6) δ 7.59 (dd, J = 5.2, 8.4 Hz, 1H), 7.39 (dd, J = 8.8, 10.0 Hz, 1H), 4.94 (s, 2H), 4.16 (s, 2H), 1.69-1.62 (m, 2H), 1.34-1.25 (m, 2H). (MH⁺ 427.0) |
| I-44 | 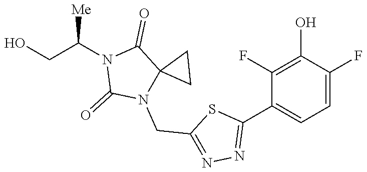 [SMILES: C[C@H](CO)N1C(=O)N(Cc2nnc(-c3ccc(F)c(O)c3F)s2)C2(CC2)C1=O] | 1H NMR (400 MHz, MeOD) δ 10.97 (br s, 1H), 7.66 (ddd, J = 5.6, 7.6, 9.2 Hz, 1H), 7.29-7.21 (m, 1H), 4.88 (d, J = 2.0 Hz, 2H), 4.16-4.06 (m, 1H), 3.80 (dd, J = 9.6, 11.2 Hz, 1H), 3.56 (dd, J = 5.2, 11.2 Hz, 1H), 1.61-1.56 (m, 2H), 1.29-1.18 (m, 5H). (MH ⁺ 411.1) |
| I-45 | 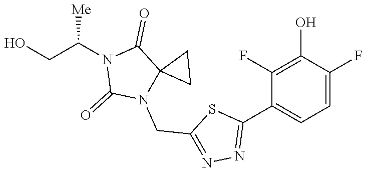 [SMILES: C[C@@H](CO)N1C(=O)N(Cc2nnc(-c3ccc(F)c(O)c3F)s2)C2(CC2)C1=O] | 1H NMR (400 MHz, MeOD) δ 7.69 (ddd, J = 6.0, 7.6, 8.8 Hz, 1H), 7.23-7.00 (m, 1H), 4.98-4.88 (m, 1H), 4.87 (d, J = 2.0 Hz, 1H), 4.31 (ddd, J = 5.2, 7.2, 9.2 Hz, 1H), 4.00 (dd, J = 9.6, 11.2 Hz, 1H), 3.66 (dd, J = 5.2, 11.2 Hz, 1H), 1.62-1.55 (m, 2H), 1.32-1.19 (m, 5H). (MH ⁺ 411.1) |
| I-46 | 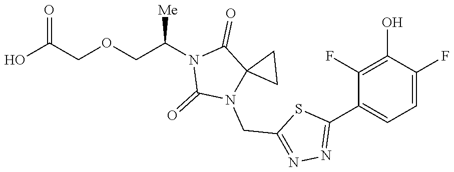 [SMILES: C[C@H](COCC(=O)O)N1C(=O)N(Cc2nnc(-c3ccc(F)c(O)c3F)s2)C2(CC2)C1=O] | 1H NMR (400 MHz, DMSO-d6) δ 10.78 (br s, 1H), 7.66 (ddd, J = 6.0, 7.6, 9.0 Hz, 1H), 7.31-7.21 (m, 1H), 4.89 (s, 2H), 4.38-4.28 (m, 1H), 4.04-3.93 (m, 2H), 3.88-3.84 (m, 1H), 3.66-3.62 (m, 1H), 1.61-1.54 (m, 2H), 1.29 (d, J = 7.2 Hz, 3H), 1.26-1.21 (m, 2H). (MH⁺ 469.1) |
| I-47 | 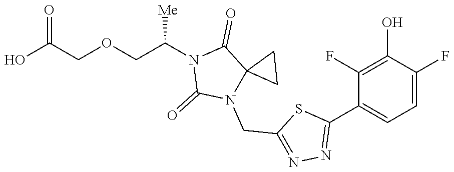 [SMILES: C[C@@H](COCC(=O)O)N1C(=O)N(Cc2nnc(-c3ccc(F)c(O)c3F)s2)C2(CC2)C1=O] | 1H NMR (400 MHz, DMSO-d6) δ 7.68-7.62 (m, 1H), 7.37-7.16 (m, 1H), 4.89 (s, 2H), 4.37-4.25 (m, 1H), 4.04-3.91 (m, 2H), 3.86 (t, J = 9.6 Hz, 1H), 3.63-3.58 (m, 1H), 1.61-1.54 (m, 2H), 1.29 (d, J = 7.2 Hz, 3H), 1.26-1.19 (m, 2H). (MH⁺ 469.1) |
| I-49 | 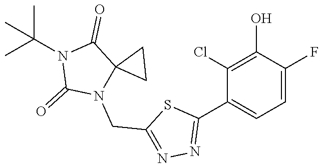 [SMILES: CC(C)(C)N1C(=O)N(Cc2nnc(-c3ccc(F)c(O)c3Cl)s2)C2(CC2)C1=O] | 1H NMR (400 MHz, DMSO-d6) δ 10.96 (br s, 1H), 7.59 (dd, J = 5.2, 8.8 Hz, 1H), 7.40 (dd, J = 8.8, 10.0 Hz, 1H), 4.85 (s, 2H), 1.56 (s, 9H), 1.53-1.48 (m, 2H), 1.22-1.16 (m, 2H). (MH⁺ 425.0) |
| I-52 | 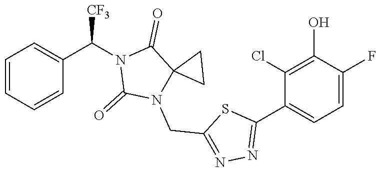 [SMILES: O=C1N([C@@H](c2ccccc2)C(F)(F)F)C(=O)C2(CC2)N1Cc1nnc(-c2ccc(F)c(O)c2Cl)s1] | 1H NMR (400 MHz, DMSO-d6) δ 10.99 (br s, 1H), 7.69-7.55 (m, 3H), 7.49-7.34 (m, 4H), 6.06 (q, J = 8.8 Hz, 1H), 4.97 (s, 2H), 1.67 (d, J = 2.4 Hz, 2H), 1.36 (s, 2H). (MH⁺ 527.0) |
| I-53 | 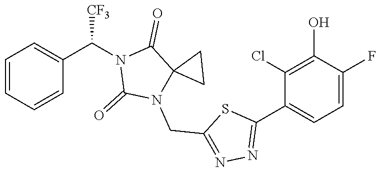 [SMILES: O=C1N([C@H](c2ccccc2)C(F)(F)F)C(=O)C2(CC2)N1Cc1nnc(-c2ccc(F)c(O)c2Cl)s1] | 1H NMR (400 MHz, DMSO-d6) δ 10.97 (br s, 1H), 7.73-7.51 (m, 3H), 7.51-7.33 (m, 4H), 6.06 (q, J = 8.8 Hz, 1H), 4.97 (s, 2H), 1.66 (s, 2H), 1.36 (s, 2H). (MH⁺ 527.0) |
| I-54 | 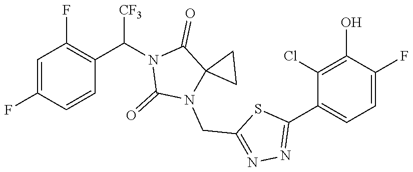 [SMILES: O=C1N(C(c2ccc(F)cc2F)C(F)(F)F)C(=O)C2(CC2)N1Cc1nnc(-c2ccc(F)c(O)c2Cl)s1] | 1H NMR (400 MHz, DMSO-d6) δ 10.97 (br s, 1H), 7.95-7.84 (m, 1H), 7.60 (dd, J = 5.6, 8.8 Hz, 1H), 7.45-7.35 (m, 2H), 7.23 (dt, J = 2.0, 8.8 Hz, 1H), 6.29 (q, J = 8.8 Hz, 1H), 4.95 (s, 2H), 1.70-1.63 (m, 2H), 1.40-1.33 (m, 2H). (MH⁺ 563.0) |
| I-55 | 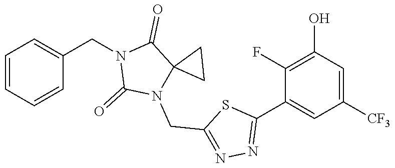 [SMILES: O=C1N(Cc2ccccc2)C(=O)C2(CC2)N1Cc1nnc(-c2cc(C(F)(F)F)cc(O)c2F)s1] | 1H NMR (400 MHz, DMSO-d6) δ 11.28 (br s, 1H), 7.90 (d, J = 3.2 Hz, 1H), 7.43 (dd, J = 2.0, 7.6 Hz, 1H), 7.39-7.24 (m, 5H), 4.97 (s, 2H), 4.68 (s, 2H), 1.69-1.55 (m, 2H), 1.37-1.22 (m, 2H). (MH ⁺ 493.0) |

We provide additional experimental details for specific examples from Table 7 as shown below.

### Route for I-31

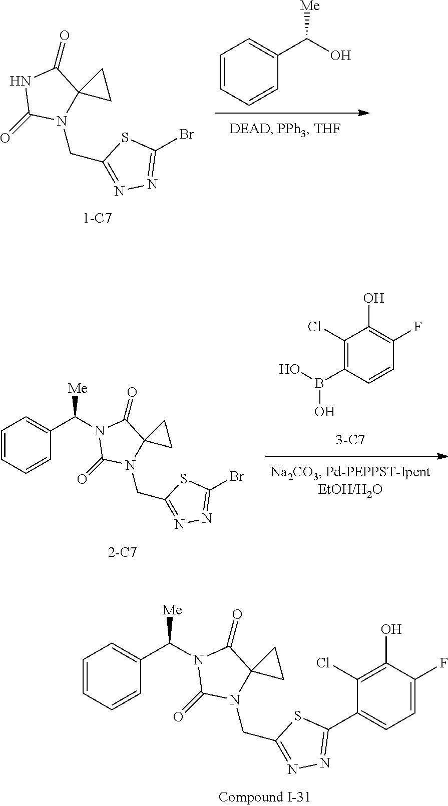 [SMILES: O=C1NC(=O)C2(CC2)N1Cc1nnc(Br)s1 ; C[C@H](O)c1ccccc1 ; C[C@H](c1ccccc1)N1C(=O)N(Cc2nnc(Br)s2)C2(CC2)C1=O ; OB(O)c1ccc(F)c(O)c1Cl ; C[C@H](c1ccccc1)N1C(=O)N(Cc2nnc(-c3ccc(F)c(O)c3Cl)s2)C2(CC2)C1=O]

### Preparation of 4-[(5-bromo-1, 3, 4-thiadiazol-2-yl) methyl]-6-[(1R)-1-phenylethyl]-4, 6-diazaspiro [2.4] heptane-5, 7-dione Compound 2-C7

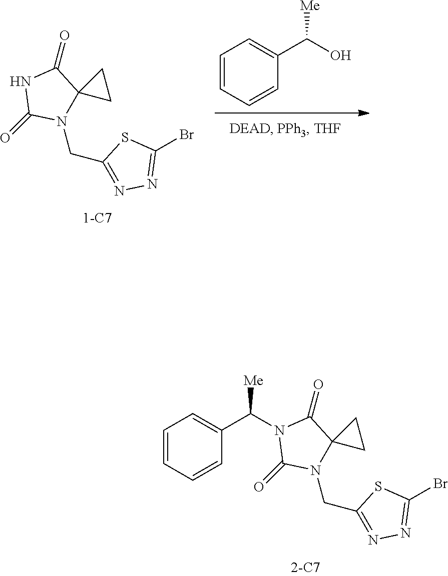 [SMILES: O=C1NC(=O)C2(CC2)N1Cc1nnc(Br)s1 ; C[C@H](O)c1ccccc1 ; C[C@H](c1ccccc1)N1C(=O)N(Cc2nnc(Br)s2)C2(CC2)C1=O]

To a solution of Compound 1-C7 (300 mg, 989.66 μmol, 1 eq) and (S)-1-phenylethan-1-ol (241.80 mg, 1.98 mmol, 238.93 μL, 2 eq) in THF (3 mL) was added diethyl azodicarboxylate (DEAD, 344.71 mg, 1.98 mmol, 359.82 μL, 2 eq) and triphenylphosphine (PPh₃, 519.15 mg, 1.98 mmol, 2 eq) at 20° C., then the reaction mixture was stirred at 20° C. for 5 h under N₂. TLC (petroleum ether:ethyl acetate=1:1, R_{f}=0.7) showed the reaction was completed. The reaction mixture was quenched by addition water (3 mL) at 20° C., and extracted with ethyl acetate (3×5 mL). The combined organic layers were washed with brine (15 mL), dried over Na₂SO₄, filtered and concentrated under reduced pressure to give a residue. The residue was purified by column chromatography (silica gel, petroleum ether:ethyl acetate=1:1) to afford Compound 2-C7 (250 mg, 580.68 μmol, 94.6% purity) as white solid.

¹H NMR (400 MHz, DMSO-d₆) δ 7.37-7.27 (m, 5H), 5.31 (d, J=7.2 Hz, 1H), 4.83 (s, 2H), 1.76 (d, J=7.6 Hz, 3H), 1.54-1.51 (m, 2H), 1.26-1.22 (m, 2H)

### Preparation of 4-[[5-(2-chloro-4-fluoro-3-hydroxy-phenyl)-1,3,4-thiadiazol-2-yl]methyl]-6-[(1R)-1-phenylethyl]-4,6-diazaspiro[2.4]heptane-5,7-dione Compound I-31

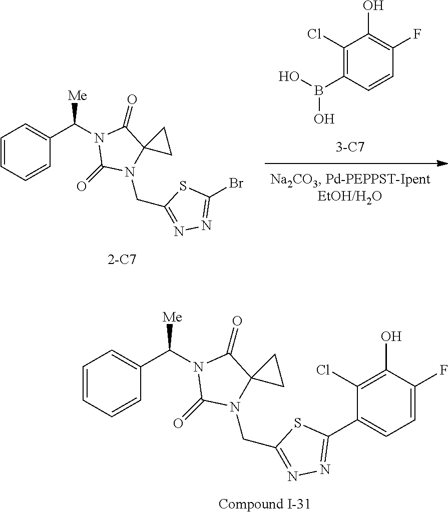 [SMILES: C[C@H](c1ccccc1)N1C(=O)N(Cc2nnc(Br)s2)C2(CC2)C1=O ; OB(O)c1ccc(F)c(O)c1Cl ; C[C@H](c1ccccc1)N1C(=O)N(Cc2nnc(-c3ccc(F)c(O)c3Cl)s2)C2(CC2)C1=O]

To a solution of Compound 2-C7 (250 mg, 613.82 μmol, 1 eq) and Compound 3-C7 (116.85 mg, 613.82 μmol, 1 eq) in EtOH (4 mL) and H₂O (1 mL) was added Na₂CO₃ (97.59 mg, 920.73 μmol, 1.5 eq) and 1,3-bis[2,6-bis(1-ethylpropyl)phenyl]-4,5-dichloro-2H-imidazol-1-ium-2-ide; 3-chloropyridine; dichloropalladium (52.82 mg, 61.38 μmol, 0.1 eq) at 25° C., then the reaction mixture was stirred at 80° C. for 1 hr under N₂. LC-MS showed the desired compound was detected. The reaction mixture was quenched by addition water (5 mL) at 20° C., and extracted with ethyl acetate (3×5 mL). The combined organic layers were washed with brine (15 mL), dried over Na₂SO₄, filtered and concentrated under reduced pressure to give a residue. The residue was purified by prep-HPLC (column: Phenomenex luna C18 100×40 mm×5 um; mobile phase: [H₂O (0.2% FA)-ACN]; gradient: 25%-65% B over 8.0 min) to afford Compound I-31 (67.75 mg, 143.26 μmol) as white solid.

¹H NMR (400 MHz, DMSO-d₆) δ 11.32-10.81 (br m, 1H), 7.58 (J=5.6, 8.8 Hz, 1H), 7.42-7.26 (m, 6H), 5.34 (q, J=7.2 Hz, 1H), 4.91 (s, 2H), 1.78 (d, J=7.2 Hz, 3H), 1.61-1.55 (m, 2H), 1.31-1.21 (m, 2H)

LCMS: RT=2.426 min, MS cal.: 472.1, MS found: [M+H]⁺=473.0

### Route for I-32

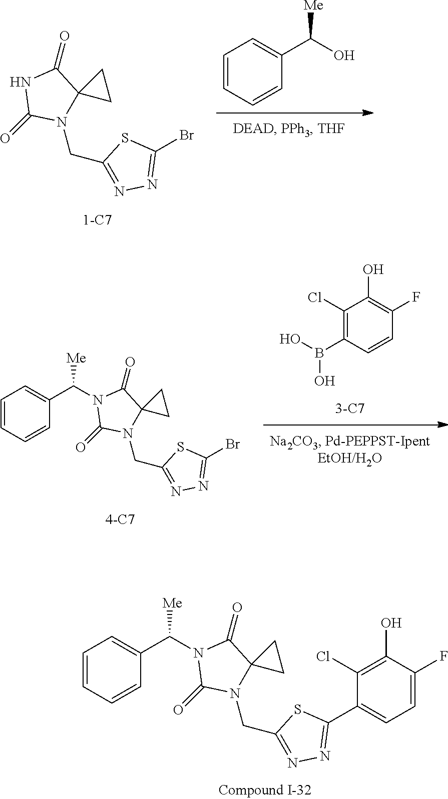 [SMILES: O=C1NC(=O)C2(CC2)N1Cc1nnc(Br)s1 ; C[C@@H](O)c1ccccc1 ; C[C@@H](c1ccccc1)N1C(=O)N(Cc2nnc(Br)s2)C2(CC2)C1=O ; OB(O)c1ccc(F)c(O)c1Cl ; C[C@@H](c1ccccc1)N1C(=O)N(Cc2nnc(-c3ccc(F)c(O)c3Cl)s2)C2(CC2)C1=O]

### Preparation of (S)-4-((5-bromo-1,3,4-thiadiazol-2-yl)methyl)-6-(1-phenylethyl)-4,6-diazaspiro[2.4]heptane-5,7-dione Compound 4-C7

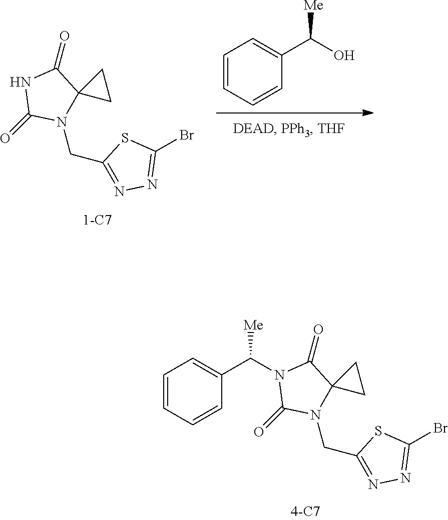 [SMILES: O=C1NC(=O)C2(CC2)N1Cc1nnc(Br)s1 ; C[C@@H](O)c1ccccc1 ; C[C@@H](c1ccccc1)N1C(=O)N(Cc2nnc(Br)s2)C2(CC2)C1=O]

To a solution of compound 1-C7 (300 mg, 989.66 μmol, 1 eq) and (R)-1-phenylethan-1-ol (145.08 mg, 1.19 mmol, 143.36 μL, 1.2 eq) in THF (5 mL) was added triphenylphosphine (PPh₃, 519.15 mg, 1.98 mmol, 2 eq). After cooled to 0° C., diethyl azodicarboxylate (DEAD, 344.70 mg, 1.98 mmol, 359.81 μL, 2 eq) was added. The mixture was stirred at 20° C. for 2 h under N₂ atmosphere. LC-MS showed compound 1-C7 was consumed completely and desired mass was detected. The mixture was concentrated under reduced pressure. The residue was purified by prep-TLC (petroleum ether:ethyl acetate=7:3) to afford compound 4-C7 (100 mg, 245.53 μmol,) as brown solid.

¹H NMR (400 MHz, CHLOROFORM-d) δ 7.50 (d, J=7.2 Hz, 2H), 7.39-7.29 (m, 3H), 5.43 (q, J=7.6 Hz, 1H), 4.70-4.59 (m, 2H), 1.89 (d, J=7.2 Hz, 3H), 1.45-1.40 (m, 4H)

### Preparation of (S)-4-((5-(2-chloro-4-fluoro-3-hydroxyphenyl)-1,3,4-thiadiazol-2-yl)methyl)-6-(1-phenylethyl)-4,6-diazaspiro[2.4]heptane-5,7-dione Compound 1-32

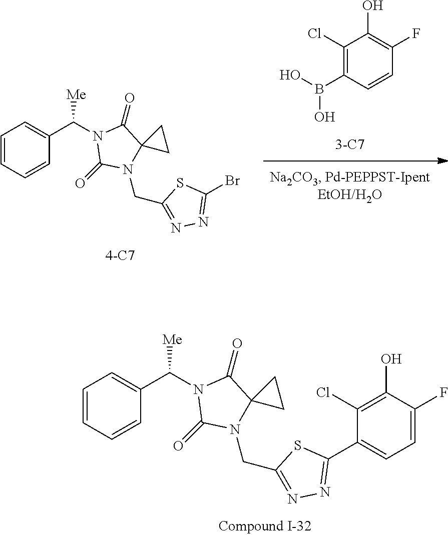 [SMILES: C[C@@H](c1ccccc1)N1C(=O)N(Cc2nnc(Br)s2)C2(CC2)C1=O ; OB(O)c1ccc(F)c(O)c1Cl ; C[C@@H](c1ccccc1)N1C(=O)N(Cc2nnc(-c3ccc(F)c(O)c3Cl)s2)C2(CC2)C1=O]

To a solution of compound 4-C7 (100 mg, 245.53 μmol, 1 eq) and compound 3-C7 (56.09 mg, 294.63 μmol, 1.2 eq) in ethanol (2 mL) and H₂O (0.5 mL) was added Na₂CO₃ (39.04 mg, 368.29 mol, 1.5 eq). After purged N₂ for three times, 1,3-bis[2,6-bis(1-ethylpropyl)phenyl]-4,5-dichloro-2H-imidazol-1-ium-2-ide; 3-chloropyridine; dichloropalladium (Pd-PEPPST-Ipent, 10.56 mg, 12.28 μmol, 0.05 eq) was added. The mixture was at 80° C. for 1 h under N₂ atmosphere. LC-MS showed reactant was consumed completely and desired mass was detected. After filtration, the filtrate was concentrated under reduced pressure. The pH of the residue was adjusted to 4 using TFA. The mixture was extracted with EtOAc (10 mL×2). The organic layer was dried, filtered and concentrated under reduced pressure. The residue was purified by prep-HPLC (column: Phenomenex Luna C18 75×30 mm×3 um; mobile phase: [H₂O (0.1% TFA)-ACN]; gradient: 35%-65% B over 8.0 min) to afford Compound I-32 (63 mg, 133.22 μmol,) as white solid.

¹H NMR (400 MHz, DMSO-d₆) δ 10.97 (br s, 1H), 7.60 (dd, J=5.6, 8.8 Hz, 1H), 7.46-7.24 (m, 6H), 5.34 (q, J=7.2 Hz, 1H), 4.91 (s, 2H), 1.78 (d, J=7.6 Hz, 3H), 1.64-1.54 (m, 2H), 1.32-1.21 (m, 2H)

LCMS: RT=2.579 min, MS cal.: 472.1, MS found: [M+H]⁺=473.0.

### Route for I-33

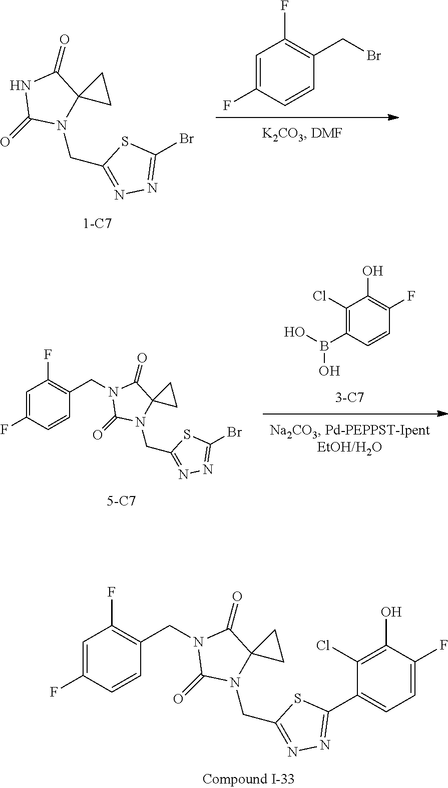 [SMILES: O=C1NC(=O)C2(CC2)N1Cc1nnc(Br)s1 ; Fc1ccc(CBr)c(F)c1 ; O=C1N(Cc2ccc(F)cc2F)C(=O)C2(CC2)N1Cc1nnc(Br)s1 ; OB(O)c1ccc(F)c(O)c1Cl ; O=C1N(Cc2ccc(F)cc2F)C(=O)C2(CC2)N1Cc1nnc(-c2ccc(F)c(O)c2Cl)s1]

### Preparation of 4-((5-bromo-1,3,4-thiadiazol-2-yl)methyl)-6-(2,4-difluorobenzyl)-4,6-diazaspiro[2.4]heptane-5,7-dione Compound 5-C7

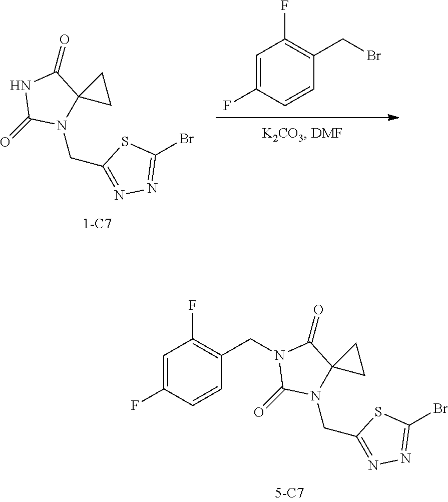 [SMILES: O=C1NC(=O)C2(CC2)N1Cc1nnc(Br)s1 ; Fc1ccc(CBr)c(F)c1 ; O=C1N(Cc2ccc(F)cc2F)C(=O)C2(CC2)N1Cc1nnc(Br)s1]

To a solution of Compound 1-C7 (0.3 g, 989.66 μmol, 1 eq) and 1-(bromomethyl)-2,4-difluorobenzene (245.85 mg, 1.19 mmol, 1.2 eq) in DMF (4.5 mL) was added K₂CO₃ (410.33 mg, 2.97 mmol, 3 eq) under N₂. Then the mixture was stirred at 60° C. for 2 h. The mixture was poured into water (10 mL) and extracted with EtOAc (15 mL×3). The combined organic layers were dried over Na₂SO₄, filtered and concentrated under reduced pressure. The residue was purified by column chromatography (SiO₂, petroleum ether:ethyl acetate=20:80) to afford Compound 5-C7 (350 mg, 815.40 μmol) as white solid.

¹H NMR (400 MHz, CDCl₃-d) δ 7.45-7.34 (m, 1H), 6.92-6.77 (m, 2H), 4.79 (s, 2H), 4.69 (s, 2H), 1.52-1.43 (m, 4H)

### Preparation of 4-((5-(2-chloro-4-fluoro-3-hydroxyphenyl)-1,3,4-thiadiazol-2-yl)methyl)-6-(2,4-difluorobenzyl)-4,6-diazaspiro[2.4]heptane-5,7-dione Compound I-33

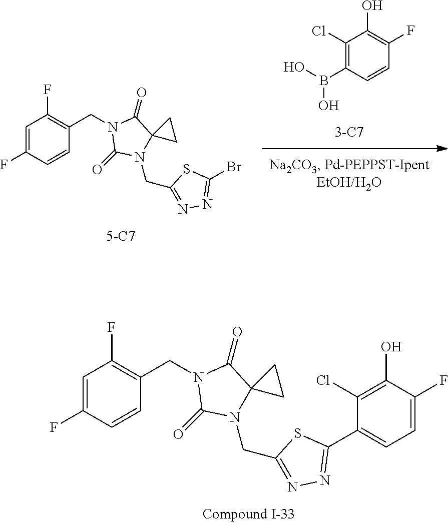 [SMILES: O=C1N(Cc2ccc(F)cc2F)C(=O)C2(CC2)N1Cc1nnc(Br)s1 ; OB(O)c1ccc(F)c(O)c1Cl ; O=C1N(Cc2ccc(F)cc2F)C(=O)C2(CC2)N1Cc1nnc(-c2ccc(F)c(O)c2Cl)s1]

To a solution of Compound 5-C7 (100 mg, 232.97 μmol, 1 eq) and Compound 3-C7 (53.22 mg, 279.56 μmol, 1.2 eq) in EtOH (1.6 mL) and H₂O (0.4 mL) was added Na₂CO₃ (37.04 mg, 349.46 mol, 1.5 eq) and 1,3-bis[2,6-bis(1-ethylpropyl)phenyl]-4,5-dichloro-2H-imidazol-1-ium-2-ide; 3-chloropyridine; dichloropalladium (10.02 mg, 11.65 μmol, 0.05 eq). The mixture was stirred at 80° C. for 1 h under N₂. The mixture was poured into water (10 mL) and extracted with ethyl acetate (15 mL×3). The combined organic layers were dried over Na₂SO₄, filtered and concentrated under reduced pressure. The residue was purified by prep-HPLC (column: Phenomenex Luna C18 80×30 mm×3 um; mobile phase: [H₂O (0.1% TFA)-ACN]; gradient: 25%-55% B over 8.0 min) to afford Compound I-33 (87.2 mg, 175.71 μmol, 99.72% purity) as white solid.

¹H NMR (400 MHz, DMSO-d₆) δ 10.96 (br s, 1H), 7.61 (dd, J=5.2, 8.8 Hz, 1H), 7.46-7.34 (m, 2H), 7.30-7.20 (m, 1H), 7.07 (dt, J=1.6, 8.4 Hz, 1H), 4.93 (s, 2H), 4.70 (s, 2H), 1.66-1.57 (m, 2H), 1.34-1.26 (m, 2H)

LCMS: RT=2.505 min, MS cal.: 494.0, MS found: [M+H]⁺=495.0.

### Route for I-41

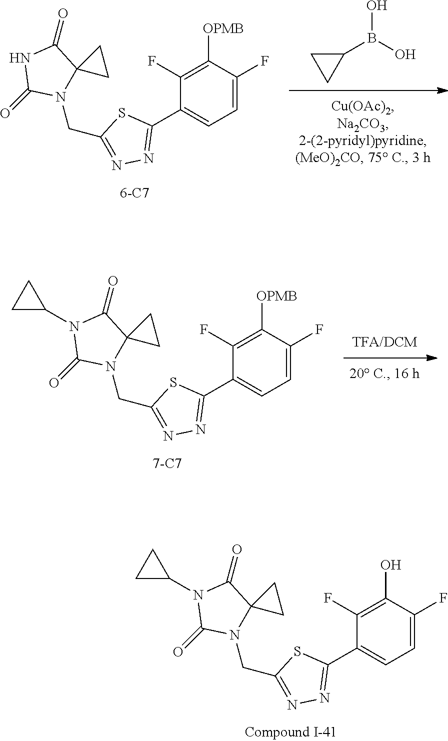

### Preparation of 6-cyclopropyl-4-[[5-[2,4-difluoro-3-[(4-methoxyphenyl)methoxy]phenyl]-1,3,4-thiadiazol-2-yl]methyl]-4,6-diazaspiro[2.4]heptane-5,7-dione Compound 7-C7

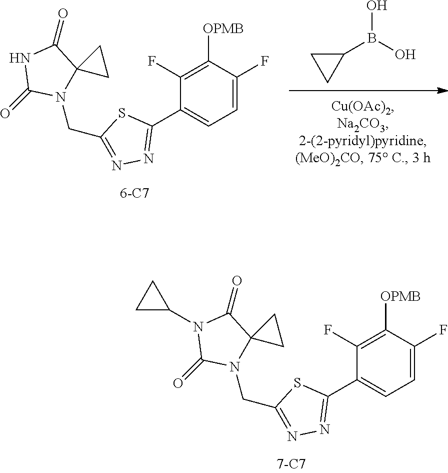

A mixture of 6-C7 (250 mg, 529.14 μmol, 1 eq), cyclopropylboronic acid (136.36 mg, 1.59 mmol, 3 eq), Na₂CO₃ (112.17 mg, 1.06 mmol, 2 eq), Cu(OAc)₂ (19.22 mg, 105.83 μmol, 0.2 eq) and 2-(2-pyridyl)pyridine (16.53 mg, 105.83 μmol, 0.2 eq) in dimethyl carbonate (1.5 mL) was degassed and purged with 02 for 3 times, and then the mixture was stirred at 75° C. for 3 h under 02 atmosphere. TLC (petroleum ether:ethyl acetate=1:1, Rt=0.43) showed the starting material was consumed completely. The reaction mixture was filtered. The reaction mixture was quenched by addition H₂O (50 mL×2) at 20° C., and extracted with ethyl acetate (50 mL×2). The combined organic layers were washed with brine (50 mL×2), dried over Na₂SO₄, filtered and concentrated under reduced pressure to give a residue. The residue was purified by prep-TLC (SiO₂, petroleum ether:ethyl acetate=1:1) to give compound 7-C7 (140 mg, 273.16 μmol,) as white solid.

¹H NMR (400 MHz, CDCl₃) δ 8.01 (ddd, J=6.0, 7.6, 9.2 Hz, 1H), 7.37 (d, J=8.4 Hz, 2H), 7.04 (dt, J=1.6, 9.6 Hz, 1H), 6.93-6.86 (m, 2H), 5.17 (s, 2H), 4.77 (s, 2H), 3.82 (s, 3H), 2.74-2.65 (m, 1H), 1.53-1.49 (m, 2H), 1.43-1.38 (m, 2H), 1.04-0.99 (m, 4H).

### Preparation of 6-cyclopropyl-4-[[5-(2,4-difluoro-3-hydroxy-phenyl)-1,3,4-thiadiazol-2-yl]methyl]-4,6-diazaspiro[2.4]heptane-5,7-dione Compound I-41

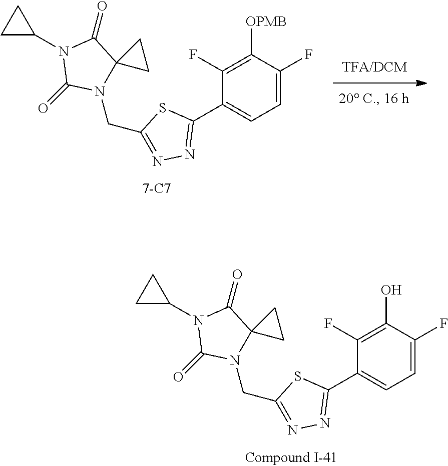

To a solution of compound 7-C7 (120 mg, 234.13 μmol, 1 eq) in trifluoroacetic acid (1.5 mL) and dichloromethane (1.5 mL). The mixture was stirred at 20° C. for 16 h. LC-MS showed the reaction was completed. The reaction mixture was concentrated under reduced pressure to give a residue. The residue was purified by prep-HPLC (column: Phenomenex Luna C18 100×40 mm×3 um; mobile phase: [H₂O (0.1% TFA)-ACN]; gradient: 15%-50% B over 8.0 min) to give Compound I-41 (57.6 mg, 146.80 μmol, 100% purity) as white solid.

¹H NMR (400 MHz, DMSO-d₆) δ 10.90-10.68 (br m, 1H), 7.65 (ddd, J=6.0, 7.6, 9.6 Hz, 1H), 7.26 (dt, J=2.0, 9.6 Hz, 1H), 4.86 (s, 2H), 2.69-2.60 (m, 1H), 1.58-1.52 (m, 2H), 1.23-1.18 (m, 2H), 0.88-0.82 (m, 4H).

LCMS: RT=2.153 min, MS cal.: 392.1, MS found: [M+H]+=393.0.

### Route for I-49

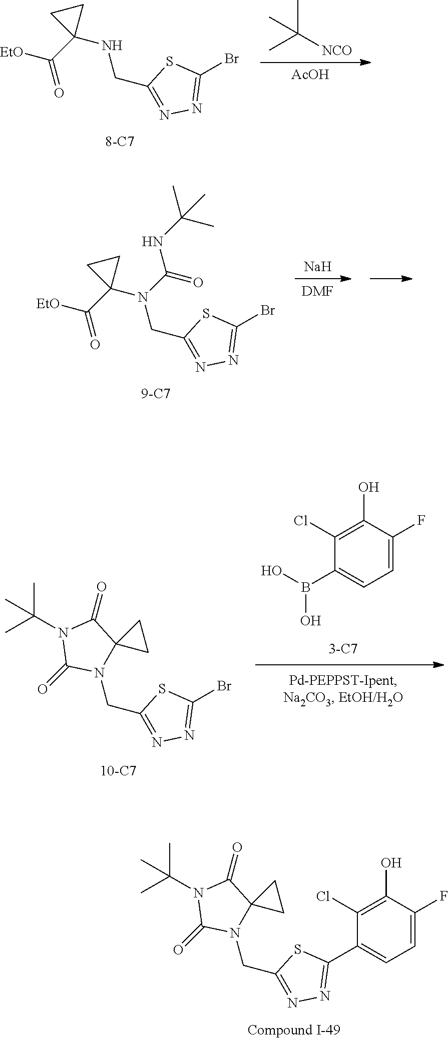

### Preparation of ethyl 1-(1-((5-bromo-1,3,4-thiadiazol-2-yl)methyl)-3-(tert-butyl)ureido)cyclopropane-1-carboxylate Compound 9-C7

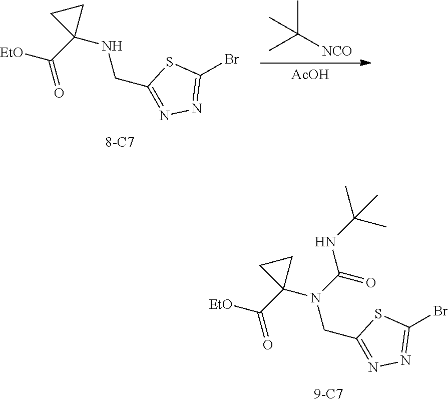

To a solution of Compound 8-C7 (800 mg, 2.61 mmol, 1 eq) in AcOH (8 mL) was added t-butyl isocyanate (518 mg, 5.23 mmol, 616.70 μL, 2 eq) at 25° C. The mixture was stirred at 90° C. for 1 h. The mixture was concentrated in reduced pressure at 40° C. The residue was adjusted to pH=7 with NaHCO₃ solution. The aqueous phase was extracted with dichloromethane (10 mL×3). The combined organic phase was washed with brine (10 mL), dried with anhydrous Na₂SO₄, filtered and concentrated in vacuum. The residue was purified by column chromatography (SiO₂, petroleum ether:ethyl acetate=9:1 to 1:1) to get Compound 9-C7 (890 mg, 2.20 mmol) as white solid.

¹H NMR (400 MHz, methanol-d₄) δ 5.34 (s, 1H), 4.80 (d, J=8.0 Hz, 2H), 4.15-3.91 (m, 2H), 1.82-1.72 (m, 1H), 1.65-1.55 (m, 1H), 1.53-1.39 (m, 2H), 1.35 (s, 9H), 1.13 (t, J=7.2 Hz, 3H).

### Procedure for preparation of 4-((5-bromo-1,3,4-thiadiazol-2-yl)methyl)-6-(tert-butyl)-4,6-diazaspiro[2.4]heptane-5,7-dione Compound 10-C7

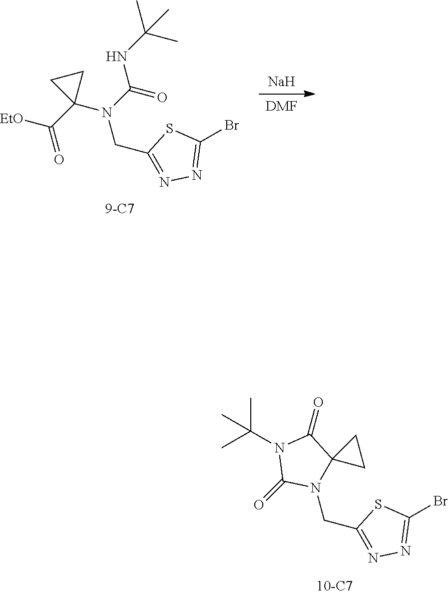

To a solution of Compound 9-C7 (630 mg, 1.55 mmol, 1 eq) in DMF (4 mL) was added NaH (93 mg, 2.33 mmol, 60% purity, 1.5 eq) at 0° C. The mixture was stirred at 15° C. for 0.5 h. The reaction mixture was quenched by addition of H₂O (5 mL) at 0° C., and then extracted with ethyl acetate (5 mL×3). The combined organic layers were dried over Na₂SO₄, filtered and concentrated under reduced pressure. The residue was purified by prep-TLC (SiO₂, petroleum ether:ethyl acetate=2:1) to get Compound 10C-7 (150 mg, 417.55 μmol) as yellow oil.

¹H NMR (400 MHz, methanol-d₄) δ 4.73 (s, 2H), 1.62 (s, 9H), 1.50-1.43 (m, 2H), 1.29-1.25 (m, 2H).

### Procedure for preparation of 6-(tert-butyl)-4-((5-(2-chloro-4-fluoro-3-hydroxyphenyl)-1,3,4-thiadiazol-2-yl)methyl)-4,6-diazaspiro[2.4]heptane-5,7-dione Compound 1-49

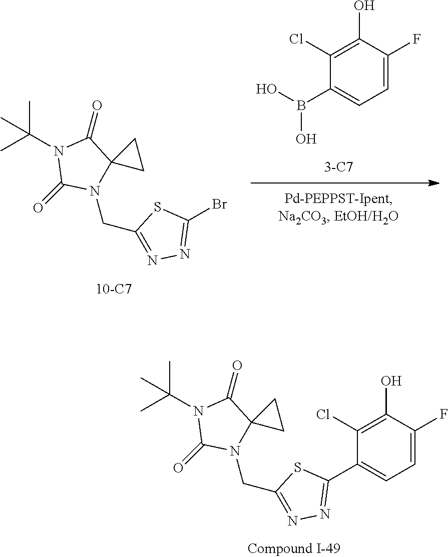 [SMILES: CC(C)(C)N1C(=O)N(Cc2nnc(Br)s2)C2(CC2)C1=O ; OB(O)c1ccc(F)c(O)c1Cl ; CC(C)(C)N1C(=O)N(Cc2nnc(-c3ccc(F)c(O)c3Cl)s2)C2(CC2)C1=O]

To a solution of Compound 10-C7 (100 mg, 278 μmol, 1 eq) in ethanol (0.8 mL), H₂O (0.2 mL) was added Compound 3-C7 (105 mg, 556 μmol, 2 eq), 1,3-bis[2,6-bis(1-ethylpropyl)phenyl]-4,5-dichloro-2H-imidazol-1-ium-2-ide; 3-chloropyridine; dichloropalladium (Pd-PEPPST-Ipent, 11.98 mg, 13.92 μmol, 0.05 eq) and Na₂CO₃ (44.2 mg, 417 μmol, 1.5 eq). The mixture was stirred at 80° C. for 1 h. The mixture was poured into water (3 mL) and extracted with EtOAc (3 mL×3). The combined organic layers were dried over Na₂SO₄, filtered and concentrated under reduced pressure. The residue was purified by prep-HPLC (column: Phenomenex Luna C18 80×30 mm×3 um; mobile phase: [H₂O (0.1% TFA)-ACN]; gradient: 40%-70% B over 8.0 min) to obtain Compound I-49 (56.9 mg, 131.71 μmol, 98.35% purity) as white solid.

¹H NMR (400 MHz, DMSO-d₆) δ 10.96 (br s, 1H), 7.59 (dd, J=5.2, 8.8 Hz, 1H), 7.40 (dd, J=8.8, 10.0 Hz, 1H), 4.85 (s, 2H), 1.56 (s, 9H), 1.53-1.48 (m, 2H), 1.22-1.16 (m, 2H).

LCMS: RT=2.351 min, MS cal.: 424.1, MS found: [M+H]⁺=425.0.

### Route for I-52

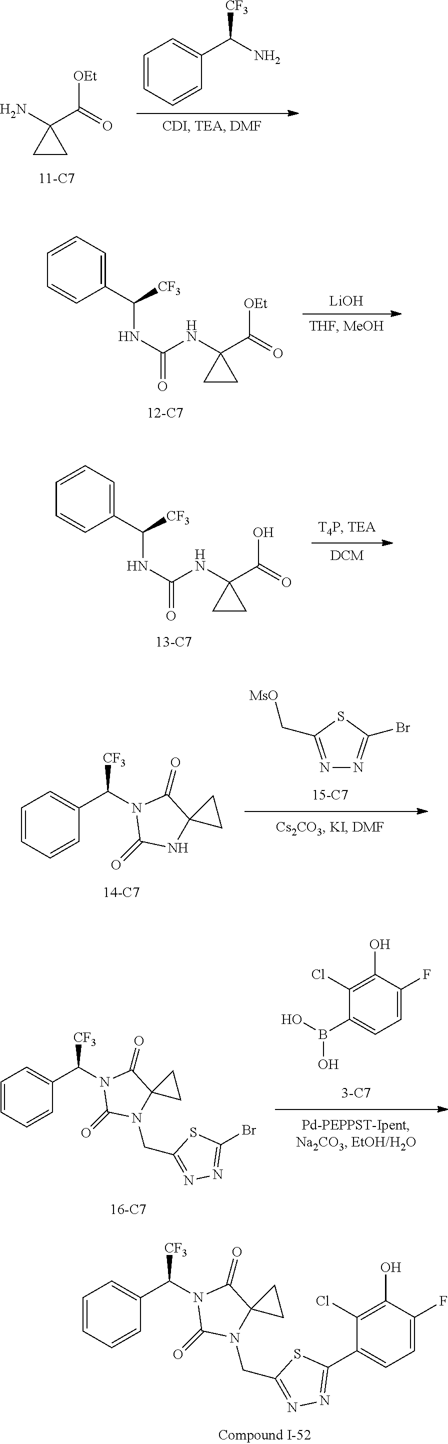

### Preparation of ethyl (S)-1-(3-(2,2,2-trifluoro-1-phenylethyl)ureido)cyclopropane-1-carboxylate Compound 12-C7

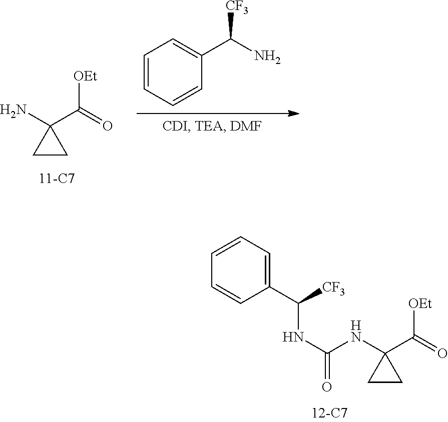

To a solution of Compound 11-C7 (499.05 mg, 2.85 mmol, 0.8 eq) in DMF (5 mL) was added TEA (1.80 g, 17.81 mmol, 2.48 mL, 5 eq) and CDI (606.38 mg, 3.74 mmol, 1.05 eq). The mixture was stirred at 25° C. for 10 min under N₂. Then (S)-2,2,2-trifluoro-1-phenylethan-1-amine (460 mg, 3.56 mmol, 1 eq) was added at 25° C. The mixture was stirred at 60° C. for 1 h under N₂. The residue was triturated with water (5 mL), filtered and the filter cake was concentrated under reduced pressure to afford Compound 12-C7 (800 mg, 2.42 mmol) as white solid.

¹H NMR (400 MHz, DMSO-d₆) δ 7.55-7.31 (m, 6H), 6.66 (s, 1H), 5.53 (quin, J=8.8 Hz, 1H), 4.05-3.91 (m, 2H), 1.35-1.26 (m, 2H), 1.10-0.98 (m, 5H)

### Preparation of (S)-1-(3-(2,2,2-trifluoro-1-phenylethyl)ureido)cyclopropane-1-carboxylic acid Compound 13-C7

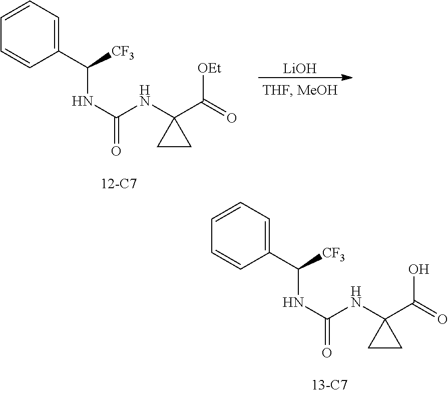 [SMILES: CCOC(=O)C1(NC(=O)N[C@@H](c2ccccc2)C(F)(F)F)CC1 ; "LiOH" ; O=C(N[C@@H](c1ccccc1)C(F)(F)F)NC1(C(=O)O)CC1]

To a solution of Compound 12-C7 (600 mg, 1.82 mmol, 1 eq) in THF (3 mL) and MeOH (1 mL) was added LiOH·H₂O (1 M, 1.82 mL, 1 eq). The mixture was stirred at 25° C. for 16 h under N₂. The mixture was poured into water (5 mL) and extracted with EtOAc (5 mL×3). The aqueous phase was adjusted to pH ˜4 with citric acid and extracted with EtOAc (5 mL×2). The combined organic layers were dried over Na₂SO₄, filtered and concentrated under reduced pressure to afford Compound 13-C7 (480 mg, 1.59 mmol,) as white solid.

¹H NMR (400 MHz, DMSO-d₆) δ 12.27 (br s, 1H), 7.52-7.37 (m, 5H), 7.33 (d, J=9.6 Hz, 1H), 6.60 (s, 1H), 5.52 (quin, J=8.8 Hz, 1H), 1.38-1.21 (m, 2H), 1.04-0.89 (m, 2H)

### Preparation of (S)-6-(2,2,2-trifluoro-1-phenylethyl)-4,6-diazaspiro[2.4]heptane-5,7-dione Compound 14-C7

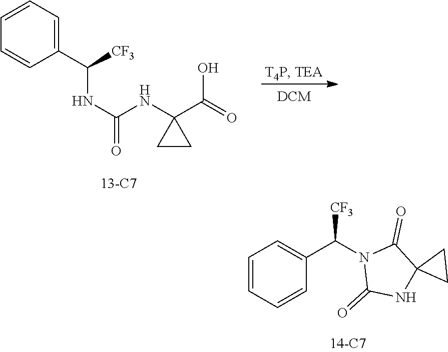 [SMILES: O=C(N[C@@H](c1ccccc1)C(F)(F)F)NC1(C(=O)O)CC1 ; "T4P, TEA" ; O=C1NC2(CC2)C(=O)N1[C@@H](c1ccccc1)C(F)(F)F]

To a solution of Compound 13-C7 (480 mg, 1.59 mmol, 1 eq) in DCM (5 mL) was added T₄P (2.29 g, 3.18 mmol, 50% purity, 2 eq) and TEA (803.49 mg, 7.94 mmol, 1.11 mL, 5 eq) at 0° C. The mixture was stirred at 15° C. for 1 hr under N₂. The mixture was poured into water (5 mL) and extracted with ethyl acetate (5 mL×3). The combined organic layers were dried over Na₂SO₄, filtered and concentrated under reduced pressure. The residue was purified by prep-TLC (SiO₂, petroleum ether:ethyl acetate=3:1, R_{f}=0.43) to afford Compound 14-C7 (280 mg, 985.11 μmol) as colorless oil.

¹H NMR (400 MHz, CDCl₃) δ 7.50-7.37 (m, 5H), 5.36 (q, J=7.6 Hz, 1H), 1.61-1.46 (m, 4H)

### Preparation of (S)-4-((5-bromo-1,3,4-thiadiazol-2-yl)methyl)-6-(2,2,2-trifluoro-1-phenylethyl)-4,6-diazaspiro[2.4]heptane-5,7-dione Compound 16-C7

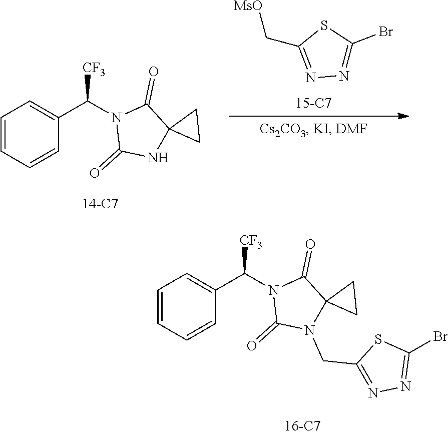 [SMILES: O=C1NC2(CC2)C(=O)N1[C@@H](c1ccccc1)C(F)(F)F ; CS(=O)(=O)OCc1nnc(Br)s1 ; O=C1N([C@@H](c2ccccc2)C(F)(F)F)C(=O)C2(CC2)N1Cc1nnc(Br)s1]

To a solution of Compound 14-C7 (280 mg, 985.11 μmol, 1 eq) and Compound 15-C7 (322.87 mg, 1.18 mmol, 1.2 eq) in DMF (3 mL) was added KI (163.53 mg, 985.11 μmol, 1 eq) and Cs₂CO₃ (641.93 mg, 1.97 mmol, 2 eq). The mixture was stirred at 25° C. for 1 h under N₂. LCMS showed Reactant was consumed completely and one main peak with desired mass was detected. The reaction mixture was diluted with H₂O (20 mL) and extracted with ethyl acetate (20 mL×2). Dried over Na₂SO₄, filtered and concentrated under reduced pressure to give a residue. The residue was purified by column chromatography (SiO₂, petroleum ether:ethyl acetate=1:0 to 1:1) to afford compound 16-C7 (350 mg, 758.80 μmol) as yellow oil.

LCMS: RT=1.321 min, MS cal.: 460.0, MS found: [M+H]⁺=461.0.

¹H NMR (400 MHz, DMSO-d₆) δ 7.71-7.56 (m, 2H), 7.36-7.51 (m, 3H), 6.04 (q, J=9.2 Hz, 1H), 4.89 (d, J=1.2 Hz, 2H), 1.56-1.67 (m, 2H), 1.28-1.39 (m, 2H).

### Preparation of 4-[[5-(2-chloro-4-fluoro-3-hydroxy-phenyl)-1,3,4-thiadiazol-2-yl]methyl]-6-[(1S)-2,2,2-trifluoro-1-phenyl-ethyl]-4,6-diazaspiro[2.4]heptane-5,7-dione Compound I-52

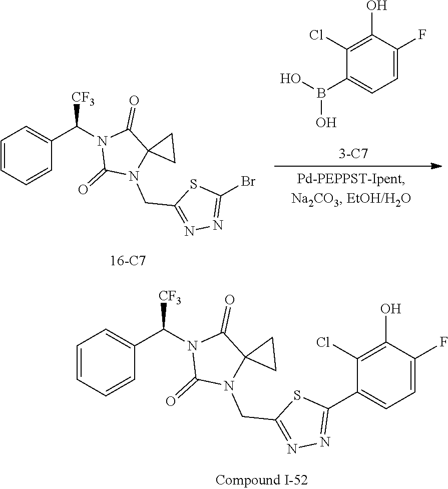 [SMILES: O=C1N([C@@H](c2ccccc2)C(F)(F)F)C(=O)C2(CC2)N1Cc1nnc(Br)s1 ; OB(O)c1ccc(F)c(O)c1Cl ; O=C1N([C@@H](c2ccccc2)C(F)(F)F)C(=O)C2(CC2)N1Cc1nnc(-c2ccc(F)c(O)c2Cl)s1]

A mixture of compound 16-C7 (50 mg, 108.40 μmol, 1 eq), compound 3-C7 (24.76 mg, 130.08 mol, 1.2 eq), 1,3-bis[2,6-bis(1-ethylpropyl) phenyl]-4,5-dichloro-2H-imidazol-1-ium-2-ide; 3-chloropyridine; dichloropalladium (Pd-PEPPST-Ipent, 4.66 mg, 5.42 μmol, 0.05 eq), Na₂CO₃ (17.23 mg, 162.60 μmol, 1.5 eq) in EtOH (0.8 mL) and H₂O (0.2 mL) was degassed and purged with Ar for 3 times, and then the mixture was stirred at 80° C. for 1 h under Ar atmosphere. LC-MS showed that reactant was consumed completely and one main peak with desired mass was detected. The reaction mixture was diluted with H₂O 10 mL and extracted with Ethyl acetate (10 mL×2). Dried over Na₂SO₄, filtered and concentrated under reduced pressure to give a residue. The residue was purified by prep-HPLC (column: Phenomenex luna C18 100×40 mm×5 um; mobile phase: [H₂O (0.2% FA)-ACN]; gradient: 45%-75% B over 8.0 min) to afford Compound I-52 (30.81 mg, 58.48 μmol) as white solid.

¹H NMR (400 MHz, DMSO-d₆) δ ppm 10.99 (br s, 1H), 7.55-7.69 (m, 3H), 7.49-7.34 (m, 4H), 6.06 (q, J=9.2 Hz, 1H), 4.97 (s, 2H), 1.67 (d, J=2.4 Hz, 2H), 1.36 (s, 2H).

LCMS: RT=2.692 min, MS cal.: 526.05, MS found: [M+H]⁺=527.0.

### Route for I-53

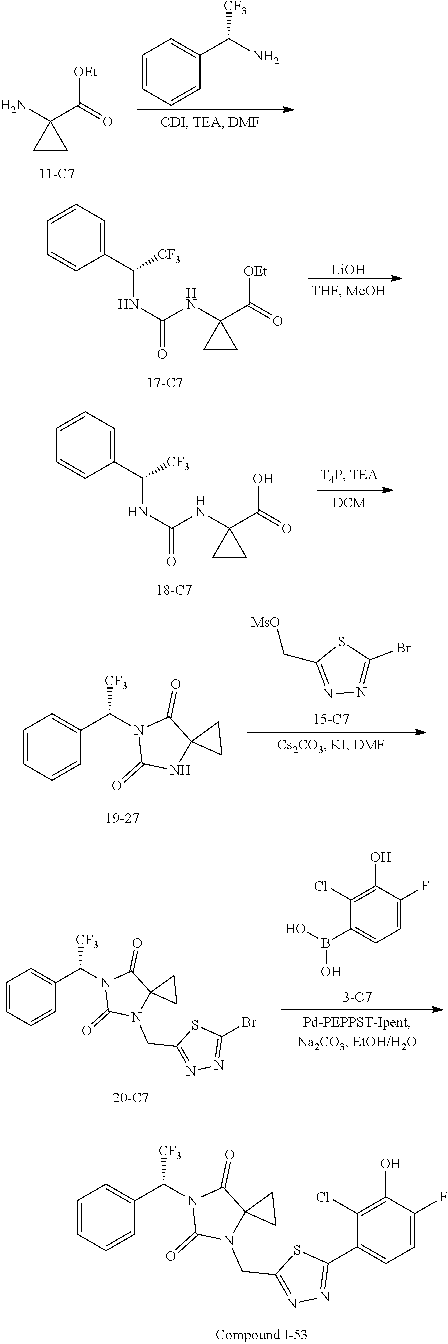

### Preparation of ethyl 1-[[(1R)-2,2,2-trifluoro-1-phenyl-ethyl]carbamoylamino]cyclopropanecarboxylate Compound 17-C7

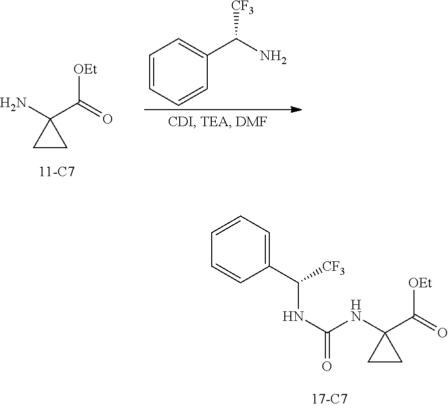 [SMILES: CCOC(=O)C1(N)CC1 ; N[C@H](c1ccccc1)C(F)(F)F ; CCOC(=O)C1(NC(=O)N[C@H](c2ccccc2)C(F)(F)F)CC1]

To a solution of compound 11-C7 (368.70 mg, 2.85 mmol, 1 eq) in DMF (5 mL) was added TEA (1.44 g, 14.27 mmol, 1.99 mL, 5 eq) and CDI (486.03 mg, 3.00 mmol, 1.05 eq) under N₂. The mixture was stirred at 15° C. for 0.5 hr under N₂. Then (R)-2,2,2-trifluoro-1-phenylethan-1-amine (500 mg, 2.85 mmol, 1 eq) was added. The mixture was stirred at 60° C. for 16 h under N₂. LCMS showed the reaction was completed. The reaction mixture was quenched by addition H₂O (20 mL) at 20° C., and filtered to afford compound 17-C7 (630 mg, 1.91 mmol) as white solid.

¹H NMR (400 MHz, DMSO-d₆) δ 7.49-7.38 (m, 6H), 6.66 (br s, 1H), 5.63-5.43 (m, 1H), 3.98 (dq, J=1.6, 6.8 Hz, 2H), 1.35-1.26 (m, 2H), 1.10-1.00 (m, 5H).

### Preparation of 1-[[(1R)-2,2,2-trifluoro-1-phenyl-ethyl]carbamoylamino]cyclopropanecarboxylic acid Compound 18-C7

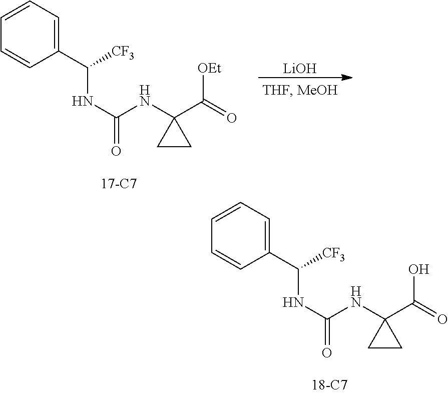 [SMILES: CCOC(=O)C1(NC(=O)N[C@H](c2ccccc2)C(F)(F)F)CC1 ; "LiOH" ; O=C(N[C@H](c1ccccc1)C(F)(F)F)NC1(C(=O)O)CC1]

A mixture of compound 17-C7 (630.00 mg, 1.91 mmol, 1 eq), LiOH·H₂O (1 M, 2.00 mL, 1.05 eq) in THF (6 mL), MeOH (2 mL) was degassed and purged with N₂ for 3 times, and then the mixture was stirred at 25° C. for 16 h under N₂ atmosphere. LCMS showed the reaction was completed. The reaction mixture was quenched by addition H₂O (30 mL×2) at 20° C., and extracted with EtOAc (30 mL×2). The aqueous phase was adjusted to pH=3 with citric acid solution, and extracted with EA (30 mL×2). The combined organic layers were washed with brine (30 mL×2), dried over Na₂SO₄, filtered and concentrated under reduced pressure to afford compound 18-C7 (490 mg, 1.62 mmol) as white solid.

¹H NMR (400 MHz, DMSO-d₆) δ 12.35-12.22 (br m, 1H), 7.49-7.39 (m, 5H), 7.33 (br d, J=10.0 Hz, 1H), 6.60 (br s, 1H), 5.52 (t, J=8.8 Hz, 1H), 1.34-1.24 (m, 2H), 1.02-0.95 (m, 2H)

### Preparation of 6-[(1R)-2,2,2-trifluoro-1-phenyl-ethyl]-4,6-diazaspiro[2.4]heptane-5,7-dione Compound 19-C7

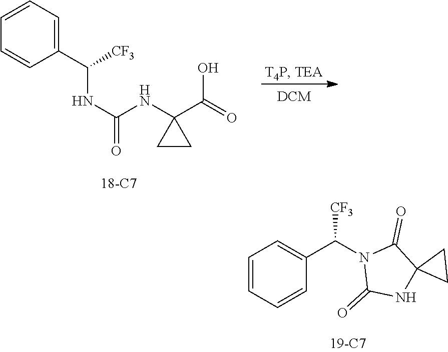 [SMILES: O=C(N[C@H](c1ccccc1)C(F)(F)F)NC1(C(=O)O)CC1 ; "T4P, TEA" ; O=C1NC2(CC2)C(=O)N1[C@H](c1ccccc1)C(F)(F)F]

A mixture of compound 18-C7 (490 mg, 1.62 mmol, 1 eq), triethylamine (1.31 g, 12.97 mmol, 1.81 mL, 8 eq) in DCM (5 mL) was degassed and purged with N₂ for 3 times, T₄P (3.50 g, 4.86 mmol, 50% purity, 3 eq) was added at 0° C. and then the mixture was stirred at 15° C. for 3 h under N₂ atmosphere. TLC (petroleum ether:ethyl acetate=2:1, Rt=0.8) showed the starting material was consumed completely. The reaction mixture was quenched by addition H₂O (20 mL×2) at 20° C., and extracted with dichloromethane (20 mL×2). The combined organic layers were washed with brine (20 mL×2), dried over Na₂SO₄, filtered and concentrated under reduced pressure to give a residue. The residue was purified by column chromatography (SiO₂, petroleum ether:ethyl acetate=85:15) to afford compound 19-C7 (115 mg, 404.60 μmol) as white oil.

¹H NMR (400 MHz, CDCl₃) δ 7.47-7.39 (m, 5H), 5.36 (q, J=7.2 Hz, 1H), 1.59-1.47 (m, 4H).

### Preparation of 4-[(5-bromo-1,3,4-thiadiazol-2-yl)methyl]-6-[(1R)-2,2,2-trifluoro-1-phenyl-ethyl]-4,6-diazaspiro[2.4]heptane-5,7-dione Compound 20-C7

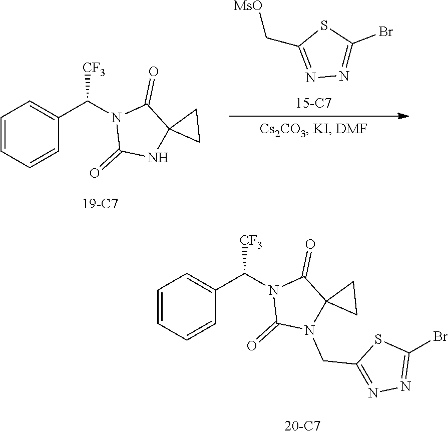 [SMILES: O=C1NC2(CC2)C(=O)N1[C@H](c1ccccc1)C(F)(F)F ; CS(=O)(=O)OCc1nnc(Br)s1 ; O=C1N([C@H](c2ccccc2)C(F)(F)F)C(=O)C2(CC2)N1Cc1nnc(Br)s1]

A mixture of compound 19-C7 (115 mg, 404.60 μmol, 1 eq), compound 15-C7 (132.61 mg, 485.52 mol, 1.2 eq), Cs₂CO₃ (263.65 mg, 809.19 μmol, 2 eq), KI (67.16 mg, 404.60 μmol, 1 eq) in DMF (2 mL) was degassed and purged with N₂ for 3 times, and then the mixture was stirred at 15° C. for 4 h under N₂ atmosphere. TLC (petroleum ether:ethyl acetate=2:1, Rt=0.51) showed the starting material was remained. The reaction mixture was quenched by addition H₂O (10 mL×2) at 20° C., and extracted with Ethyl acetate (10 mL×2). The combined organic layers were washed with brine (10 mL×2), dried over Na₂SO₄, filtered and concentrated under reduced pressure to give a residue. The residue was purified by prep-TLC (SiO₂, petroleum ether/ethyl acetate=2:1) to afford compound 20-C7 (73.8 mg, 159.03 μmol) as white oil.

¹H NMR (400 MHz, CDCl₃) δ 7.70-7.61 (m, 2H), 7.47-7.39 (m, 3H), 5.81 (q, J=8.8 Hz, 1H), 4.76-4.62 (m, 2H), 1.56-1.48 (m, 4H).

### Preparation of 4-[[5-(2-chloro-4-fluoro-3-hydroxy-phenyl)-1,3,4-thiadiazol-2-yl]methyl]-6-[(S)-(difluoromethyl-fluoranyl)-phenyl-methyl]-4,6-diazaspiro[2.4]heptane-5,7-dione Compound I-53

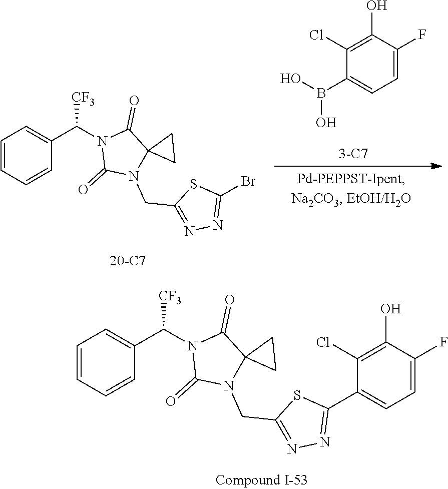 [SMILES: O=C1N([C@H](c2ccccc2)C(F)(F)F)C(=O)C2(CC2)N1Cc1nnc(Br)s1 ; OB(O)c1ccc(F)c(O)c1Cl ; O=C1N([C@H](c2ccccc2)C(F)(F)F)C(=O)C2(CC2)N1Cc1nnc(-c2ccc(F)c(O)c2Cl)s1]

A mixture of compound 20-C7 (16.10 mg, 84.55 μmol, 1.3 eq), compound 3-C7 (30 mg, 65.04 mol, 1 eq), Pd-PEPPST-Ipent (2.80 mg, 3.25 μmol, 0.05 eq), Na₂CO₃ (10.34 mg, 97.56 μmol, 1.5 eq) in EtOH (0.8 mL) and H₂O (0.2 mL) was degassed and purged with N₂ three times, and then the mixture was stirred at 80° C. for 1 h under N₂ atmosphere. LCMS showed the reaction was completed. The aqueous phase was adjusted to pH=3 with citric acid solution, and extracted with ethyl acetate (10 mL xx 2). The combined organic layers were washed with brine (10 mL×2), dried over Na₂SO₄, filtered and concentrated under reduced pressure to give a residue. The residue was purified by prep-HPLC (column: Phenomenex Luna C18 100×40 mm×3 um; mobile phase: [H₂O (0.1% TFA)-ACN]; gradient: 30%-70% B over 8.0 min) to afford Compound I-53 (14.57 mg, 27.56 μmol, 99.65% purity) as white solid.

¹H NMR (400 MHz, DMSO-d₆) δ 10.97 (br s, 1H), 7.73-7.51 (m, 3H), 7.51-7.33 (m, 4H), 6.06 (q, J=8.8 Hz, 1H), 4.97 (s, 2H), 1.66 (s, 2H), 1.36 (s, 2H).

LCMS: RT=2.724 min, MS cal.: 526.05, MS found: [M+H]⁺=527.0.

### Route for I-54

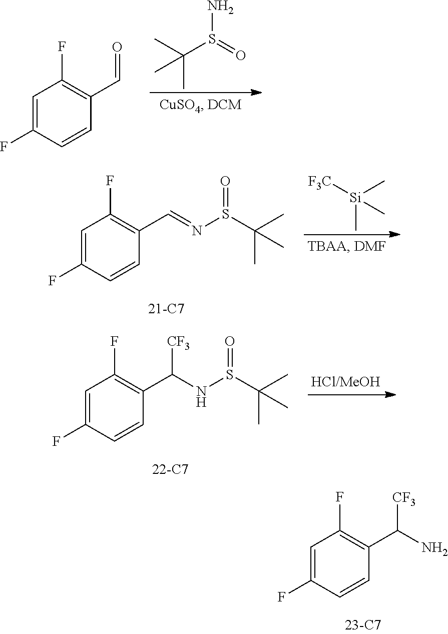

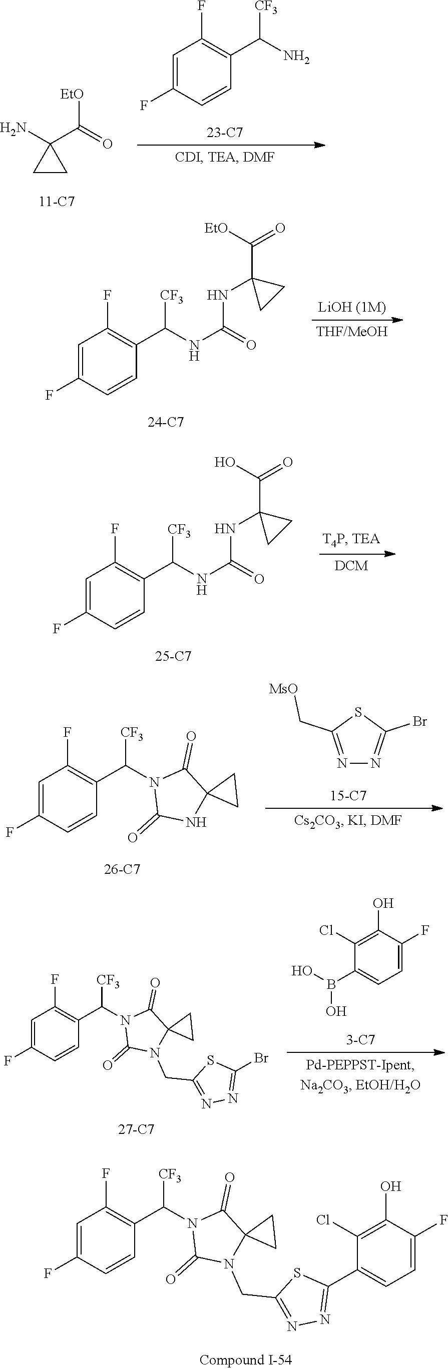

### Preparation of N-(2,4-difluorobenzylidene)-2-methylpropane-2-sulfinamide Compound 21-C7

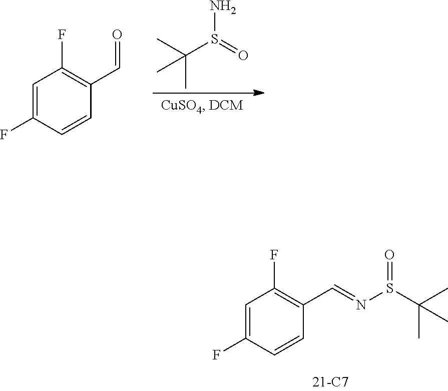

To a solution of 2,4-difluorobenzaldehyde (5 g, 35.19 mmol, 3.83 mL, 1 eq) in dichloromethane (60 mL) was added compound 2-methylpropane-2-sulfinamide (7.25 g, 59.82 mmol, 1.7 eq), CuSO₄ (61.78 g, 387.04 mmol, 59.40 mL, 11 eq). The mixture was stirred at 40° C. for 24 h. The reaction mixture was concentrated under reduced pressure to remove dichloromethane. The residue was purified by column chromatography (SiO₂, petroleum ether:ethyl acetate=99:1 to 9:1) to afford compound 21-C7 (6.58 g, 26.83 mmol) as colorless oil.

¹H NMR (400 MHz, CDCl₃) δ 8.83 (s, 1H), 8.02 (dt, J=6.8, 8.0 Hz, 1H), 7.01-6.95 (m, 1H), 6.90 (ddd, J=1.6, 8.4, 10.8 Hz, 1H), 1.27 (s, 9H).

### Procedure for preparation of N-(1-(2,4-difluorophenyl)-2,2,2-trifluoroethyl)-2-methylpropane-2-sulfinamide Compound 22-C7

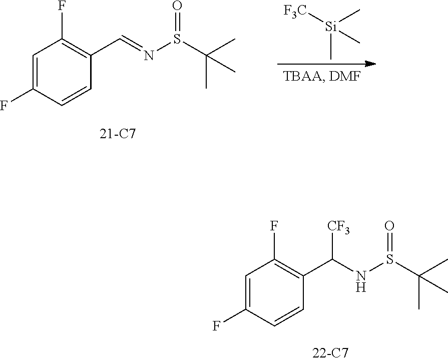

To a solution of compound 21-C7 (5 g, 20.38 mmol, 1 eq.) in DMF (50 mL) was added tetrabutylammonium acetate (6.15 g, 20.38 mmol, 6.21 mL, 1 eq.) and trimethyl(trifluoromethyl)silane (8.70 g, 61.15 mmol, 3 eq.) at 0° C. The mixture was stirred at 30° C. for 3 h. The mixture was poured into water (20 mL) and extracted with ethyl acetate (20 mL×3). The combined organic layers were dried over Na₂SO₄, filtered and concentrated under reduced pressure. The residue was purified by column chromatography (SiO₂, Petroleum ether:Ethyl acetate=9:1 to 1:1) to afford compound 22-C7 (4 g, 12.69 mmol) as white solid.

¹H NMR (400 MHz, CDCl₃) δ 7.39 (d, J=6.4 Hz, 1H), 6.99-6.87 (m, 2H), 5.09 (t, J=7.6 Hz, 1H), 3.89 (d, J=8.0 Hz, 1H), 1.26 (s, 9H).

### Procedure for preparation of 1-(2,4-difluorophenyl)-2,2,2-trifluoroethan-1-amine Compound 23-C7

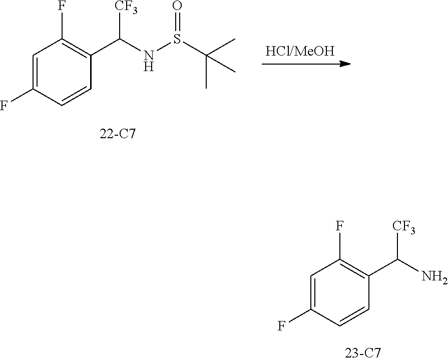 [SMILES: CC(C)(C)S(=O)NC(c1ccc(F)cc1F)C(F)(F)F ; NC(c1ccc(F)cc1F)C(F)(F)F]

A mixture of compound 22-C7 (3.76 g, 11.93 mmol, 1 eq) with the solution of HCl in MeOH (4 M, 60 mL, 20.13 eq.) was degassed and purged with N₂ for 3 times, and then the mixture was stirred at 15° C. for 2 h under N₂ atmosphere. The reaction mixture was concentrated under reduced pressure to give a residue to afford crude compound 23-C7 (3.3 g) as white solid.

¹H NMR (400 MHz, methanol-d₄) δ 7.76-7.65 (m, 1H), 7.32-7.17 (m, 2H), 5.64 (q, J=7.2 Hz, 1H).

### Preparation of ethyl 1-(3-(1-(2,4-difluorophenyl)-2,2,2-trifluoroethyl)ureido)cyclopropane-1-carboxylate Compound 24-C7

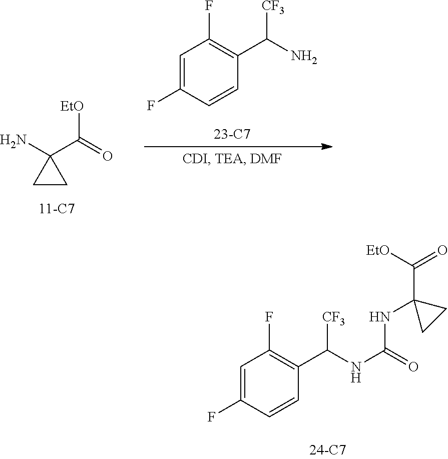 [SMILES: NC(c1ccc(F)cc1F)C(F)(F)F ; CCOC(=O)C1(N)CC1 ; CCOC(=O)C1(NC(=O)NC(c2ccc(F)cc2F)C(F)(F)F)CC1]

To a solution of compound 11-C7 (2.07 g, 15.99 mmol, 1.2 eq) in DMF (15 mL) was added TEA (10.79 g, 106.63 mmol, 14.84 mL, 8 eq.), CDI (2.27 g, 13.99 mmol, 1.05 eq.) at 15° C. for 0.5 h, then compound 23-C7 (3.3 g, 13.33 mmol, 1 eq., HCl) in DMF (15 mL) added to the above reaction mixture. The mixture was stirred at 60° C. for 12 h. The mixture was poured into water (20 mL) and extracted with EtOAc (20 mL×3). The combined organic layers were dried over Na₂SO₄, filtered and concentrated under reduced pressure. The residue was purified by column chromatography (SiO₂, petroleum ether:ethyl acetate=93:7 to 4:1) to afford compound 24-C7 (2.47 g, 6.74 mmol) as colorless oil.

¹H NMR (400 MHz, CDCl₃) δ 7.40-7.30 (m, 1H), 6.92-6.76 (m, 2H), 6.69 (br d, J=8.8 Hz, 1H), 6.17 (br s, 1H), 5.91-5.76 (m, 1H), 4.07 (q, J=7.2 Hz, 2H), 1.51 (q, J=3.6 Hz, 2H), 1.19-1.08 (m, 5H).

### Preparation of 1-(3-(1-(2,4-difluorophenyl)-2,2,2-trifluoroethyl)ureido)cyclopropane-1-carboxylic acid Compound 25-C7

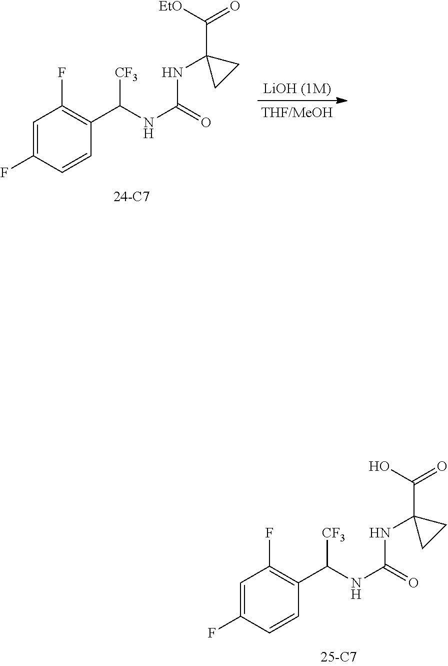 [SMILES: CCOC(=O)C1(NC(=O)NC(c2ccc(F)cc2F)C(F)(F)F)CC1 ; O=C(NC(c1ccc(F)cc1F)C(F)(F)F)NC1(C(=O)O)CC1]

To a solution of compound 24-C7 (2.47 g, 6.74 mmol, 1 eq) in THF (30 mL), MeOH (10 mL) was added LiOH·H₂O (1 M, 7.08 mL, 1.05 eq). The mixture was stirred at 25° C. for 12 h. LCMS showed the reaction was completed. The reaction mixture was quenched by addition H₂O (50 mL×2) at 20° C., and extracted with ethyl acetate (50 mL×2). The aqueous phase was adjusted to pH=3 with citric acid solution, and extracted with ethyl acetate (50 mL×2). The combined organic layers were washed with brine (50 mL×2), dried over Na₂SO₄, filtered and concentrated under reduced pressure to afford compound 25-C7 (1.72 g, 5.09 mmol) as white solid.

¹H NMR (400 MHz, DMSO-d₆) δ 12.28 (br s, 1H), 7.66-7.57 (m, 1H), 7.46 (d, J=9.6 Hz, 1H), 7.43-7.33 (m, 1H), 7.24 (br dt, J=2.0, 8.4 Hz, 1H), 6.72 (br s, 1H), 5.79 (quin, J=8.4 Hz, 1H), 1.33-1.25 (m, 2H), 0.99 (d, J=3.6 Hz, 2H).

### Preparation of 6-[1-(2,4-difluorophenyl)-2,2,2-trifluoro-ethyl]-4,6-diazaspiro[2.4]heptane-5,7-dione Compound 26-C7

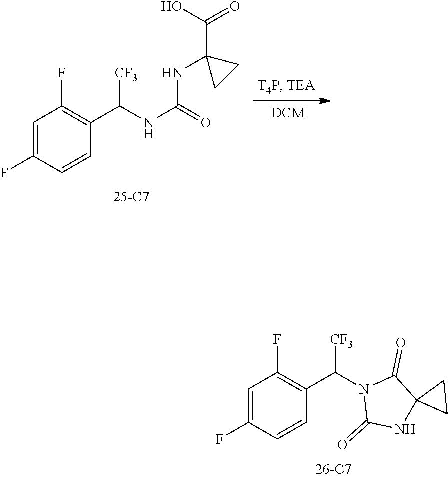 [SMILES: O=C(NC(c1ccc(F)cc1F)C(F)(F)F)NC1(C(=O)O)CC1 ; O=C1NC2(CC2)C(=O)N1C(c1ccc(F)cc1F)C(F)(F)F]

A mixture of compound 25-C7 (1.72 g, 5.09 mmol, 1 eq), triethylamine (2.57 g, 25.43 mmol, 3.54 mL, 5 eq) in dichloromethane (20 mL) was degassed and purged with N₂ for 3 times, T₄P (7.33 g, 10.17 mmol, 50% purity, 2 eq) was added at 0° C. and then the mixture was stirred at 15° C. for 2 h under N₂ atmosphere. TLC (petroleum ether:ethyl acetate=2:1, Rt=0.7) showed the starting material was consumed. The reaction mixture was quenched by addition H₂O (50 mL) at 20° C., and extracted with dichloromethane (50 mL×2). The combined organic layers were washed with brine (50 mL×2), dried over Na₂SO₄, filtered and concentrated under reduced pressure to give a residue. The residue was purified by column chromatography (SiO₂, petroleum ether:ethyl acetate=8:1) to afford compound 26-C7 (420 mg, 1.31 mmol) as white oil.

¹H NMR (400 MHz, CDCl₃) δ 7.42-7.34 (m, 1H), 7.02-6.89 (m, 2H), 5.60 (q, J=7.6 Hz, 1H), 1.59-1.49 (m, 4H).

### Preparation of 4-[(5-bromo-1,3,4-thiadiazol-2-yl)methyl]-6-[1-(2,4-difluorophenyl)-2,2,2-trifluoro-ethyl]-4,6-diazaspiro[2.4]heptane-5,7-dione Compound 27-C7

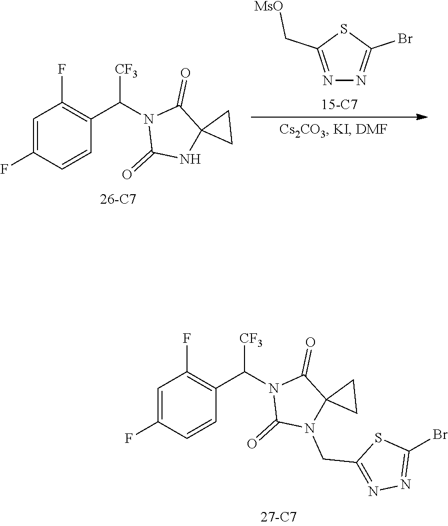 [SMILES: O=C1NC2(CC2)C(=O)N1C(c1ccc(F)cc1F)C(F)(F)F ; CS(=O)(=O)OCc1nnc(Br)s1 ; O=C1N(C(c2ccc(F)cc2F)C(F)(F)F)C(=O)C2(CC2)N1Cc1nnc(Br)s1]

A mixture of compound 26-C7 (420 mg, 1.31 mmol, 1 eq), compound 15-C7 (429.89 mg, 1.57 mmol, 1.2 eq), KI (217.73 mg, 1.31 mmol, 1 eq), Cs₂CO₃ (854.70 mg, 2.62 mmol, 2 eq) in DMF (4 mL) was degassed and purged with N₂ for 3 times, and then the mixture was stirred at 15° C. for 2 h under N₂ atmosphere. TLC (petroleum ether:ethyl acetate=2:1, Rt=0.51) showed the starting material was consumed completely. The reaction mixture was quenched by addition of H₂O (20 mL×2) at 20° C., and extracted with ethyl acetate (20 mL×2). The combined organic layers were washed with brine (20 mL×2), dried over Na₂SO₄, filtered and concentrated under reduced pressure to give a residue. The residue was purified by column chromatography (SiO₂, petroleum ether:ethyl acetate=4:1) to afford compound 27-C7 (450 mg, 905.00 μmol) as white oil.

¹H NMR (400 MHz, CDCl₃) δ 8.08-7.97 (m, 1H), 6.98 (tt, J=1.2, 7.8 Hz, 1H), 6.88 (ddd, J=2.4, 8.4, 10.4 Hz, 1H), 6.17 (q, J=8.4 Hz, 1H), 4.78-4.61 (m, 2H), 1.57-1.45 (m, 4H).

### Preparation of 4-[[5-(2-chloro-4-fluoro-3-hydroxy-phenyl)-1,3,4-thiadiazol-2-yl]methyl]-6-[1-(2,4-difluorophenyl)-2,2,2-trifluoro-ethyl]-4,6-diazaspiro[2.4]heptane-5,7-dione Compound I-54

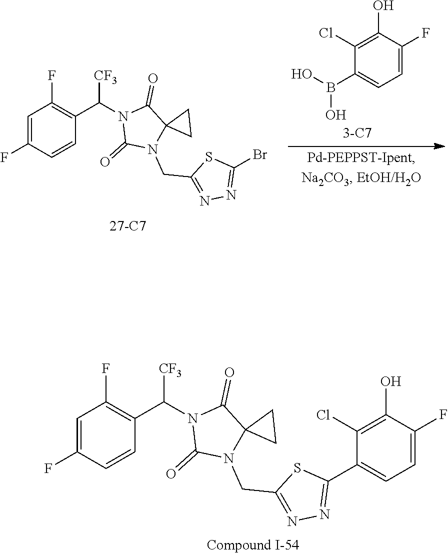 [SMILES: O=C1N(C(c2ccc(F)cc2F)C(F)(F)F)C(=O)C2(CC2)N1Cc1nnc(Br)s1 ; OB(O)c1ccc(F)c(O)c1Cl ; O=C1N(C(c2ccc(F)cc2F)C(F)(F)F)C(=O)C2(CC2)N1Cc1nnc(-c2ccc(F)c(O)c2Cl)s1]

A mixture of compound 27-C7 (150 mg, 301.67 μmol, 1 eq), compound 3-C7 (74.65 mg, 392.17 mol, 1.3 eq), Na₂CO₃ (47.96 mg, 452.50 μmol, 1.5 eq), Pd-PEPPST-Ipent (12.98 mg, 15.08 μmol, 0.05 eq) in ethanol (1.6 mL) and H₂O (0.4 mL) was degassed and purged with N₂ for 3 times, and then the mixture was stirred at 80° C. for 1 h under N₂ atmosphere. LCMS showed the reaction was completed. The aqueous phase was adjusted to pH=3 with citric acid solution, and extracted with ethyl acetate (15 mL×2). The combined organic layers were washed with brine (15 mL×2), dried over Na₂SO₄, filtered and concentrated under reduced pressure to give a residue. The residue was purified by prep-HPLC (column: Phenomenex Luna C18 100×40 mm×3 um; mobile phase: [H₂O (0.1% TFA)-ACN]; gradient: 35%-75% B over 8.0 min) to afford Compound I-54 (82.75 mg, 147.01 μmol, 100% purity) as white solid.

¹H NMR (400 MHz, DMSO-d₆) δ 10.97 (br s, 1H), 7.95-7.84 (m, 1H), 7.60 (dd, J=5.6, 8.8 Hz, 1H), 7.45-7.35 (m, 2H), 7.23 (dt, J=2.0, 8.8 Hz, 1H), 6.29 (q, J=8.8 Hz, 1H), 4.95 (s, 2H), 1.70-1.63 (m, 2H), 1.40-1.33 (m, 2H).

LCMS: RT=2.801 min, MS cal.: 562.0, MS found: [M+H]⁺=563.0.

### Route for I-55

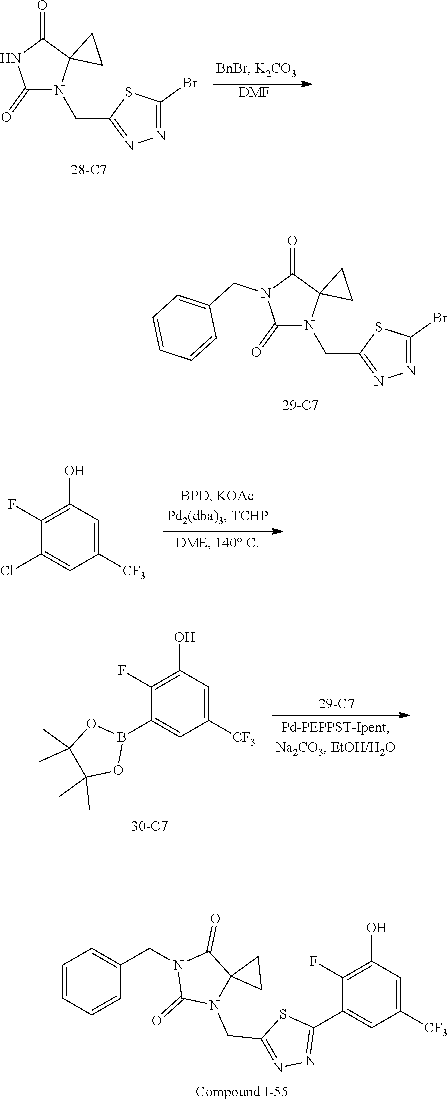

### Preparation of 6-benzyl-4-((5-bromo-1,3,4-thiadiazol-2-yl)methyl)-4,6-diazaspiro[2.4]heptane-5,7-dione Compound 29-C7

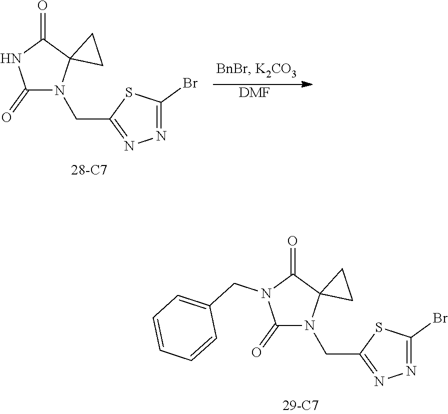

To a solution of compound 28-C7 (1 g, 3.30 mmol, 1 eq) and bromomethylbenzene (677.06 mg, 3.96 mmol, 470.18 μL, 1.2 eq) in DMF (10 mL) was added K₂CO₃ (1.37 g, 9.90 mmol, 3 eq). The mixture was heated to 60° C. for 3 h. LCMS indicated reactant was consumed completely. The mixture was poured into water (20 mL) and extracted with ethyl acetate (20 mL×3). The combined organic layers were dried over Na₂SO₄, filtered and concentrated under reduced pressure. The residue was purified by column chromatography (SiO₂, petroleum ether:ethyl acetate=9:1 to 1:1) to give compound 29-C7 (1 g, 2.54 mmol) as white solid.

¹H NMR (400 MHz, DMSO-d₆) δ ppm 7.39-7.25 (m, 5H), 4.86 (s, 2H), 4.65 (s, 2H), 1.61-1.51 (m, 2H), 1.32-1.24 (m, 2H)

### Preparation of 2-fluoro-3-(4,4,5,5-tetramethyl-1,3,2-dioxaborolan-2-yl)-5-(trifluoromethyl)phenol Compound 30-C7

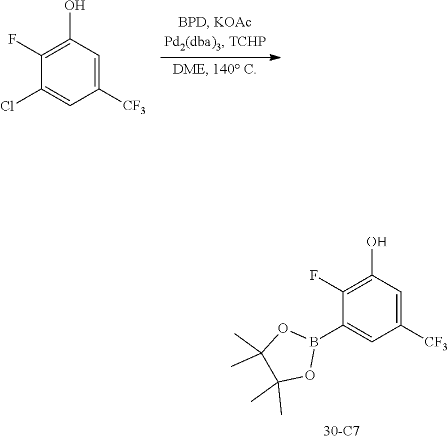 [SMILES: Oc1cc(C(F)(F)F)cc(Cl)c1F ; CC1(C)OB(c2cc(C(F)(F)F)cc(O)c2F)OC1(C)C]

To a solution of compound 30-C7 (0.5 g, 2.33 mmol, 1 eq.) in 1,2-dimethoxyethane (5 mL) was added 4,4,5,5-tetramethyl-2-(4,4,5,5-tetramethyl-1,3,2-dioxaborolan-2-yl)-1,3,2-dioxaborolane (BPD, 1.18 g, 4.66 mmol, 2 eq.) and KOAc (343.08 mg, 3.50 mmol, 1.5 eq). After purged N₂ for three times, Pd₂(dba)₃ (213.41 mg, 233.05 μmol, 0.1 eq) and P(Cy)₃ (261.42 mg, 932.21 μmol, 302.22 μL, 0.4 eq.) was added. The mixture was heated to 140° C. for 6 h under microwave. LC-MS showed reactant was consumed completely and one main peak with desired m/z. The reaction mixture was quenched by addition of water (10 mL), and extracted with ethyl acetate (10 mL×3). The combined organic layers were washed with brine (3×10 mL), dried over Na₂SO₄, filtered and concentrated under reduced pressure to give compound 2 (0.5 g, 1.63 mmol) as a white solid.

¹H NMR (400 MHz, DMSO-d₆) δ ppm 10.59 (s, 1H), 7.34 (dd, J=2.4, 7.6 Hz, 1H), 7.29-7.25 (m, 1H), 1.30 (s, 12H)

### Preparation of 6-benzyl-4-((5-(2-fluoro-3-hydroxy-5-(trifluoromethyl)phenyl)-1,3,4-thiadiazol-2-yl)methyl)-4,6-diazaspiro[2.4]heptane-5,7-dione Compound I-55

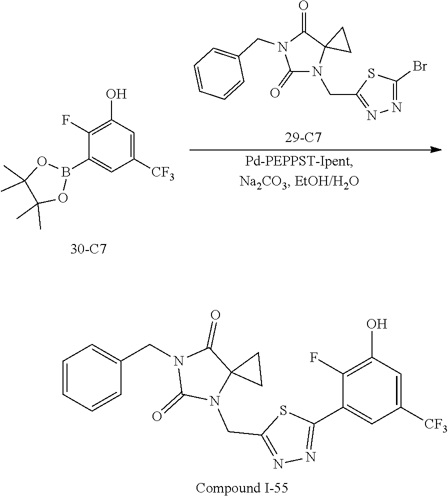 [SMILES: CC1(C)OB(c2cc(C(F)(F)F)cc(O)c2F)OC1(C)C ; O=C1N(Cc2ccccc2)C(=O)C2(CC2)N1Cc1nnc(Br)s1 ; O=C1N(Cc2ccccc2)C(=O)C2(CC2)N1Cc1nnc(-c2cc(C(F)(F)F)cc(O)c2F)s1]

To a solution of compound 30-C7 (250 mg, 635.72 μmol, 1 eq) in ethanol (4 mL) and H₂O (1 mL) was added compound 2 (194.57 mg, 635.72 μmol, 1 eq) and Na₂CO₃ (101.07 mg, 953.57 μmol, 1.5 eq). After purged N₂ for three times, 1,3-bis[2,6-bis(1-ethylpropyl)phenyl]-4,5-dichloro-2H-imidazol-1-ium-2-ide; 3-chloropyridine; dichloropalladium (Pd-PEPPST-Ipent, 54.71 mg, 63.57 mol, 0.1 eq.) was added. The mixture was heated to 80° C. for 1 h under N₂ atmosphere. LC-MS showed Reactant was consumed completely and one main peak with desired m/z. The reaction mixture was poured into water (5 mL), and then adjusted the pH=4 using citric acid. The mixture was extracted with ethyl acetate (5 mL×2). The organic layer was dried, filtered and concentrated to give a residue. The residue was purified by prep-HPLC (column: Phenomenex Luna C18 100×30 mm×3 um; mobile phase: [H₂O (0.2% FA)-ACN]; gradient: 40%-70% B over 8.0 min) to give Compound I-55 (126 mg, 255.87 μmol) as white solid.

¹H NMR (400 MHz, DMSO-d₆) δ ppm 11.28 (br s, 1H), 7.90 (d, J=3.2 Hz, 1H), 7.43 (dd, J=2.0, 7.6 Hz, 1H), 7.39-7.24 (m, 5H), 4.97 (s, 2H), 4.68 (s, 2H), 1.69-1.55 (m, 2H), 1.37-1.22 (m, 2H)

LCMS: RT=2.689 min, MS cal.: 492.1, MS found: [M+H]⁺=493.1.

The following examples in Table 8 can be made in a similar manner to the procedures outlined above.

TABLE 8

| Compound | Structure | Name |
|---|---|---|
| I-56 | 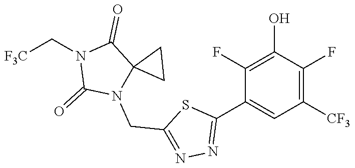 [SMILES: O=C1N(CC(F)(F)F)C(=O)C2(CC2)N1Cc1nnc(-c2cc(C(F)(F)F)c(F)c(O)c2F)s1] | 1H NMR (400 MHz, DMSO- d6) δ 7.95-7.75 (m, 1H), 4.99 (s, 2H), 4.35 (d, J = 9.2 Hz, 2H), 1.68 (d, J = 2.8 Hz, 2H), 1.35 (d, J = 2.8 Hz, 2H). (MH⁺ 503.0) |
| I-57 | 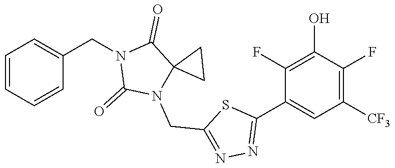 [SMILES: O=C1N(Cc2ccccc2)C(=O)C2(CC2)N1Cc1nnc(-c2cc(C(F)(F)F)c(F)c(O)c2F)s1] | 1H NMR (400 MHz, DMSO- d6) δ 7.89-7.74 (m, 1H), 7.40-7.21 (m, 5H), 4.96 (s, 2H), 4.68 (s, 2H), 1.61 (d, J = 2.8 Hz, 2H), 1.29 (d, J = 2.8 Hz, 2H). (MH⁺ 511.1) |
| I-58 | 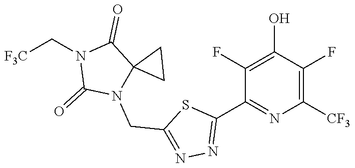 [SMILES: O=C1N(CC(F)(F)F)C(=O)C2(CC2)N1Cc1nnc(-c2nc(C(F)(F)F)c(F)c(O)c2F)s1] | 1H NMR (400 MHz, DMSO- d6) δ 5.04-4.87 (m, 2H), 4.34 (q, J = 9.6 Hz, 2H), 1.74-1.58 (m, 2H), 1.45-1.26 (m, 2H). (MH⁺ 504.0) |
| I-59 | 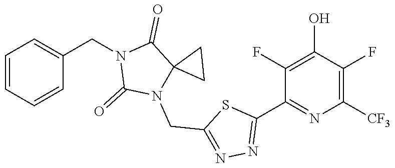 [SMILES: O=C1N(Cc2ccccc2)C(=O)C2(CC2)N1Cc1nnc(-c2nc(C(F)(F)F)c(F)c(O)c2F)s1] | 1H NMR (400 MHz, DMSO- d6) δ 7.43-7.18 (m, 5H), 4.98-4.88 (m, 2H), 4.70-4.63 (m, 2H), 1.64-1.57 (m, 2H), 1.33-1.27 (m, 2H). (MH⁺ 512.0) |
| I-60 | 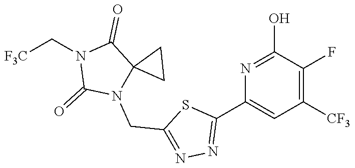 [SMILES: O=C1N(CC(F)(F)F)C(=O)C2(CC2)N1Cc1nnc(-c2cc(C(F)(F)F)c(F)c(O)n2)s1] | ¹H NMR (400 MHz, DMSO- d₆) δ 13.34-13.13 (br m, 1H), 7.87-7.77 (m, 1H), 4.93 (s, 2H), 4.34 (q, J = 9.2 Hz, 2H), 1.71-1.63 (m, 2H), 1.38-1.31 (m, 2H). (MH⁺ 486.0) |
| I-61 | 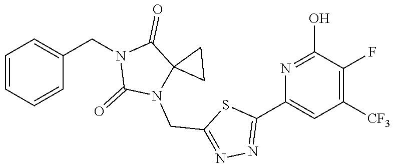 [SMILES: O=C1N(Cc2ccccc2)C(=O)C2(CC2)N1Cc1nnc(-c2cc(C(F)(F)F)c(F)c(O)n2)s1] | ¹H NMR (400 MHz, DMSO- d6) δ 13.41-13.05 (br m, 1H), 7.83 (d, J = 2.4 Hz, 1H), 7.40-7.24 (m, 5H), 4.91 (s, 2H), 4.67 (s, 2H), 1.64-1.58 (m, 2H), 1.33-1.26 (m, 2H). (MH⁺ 494.0) |
| I-62 | 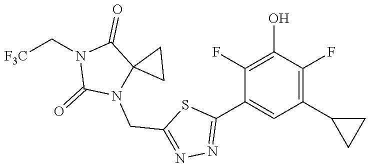 [SMILES: O=C1N(CC(F)(F)F)C(=O)C2(CC2)N1Cc1nnc(-c2cc(C3CC3)c(F)c(O)c2F)s1] | 1H NMR (400 MHz, DMSO- d6) 8 8.25 (br s, 1H), 7.16 (t, J = 7.6 Hz, 1H), 4.94 (s, 2H), 4.34 (q, J = 9.2 Hz, 2H), 2.00-2.09 (m, 1H), 1.64-1.70 (m, 2H), 1.29-1.37 (m, 2H), 0.95-1.02 (m, 2H), 0.70-0.77 (m, 2H) (MH⁺ 475.1) |
| I-63 | 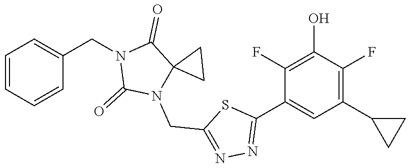 [SMILES: O=C1N(Cc2ccccc2)C(=O)C2(CC2)N1Cc1nnc(-c2cc(C3CC3)c(F)c(O)c2F)s1] | 1H NMR (400 MHz, DMSO- d6) δ 7.26-7.38 (m, 5H), 7.19 (t, J = 7.6 Hz, 1H), 4.93 (s, 2H), 4.67 (s, 2H), 1.99-2.11 (m, 1H), 1.56-1.66 (m, 2H), 1.23-1.34 (m, 2H), 1.00 (dd, J = 8.4, 1.88 Hz, 2H), 0.69-0.79 (m, 2H). (MH⁺ 483.1) |
| I-64 | 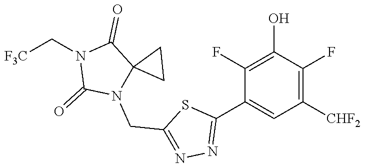 [SMILES: O=C1N(CC(F)(F)F)C(=O)C2(CC2)N1Cc1nnc(-c2cc(C(F)F)c(F)c(O)c2F)s1] | 1H NMR (400 MHz, DMSO- d6) δ 6.63-7.36 (m, 2H), 4.92 (br s, 2H), 4.34 (br d, J = 8.76 Hz, 2H), 1.67 (br s, 2H), 1.23-1.42 (m, 2H). (MH⁺ 483.0) |
| I-65 | 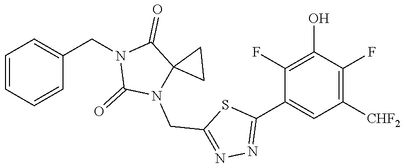 [SMILES: O=C1N(Cc2ccccc2)C(=O)C2(CC2)N1Cc1nnc(-c2cc(C(F)F)c(F)c(O)c2F)s1] | 1H NMR (400 MHz, DMSO- d6) δ ppm 7.21-7.42 (m, 5H), 6.89-7.17 (m, 2H), 4.90 (s, 2H), 4.68 (s, 2H), 1.53-1.68 (m, 2H), 1.28 (s, 2H). (MH⁺ 491.0) |
| I-68 | 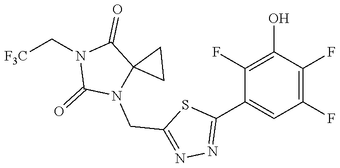 [SMILES: O=C1N(CC(F)(F)F)C(=O)C2(CC2)N1Cc1nnc(-c2cc(F)c(F)c(O)c2F)s1] | 1H NMR (400 MHz, DMSO- d6) δ 6.79-6.99 (m, 1H), 4.92 (s, 2H), 4.34 (q, J = 9.2 Hz, 2H), 1.62-1.72 (m, 2H), 1.28-1.38 (m, 2H). (MH⁺ 451.0) |
| I-69 | 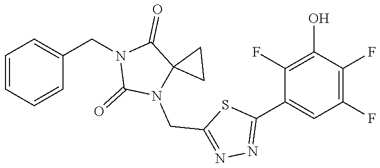 [SMILES: O=C1N(Cc2ccccc2)C(=O)C2(CC2)N1Cc1nnc(-c2cc(F)c(F)c(O)c2F)s1] | 1H NMR: (400 MHz, DMSO- d6) 8 7.65 (ddd, J = 10.8, 8.4, 6.0 Hz, 1H), 7.25-7.40 (m, 5H), 4.94 (s, 2H), 4.67 (s, 2H), 1.56-1.65 (m, 2H), 1.25-1.34 (m, 2H). (MH⁺ 459.0) |
| I-72 Metabolite | 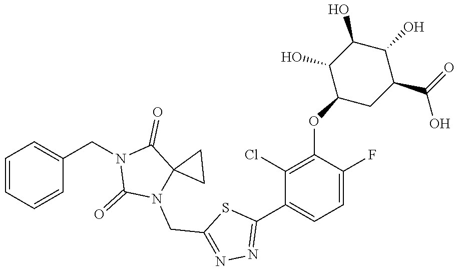 [SMILES: O=C(O)[C@H]1C[C@@H](Oc2c(F)ccc(-c3nnc(CN4C(=O)N(Cc5ccccc5)C(=O)C45CC5)s3)c2Cl)[C@H](O)[C@@H](O)[C@@H]1O] | 1H NMR (400 MHz, DMSO- d6) δ 12.74 (br s, 1H), 7.96-7.93 (dd, 1H), 7.55-7.50 (t, 1H), 7.37-7.29 (m, 5H), 5.59 (br s, 1H), 5.32 (br s, 1H), 5.06-5.04 (d, 1H), 4.95 (s, 2H), 4.68 (s, 2H), 3.71-3.68 (d, 1H), 3.44-3.42 (m, 1H), 3.39 (m, 1H), 3.29 (s, 1H), 3.26 (s, 1H), 1.63-1.59 (m, 2H), 1.31-1.28 (m, 2H). (MH⁺ 635.1) |
| I-73 | 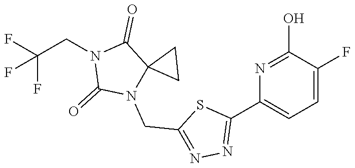 [SMILES: O=C1N(CC(F)(F)F)C(=O)C2(CC2)N1Cc1nnc(-c2ccc(F)c(O)n2)s1] | 1HNMR (400 MHz, DMSO- d6) δ 12.39-12.13 (br m, 1H), 7.84-7.52 (m, 2H), 4.90 (s, 2H), 4.34 (q, J = 9.2 Hz, 2H), 1.73-1.61 (m, 2H), 1.41-1.29 (m, 2H). (MH⁺ 418.1) |
| I-74 | 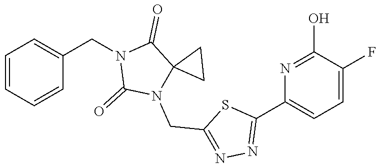 [SMILES: O=C1N(Cc2ccccc2)C(=O)C2(CC2)N1Cc1nnc(-c2ccc(F)c(O)n2)s1] | 1H NMR (400 MHz, DMSO- d6) δ 12.40-12.09 (br m, 1H), 7.81-7.59 (m, 2H), 7.42-7.23 (m, 5H), 4.88 (s, 2H), 4.67 (s, 2H), 1.65-1.58 (m, 2H), 1.34-1.25 (m, 2H). (MH⁺ 426.0) |
| I-75 | 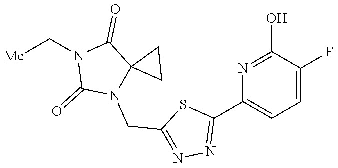 [SMILES: CCN1C(=O)N(Cc2nnc(-c3ccc(F)c(O)n3)s2)C2(CC2)C1=O] | 1H NMR (400 MHz, DMSO- d6) δ 12.25 (br s, 1H), 7.83-7.71 (m, 1H), 7.66 (s, 1H), 7.82-7.57 (m, 1H), 4.85 (s, 2H), 3.55-3.48 (m, 2H), 1.63-1.51 (m, 2H), 1.31-1.20 (m, 2H), 1.13 (t, J = 7.2 Hz, 3H) (MH⁺ 364.0) |
| I-76 | 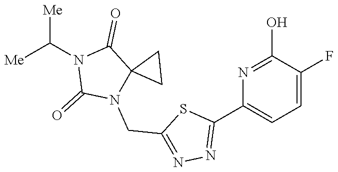 [SMILES: CC(C)N1C(=O)N(Cc2nnc(-c3ccc(F)c(O)n3)s2)C2(CC2)C1=O] | 1HNMR (400 MHz, DMSO- d6) δ 10.81 (br s, 1H), 7.66 (ddd, J = 8.69, 7.50, 6.07 Hz, 1H), 7.31-7.43 (m, 4H), 7.21-7.31 (m, 2H), 5.34 (q, J = 7.13 Hz, 1H), 4.91 (s, 2H), 1.78 (d, J = 7.25 Hz, 3H), 1.53-1.63 (m, 2H), 1.21-1.31 (m, 2H). (MH⁺ 378.0) |
| I-77 | 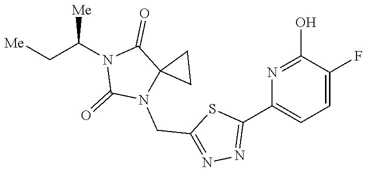 [SMILES: CC[C@@H](C)N1C(=O)N(Cc2nnc(-c3ccc(F)c(O)n3)s2)C2(CC2)C1=O] | 1HNMR (400 MHz, DMSO- d6) δ 7.25-7.13 (m, 1H), 7.01 (d, J = 6.0 Hz, 1H), 4.78 (s, 2H), 4.16-3.92 (m, 1H), 1.98-1.83 (m, 1H), 1.73-1.60 (m, 1H), 1.58-1.50 (m, 2H), 1.35 (d, J = 6.8 Hz, 3H), 1.26- 1.20 (m, 2H), 0.81 (t, J = 7.2 Hz, 3H). (MH⁺ 392.1) |
| I-78 | 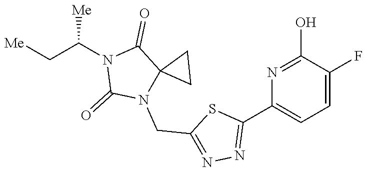 [SMILES: CC[C@H](C)N1C(=O)N(Cc2nnc(-c3ccc(F)c(O)n3)s2)C2(CC2)C1=O] | 1HNMR (400 MHz, DMSO- d6) δ 7.25-7.13 (m, 1H), 7.01 (d, J = 6.0 Hz, 1H), 4.78 (s, 2H), 4.16-3.92 (m, 1H), 1.98-1.83 (m, 1H), 1.73-1.60 (m, 1H), 1.58-1.50 (m, 2H), 1.35 (d, J = 6.8 Hz, 3H), 1.26-1.20 (m, 2H), 0.81 (t, J = 7.2 Hz, 3H). (MH⁺ 392.1) |
| I-79 | 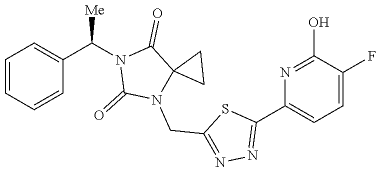 [SMILES: C[C@H](c1ccccc1)N1C(=O)N(Cc2nnc(-c3ccc(F)c(O)n3)s2)C2(CC2)C1=O] | 1H NMR (400 MHz, DMSO- d6) δ 12.13-12.46 (br m, 1H), 7.63-8.00 (m, 2H), 7.07-7.50 (m, 5H), 5.22-5.49 (m, 1H), 4.62-4.97 (m, 2H), 1.72-1.85 (m, 3H), 1.46-1.65 (m, 2H), 1.13-1.35 (m, 2H). (MH⁺ 440.1) |
| I-80 | 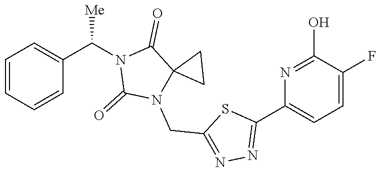 [SMILES: C[C@@H](c1ccccc1)N1C(=O)N(Cc2nnc(-c3ccc(F)c(O)n3)s2)C2(CC2)C1=O] | 1H NMR (400 MHz, DMSO- d6) δ 7.56-7.69 (m, 1H), 7.43-7.53 (m, 1H), 7.31-7.40 (m, 4H), 7.23-7.30 (m, 1H), 5.18-5.47 (m, 1H), 4.68-4.98 (m, 2H), 1.68-1.85 (m, 3H), 1.44-1.64 (m, 2H), 1.08-1.31 (m, 2H). (MH⁺ 440.1) |
| I-81 | 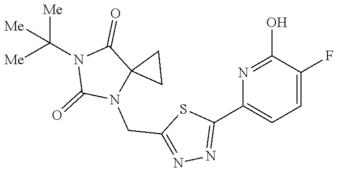 [SMILES: CC(C)(C)N1C(=O)N(Cc2nnc(-c3ccc(F)c(O)n3)s2)C2(CC2)C1=O] | 1H NMR (400 MHz, DMSO- d6) δ 11.97-12.31 (br m, 1H), 7.43-8.03 (m, 2H), 4.54-4.96 (m, 2H), 1.54-1.60 (m, δ H), 1.54-1.69 (m, 9H), 1.46-1.54 (m, 2H), 1.16-1.23 (m, 2H). (MH⁺ 392.0) |
| I-82 | 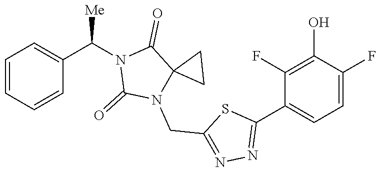 [SMILES: C[C@H](c1ccccc1)N1C(=O)N(Cc2nnc(-c3ccc(F)c(O)c3F)s2)C2(CC2)C1=O] | 1H NMR (400 MHz, DMSO- d6) δ 10.81 (br s, 1H), 7.66 (ddd, J = 8.8, 7.6, 6.0 Hz, 1H), 7.31-7.43 (m, 4H), 7.21-7.31 (m, 2H), 5.34 (q, J = 7.13 Hz, 1H), 4.91 (s, 2H), 1.78 (d, J = 7.2 Hz, 3H), 1.53-1.63 (m, 2H), 1.21-1.31 (m, 2H). (MH⁺ 457.0) |
| I-83 | 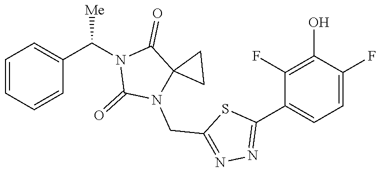 [SMILES: C[C@@H](c1ccccc1)N1C(=O)N(Cc2nnc(-c3ccc(F)c(O)c3F)s2)C2(CC2)C1=O] | 1H NMR (400 MHz, DMSO- d6) δ 7.56-7.75 (m, 1H), 7.17-7.44 (m, 6H), 5.25-5.40 (m, 1H), 4.83-4.97 (m, 2H), 1.74-1.81 (m, 3H), 1.53-1.63 (m, 2H), 1.22-1.29 (m, 2H). (MH⁺ 457.0) |
| I-84 | 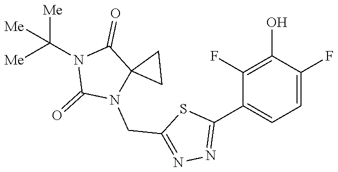 [SMILES: CC(C)(C)N1C(=O)N(Cc2nnc(-c3ccc(F)c(O)c3F)s2)C2(CC2)C1=O] | 1H NMR (400 MHz, DMSO- d6) δ 7.70-7.60 (m, 1H), 7.31-7.19 (m, 1H), 4.85 (s, 2H), 2.07 (s, 1H), 1.56 (s, 9H), 1.53-1.47 (m, 2H), 1.22-1.14 (m, 2H). (MH⁺ 409.1) |
| I-85 | 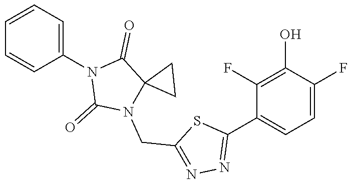 [SMILES: O=C1N(c2ccccc2)C(=O)C2(CC2)N1Cc1nnc(-c2ccc(F)c(O)c2F)s1] | 1H NMR (400 MHz, DMSO- d6) δ 10.84 (br s, 1H), 7.66 (ddd, J = 6.0, 7.6, 8.8 Hz, 1H), 7.55-7.36 (m, 5H), 7.30-7.21 (m, 1H), 4.99 (s, 2H), 1.80-1.65 (m, 2H), 1.45-1.30 (m, 2H). (MH⁺ 429.0) |
| I-86 | 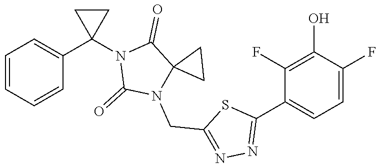 [SMILES: O=C1N(Cc2nnc(-c3ccc(F)c(O)c3F)s2)C2(CC2)C(=O)N1C1(c2ccccc2)CC1] | 1H NMR (400 MHz, DMSO- d6) δ 10.83 (br s, 1H), 7.73-7.59 (m, 1H), 7.34-7.19 (m, 4H), 7.19-7.13 (m, 2H), 4.92 (s, 2H), 1.63-1.56 (m, 2H), 1.43 (d, J = 4.8 Hz, 4H), 1.31-1.24 (m, 2H). (MH⁺ 469.1) |
| I-87 | 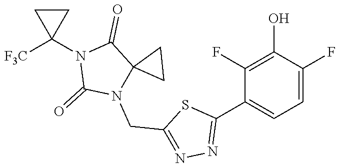 [SMILES: O=C1N(Cc2nnc(-c3ccc(F)c(O)c3F)s2)C2(CC2)C(=O)N1C1(C(F)(F)F)CC1] | 1H NMR (400 MHz, DMSO- d6) δ 10.83 (br s, 1H), 7.68-7.63 (m, 1H), 7.30-7.20 (m, 1H), 4.91 (s, 2H), 1.69-1.59 (m, 2H), 1.53-1.44 (m, 2H), 1.40-1.32 (m, 2H), 1.32-1.24 (m, 2H). (MH⁺ 461.0) |
| I-88 | 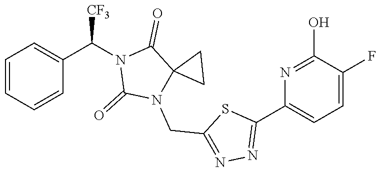 [SMILES: O=C1N([C@@H](c2ccccc2)C(F)(F)F)C(=O)C2(CC2)N1Cc1nnc(-c2ccc(F)c(O)n2)s1] | 1H NMR (400 MHz, DMSO- d6) δ ppm 12.50-12.05 (br m, 1H), 7.88-7.54 (m, 4H), 7.45 (s, 3H), 6.19-5.88 (m, 1H), 5.00-4.83 (m, 2H), 1.73-1.59 (m, 2H), 1.35 (s, 2H). (MH⁺ 494.0) |
| I-89 | 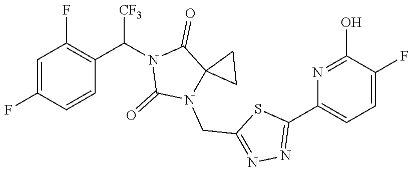 [SMILES: O=C1N(C(c2ccc(F)cc2F)C(F)(F)F)C(=O)C2(CC2)N1Cc1nnc(-c2ccc(F)c(O)n2)s1] | ¹H NMR (400 MHz, DMSO- d6) δ 12.34-12.06 (br m, 1H), 7.95-7.86 (m, 1H), 7.85-7.64 (m, 2H), 7.46-7.36 (m, 1H), 7.23 (dt, J = 2.0, 8.8 Hz, 1H), 6.28 (q, J = 8.8 Hz, 1H), 5.03-4.78 (m, 2H), 1.75-1.58 (m, 2H), 1.42-1.25 (m, 2H). (MH⁺ 530.0) |
| I-90A | 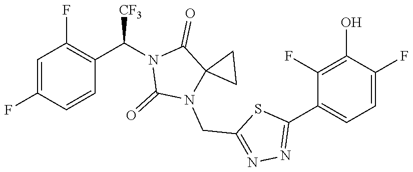 [SMILES: O=C1N([C@@H](c2ccc(F)cc2F)C(F)(F)F)C(=O)C2(CC2)N1Cc1nnc(-c2ccc(F)c(O)c2F)s1] | 1H NMR (400 MHz, DMSO- d6) δ 10.80 (br s, 1H), 7.97-7.84 (m, 1H), 7.66 (ddd, J = 6.0, 7.6, 8.8 Hz, 1H), 7.41 (ddd, J = 2.4, 8.8, 11.2 Hz, 1H), 7.32-7.15 (m, 2H), 6.29 (q, J = 8.8 Hz, 1H), 5.02-4.89 (m, 2H), 1.71-1.60 (m, 2H), 1.37-1.28 (m, 2H). (MH⁺ 547.0) |
| I-90B | 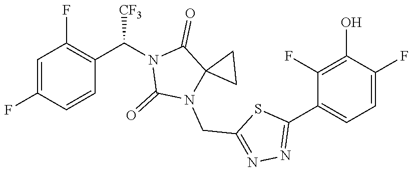 [SMILES: O=C1N([C@H](c2ccc(F)cc2F)C(F)(F)F)C(=O)C2(CC2)N1Cc1nnc(-c2ccc(F)c(O)c2F)s1] | 1H NMR (400 MHz, DMSO- d6) δ 11.12-10.63 (br m, 1H), 7.96-7.85 (m, 1H), 7.67-7.59 (m, 1H), 7.44-7.36 (m, 1H), 7.30-7.17 (m, 2H), 6.29 (q, J = 9.2 Hz, 1H), 5.02-4.88 (m, 2H), 1.71-1.60 (m, 2H), 1.40-1.28 (m, 2H). (MH⁺ 547.0) |
| I-91 | 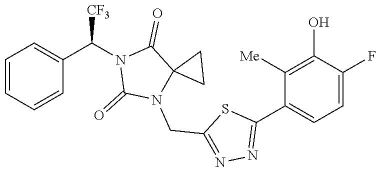 [SMILES: Cc1c(-c2nnc(CN3C(=O)N([C@@H](c4ccccc4)C(F)(F)F)C(=O)C34CC4)s2)ccc(F)c1O] | 1H NMR (400 MHz, DMSO- d6) δ 10.29 (br s, 1H), 7.63 (dd, J = 2.8, 6.4 Hz, 2H), 7.48-7.42 (m, 3H), 7.40 (dd, J = 2.0, 8.0 Hz, 1H), 7.28 (dd, J = 2.0, 5.9 Hz, 1H), 6.04 (d, J = 9.2 Hz, 1H), 4.91 (d, J = 0.8 Hz, 2H), 2.27 (d, J = 2.0 Hz, 3H), 1.70-1.62 (m, 2H), 1.39-1.31 (m, 2H). (MH⁺ 507.1) |
| I-92 | 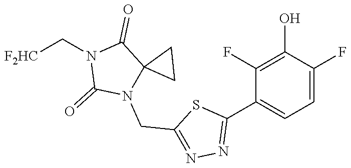 [SMILES: O=C1N(CC(F)F)C(=O)C2(CC2)N1Cc1nnc(-c2ccc(F)c(O)c2F)s1] | 1H NMR (400 MHz, DMSO- d6) δ 10.76 (s, 1H), 7.78-7.54 (m, 1H), 7.39-7.13 (m, 1H), 6.23 (tt, J = 3.6, 54.8 Hz, 1H), 4.94 (s, 2H), 3.92 (dt, J = 3.2, 15.2 Hz, 2H), 1.73-1.57 (m, 2H), 1.39-1.23 (m, 2H). (MH⁺ 417.0) |
| I-93 | 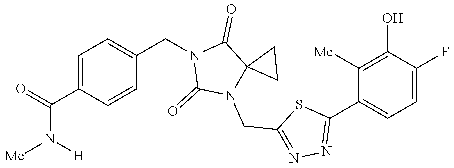 [SMILES: CNC(=O)c1ccc(CN2C(=O)N(Cc3nnc(-c4ccc(F)c(O)c4C)s3)C3(CC3)C2=O)cc1] | ¹H NMR (400 MHz, DMSO- d₆) δ 10.76 (s, 1H), 8.40 (q, J = 4.4 Hz, 1H), 7.79 (d, J = 8.4 Hz, 2H), 7.70-7.60 (m, 1H), 7.36 (d, J = 8.4 Hz, 2H), 7.31-7.21 (m, 1H), 4.94 (s, 2H), 4.72 (s, 2H), 2.77 (d, J = 4.4 Hz, 3H), 1.69-1.54 (m, 2H), 1.38-1.23 (m, 2H). (MH⁺ 500.1) |
| I-94 | 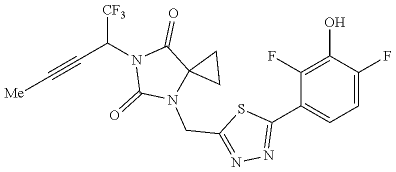 [SMILES: CC#CC(N1C(=O)N(Cc2nnc(-c3ccc(F)c(O)c3F)s2)C2(CC2)C1=O)C(F)(F)F] | ¹H NMR (400 MHz, METHANOL-d₄) δ 7.76-7.65 (m, 1H), 7.19-7.07 (m, 1H), 5.72-5.57 (m, 1H), 4.92 (s, 2H), 1.97-1.95 (m, 1H), 1.89 (d, J = 2.4 Hz, 2H), 1.72-1.64 (m, 2H), 1.41 (s, 2H). (MH⁺ 473.1) |
| I-95 | 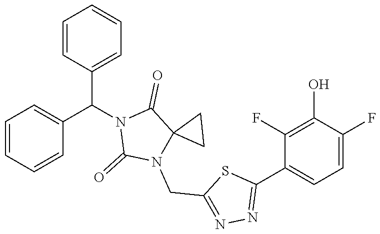 [SMILES: O=C1N(C(c2ccccc2)c2ccccc2)C(=O)C2(CC2)N1Cc1nnc(-c2ccc(F)c(O)c2F)s1] | 1H NMR (400 MHz, DMSO- d6) δ 10.84 (s, 1H), 7.72-7.58 (m, 1H), 7.46-7.18 (m, 11H), 6.52 (s, 1H), 4.93 (s, 2H), 1.68-1.53 (m, 2H), 1.36-1.22 (m, 2H). (MH⁺ 519.1) |
| I-96 | 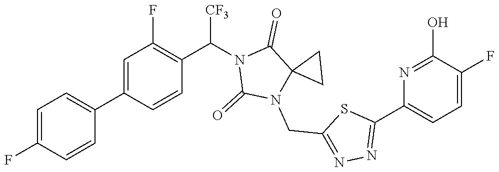 [SMILES: O=C1N(C(c2ccc(-c3ccc(F)cc3)cc2F)C(F)(F)F)C(=O)C2(CC2)N1Cc1nnc(-c2ccc(F)c(O)n2)s1] | 1H NMR (400 MHz, DMSO- d6) δ 12.35-12.05 (br m, 1H), 7.89 (t, J = 8.0 Hz, 1H), 7.84-7.74 (m, 4H), 7.69-7.60 (m, 2H), 7.32 (t, J = 8.8 Hz, 2H), 6.33 (q, J = 8.8 Hz, 1H), 4.99-4.84 (m, 2H), 1.74-1.60 (m, 2H), 1.42-1.28 (m, 2H). (MH⁺ 606.0) |
| I-97 | 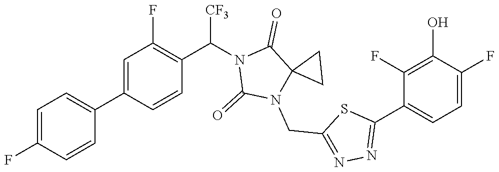 [SMILES: O=C1N(C(c2ccc(-c3ccc(F)cc3)cc2F)C(F)(F)F)C(=O)C2(CC2)N1Cc1nnc(-c2ccc(F)c(O)c2F)s1] | 1H NMR (400 MHz, DMSO- d6) δ 10.84 (br s, 1H), 7.91 (br t, J = 7.6 Hz, 1H), 7.85-7.75 (m, 2H), 7.75-7.56 (m, 3H), 7.18-7.41 (m, 3H), 6.35 (q, J = 8.8 Hz, 1H), 5.04-4.87 (m, 2H), 1.77-1.61 (m, 2H), 1.43-1.27 (m, 2H). (MH⁺ 623.1) |
| I-99A | 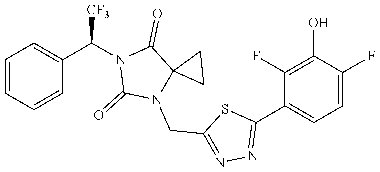 [SMILES: O=C1N([C@@H](c2ccccc2)C(F)(F)F)C(=O)C2(CC2)N1Cc1nnc(-c2ccc(F)c(O)c2F)s1] | 1H NMR (400 MHz, DMSO- d6) δ 10.58 (br s, 1H), 7.69-7.61 (m, 3H), 7.48-7.42 (m, 3H), 7.26 (dt, J = 2.0, 9.6 Hz, 1H), 6.12-6.00 (m, 1H), 4.97 (s, 2H), 1.69-1.63 (m, 2H), 1.39-1.32 (m, 2H). (MH⁺ 511.0) |
| I-100 Metabolite | 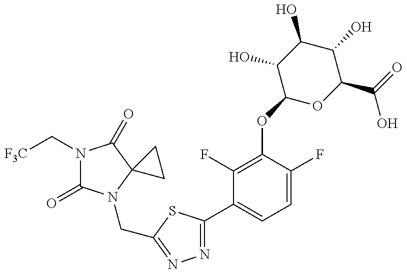 [SMILES: O=C(O)[C@H]1O[C@@H](Oc2c(F)ccc(-c3nnc(CN4C(=O)N(CC(F)(F)F)C(=O)C45CC5)s3)c2F)[C@H](O)[C@@H](O)[C@@H]1O] | 1H NMR (400 MHz, DMSO- d6) 8 8.06-7.97 (m, 1H), 7.39 (t, J = 8.8 Hz, 1H), 5.59 (br d, J = 4.8 Hz, 1H), 5.24 (br d, J = 4.4 Hz, 1H), 4.99-4.94 (m, 3H), 4.35 (q, J = 9.2 Hz, 2H), 3.67 (d, J = 10.0 Hz, 1H), 3.44-3.37 (m, 1H), 3.27 (s, 2H), 1.75-1.61 (m, 2H), 1.42-1.28 (m, 2H). (MH⁺ 611.0) |
| I-101 | 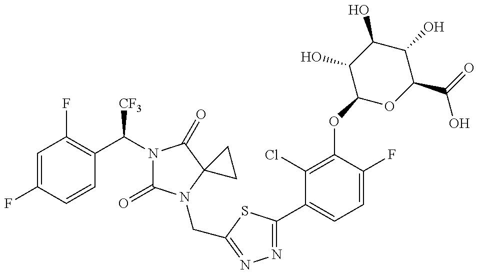 [SMILES: O=C(O)[C@H]1O[C@@H](Oc2c(F)ccc(-c3nnc(CN4C(=O)N([C@@H](c5ccc(F)cc5F)C(F)(F)F)C(=O)C45CC5)s3)c2Cl)[C@H](O)[C@@H](O)[C@@H]1O] | 1H NMR (400 MHz, DMSO- d6) δ 12.98-12.51 (br m, 1H), 8.02-7.84 (m, 2H), 7.52 (t, J = 9.6 Hz, 1H), 7.45-7.36 (m, 1H), 7.23 (dt, J = 2.0, 8.8 Hz, 1H), 6.29 (q, J = 8.4 Hz, 1H), 5.70-5.49 (br m, 1H), 5.47-5.12 (br m, 2H), 5.05 (d, J = 7.6 Hz, 1H), 5.02-4.91 (m, 2H), 3.69 (d, J = 9.6 Hz, 1H), 3.45-3.39 (m, 2H), 3.29-3.26 (m, 1H), 1.72-1.60 (m, 2H), 1.41-1.28 (m, 2H). (MH⁺ 739.0) |
| I-102 | 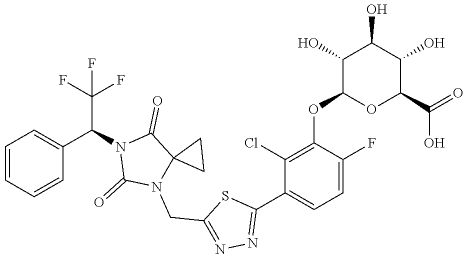 [SMILES: O=C(O)[C@H]1O[C@@H](Oc2c(F)ccc(-c3nnc(CN4C(=O)N([C@@H](c5ccccc5)C(F)(F)F)C(=O)C45CC5)s3)c2Cl)[C@H](O)[C@@H](O)[C@@H]1O] | 1H NMR (400 MHz, DMSO- d6) δ ppm 12.73 (br s, 1H), 7.94 (dd, J = 8.8, 5.6 Hz, 1H), 7.63 (dd, J = 5.6, 3.6 Hz, 2H), 7.52 (t, J = 9.6 Hz, 1H), 7.43-7.48 (m, 3H), 6.06 (q, J = 9.2 Hz, 1H), 5.59 (br d, J = 5.2 Hz, 1H), 5.26 (br d, J = 5.2 Hz, 1H), 5.04 (d, J = 7.6 Hz, 1H), 4.98 (s, 2H), 3.67 (d, J = 9.6 Hz, 1H), 3.39 (d, J = 9.6 Hz, 2H), 3.25-3.30 (m, 2H), 1.63-1.70 (m, 2H), 1.32-1.40 (m, 2H). (MH⁺ 703.0) |
| I-103 | 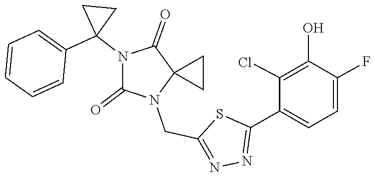 [SMILES: O=C1N(Cc2nnc(-c3ccc(F)c(O)c3Cl)s2)C2(CC2)C(=O)N1C1(c2ccccc2)CC1] | 1H NMR (400 MHz, DMSO- d6) δ ppm 9.78 (br s, 1H), 7.57-7.48 (m, 1H), 7.40-7.26 (m, 3H), 7.24-7.19 (m, 1H), 7.18-7.12 (m, 2H), 4.92 (s, 2H), 1.64-1.55 (m, 2H), 1.43 (d, J = 6.4 Hz, 4H), 1.32-1.25 (m, 2H). (MH⁺ 485.0) |
| I-104 | 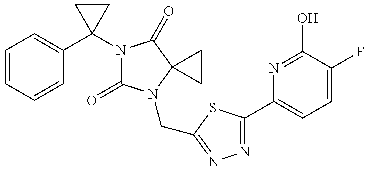 [SMILES: O=C1N(Cc2nnc(-c3ccc(F)c(O)n3)s2)C2(CC2)C(=O)N1C1(c2ccccc2)CC1] | 1H NMR (400 MHz, DMSO- d6) δ ppm 12.23 (br s, 1H), 7.83-7.74 (m, 1H), 7.68 (s, 1H), 7.35-7.27 (m, 2H), 7.24-7.19 (m, 1H), 7.18-7.13 (m, 2H), 4.86 (s, 2H), 1.64-1.57 (m, 2H), 1.43 (d, J = 1.6 Hz, 4H), 1.32-1.25 (m, 2H). (MH⁺ 452.1) |
| I-105 | 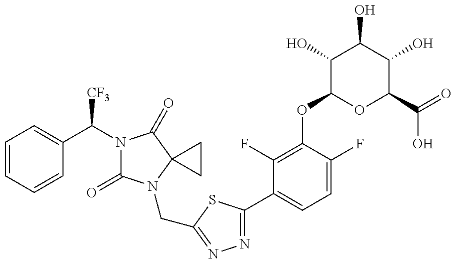 [SMILES: O=C(O)[C@H]1O[C@@H](Oc2c(F)ccc(-c3nnc(CN4C(=O)N([C@@H](c5ccccc5)C(F)(F)F)C(=O)C45CC5)s3)c2F)[C@H](O)[C@@H](O)[C@@H]1O] | 1H NMR (400 MHz, DMSO- d6) δ 12.66 (br s, 1H), 8.06-7.93 (m, 1H), 7.63 (d, J = 3.6 Hz, 2H), 7.48-7.43 (m, 3H), 7.39 (t, J = 9.6 Hz, 1H), 6.12-5.99 (m, 1H), 5.61 (br d, J = 4.4 Hz, 1H), 5.26 (br d, J = 4.4 Hz, 1H), 5.04-4.91 (m, 3H), 3.69 (d, J = 9.6 Hz, 1H), 3.40 (t, J = 8.8 Hz, 1H), 3.31-3.24 (m, 3H), 1.66 (d, J = 2.0 Hz, 2H), 1.35 (s, 2H). (MH⁺ 687.1) |

We provide additional experimental details for specific examples from Table 8 as shown below.

### Route of Compound I-88

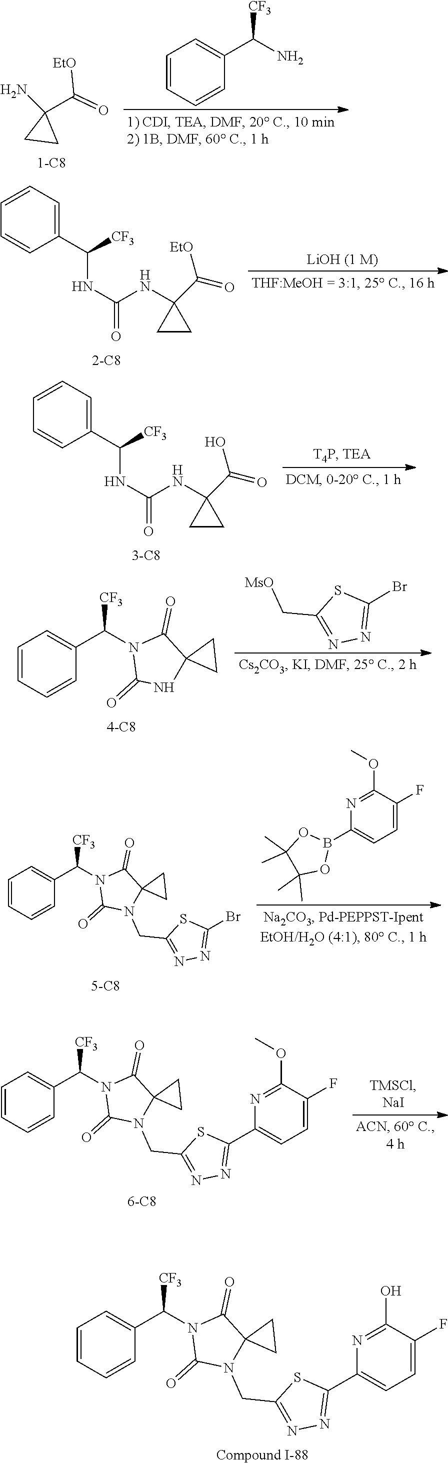

### Procedure for preparation of (S)-ethyl 1-(3-(2,2,2-trifluoro-1-phenylethyl)ureido)cyclopropanecarboxylate Compound 2-C8

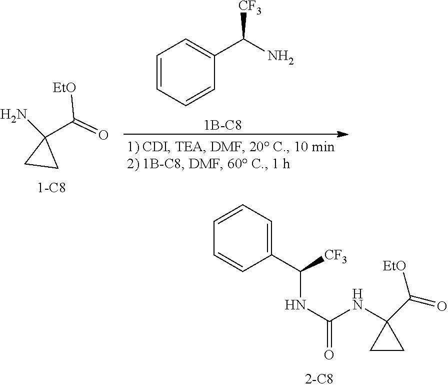 [SMILES: CCOC(=O)C1(N)CC1 ; N[C@@H](c1ccccc1)C(F)(F)F ; CCOC(=O)C1(NC(=O)N[C@@H](c2ccccc2)C(F)(F)F)CC1]

A mixture of Compound 1-C8 (600.00 mg, 4.65 mmol, 1 eq), CDI (790.93 mg, 4.88 mmol, 1.05 eq), TEA (2.35 g, 23.23 mmol, 3.23 mL, 5 eq) in DMF (6 mL) was degassed and purged with N₂ for 3 times, and (1S)-2,2,2-trifluoro-1-phenyl-ethanamine (650.93 mg, 3.72 mmol, 0.8 eq) was added 10 min later. Then the mixture was stirred at 60° C. for 1 hr under N₂ atmosphere. TLC indicated reactant was consumed completely and one new spot formed. The reaction mixture was purified by re-crystallization from H₂O 15 mL, filtered and concentrated under reduced pressure to afford Compound 2-C8 (780 mg, 2.36 mmol) as white solid.

¹H NMR (400 MHz, DMSO-d₆) δ ppm 7.48-7.40 (m, 6H), 6.66 (s, 1H), 5.64-5.46 (m, 1H), 4.06-3.91 (m, 2H), 1.35-1.26 (m, 2H), 1.14-0.96 (m, 5H).

### Preparation of (S)-1-(3-(2,2,2-trifluoro-1-phenylethyl)ureido)cyclopropanecarboxylic acid Compound 3-C8

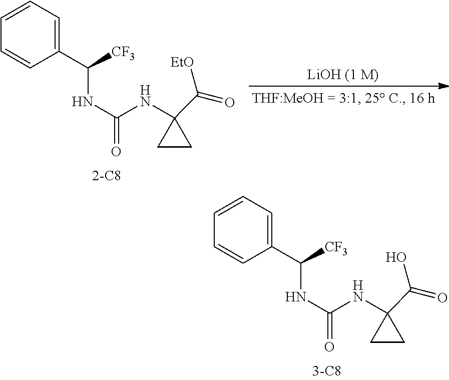 [SMILES: CCOC(=O)C1(NC(=O)N[C@@H](c2ccccc2)C(F)(F)F)CC1 ; O=C(N[C@@H](c1ccccc1)C(F)(F)F)NC1(C(=O)O)CC1]

To a solution of Compound 2-C8 (780 mg, 2.36 mmol, 1 eq) in THF (6 mL) and MeOH (2 mL) was added LiOH·H₂O (1 M, 2.36 mL, 1 eq). The mixture was stirred at 25° C. for 16 hr. TLC indicated reactant was consumed completely and one new spot formed. The reaction mixture was diluted with H₂O 40 mL and extracted with EA (20 mL*2). The aqueous phase was adjusted to pH 3-4 with 1M HCl and extracted with EA (40 mL×2). Dried over Na₂SO₄, filtered and concentrated under reduced pressure to afford Compound 3-C8 (500 mg, 1.65 mmol) as a white solid.

¹H NMR (400 MHz, DMSO-d₆) δ ppm 12.27 (br s, 1H), 7.51-7.37 (m, 5H), 7.31 (d, J=9.6 Hz, 1H), 6.59 (s, 1H), 5.52 (quin, J=8.8 Hz, 1H), 1.36-1.24 (m, 2H), 1.04-0.94 (m, 2H).

### Preparation of (S)-6-(2,2,2-trifluoro-1-phenylethyl)-4,6-diazaspiro[2.4]heptane-5,7-dione Compound 4-C8

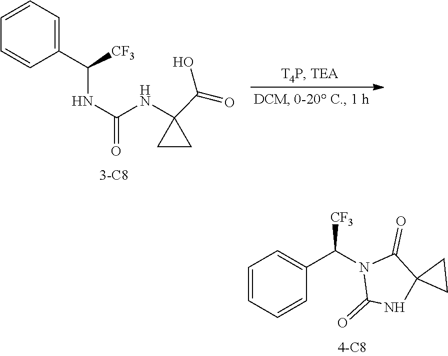 [SMILES: O=C(N[C@@H](c1ccccc1)C(F)(F)F)NC1(C(=O)O)CC1 ; O=C1NC2(CC2)C(=O)N1[C@@H](c1ccccc1)C(F)(F)F]

A mixture of Compound 3-C8 (500 mg, 1.65 mmol, 1 eq), TEA (836.97 mg, 8.27 mmol, 1.15 mL, 5 eq) in DCM (10 mL) was degassed and purged with N₂ for 3 times, and added T₄P (2.38 g, 3.31 mmol, 50% purity, 2 eq) at 0° C. Then the mixture was stirred at 20° C. for 1 hr under N₂ atmosphere. TLC indicated Reactant was consumed completely and one new spot formed. The reaction mixture was diluted with H₂O 40 mL and extracted with DCM (20 mL*2). Dried over Na₂SO₄, filtered and concentrated under reduced pressure to give a residue. The residue was purified by column chromatography (SiO₂, petroleum ether/ethyl acetate=1/0 to 3/1) to afford Compound 4-C8 (300 mg, 1.06 mmol) as a colorless oil.

¹H NMR (400 MHz, DMSO-d₆) δ ppm 9.01 (br d, J=10.0 Hz, 1H), 7.59 (d, J=4.4 Hz, 2H), 7.43 (dd, J=5.2, 1.6 Hz, 3H), 5.62-5.40 (m, 1H), 1.54-1.30 (m, 4H)

### Procedure for preparation of (S)-4-((5-bromo-1,3,4-thiadiazol-2-yl)methyl)-6-(2,2,2-trifluoro-1-phenylethyl)-4,6-diazaspiro[2.4]heptane-5,7-dione Compound 5-C8

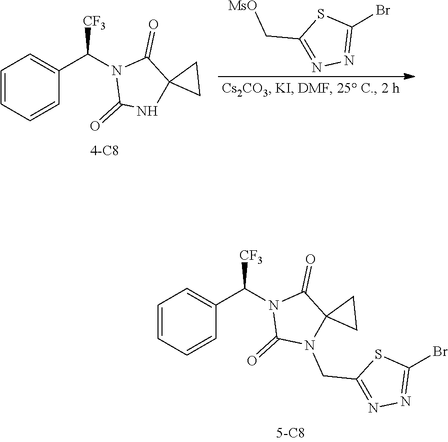 [SMILES: O=C1NC2(CC2)C(=O)N1[C@@H](c1ccccc1)C(F)(F)F ; CS(=O)(=O)OCc1nnc(Br)s1 ; O=C1N([C@@H](c2ccccc2)C(F)(F)F)C(=O)C2(CC2)N1Cc1nnc(Br)s1]

A mixture of Compound 4-C8 (300 mg, 1.06 mmol, 1 eq), (5-bromo-1,3,4-thiadiazol-2-yl)methyl methanesulfonate (345.93 mg, 1.27 mmol, 1.2 eq), Cs₂CO₃ (687.78 mg, 2.11 mmol, 2 eq), KI (175.21 mg, 1.06 mmol, 1 eq) in DMF (5 mL) was degassed and purged with N₂ for 3 times, and then the mixture was stirred at 25° C. for 2 hr under N₂ atmosphere. LC-MS showed reactant was consumed completely and one main peak with desired mass was detected. The reaction mixture was diluted with H₂O 20 mL and extracted with EA (20 mL*2). Dried over Na₂SO₄, filtered and concentrated under reduced pressure to give a residue. The residue was purified by column chromatography (SiO₂, petroleum ether/ethyl acetate=1/0 to 1/1) to afford Compound 5-C8 (280 mg, 607.04 μmol) as yellow oil.

¹H NMR (400 MHz, DMSO-d₆) δ ppm 7.71-7.56 (m, 2H) 7.51-7.35 (m, 3H) 6.03 (d, J=9.2 Hz, 1H) 4.89 (d, J=1.6 Hz, 2H) 1.65-1.54 (m, 2H) 1.37-1.29 (m, 2H)

### Preparation of (S)-4-((5-(5-fluoro-6-methoxypyridin-2-yl)-1,3,4-thiadiazol-2-yl)methyl)-6-(2,2,2-trifluoro-1-phenylethyl)-4,6-diazaspiro[2.4]heptane-5,7-dione Compound 6-C8

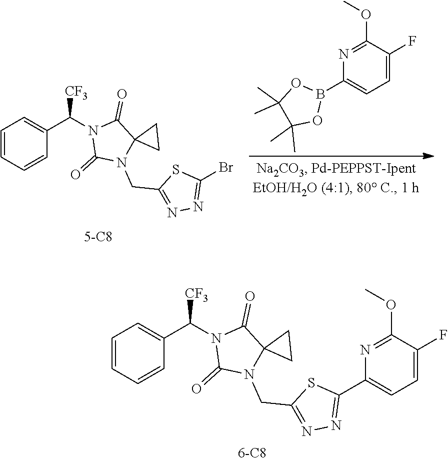 [SMILES: O=C1N([C@@H](c2ccccc2)C(F)(F)F)C(=O)C2(CC2)N1Cc1nnc(Br)s1 ; COc1nc(B2OC(C)(C)C(C)(C)O2)ccc1F ; COc1nc(-c2nnc(CN3C(=O)N([C@@H](c4ccccc4)C(F)(F)F)C(=O)C34CC4)s2)ccc1F]

A mixture of Compound 5-C8 (280 mg, 607.04 μmol, 1 eq), 3-fluoro-2-methoxy-6-(4,4,5,5-tetramethyl-1,3,2-dioxaborolan-2-yl)pyridine (184.35 mg, 728.45 μmol, 1.2 eq), 1,3-bis[2,6-bis(1-ethylpropyl)phenyl]-4,5-dichloro-2H-imidazol-1-ium-2-ide; 3-chloropyridine; dichloropalladium (26.12 mg, 30.35 μmol, 0.05 eq), Na₂CO₃ (96.51 mg, 910.56 μmol, 1.5 eq) in ethanol (4 mL) and H₂O (1 mL) was degassed and purged with Ar for 3 times, and then the mixture was stirred at 80° C. for 1 hr under argon atmosphere. LC-MS showed reactant was consumed completely and one main peak with desired mass was detected. The reaction mixture was diluted with H₂O 20 mL and extracted with EA (40 mL*2). Dried over Na₂SO₄, filtered and concentrated under reduced pressure to give a residue. The residue was purified by column chromatography (SiO₂, petroleum ether/ethyl acetate=1/0 to 1/1) to afford Compound 6-C8 (260 mg, 512.36 μmol) as yellow oil.

¹H NMR (400 MHz, DMSO-d₆) δ ppm 7.98-7.80 (m, 2H) 7.62 (d, J=2.8 Hz, 2H) 7.51-7.36 (m, 3H) 6.06 (d, J=9.2 Hz, 1H) 4.93 (s, 2H) 4.03-3.98 (m, 3H) 1.72-1.58 (m, 2H) 1.39-1.30 (m, 2H)

### Procedure for preparation of (S)-4-((5-(5-fluoro-6-hydroxypyridin-2-yl)-1,3,4-thiadiazol-2-yl)methyl)-6-(2,2,2-trifluoro-1-phenylethyl)-4,6-diazaspiro[2.4]heptane-5,7-dione Compound I-88

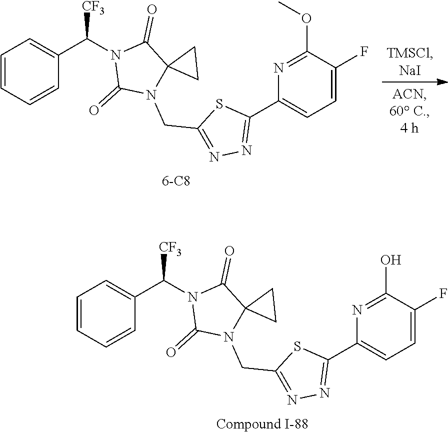 [SMILES: COc1nc(-c2nnc(CN3C(=O)N([C@@H](c4ccccc4)C(F)(F)F)C(=O)C34CC4)s2)ccc1F ; O=C1N([C@@H](c2ccccc2)C(F)(F)F)C(=O)C2(CC2)N1Cc1nnc(-c2ccc(F)c(O)n2)s1]

A mixture of Compound 6-C8 (240 mg, 472.94 μmol, 1 eq), TMSCl (513.82 mg, 4.73 mmol, 600.25 μL, 10 eq), NaI (354.46 mg, 2.36 mmol, 5 eq) in ACN (5 mL) was degassed and purged with N₂ three times, and then the mixture was stirred at 60° C. for 4 hr under N₂ atmosphere. LC-MS showed reactant was consumed completely and one main peak with desired mass was detected. The reaction mixture was diluted with H₂O 20 mL and extracted with EA (20 mL*2). Dried over Na₂SO₄, filtered and concentrated under reduced pressure to give a residue. The residue was purified by prep-HPLC (column: Phenomenex luna C18 100*40 mm*5 um; mobile phase: [H₂O (0.2% FA)-ACN]; gradient: 40%-75% B over 8.0 min) to afford Compound I-88 (60 mg, 121.60 μmol, 100% purity) as pink solid.

¹H NMR (400 MHz, DMSO-d₆) δ ppm 12.50-12.05 (br m, 1H), 7.88-7.54 (m, 4H), 7.45 (s, 3H), 6.19-5.88 (m, 1H), 5.00-4.83 (m, 2H), 1.73-1.59 (m, 2H), 1.35 (s, 2H).

LCMS: RT=2.473 min, MS cal.: 493.08, MS found: [M+H]⁺=494.0.

HPLC: RT=3.436 min, purity: 100%

### Route of Compound I-99A

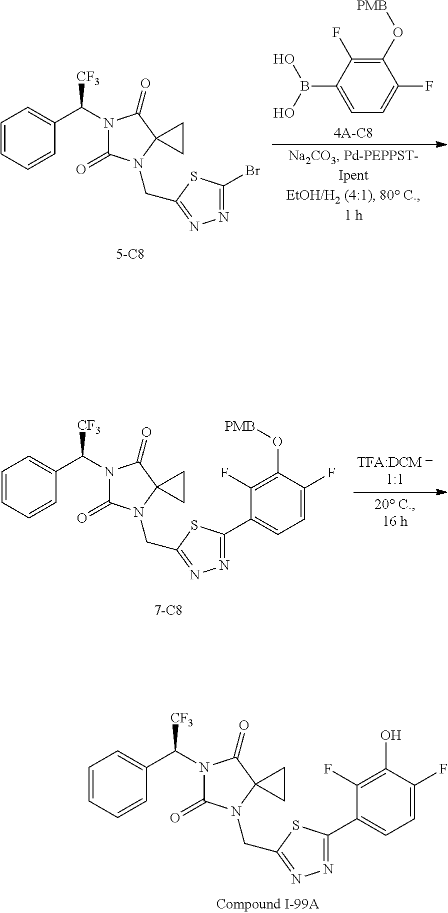

### Procedure for preparation of 4-[[5-[2,4-difluoro-3-[(4-methoxyphenyl)methoxy]phenyl]-1,3,4-thiadiazol-2-yl]methyl]-6-[(1S)-2,2,2-trifluoro-1-phenyl-ethyl]-4,6-diazaspiro[2.4]heptane-5,7-dione Compound 7-C8

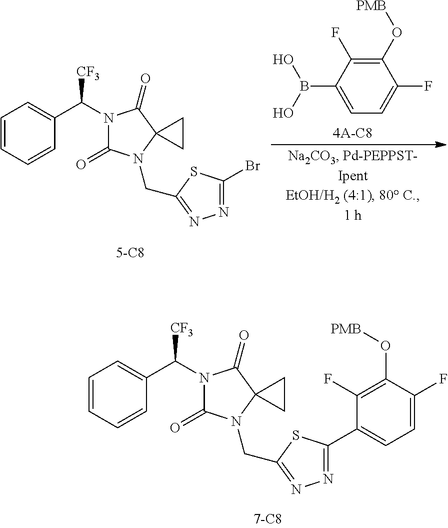

A mixture of compound 5-C8 (80 mg, 173.44 μmol, 1 eq), compound 4A-C8 (66.30 mg, 225.47 mol, 1.3 eq), Na₂CO₃ (27.57 mg, 260.16 μmol, 1.5 eq), Pd-PEPPST-Ipent (7.46 mg, 8.67 μmol, 0.05 eq) in ethanol (0.8 mL), H₂O (0.2 mL) was degassed and purged with N₂ for 3 times, and then the mixture was stirred at 80° C. for 1 hr under nitrogen atmosphere. LC-MS showed the reaction was completed. The reaction mixture was quenched by addition H₂O (10 mL×2) at 20° C., and extracted with ethyl acetate (10 mL×2). The combined organic layers were washed with brine (10 mL×2), dried over Na₂SO₄, filtered and concentrated under reduced pressure to afford compound 2 (110 mg, crude) as yellow oil.

### Procedure for preparation of 4-[[5-(2,4-difluoro-3-hydroxy-phenyl)-1,3,4-thiadiazol-2-yl]methyl]-6-[(1S)-2,2,2-trifluoro-1-phenyl-ethyl]-4,6-diazaspiro[2.4]heptane-5,7-dione Compound I-99A

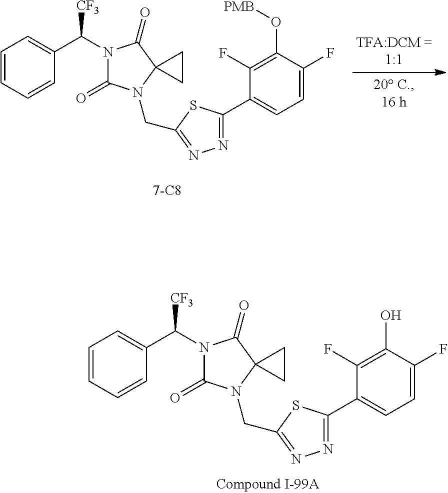

To a solution of compound 7-C8 (110 mg, 174.44 μmol, 1 eq) in DCM (2 mL) was added TFA (2 mL). The mixture was stirred at 20° C. for 16 hr. LC-MS showed the reaction was completed. The reaction mixture was concentrated under reduced pressure to give a residue. The residue was purified by prep-HPLC (column: Phenomenex luna C18 100*40 mm*3 um; mobile phase: [H₂O (0.1% TFA)-ACN]; gradient: 35%-65% B over 8.0 min) to afford Compound I-99A (35.19 mg, 68.41 μmol, 99.233% purity) as white solid.

¹H NMR (400 MHz, DMSO-d₆) δ10.80 (br d, J=1.2 Hz, 1H), 7.69-7.61 (m, 3H), 7.48-7.42 (m, 3H), 7.26 (dt, J=2.0, 9.6 Hz, 1H), 6.12-6.00 (m, 1H), 4.97 (s, 2H), 1.69-1.63 (m, 2H), 1.39-1.32 (m, 2H).

LCMS: RT=2.527 min, MS calc.: 510.0, MS found: [M+H]⁺=511.0.

### Route of Compounds I-54A & I-54B

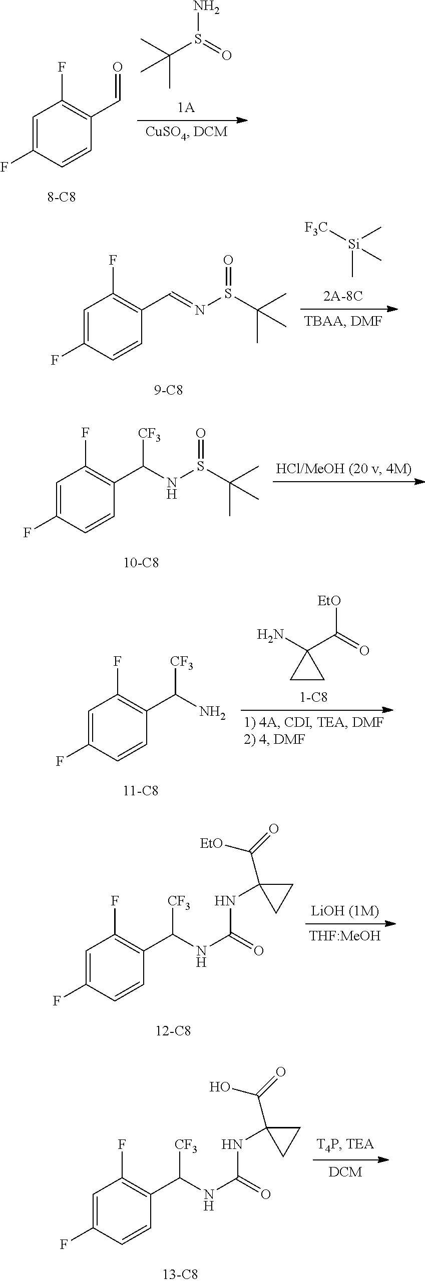

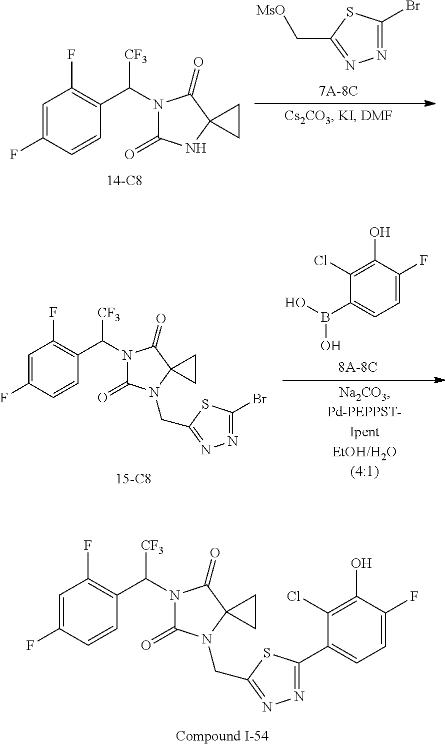 [SMILES: O=C1NC2(CC2)C(=O)N1C(c1ccc(F)cc1F)C(F)(F)F ; CS(=O)(=O)OCc1nnc(Br)s1 ; O=C1N(C(c2ccc(F)cc2F)C(F)(F)F)C(=O)C2(CC2)N1Cc1nnc(Br)s1 ; OB(O)c1ccc(F)c(O)c1Cl ; O=C1N(C(c2ccc(F)cc2F)C(F)(F)F)C(=O)C2(CC2)N1Cc1nnc(-c2ccc(F)c(O)c2Cl)s1]

### Procedure for preparation of N-(2,4-difluorobenzylidene)-2-methylpropane-2-sulfinamide Compound 9-C8

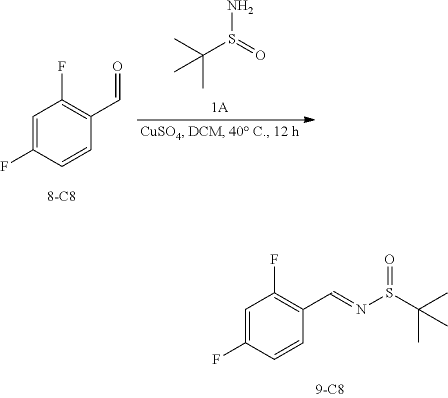

To a solution of compound 8-C8 (5 g, 35.19 mmol, 3.83 mL, 1 eq) in DCM (60 mL) was added compound 1A (7.25 g, 59.82 mmol, 1.7 eq), CuSO₄ (61.78 g, 387.04 mmol, 59.40 mL, 11 eq). The mixture was stirred at 40° C. for 24 hrs. The reaction mixture was concentrated under reduced pressure to remove DCM. The residue was purified by column chromatography (SiO₂, Petroleum ether:Ethyl acetate=99:1 to 9:1) to afford compound 9-C8 (6.58 g, 26.83 mmol) as colorless oil.

¹H NMR (400 MHz, CDCl₃) δ 8.83 (s, 1H), 8.02 (dt, J=6.8, 8.0 Hz, 1H), 7.01-6.95 (m, 1H), 6.90 (ddd, J=1.6, 8.4, 10.8 Hz, 1H), 1.27 (s, 9H).

### Procedure for preparation of N-(1-(2,4-difluorophenyl)-2,2,2-trifluoroethyl)-2-methylpropane-2-sulfinamide Compound 10-C8

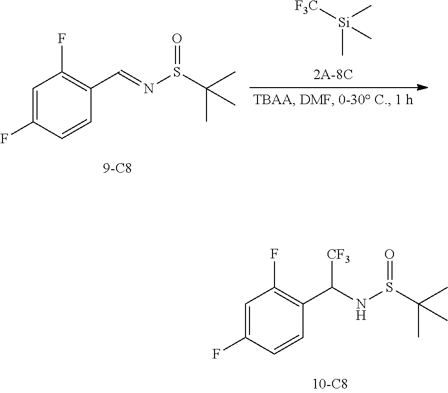

To a solution of compound 9-C8 (5 g, 20.38 mmol, 1 eq) in DMF (50 mL) was added tetrabutylammonium; acetate (6.15 g, 20.38 mmol, 6.21 mL, 1 eq) and compound 2A-8C (8.70 g, 61.15 mmol, 3 eq) at 0° C. The mixture was stirred at 30° C. for 3 hrs. The mixture was poured into water (20 mL) and extracted with EtOAc (20 mL×3). The combined organic layers were dried over Na₂SO₄, filtered and concentrated under reduced pressure. The residue was purified by column chromatography (SiO₂, petroleum ether:ethyl acetate=9:1 to 1:1) to afford compound 10-C8 (4 g, 12.69 mmol) as white solid.

¹H NMR (400 MHz, CDCl₃) δ 7.39 (d, J=6.4 Hz, 1H), 6.99-6.87 (m, 2H), 5.09 (t, J=7.6 Hz, 1H), 3.89 (d, J=8.0 Hz, 1H), 1.26 (s, 9H).

### Procedure for preparation of 1-(2,4-difluorophenyl)-2,2,2-trifluoroethan-1-amine

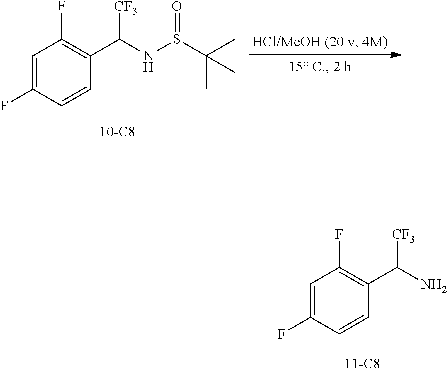 [SMILES: CC(C)(C)S(=O)NC(c1ccc(F)cc1F)C(F)(F)F ; NC(c1ccc(F)cc1F)C(F)(F)F]

A mixture of compound 10-C8 (3.76 g, 11.93 mmol, 1 eq) in HCl/MeOH (4 M, 60 mL, 20.13 eq) was degassed and purged with N₂ for 3 times, and then the mixture was stirred at 15° C. for 2 hrs under N₂ atmosphere. The reaction mixture was concentrated under reduced pressure to give a residue to afford compound 11-C8 (3.3 g, crude) as white solid.

¹H NMR (400 MHz, MeOD) δ 7.76-7.65 (m, 1H), 7.32-7.17 (m, 2H), 5.64 (q, J=7.2 Hz, 1H).

### Procedure for preparation of ethyl 1-(3-(1-(2,4-difluorophenyl)-2,2,2-trifluoroethyl)ureido)cyclopropane-1-carboxylate Compound 12-C8

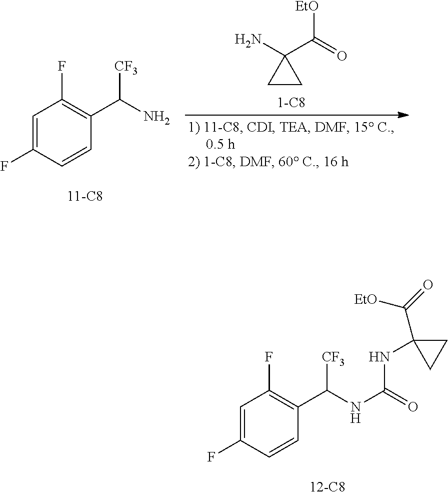 [SMILES: NC(c1ccc(F)cc1F)C(F)(F)F ; CCOC(=O)C1(N)CC1 ; CCOC(=O)C1(NC(=O)NC(c2ccc(F)cc2F)C(F)(F)F)CC1]

To a solution of compound 11-C8 (2.07 g, 15.99 mmol, 1.2 eq) in DMF (15 mL) was added TEA (10.79 g, 106.63 mmol, 14.84 mL, 8 eq), CDI (2.27 g, 13.99 mmol, 1.05 eq) at 15° C. for 0.5 hr, then added compound 4 (3.3 g, 13.33 mmol, 1 eq, HCl) in DMF (15 mL). The mixture was stirred at 60° C. for 12 hrs. The mixture was poured into water (20 mL) and extracted with EtOAc (20 mL×3). The combined organic layers were dried over Na₂SO₄, filtered and concentrated under reduced pressure. The residue was purified by column chromatography (SiO₂, Petroleum ether:ethyl acetate=93:7 to 4:1) to afford compound 12-C8 (2.47 g, 6.74 mmol) as colorless oil.

¹H NMR (400 MHz, CDCl₃) δ 7.40-7.30 (m, 1H), 6.92-6.76 (m, 2H), 6.69 (br d, J=8.8 Hz, 1H), 6.17 (br s, 1H), 5.91-5.76 (m, 1H), 4.07 (q, J=7.2 Hz, 2H), 1.51 (q, J=3.6 Hz, 2H), 1.19-1.08 (m, 5H).

### Procedure for preparation of 1-(3-(1-(2,4-difluorophenyl)-2,2,2-trifluoroethyl)ureido)cyclopropane-1-carboxylic acid Compound 13-C8

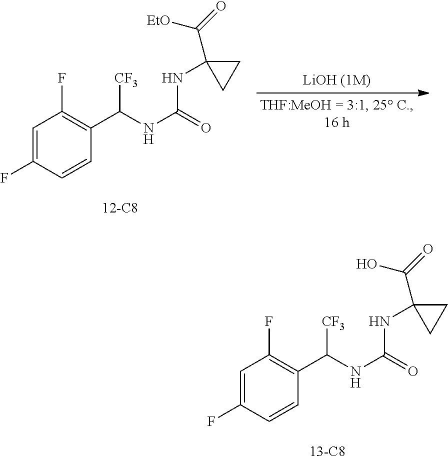 [SMILES: CCOC(=O)C1(NC(=O)NC(c2ccc(F)cc2F)C(F)(F)F)CC1 ; O=C(NC(c1ccc(F)cc1F)C(F)(F)F)NC1(C(=O)O)CC1]

To a solution of compound 12-C8 (2.47 g, 6.74 mmol, 1 eq) in THF (30 mL), MeOH (10 mL) was added LiOH·H₂O (1 M, 7.08 mL, 1.05 eq). The mixture was stirred at 25° C. for 12 hrs. LCMS showed the reaction was completed. The reaction mixture was quenched by addition H₂O (50 mL×2) at 20° C., and extracted with EtOAc (50 mL×2). The aqueous phase was adjusted to PH=3 with citric acid solution, and extracted with EtOAc (50 mL×2). The combined organic layers were washed with brine (50 mL×2), dried over Na₂SO₄, filtered and concentrated under reduced pressure to afford compound 13-C8 (1.72 g, 5.09 mmol) as white solid.

¹H NMR (400 MHz, DMSO-d₆) δ 12.28 (br s, 1H), 7.66-7.57 (m, 1H), 7.46 (d, J=9.6 Hz, 1H), 7.43-7.33 (m, 1H), 7.24 (br dt, J=2.0, 8.4 Hz, 1H), 6.72 (br s, 1H), 5.79 (quin, J=8.4 Hz, 1H), 1.33-1.25 (m, 2H), 0.99 (d, J=3.6 Hz, 2H).

### Procedure for preparation of 6-[1-(2,4-difluorophenyl)-2,2,2-trifluoro-ethyl]-4,6-diazaspiro[2.4]heptane-5,7-dione Compound 14-C8

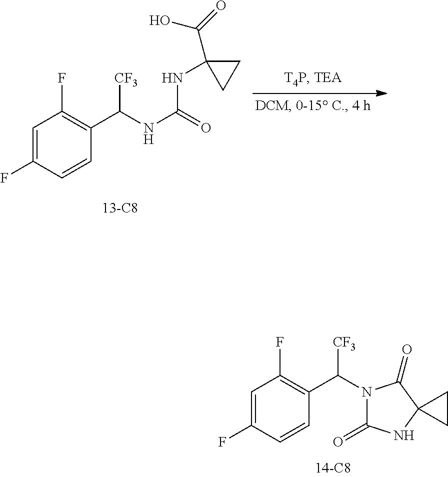 [SMILES: O=C(NC(c1ccc(F)cc1F)C(F)(F)F)NC1(C(=O)O)CC1 ; O=C1NC2(CC2)C(=O)N1C(c1ccc(F)cc1F)C(F)(F)F]

A mixture of compound 13-C8 (1.72 g, 5.09 mmol, 1 eq), TEA (2.57 g, 25.43 mmol, 3.54 mL, 5 eq) in DCM (20 mL) was degassed and purged with nitrogen for 3 times, T₄P (7.33 g, 10.17 mmol, 50% purity, 2 eq) was added at 0° C. and then the mixture was stirred at 15° C. for 2 hr under N₂ atmosphere. TLC (petroleum ether:ethyl acetate=2:1, Rt=0.7) showed the starting material was consumed. The reaction mixture was quenched by addition H₂O (50 mL×2) at 20° C., and extracted with DCM (50 mL×2). The combined organic layers were washed with brine (50 mL×2), dried over Na₂SO₄, filtered and concentrated under reduced pressure to give a residue. The residue was purified by column chromatography (SiO₂, petroleum ether:ethyl acetate=89:11) to afford compound 14-C8 (420 mg, 1.31 mmol) as white oil.

¹H NMR (400 MHz, CDCl₃) δ 7.42-7.34 (m, 1H), 7.02-6.89 (m, 2H), 5.60 (q, J=7.6 Hz, 1H), 1.59-1.49 (m, 4H).

### Procedure for preparation of 4-[(5-bromo-1,3,4-thiadiazol-2-yl)methyl]-6-[1-(2,4-difluorophenyl)-2,2,2-trifluoro-ethyl]-4,6-diazaspiro[2.4]heptane-5,7-dione Compound 15-C8

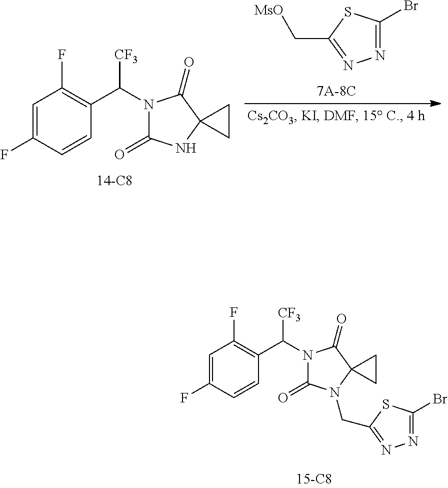 [SMILES: O=C1NC2(CC2)C(=O)N1C(c1ccc(F)cc1F)C(F)(F)F ; CS(=O)(=O)OCc1nnc(Br)s1 ; O=C1N(C(c2ccc(F)cc2F)C(F)(F)F)C(=O)C2(CC2)N1Cc1nnc(Br)s1]

A mixture of compound 14-C8 (420 mg, 1.31 mmol, 1 eq), compound 7A-8C (429.89 mg, 1.57 mmol, 1.2 eq), KI (217.73 mg, 1.31 mmol, 1 eq), Cs₂CO₃ (854.70 mg, 2.62 mmol, 2 eq) in DMF (4 mL) was degassed and purged with N₂ for 3 times, and then the mixture was stirred at 15° C. for 2 hr under N₂ atmosphere. TLC (petroleum ether:ethyl acetate=2:1, Rt=0.51) showed the starting material was consumed completely. The reaction mixture was quenched by addition H₂O (20 mL×2) at 20° C., and extracted with ethyl acetate (20 mL×2). The combined organic layers were washed with brine (20 mL×2), dried over Na₂SO₄, filtered and concentrated under reduced pressure to give a residue. The residue was purified by column chromatography (SiO₂, petroleum ether:ethyl acetate=82:18) to afford compound 15-C8 (450 mg, 905.00 μmol) as white oil.

¹H NMR (400 MHz, CDCl₃) δ 8.08-7.97 (m, 1H), 6.98 (tt, J=1.2, 7.8 Hz, 1H), 6.88 (ddd, J=2.4, 8.4, 10.4 Hz, 1H), 6.17 (q, J=8.4 Hz, 1H), 4.78-4.61 (m, 2H), 1.57-1.45 (m, 4H).

### Procedure for preparation of 4-[[5-(2-chloro-4-fluoro-3-hydroxy-phenyl)-1,3,4-thiadiazol-2-yl]methyl]-6-[1-(2,4-difluorophenyl)-2,2,2-trifluoro-ethyl]-4,6-diazaspiro[2.4]heptane-5,7-dione Compound I-54

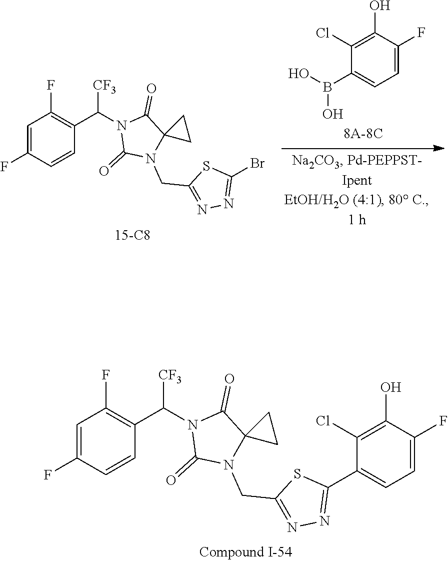 [SMILES: O=C1N(C(c2ccc(F)cc2F)C(F)(F)F)C(=O)C2(CC2)N1Cc1nnc(Br)s1 ; OB(O)c1ccc(F)c(O)c1Cl ; O=C1N(C(c2ccc(F)cc2F)C(F)(F)F)C(=O)C2(CC2)N1Cc1nnc(-c2ccc(F)c(O)c2Cl)s1]

A mixture of compound 15-C8 (150 mg, 301.67 μmol, 1 eq), compound 8A-8C (74.65 mg, 392.17 mol, 1.3 eq), Na₂CO₃ (47.96 mg, 452.50 μmol, 1.5 eq), Pd-PEPPST-Ipent (12.98 mg, 15.08 μmol, 0.05 eq) in EtOH (1.6 mL) and H₂O (0.4 mL) was degassed and purged with N₂ for 3 times, and then the mixture was stirred at 80° C. for 1 hr under N₂ atmosphere. LCMS showed the reaction was completed. The aqueous phase was adjusted to pH=3 with citric acid solution, and extracted with ethyl acetate (15 mL×2). The combined organic layers were washed with brine (15 mL×2), dried over Na₂SO₄, filtered and concentrated under reduced pressure to give a residue. The residue was purified by prep-HPLC (column: Phenomenex luna C18 100*40 mm*3 um; mobile phase: [H₂O (0.1% TFA)-ACN]; gradient: 35%-75% B over 8.0 min) to afford Compound I-54 (82.75 mg, 147.01 μmol, 100% purity) as white solid.

¹H NMR (400 MHz, DMSO-d₆) δ 10.97 (br s, 1H), 7.95-7.84 (m, 1H), 7.60 (dd, J=5.6, 8.8 Hz, 1H), 7.45-7.35 (m, 2H), 7.23 (dt, J=2.0, 8.8 Hz, 1H), 6.29 (q, J=8.8 Hz, 1H), 4.95 (s, 2H), 1.70-1.63 (m, 2H), 1.40-1.33 (m, 2H).

### Procedure for preparation of (S)-4-((5-(2-chloro-4-fluoro-3-hydroxyphenyl)-1,3,4-thiadiazol-2-yl)methyl)-6-(1-(2,4-difluorophenyl)-2,2,2-trifluoroethyl)-4,6-diazaspiro[2.4]heptane-5,7-dione Compound I-54A and (R)-4-((5-(2-chloro-4-fluoro-3-hydroxyphenyl)-1,3,4-thiadiazol-2-yl)methyl)-6-(1-(2,4-difluorophenyl)-2,2,2-trifluoroethyl)-4,6-diazaspiro[2.4]heptane-5,7-dione Compound I-54B

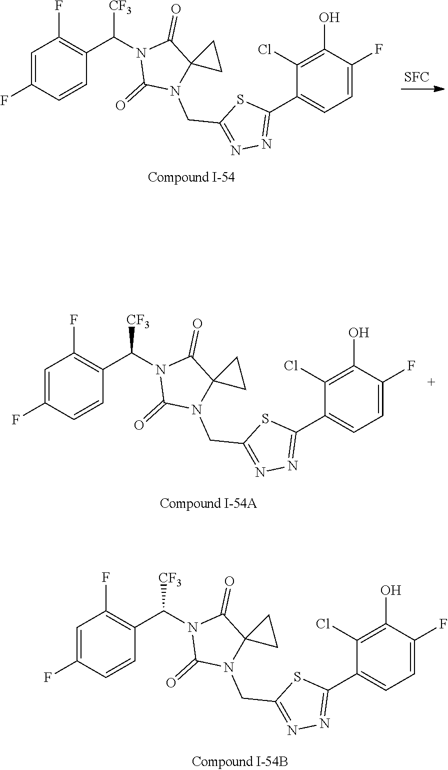

A mixture of Compound I-54 (60 mg, 111.67 μmol, 1 eq) was purified by prep-SFC (column: ChiralPak IH, 250*30 mm, 10 um; mobile phase: [CO₂-MeOH (0.1% NH₃·H₂O)]; B %: 0%, isocratic elution mode) to afford Compound I-54A (18.2 mg, 99.866% purity) as white solid and Compound I-54B (18.0 mg, 100% purity) as white solid.

### Compound I-54A:

LCMS: RT=2.717 min, MS cal.: 562.1, MS found: [M+H]⁺=563.0.

¹H NMR (400 MHz, DMSO-d₆) δ=11.00 (br s, 1H), 7.97-7.84 (m, 1H), 7.58 (dd, J=5.6, 8.8 Hz, 1H), 7.45-7.35 (m, 2H), 7.23 (dt, J=2.0, 8.4 Hz, 1H), 6.29 (q, J=8.8 Hz, 1H), 5.03-4.88 (m, 2H), 1.72-1.61 (m, 2H), 1.40-1.28 (m, 2H)

### Compound I-54B:

LCMS: RT=2.717 min, MS cal.: 562.1, MS found: [M+H]⁺=563.0.

¹H NMR (400 MHz, DMSO-d₆) δ=11.00 (br d, J=1.2 Hz, 1H), 8.00-7.84 (m, 1H), 7.58 (dd, J=5.6, 8.4 Hz, 1H), 7.48-7.32 (m, 2H), 7.30-7.15 (m, 1H), 6.29 (q, J=8.8 Hz, 1H), 5.03-4.86 (m, 2H), 1.74-1.61 (m, 2H), 1.41-1.28 (m, 2H)

### Route of Compounds I-90B & I-90A

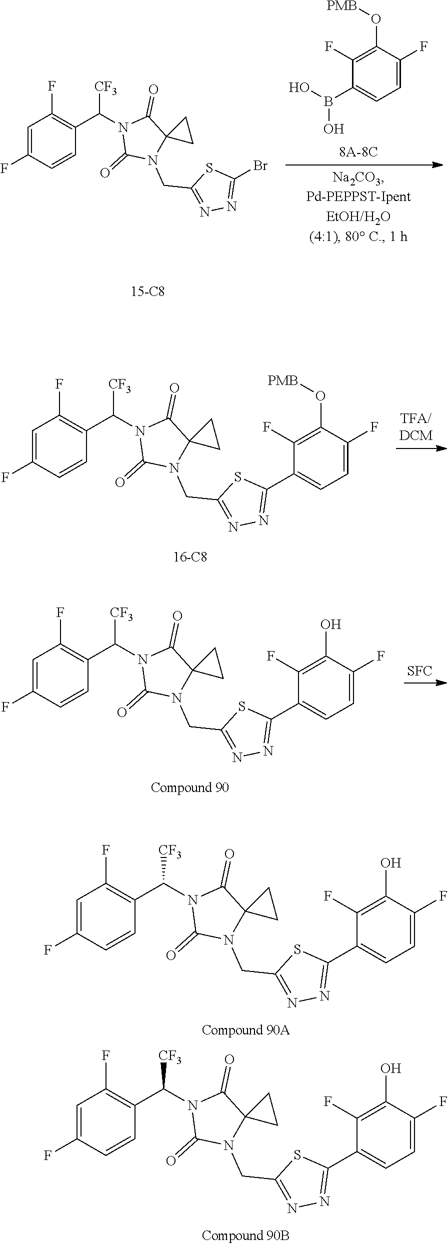

### Procedure for preparation of 4-((5-(2,4-difluoro-3-((4-methoxybenzyl)oxy)phenyl)-1,3,4-thiadiazol-2-yl)methyl)-6-(1-(2,4-difluorophenyl)-2,2,2-trifluoroethyl)-4,6-diazaspiro[2.4]heptane-5,7-dione Compound 90

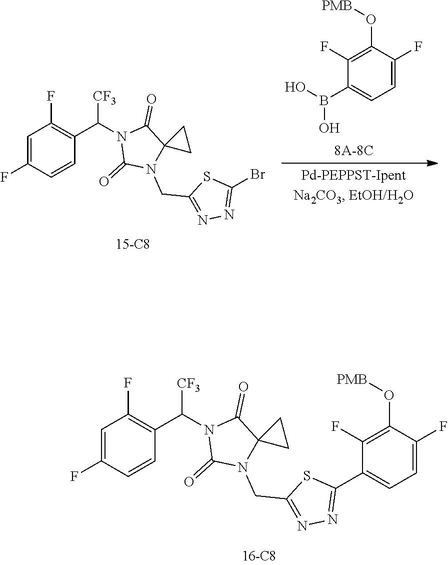

To a solution of compound 15-C8 (150 mg, 301.67 μmol, 1 eq) and [compound 8A-8C (115.32 mg, 392.17 μmol, 1.3 eq) in EtOH (1.5 mL) and H₂O (0.3 mL) was added Na₂CO₃ (47.96 mg, 452.50 μmol, 1.5 eq) and 1,3-bis[2,6-bis(1-ethylpropyl)phenyl]-4,5-dichloro-2H-imidazol-1-ium-2-ide; 3-chloropyridine; dichloropalladium (12.98 mg, 15.08 μmol, 0.05 eq) at 15° C. under nitrogen. Then the mixture was stirred at 80° C. for 1 h. The mixture was cooled to 20° C. The residue was poured into ice-water (1 mL). The aqueous phase was extracted with ethyl acetate (2 mL×3). The combined organic phase was washed with brine (2 mL), dried with anhydrous Na₂SO₄, filtered and concentrated in vacuum. The residue was purified by silica gel chromatography (column height: 250 mm, diameter: 100 mm, 100-200 mesh silica gel, petroleum ether:ethyl acetate=5:1, 2:1) to afford compound 3 (150 mg, 225.03 μmol) as a white solid.

¹H NMR (400 MHz, DMSO-d₆) δ 7.98-7.85 (m, 2H), 7.46-7.29 (m, 4H), 7.24 (dt, J=2.0, 8.8 Hz, 1H), 6.98-6.88 (m, 2H), 6.34-6.22 (m, 1H), 5.17 (s, 2H), 4.96 (d, J=1.2 Hz, 2H), 3.74 (s, 3H), 1.72-1.61 (m, 2H), 1.39-1.28 (m, 2H)

### Preparation of (R)-4-((5-(2,4-difluoro-3-hydroxyphenyl)-1,3,4-thiadiazol-2-yl)methyl)-6-(1-(2,4-difluorophenyl)-2,2,2-trifluoroethyl)-4,6-diazaspiro[2.4]heptane-5,7-dione Compound 90A and (S)-4-((5-(2,4-difluoro-3-hydroxyphenyl)-1,3,4-thiadiazol-2-yl)methyl)-6-(1-(2,4-difluorophenyl)-2,2,2-trifluoroethyl)-4,6-diazaspiro[2.4]heptane-5,7-dione Compound 90B

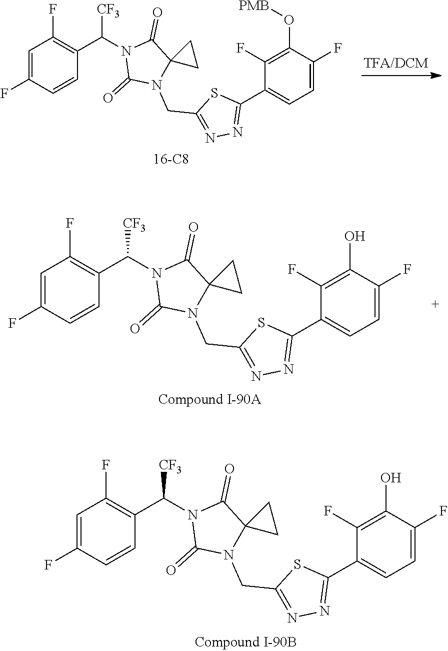

To a solution of compound 16-C8 (240 mg, 360.05 μmol, 1 eq) in DCM (2 mL) was added TFA (2 mL) under nitrogen. Then the mixture was stirred at 15° C. for 2 h. The mixture was concentrated under reduced pressure to give a residue. The residue was purified by SFC (column: DAICEL CHIRALPAK IG (250 mm*30 mm, 10 um); mobile phase: [CO₂-MeOH (0.1% NH₃H₂O)]; B %: 17%, isocratic elution mode) to afford Compound I-90A (48 mg, 86.97 μmol, 99% purity) as white solid.

LCMS: RT=2.717 min, MS cal.: 546.1, MS found: [M+H]⁺=547.0.

¹H NMR (400 MHz, DMSO-d₆) δ=11.12-10.63 (br m, 1H), 7.96-7.85 (m, 1H), 7.67-7.59 (m, 1H), 7.44-7.36 (m, 1H), 7.30-7.17 (m, 2H), 6.29 (q, J=9.2 Hz, 1H), 5.02-4.88 (m, 2H), 1.71-1.60 (m, 2H), 1.40-1.28 (m, 2H)

To get Compound I-90B (42 mg, 73.79 μmol, 96% purity) as white solid.

LCMS: RT=2.711 min, MS cal.: 546.1, MS found: [M+H]⁺=547.0.

¹H NMR (400 MHz, DMSO-d₆) δ 10.80 (br s, 1H), 7.97-7.84 (m, 1H), 7.66 (ddd, J=6.0, 7.6, 8.8 Hz, 1H), 7.41 (ddd, J=2.4, 8.8, 11.2 Hz, 1H), 7.32-7.15 (m, 2H), 6.29 (q, J=8.8 Hz, 1H), 5.02-4.89 (m, 2H), 1.71-1.60 (m, 2H), 1.37-1.28 (m, 2H)

### Route to Compound I-97 and Compound I-96

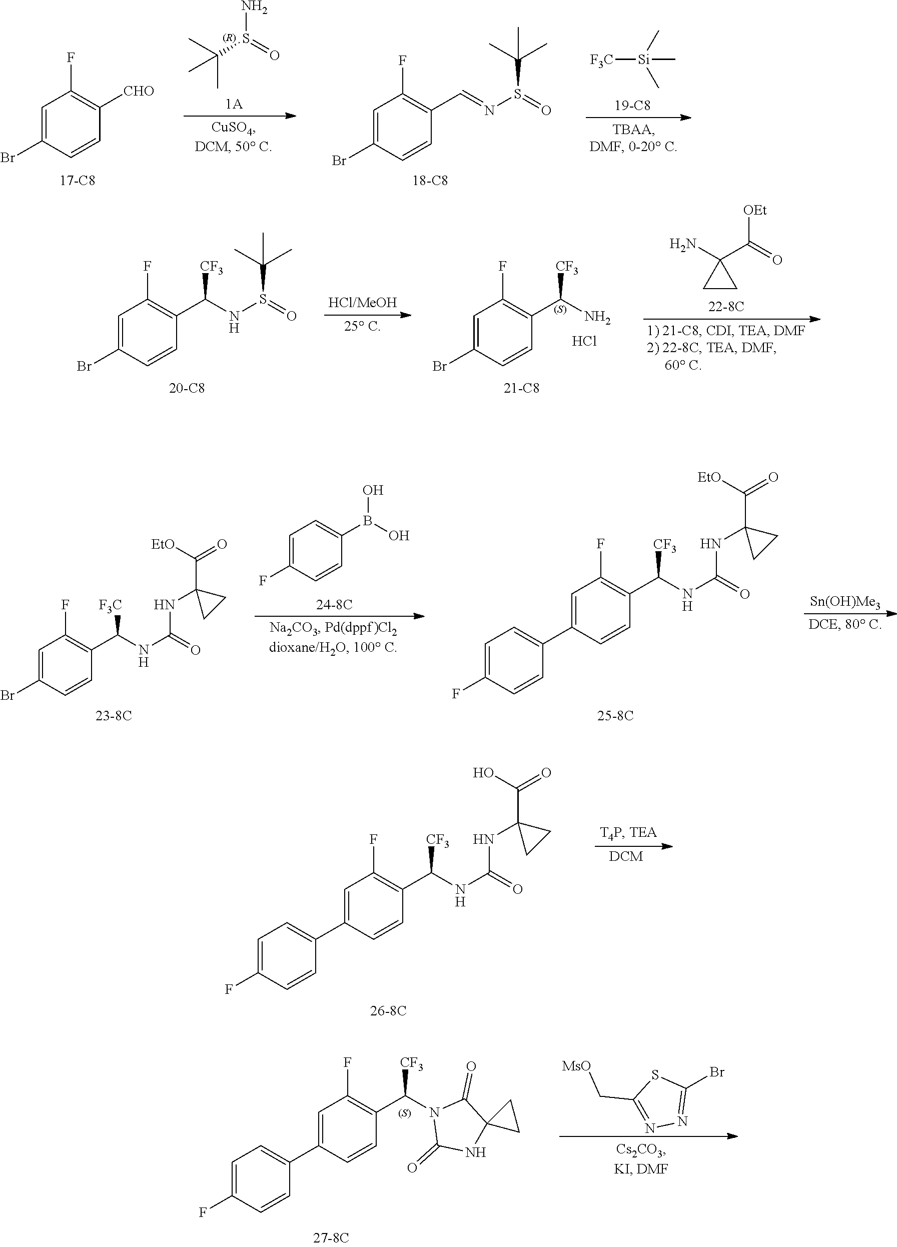

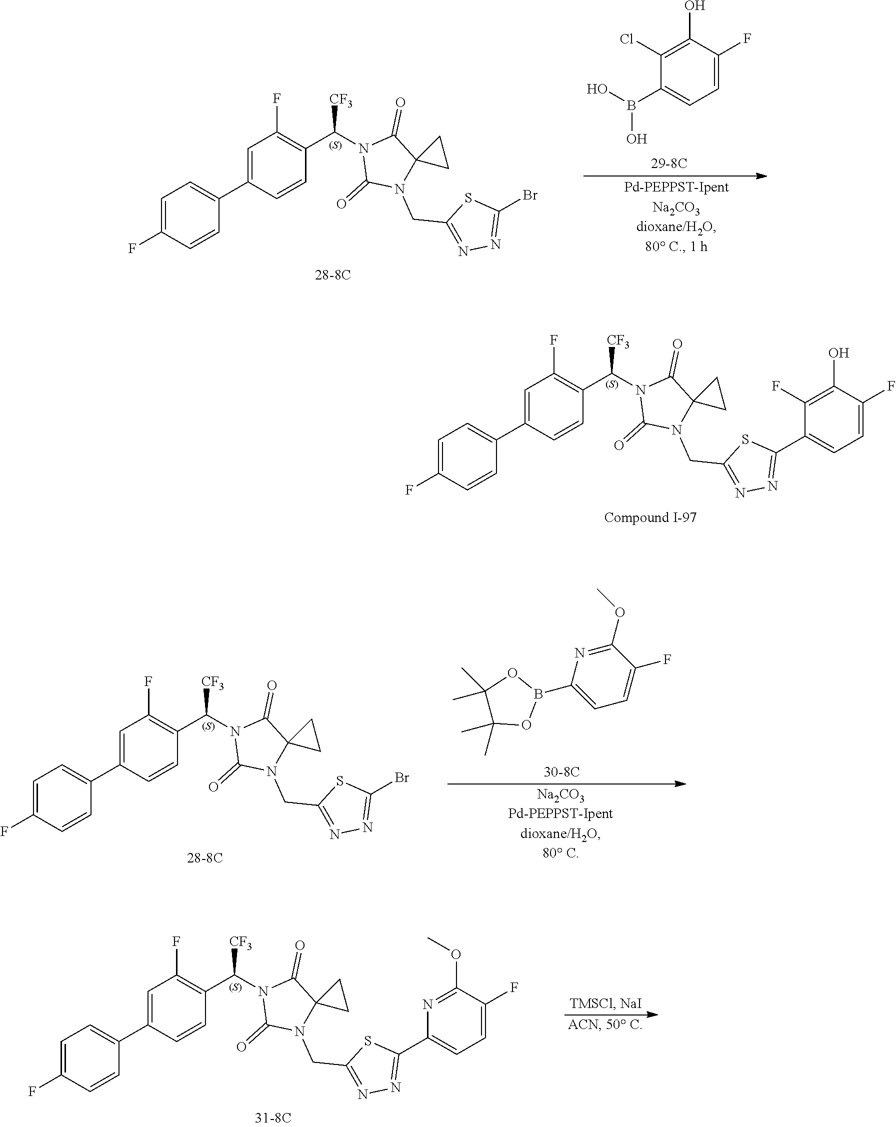

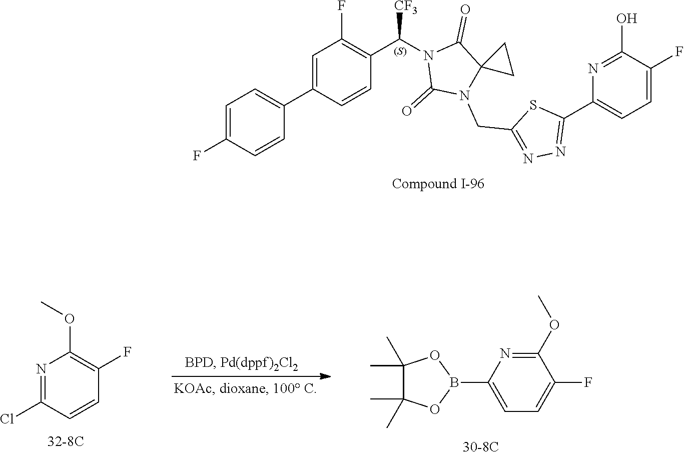

### Preparation of (R)—N-(4-bromo-2 fluorobenzylidene)-2-methylpropane-2-sulfinamide Compound 18-C8

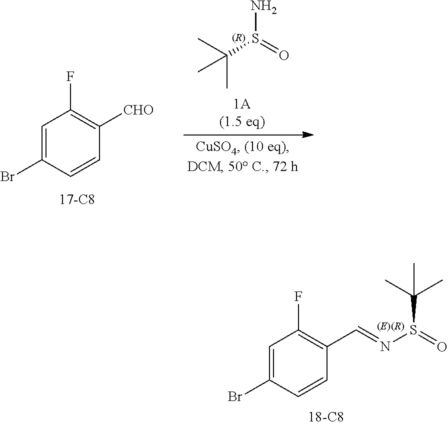

To a mixture of Compound 17-C8 (20 g, 98.5 mmol, 1 eq) and Compound 1A (17.9 g, 147 mmol, 1.5 eq) in DCM (300 mL) was added CuSO₄ (157 g, 985 mmol, 151 mL, 10 eq) in one portion at 25° C. The mixture was stirred at 50° C. for 72 hours. The mixture was filtered. The residue was poured into water (100 mL). The aqueous phase was extracted with DCM (50 mL×2). The combined organic phase was washed with brine (150 mL), dried with anhydrous Na₂SO₄, filtered and concentrated in vacuum. The residue was purified by silica gel chromatography (petroleum ether/ethyl acetate=50:1, to 5:1) to afford Compound 18-C8 (21.4 g, 69.8 mmol) as yellow solid.

¹H NMR (400 MHz, CDCl₃) δ 8.76 (s, 1H), 8.05 (dt, J=8.4 Hz, 1H), 7.35-7.24 (m, 2H), 1.19 (s, 9H).

### Preparation of (R)—N—((S)-1-(4-bromo-2-fluorophenyl)-2,2,2-trifluoroethyl)-2-methylpropane-2-sulfinamide Compound 20-C8

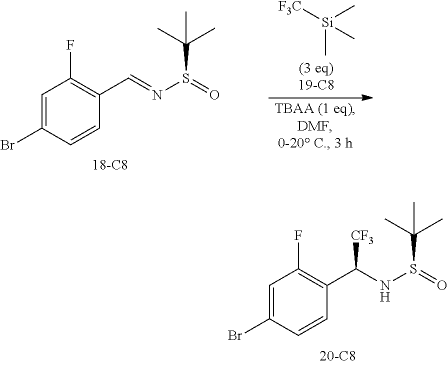

To a mixture of Compound 18-C8 (21.4 g, 69.8 mmol, 1 eq) in DMF (220 mL) was added trimethyl(trifluoromethyl)silane compound 19-C8 (29.8 g, 209.7 mmol, 3 eq) and TBAA (21.1 g, 69.9 mmol, 21.3 mL, 1 eq) in one portion at 0° C. The mixture was stirred at 20° C. for 3 hours. The mixture was poured into water (200 mL). The aqueous phase was extracted with ethyl acetate (100 mL×3). The combined organic phase was washed with brine (200 mL), dried with anhydrous Na₂SO₄, filtered and concentrated in vacuum. The residue was purified by silica gel chromatography (petroleum ether/ethyl acetate=30:1, to 3:1), and recrystallization from PE (200 mL) and EtOAc (10 mL) at 0-20° C. to afford Compound 20-C8 (17 g, 45.2 mmol) as white solid.

¹H NMR (400 MHz, CDCl₃) δ 7.39-7.34 (m, 2H), 7.30-7.26 (m, 1H), 5.11-5.05 (m, 1H), 3.92 (d, J=7.6 Hz, 1H), 1.27 (s, 9H).

### Preparation of (S)-1-(4-bromo-2-fluorophenyl)-2,2,2-trifluoroethan-1-amine Compound 21-C8

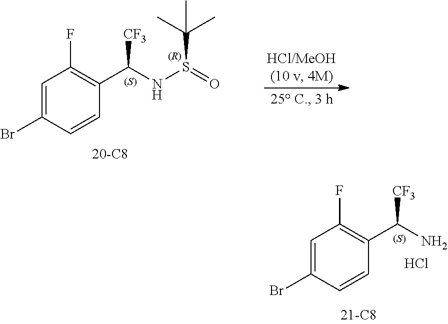

A mixture of Compound 20-C8 (17 g, 45.2 mmol, 1 eq) in 4M HCl/MeOH (170 mL) was stirred at 25° C. for 3 hours. The mixture was concentrated in reduced pressure at 40° C. to afford crude Compound 21-C8 (13.8 g, 44.7 mmol, HCl) as white solid, which was used to next step directly.

¹H NMR (400 MHz, DMSO-d₆) δ 9.53 (brs, 2H), 7.76 (m, 2H), 7.68-7.65 (m, 1H), 5.71-5.65 (m, 1H).

### Preparation of ethyl (S)-1-(3-(1-(4-bromo-2-fluorophenyl)-2,2,2-trifluoroethyl)ureido)cyclopropane-1-carboxylate Compound 23-C8

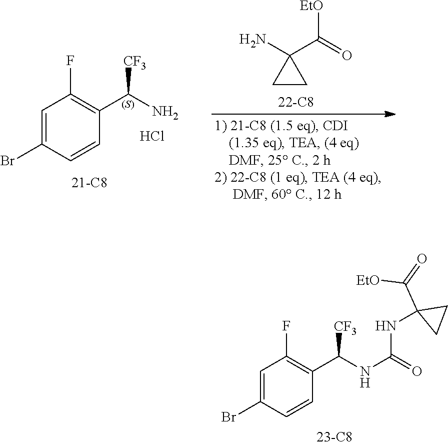

To a mixture of Compound 21-C8 (7.66 g, 59.3 mmol, 1.5 eq) and TEA (16.0 g, 158 mmol, 22.0 mL, 4 eq) in DMF (100 mL) was added CDI (8.66 g, 53.4 mmol, 1.35 eq) in one portion at 25° C. under N₂. The mixture was stirred at 25° C. for 2 hrs, then Compound 22-C8 (12.2 g, 39.5 mmol, 1 eq, HCl) and TEA (16.0 g, 158 mmol, 22.0 mL, 4 eq) in DMF (100 mL) was added and stirred for 12 hours at 60° C. The mixture was poured into water (200 mL). The aqueous phase was extracted with ethyl acetate (100 mL×3). The combined organic phase was washed with 1N HCl solution (100 mL×5), brine (150 mL), dried with anhydrous Na₂SO₄, filtered and concentrated in vacuum to afford Compound 23-C8 (14.4 g, 33.7 mmol) as white solid, which was used to next step directly.

¹H NMR (400 MHz, CDCl₃) δ 7.27-7.22 (m, 2H), 7.16-7.12 (m, 1H), 5.75 (br d, J=8 Hz, 1H), 5.71-5.67 (m, 1H), 5.38 (brs, 1H), 4.09-4.03 (m, 2H), 1.56-1.53 (m, 2H), 1.16-1.09 (m, 5H).

### Preparation of ethyl (S)-1-(3-(1-(3,4′-difluoro-[1,1′-biphenyl]-4-yl)-2,2,2-trifluoroethyl)ureido)cyclopropane-1-carboxylate Compound 25-8C

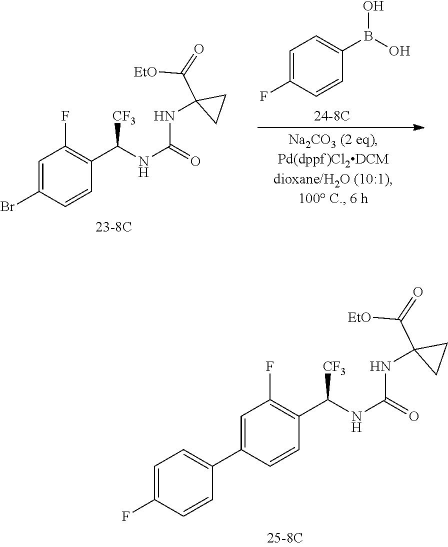

To a mixture of Compound 23-8C (13 g, 30.4 mmol, 1 eq) and Compound 24-8C (5.11 g, 36.5 mmol, 1.2 eq) in dioxane (300 mL) and H₂O (30 mL) was added Pd(dppf)Cl₂·CH₂Cl₂ (2.49 g, 3.04 mmol, 0.1 eq), Na₂CO₃ (6.45 g, 60.8 mmol, 2 eq) in one portion at 25° C. under N₂. The mixture was stirred at 100° C. for 6 hours. The mixture was filtered. The residue was poured into water (200 mL). The aqueous phase was extracted with ethyl acetate (100 mL×3). The combined organic phase was washed with brine (150 mL), dried with anhydrous Na₂SO₄, filtered and concentrated in vacuum. The residue was purified by silica gel chromatography (petroleum ether/ethyl acetate=30:1 to 3:1) to afford Compound 25-8C (10.7 g, 24.1 mmol) as yellow solid.

¹H NMR (400 MHz, CDCl₃) δ 7.45-7.42 (m, 2H), 7.27-7.19 (m, 3H), 7.09-7.05 (m, 2H), 5.96-5.94 (m, 1H), 5.82-5.76 (m, 1H), 5.45 (br s, 1H), 4.09-4.03 (m, 2H), 1.55-1.52 (m, 2H), 1.19-1.09 (m, 5H).

### Preparation of (S)-1-(3-(1-(3,4′-difluoro-[1,1′-biphenyl]-4-yl)-2,2,2-trifluoroethyl)ureido)cyclopropane-1-carboxylic acid Compound 26-8C

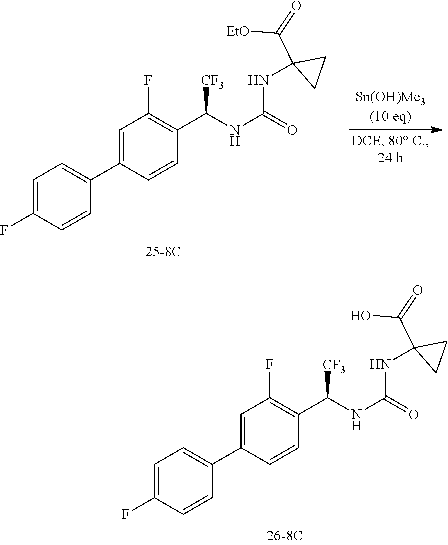 [SMILES: CCOC(=O)C1(NC(=O)N[C@@H](c2ccc(-c3ccc(F)cc3)cc2F)C(F)(F)F)CC1 ; "Sn(OH)Me3(10 eq)" ; O=C(N[C@@H](c1ccc(-c2ccc(F)cc2)cc1F)C(F)(F)F)NC1(C(=O)O)CC1]

To a mixture of Compound 25-8C (9.7 g, 21.9 mmol, 1 eq) in DCE (450 mL) was added hydroxy(trimethyl)stannane (39.65 g, 219.27 mmol, 10 eq) in one portion at 25° C. under N₂. The mixture was stirred at 80° C. for 24 hours. The mixture was adjusted to pH=6 with 1N HCl solution. The aqueous phase was extracted with DCM (100 mL×3). The combined organic phase was washed with brine (150 mL), dried with anhydrous Na₂SO₄, filtered and concentrated in vacuum. The residue was purified by silica gel chromatography (Petroleum ether/Ethyl acetate=20/1, 0/1) to afford Compound 26-8C (8.3 g, 20.0 mmol) as yellow solid.

¹H NMR (400 MHz, CDCl₃) δ 7.69-7.66 (m, 2H), 7.54-7.42 (m, 3H), 7.26-7.13 (m, 2H), 5.92-5.86 (m, 1H), 1.49-1.40 (m, 2H), 1.12-1.05 (m, 2H).

### Preparation of (S)-6-(1-(3,4′-difluoro-[1,1′-biphenyl]-4-yl)-2,2,2-trifluoroethyl)-4,6-diazaspiro[2.4]heptane-5,7-dione Compound 27-8C

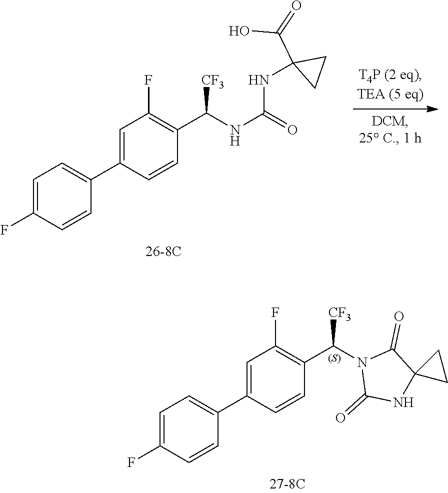

Four batches in parallel: To a mixture of Compound 26-8C (500 mg, 1.21 mmol, 1 eq) and TEA (610 mg, 6.03 mmol, 839 μL, 5 eq) in DCM (10 mL) was added T₄P [CAS #163755-62-2] (1.74 g, 2.41 mmol, 50% purity, 2 eq) in one portion at 25° C. under N₂. The mixture was stirred at 25° C. for 1 hour. The mixture was poured into water (10 mL). The aqueous phase was extracted with DCM (10 mL×3). The combined organic phase was washed with brine (15 mL), dried with anhydrous Na₂SO₄, filtered and concentrated in vacuum to afford crude Compound 27-8C (1.91 g, crude) as yellow solid, which was used in next step directly without storing.

### Preparation of (S)-4-((5-bromo-1,3,4-thiadiazol-2-yl)methyl)-6-(1-(3,4′-difluoro-[1,1′-biphenyl]-4-yl)-2,2,2-trifluoroethyl)-4,6-diazaspiro[2.4]heptane-5,7-dione Compound 28 8C

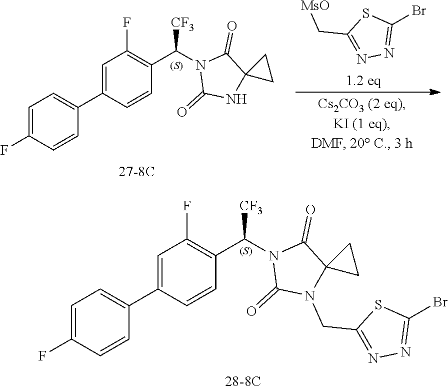

Four batches in parallel: To a mixture of Compound 27-8C (478 mg, 1.21 mmol, 1 eq) and (5-bromo-1,3,4-thiadiazol-2-yl)methyl methanesulfonate (395 mg, 1.45 mmol, 1.2 eq) in DMF (5 mL) was added Cs₂CO₃ (785 mg, 2.41 mmol, 2 eq) and KI (200 mg, 1.21 mmol, 1 eq) in one portion at 25° C. under N₂. The mixture was stirred at 25° C. for 3 hours. Combine the four batches. The mixture was poured into water (20 mL). The aqueous phase was extracted with ethyl acetate (20 mL×2). The combined organic phase was washed with brine (30 mL), dried with anhydrous Na₂SO₄, filtered and concentrated in vacuum. The residue was purified by silica gel chromatography (Petroleum ether/Ethyl acetate=30:1, to 3:1) to afford Compound 28-8C (1.6 g, 2.79 mmol) as yellow solid.

¹H NMR (400 MHz, CDCl₃) δ 7.98-7.95 (m, 1H), 7.48-7.45 (m, 2H), 7.35-7.32 (m, 1H), 7.23-7.20 (m, 1H), 7.09-7.05 (m, 2H), 6.18-6.12 (m, 1H), 4.68-4.55 (m, 2H), 1.48-1.41 (m, 4H).

### Preparation of (S)-4-((5-(2,4-difluoro-3-hydroxyphenyl)-1,3,4-thiadiazol-2-yl)methyl)-6-(1-(3,4′-difluoro-[1,1′-biphenyl]-4-yl)-2,2,2-trifluoroethyl)-4,6-diazaspiro[2.4]heptane-5,7-dione Compound I-97

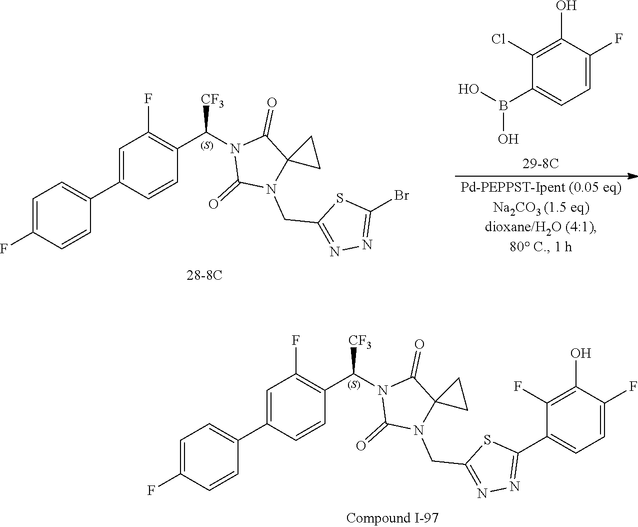

To a mixture of Compound 28-8C (0.4 g, 697 μmol, 1 eq) and (2,4-difluoro-3-hydroxyphenyl)boronic acid 29-8C (145 mg, 837 mol, 1.2 eq) in dioxane (4 mL) and H₂O (1 mL) was added Na₂CO₃ (110 mg, 1.05 mmol, 1.5 eq) and Pd-PEPPST-Ipent (45.0 mg, 52.3 μmol, 0.075 eq) in one portion at 25° C. under N₂. The mixture was stirred at 80° C. for 1 hour. The mixture was filtered. The residue was poured into water (5 mL). The aqueous phase was extracted with ethyl acetate (10 mL×3). The combined organic phase was washed with brine (15 mL), dried with anhydrous Na₂SO₄, filtered and concentrated in vacuum. The residue was purified by prep-HPLC (column: Waters Xbridge BEH C18 100×30 mm×10 um; mobile phase: [H₂O (10 mM NH₄HCO₃)-ACN]; gradient: 45%-75% B over 8.0 min) to afford crude product (180 mg, SFC purity: 96.18%), this crude mixture was purified by prep-SFC (column: DAICEL CHIRALPAK IG (250 mm×30 mm, 10 μm); mobile phase: [CO₂-MeOH (0.1% NH₃H₂O)]; B %: 35%, isocratic elution mode) to afford Compound I-97 (120 mg, 193 μmol, 99.8% purity) as white solid.

¹H NMR (400 MHz, DMSO-d₆) δ 10.84 (br s, 1H), 7.91 (br t, J=7.87 Hz, 1H), 7.75-7.85 (m, 2H), 7.56-7.75 (m, 3H), 7.18-7.41 (m, 3H), 6.35 (q, J=8.90 Hz, 1H), 4.87-5.04 (m, 2H), 1.61-1.77 (m, 2H), 1.27-1.43 (m, 2H).

### Preparation of (S)-6-(1-(3,4′-difluoro-[1,1′-biphenyl]-4-yl)-2,2,2-trifluoroethyl)-4-((5-(5-fluoro-6-methoxypyridin-2-yl)-1,3,4-thiadiazol-2-yl)methyl)-4,6-diazaspiro[2.4]heptane-5,7-dione Compound 31-8C

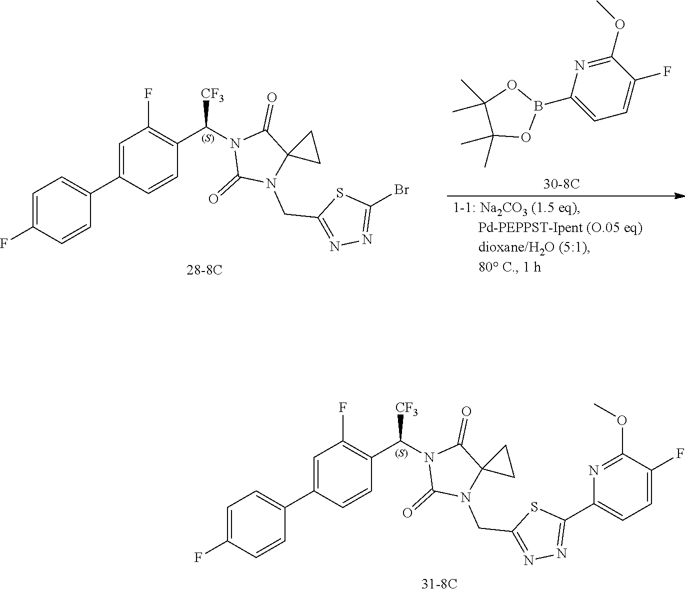

To a mixture of Compound 28-8C (1.5 g, 2.62 mmol, 1 eq) and Compound 30-8C (794 mg, 3.14 mmol, 1.2 eq) in dioxane (30 mL) and H₂O (6 mL) was added Na₂CO₃ (554 mg, 5.23 mmol, 2 eq) and Pd-PEPPST-Ipent (225 mg, 261 μmol, 0.1 eq) in one portion at 25° C. under N₂. The mixture was stirred at 80° C. for 1 hour. The mixture was filtered and concentrated in reduced pressure at 40° C. The residue was poured into water (20 mL). The aqueous phase was extracted with ethyl acetate (20 mL×3). The combined organic phase was washed with brine (30 mL), dried with anhydrous Na₂SO₄, filtered and concentrated in vacuum. The residue was purified by silica gel chromatography (petroleum ether/ethyl acetate=30:1, to 3:1) to afford Compound 31-8C (1.6 g, 2.58 mmol) as yellow solid.

¹H NMR (400 MHz, DMSO-d₆) δ 7.98 (q, J=7.87 Hz, 1H), 7.84-7.81 (m, 1H), 7.47-7.39 (m, 4H), 7.25 (m, 1H), 7.07 (q, J=8.80 Hz, 1H), 6.22-6.16 (m, 1H), 4.75-4.64 (m, 2H), 3.97 (s, 3H), 1.54-1.52 (m, 2H), 1.51-1.49 (m, 2H).

### Preparation of (S)-6-(1-(3,4′-difluoro-[1,1′-biphenyl]-4-yl)-2,2,2-trifluoroethyl)-4-((5-(5-fluoro-6-hydroxypyridin-2-yl)-1,3,4-thiadiazol-2-yl)methyl)-4,6-diazaspiro[2.4]heptane-5,7-dione Compound I-96

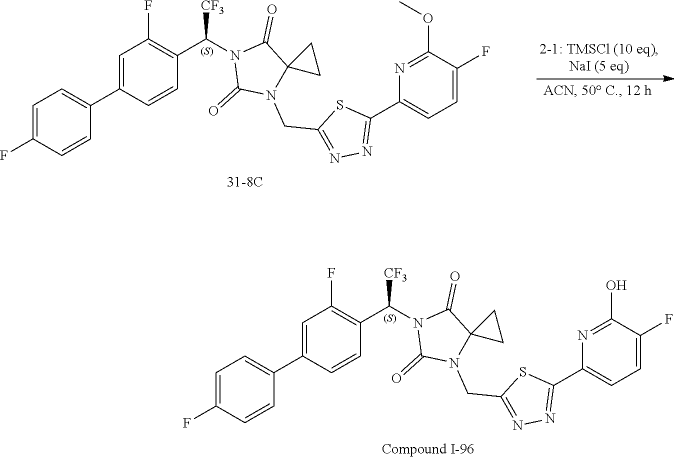

To a mixture of Compound 31-8C (400 mg, 645 μmol, 1 eq) in CH₃CN (8 mL) was added TMSCl (701 mg, 6.46 mmol, 819 μL, 10 eq) and NaI (483 mg, 3.23 mmol, 5 eq) in one portion at 25° C. under N₂. The mixture was stirred at 50° C. for 12 hours. The mixture was filtered and concentrated in reduced pressure at 40° C. The residue was purified by prep-HPLC (column: Phenomenex luna C18 100×40 mm×5 um; mobile phase: [H₂O (0.2% FA)-ACN]; gradient: 45%-75% B over 8.0 min) to afford Compound I-96 (144.88 mg, 239.03 μmol, 99.9% purity) as yellow solid.

¹H NMR (400 MHz, DMSO-d₆) δ 12.35-12.05 (br m, 1H), 7.89 (t, J=8.0 Hz, 1H), 7.84-7.74 (m, 4H), 7.69-7.60 (m, 2H), 7.32 (t, J=8.8 Hz, 2H), 6.33 (q, J=8.8 Hz, 1H), 4.99-4.84 (m, 2H), 1.74-1.60 (m, 2H), 1.42-1.28 (m, 2H).

### Preparation of 3-fluoro-2-methoxy-6-(4,4,5,5-tetramethyl-1,3,2-dioxaborolan-2-yl)pyridine Compound 30-8C

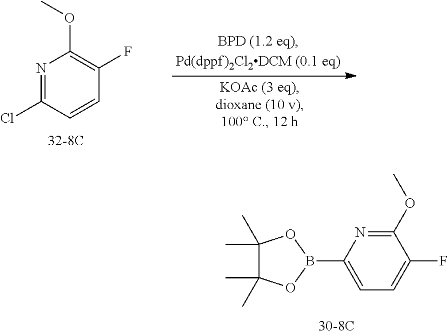

To a mixture of Compound 32-8C (0.9 g, 5.57 mmol, 1 eq) in dioxane (9 mL) was added BPD (2.12 g, 8.36 mmol, 1.5 eq) and Pd(dppf)Cl₂·DCM (407 mg, 557 μmol, 0.1 eq) KOAc (1.64 g, 16.7 mmol, 3 eq) in one portion at 20° C. under N₂. The mixture was stirred at 100° C. for 12 hours. The mixture was filtered and concentrated in reduced pressure at 40° C. The residue was poured into water (10 mL). The aqueous phase was extracted with ethyl acetate (10 mL×3). The combined organic phase was washed with brine (15 mL), dried with anhydrous Na₂SO₄, filtered and concentrated in vacuum. The residue was purified by silica gel chromatography (petroleum ether/ethyl acetate=15:1, to 1:1) to afford Compound 30-8C (1.9 g, crude) as yellow solid.

¹H NMR (400 MHz, CDCl₃) δ 7.38-7.35 (m, 1H), 7.21-7.16 (m, 1H), 4.04 (s, 3H), 1.29 (s, 12H).

The following prophetic examples in Table 9 can be made using the procedures outlined above.

TABLE 9

| | | |
|---|---|---|
| I-98 | 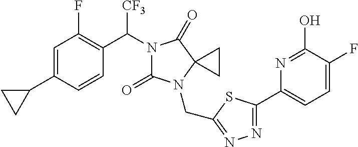 [SMILES: O=C1N(C(c2ccc(C3CC3)cc2F)C(F)(F)F)C(=O)C2(CC2)N1Cc1nnc(-c2ccc(F)c(O)n2)s1] | 6-(1-(4-cyclopropyl-2-fluoro- phenyl)-2,2,2-trifluoroethyl)- 4-((5-(5-fluoro-6-hydroxy- pyridin-2-yl)-1,3,4-thiadiazol- 2-yl)methyl)-4,6-diazaspiro- [2.4]heptane-5,7-dione |
| I-99 | 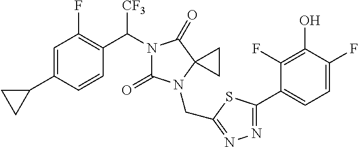 [SMILES: O=C1N(C(c2ccc(C3CC3)cc2F)C(F)(F)F)C(=O)C2(CC2)N1Cc1nnc(-c2ccc(F)c(O)c2F)s1] | 6-(1-(4-cyclopropyl-2-fluoro- phenyl)-2,2,2-trifluoroethyl)- 4-((5-(2,4-difluoro-3-hydroxy- phenyl)-1,3,4-thiadiazol-2-yl)- methyl)-4,6-diazaspiro[2.4]- heptane-5,7-dione |
| I-106 | 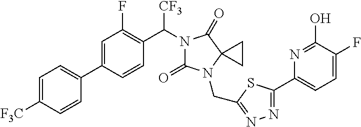 [SMILES: O=C1N(C(c2ccc(-c3ccc(C(F)(F)F)cc3)cc2F)C(F)(F)F)C(=O)C2(CC2)N1Cc1nnc(-c2ccc(F)c(O)n2)s1] | 4-((5-(5-fluoro-6-hydroxy- pyridin-2-yl)-1,3,4-thiadiazol- 2-yl)methyl)-6-(2,2,2-tri- fluoro-1-(3-fluoro-4′-(tri- fluoromethyl)-[1,1′-biphenyl]- 4-yl)ethyl)-4,6-diazaspiro- [2.4]heptane-5,7-dione |
| I-107 | 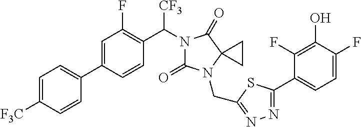 [SMILES: O=C1N(C(c2ccc(-c3ccc(C(F)(F)F)cc3)cc2F)C(F)(F)F)C(=O)C2(CC2)N1Cc1nnc(-c2ccc(F)c(O)c2F)s1] | 4-((5-(2,4-difluoro-3-hydroxy- phenyl)-1,3,4-thiadiazol-2- yl)methyl)-6-(2,2,2-trifluoro- 1-(3-fluoro-4′-(trifluorometh- yl)-[1,1′-biphenyl]-4-yl)ethyl)- 4,6-diazaspiro[2.4]heptane- 5,7-dione |
| I-108 | 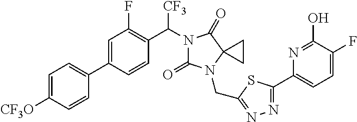 | 4-((5-(5-fluoro-6-hydroxy- pyridin-2-yl)-1,3,4-thiadiazol- 2-yl)-methyl)-6-(2,2,2-tri- fluoro-1-(3-fluoro-4′-(tri- fluoromethoxy)-[1,1′-biphen- yl]-4-yl)ethyl)-4,6-diazaspiro- [2.4]heptane-5,7-dione |
| I-109 | 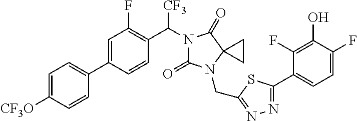 | 4-((5-(2,4-difluoro-3-hydroxy- phenyl)-1,3,4-thiadiazol-2- yl)methyl)-6-(2,2,2-trifluoro- 1-(3-fluoro-4′-(trifluorometh- oxy)-[1,1′-biphenyl]-4-yl)eth- yl)-4,6-diazaspiro[2.4]heptane- 5,7-dione |
| I-110 | 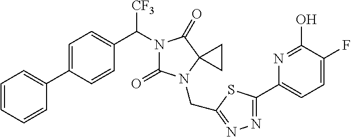 [SMILES: O=C1N(C(c2ccc(-c3ccccc3)cc2)C(F)(F)F)C(=O)C2(CC2)N1Cc1nnc(-c2ccc(F)c(O)n2)s1] | 6-(1-([1,1′-biphenyl]-4-yl)- 2,2,2-trifluoroethyl)-4-((5-(5- fluoro-6-hydroxypyridin-2-yl)- 1,3,4-thiadiazol-2-yl)methyl)- 4,6-diazaspiro[2.4]heptane- 5,7-dione |
| I-111 | 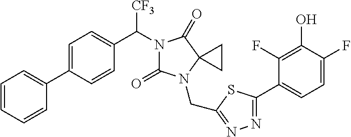 [SMILES: O=C1N(C(c2ccc(-c3ccccc3)cc2)C(F)(F)F)C(=O)C2(CC2)N1Cc1nnc(-c2ccc(F)c(O)c2F)s1] | 6-(1-([1,1'-biphenyl]-4-yl)- 2,2,2-trifluoroethyl)-4-((5-(2,4- difluoro-3-hydroxyphenyl)- 1,3,4-thiadiazol-2-yl)methyl)- 4,6-diazaspiro[2.4]heptane- 5,7-dione |

Compound I-51, a glucuronide of compound I-16, was used to develop methodology to ensure the glucuronide metabolite of the phenol could be stabilized in liver tissue to glucuronidase and other enzymatic cleavage in order to determine accurate levels of parent:glucuronide in the liver tissue. A known amount of Compound I-51 was spiked into a gram of mouse liver tissue where 2% formic acid (FA) was used with 30% acetonitrile (ACN) in water to stop all enzymatic glucuronidase activity.

Comparison of Treatment on ‘30% ACN in Water with 2% FA’ Homogenate Buffer for Compound I-51:

### Compound I-51 was Added Before Homogenate:

1). Prepared the homogenate buffer ‘30% ACN in water with 2% FA’ (including 1 ag/mL Compound 1-51), then homogenized with the liver tissue together. Due to the ratio of 1:9 (1 g tissue with 9 mL buffer), the final concentration was 900 ng/mL.

2). An aliquot of 20 μL sample were taken for analysis.
Compound I-51 was Added after Homogenate:

1). The liver tissue was homogenized with the buffer ‘30% ACN in water with 2% FA’, then 2 μL 18 g/mL Compound I-51 was spiked in 38 μL liver homogenate, the final concentration was 900 ng/mL. 2). An aliquot of 20 μL sample were taken for analysis.

A standard curve for Compound I-51 was generated for peak AUC versus known amount of Compound I-51 from 1 ng/g to 3000 ng/g using ‘30% ACN in water with 2% FA. The results of the 2 conditions are shown below:

| | | | | Analyte |
|---|---|---|---|---|
| | Analyte | Calculated | Analyte | Concentration |
| | Peak | Concentration | Peak Area | (ng/mL) |
| Conditions | Name | (ng/mL) | (counts) | Maximum |
| before | Compound | 867.785 | 7.37E+05 | 900 |
| homogenate | I-51 | (96.4%) | | |
| after | Compound | 878.783 | 7.69E+05 | 900 |
| homogenate | I-51 | (97.6%) | | |

The amount of parent was measured under these conditions Compound I-16 after generating a standard curve for Compound I-16.

| | | | Analyte |
|---|---|---|---|
| | Analyte | Calculated | Peak |
| | Peak | Concentration | Area |
| Conditions | Name | (ng/mL) | (counts) |
| before | Compound | 5.618 | 2.16E+04 |
| homogenate | I-16 | | |
| after | Compound | 5.237 | 2.08E+04 |
| homogenate | I-16 | | |

### Stability of Compound I-51 in Liver Homogenate for 24 Hours:

1). Prepared the homogenate buffer ‘30% ACN in water with 2% FA’ (including 1 μg/mL Compound I-51), then homogenized with the liver tissue together at the ratio of 1:9 (1 g tissue with 9 mL buffer). Then placed it at 4° C. for 24 hours, as T=24 h sample. An aliquot of 20 μL sample were taken for analysis.

2). Prepared the liver homogenate, then 2 μL 18 g/mL Compound I-51 was spiked in 38 μL liver homogenate, the final concentration was 900 ng/mL, as T=0 h sample. An aliquot of 20 μL sample were taken for analysis.

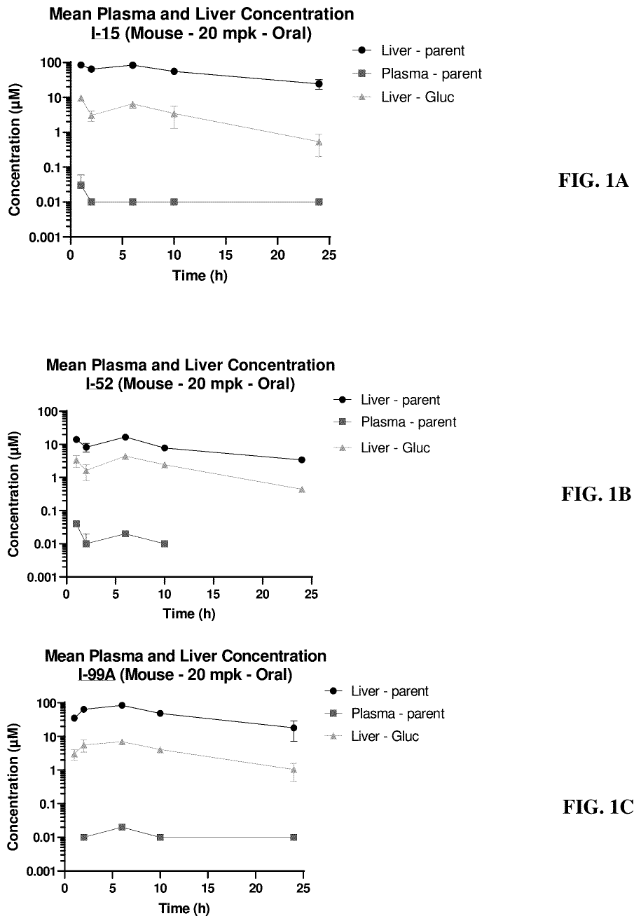

Under these conditions, Compound I-51 was stable for 24 h with 30% ACN in water with 2% FA providing 100% of expected compound I-51 at both 0 h and 24 h time points. This experiment of spiking the glucuronide in the liver tissue was conducted a second time with parent:glucuronide pair compound I-35: compound I-72 to confirm stability of the glucuronide using this methodology of ‘30% ACN in water with 2% FA’ with similar results. Compound I-15 is used as an example of methodology used in a mouse (C57/BL6 strain) pharmacokinetic study where compound I-15 was dosed PO at 20 MPK. The liver collection was conducted as follows: Liver Collection: After collection, each liver tissue was rinsed with saline and wiped dry, then divided into two parts, one was homogenized and analyzed, and the other was used for backup. Each part was weighed. For the analyzed part liver sample, cold homogenizing buffer (30% ACN in water with 2% FA) was added at the ratio of 1:9 (1 g tissue with 9 mL buffer), then homogenized on wet ice. The tissue homogenate was kept at −60° C. or lower until LC-MS/MS analysis. Compounds I-15, I-52, and I-99A were dosed at 20 MPK PO to C57/BL6 mice and liver time points were taken at 1 h, 2 h, 6 h, 10 h, and 24 h to determine the parent:glucuronide ratio and the graphs are shown in FIGS. 1A-1C.

For purposes of interpreting this specification, the following definitions will apply, and whenever appropriate, terms used in the singular will also include the plural. Terms used in the specification have the following meanings unless the context clearly indicates otherwise: As used herein, the term “subject” refers to an animal. In certain aspects, the animal is a mammal. A subject also refers to, for example, primates (e.g., humans), cows, sheep, goats, horses, dogs, cats, rabbits, rats, mice, fish, birds and the like. In certain embodiments, the subject is a human. A “patient” as used herein refers to a human subject.

As used herein, the term “inhibition” or “inhibiting” refers to the reduction or suppression of a given condition, symptom, or disorder, or disease, or a significant decrease in the baseline activity of a biological activity or process.

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. “Optionally substituted” means the group referred to can be substituted at one or more positions by any one or any combination of the radicals listed thereafter. The number, placement and selection of substituents is understood to encompass only those substitutions that a skilled chemist would expect to be reasonably stable; thus ‘oxo’ would not be a substituent on an aryl or heteroaryl ring, for example, and a single carbon atom would not have three hydroxy or amino substituents. Unless otherwise specified, optional substituents are typically up to four groups selected from halo, oxo, CN, amino, hydroxy, —C₁₋₃ alkyl, C₁-C₄ haloalkyl, —OR*, —NR*₂, —SR*, —SO₂R*, —COOR*, and —CONR*₂, where each R* is independently H, C1-4 alkyl, C1-C4 haloalkyl, or C₃₋₈ cycloalkyl with the proviso that two R* on —CONR*₂ may form a ring of C₃-C₈ carbons.

“Aryl” as used herein refers to a phenyl or naphthyl or biphenyl group unless otherwise specified.

Aryl groups unless otherwise specified may be optionally substituted with up to four groups selected from halo, CN, amino, hydroxy, C₁₋₃ alkyl, optionally substituted phenyl, optionally substituted cyclopropyl, —OR*, —NR*₂, —SR*, —SO₂R*, —COOR*, and —CONR*₂, where each R* is independently H C₁₋₄ alkyl, or C₃₋₈ cycloalkyl with the proviso that two R* on —CONR*₂ may form a ring of C₃₋₈ carbons.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

Carbocycle includes cycloalkyl and aryl.

“Halo” or “halogen”, as used herein, may be fluorine, chlorine, bromine or iodine. “C₁₋₈ alkyl” or “C₁-C₈ alkyl”, as used herein, denotes straight chain or branched alkyl having 3-8 carbon atoms. If a different number of carbon atoms is specified, such as C₅ or C₅, then the definition is to be amended accordingly, such as “C₁₋₄ alkyl” will represent methyl, ethyl, propyl, isopropyl, butyl, isobutyl, sec-butyl and tert-butyl.

Optionally substituted C₁-C₈ linear or branched alkyl unless otherwise specified, optional substituents are typically up to four groups selected from halo, oxo, CN, amino, hydroxy, —C₁₋₃ alkyl, C₁-C₄ haloalkyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the provisos that two R* on —CONR*₂ may form a ring of C₃-C₈ carbons and wherein a —CH₂— group of the C₁-C₈ linear or branched alkyl of R³ may be replaced by a spirocyclopropyl:

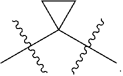 [SMILES: *C1(*)CC1]

Optionally substituted C₁-C₈ cycloalkyl (including bicyclo- or tricycloalkyl) unless otherwise specified, optional substituents are typically up to four groups selected from halo, oxo, CN, amino, hydroxy, —C₁₋₃ alkyl, C₁-C₄ haloalkyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons.

Optionally substituted phenyl unless otherwise specified, optional substituents are typically up to four groups selected from halo, oxo, CN, amino, hydroxy, —C₁₋₃ alkyl, C₁-C₄ haloalkyl, optionally substituted phenyl, optionally substituted cyclopropyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons.

Optionally substituted benzyl unless otherwise specified, optional substituents are typically up to four groups selected from halo, oxo, CN, amino, hydroxy, —C₁₋₃ alkyl, C₁-C₄ haloalkyl, optionally substituted phenyl, optionally substituted cyclopropyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons and wherein a —CH₂— group of the benzyl of R³ may be replaced by a spirocyclopropyl:

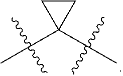 [SMILES: *C1(*)CC1]

Optionally substituted 2-phenethyl groups unless otherwise specified, optional substituents are typically up to four groups selected from halo, oxo, CN, amino, hydroxy, —C₁₋₃ alkyl, C₁-C₄ haloalkyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons.

Optionally substituted C₁-C₈ alkynyl unless otherwise specified, optional substituents are typically up to four groups selected from halo, oxo, CN, amino, hydroxy, —C₁₋₃ alkyl, C₁-C₄ haloalkyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons.

“C₁₋₁₀ alkylene” or “C₁-C₁₀ alkylene”, as used herein, denotes straight chain or branched alkyl having 1-10 carbon atoms and two open valences for connection to two other groups. If a different number of carbon atoms is specified, such as C₄ or C₃, then the definition is to be amended accordingly, such as “C₁₋₄ alkylene” will represent methylene (—CH₂—), ethylene (—CH₂CH₂—), straight chain or branched propylene (—CH₂CH₂CH₂— or —CH₂—CHMe-CH₂—), and the like.

Optionally substituted C₁-C₁₀ alkylene unless otherwise specified, optional substituents are typically up to four groups selected from halo, oxo, CN, amino, hydroxy, —C₁₋₃ alkyl, C₁-C₄ haloalkyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons and wherein a —CH₂— group of the C₁-C₁₀ linear alkyl of R³ may be replaced by a spirocyclopropyl:

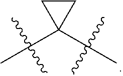 [SMILES: *C1(*)CC1]

“C₅₋₈ alkoxy”, as used herein, denotes straight chain or branched alkoxy (—O-Alkyl) having 5-8 carbon atoms. If a different number of carbon atoms is specified, such as C₄ or C₃, then the definition is to be amended accordingly, such as “C₁₋₄ alkoxy” will represent methoxy, ethoxy, propoxy, isopropoxy, butoxy, isobutoxy, sec-butoxy and tert-butoxy.

“C₁₋₄ Haloalkyl” or “C₁-C₄ haloalkyl” as used herein, denotes straight chain or branched alkyl having 1-4 carbon atoms wherein at least one hydrogen has been replaced with a halogen. The number of halogen replacements can be from one up to the number of hydrogen atoms on the unsubstituted alkyl group. If a different number of carbon atoms is specified, such as C₆ or C₃, then the definition is to be amended accordingly. Thus “C₁₋₄ haloalkyl” will represent methyl, ethyl, propyl, isopropyl, butyl, isobutyl, sec-butyl and tert-butyl that have at least one hydrogen substituted with halogen, such as where the halogen is fluorine: CF₃CF₂—, (CF₃)₂CH—, CH₃—CF₂—, CF₃CF₂—, CF₃—, CF₂H—, CF₃CF₂CH(CF₃)— or CF₃CF₂CF₂CF₂—.

“C₃₋₈ cycloalkyl” as used herein refers to a saturated monocyclic hydrocarbon ring of 3 to 8 carbon atoms. Examples of such groups include cyclopropyl, cyclobutyl, cyclopentyl and cyclohexyl. If a different number of carbon atoms is specified, such as C₃-C₆, then the definition is to be amended accordingly.

“Alkynyl” refers to any group derived from a straight or branched hydrocarbon with at least one carbon-carbon triple bond and includes those groups having one triple bond and one double bond. Examples of alkynyl groups include, but are not limited to, ethynyl (C≡CH), propargyl (—CH₂C≡CH), (E)-pent-3-en-1-ynyl, and the like. Unless otherwise specified, an alkynyl group has from 2 to about 10 carbon atoms, for example from 2 to 10 carbon atoms, for example from 2 to 6 carbon atoms, for example from 2 to 4 carbon atoms.

“4- to 8-Membered heterocyclyl”, “5- to 6-membered heterocyclyl”, “3- to 10-membered heterocyclyl”, “3- to 14-membered heterocyclyl”, “4- to 14-membered heterocyclyl” and “5- to 14-membered heterocyclyl”, refers, respectively, to 4- to 8-membered, 5- to 6-membered, 3- to 10-membered, 3- to 14-membered, 4- to 14-membered and 5- to 14-membered heterocyclic rings; unless otherwise specified, such rings contain 1 to 7, 1 to 5, or 1 to 3 heteroatoms selected from the group consisting of nitrogen, oxygen and sulfur as ring members, and the rings may be saturated, or partially saturated but not aromatic. The heterocyclic group can be attached to another group at a nitrogen or a carbon atom. The term “heterocyclyl” includes single ring groups, fused ring groups and bridged groups. Examples of such heterocyclyl include, but are not limited to pyrrolidine, piperidine, piperazine, pyrrolidinone, morpholine, tetrahydrofuran, tetrahydrothiophene, tetrahydrothiopyran, tetrahydropyran, 1,4-dioxane, 1,4-oxathiane, 8-aza-bicyclo[3.2.1]octane, 3,8-diazabicyclo[3.2.1]octane, 3-Oxa-8-aza-bicyclo[3.2.1]octane, 8-oxa-3-aza-bicyclo[3.2.1]octane, 2-oxa-5-aza-bicyclo[2.2.1]heptane, 2,5-Diaza-bicyclo[2.2.1]heptane, azetidine, ethylenedioxo, oxetane or thiazole. In certain embodiments, if not otherwise specified, heterocyclic groups have 1-2 heteroatoms selected from N, O and S as ring members, and 4-7 ring atoms, and are optionally substituted with up to four groups selected from halo, oxo, CN, amino, hydroxy, C₁₋₃ alkyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons. In particular, heterocyclic groups containing a sulfur atom are optionally substituted with one or two oxo groups on the sulfur.

“4-6 membered cyclic ether” as used herein refers to a 4 to 6 membered ring comprising one oxygen atom as a ring member. Examples include oxetane, tetrahydrofuran and tetrahydropyran. “Heteroaryl” is a completely unsaturated (aromatic) ring. The term “heteroaryl” refers to a 5-14 membered monocyclic- or bicyclic- or tricyclic-aromatic ring system, having 1 to 8 heteroatoms selected from N, O or S. Typically, the heteroaryl is a 5-10 membered ring or ring system (e.g., 5-7 membered monocyclic group or an 8-10 membered bicyclic group), often a 5-6 membered ring containing up to four heteroatoms selected from N, O and S, though often a heteroaryl ring contains no more than one divalent 0 or S in the ring. Typical heteroaryl groups include furan, isothiazole, thiadiazole, oxadiazole, indazole, indole, quinoline, 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-(1,2,4-triazolyl), 4- or 5-(1,2, 3-triazolyl), tetrazolyl, triazine, pyrimidine, 2-, 3-, or 4-pyridyl, 3- or 4-pyridazinyl, 3-, 4-, or 5-pyrazinyl, 2-pyrazinyl, and 2-, 4-, or 5-pyrimidinyl. Heteroaryl groups are optionally substituted with up to four groups selected from halo, CN, amino, hydroxy, C₁₋₃ alkyl, —OR*, —NR*₂, —SR*, —SO₂R*, —COOR*, and —CONR*₂, where each R* is independently H, C₁₋₄ alkyl, C₁-C₄ haloalkyl, or C₃₋₈ cycloalkyl with the proviso that two R* on —CONR*2 may form a ring of C₃₋₈ carbons. The term “hydroxy” or “hydroxyl” refers to the group —OH.

It is noteworthy that 2-hydroxypyridine includes a tautomeric 2-pyridone form as illustrated by:

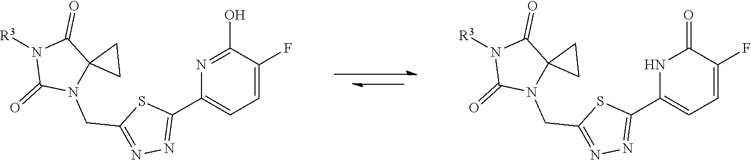

Where V=N, X¹=hydroxy, and X²=fluoro.

The term “aralkyl” refers to an aryl group covalently linked to an alkylene group, where aryl and alkylene are defined herein. “Optionally substituted aralkyl” refers to an optionally substituted aryl group covalently linked to an optionally substituted alkylene group. “Optionally substituted aralkyl” includes diarylalkyl groups like optionally substituted benzhydryl. Such aralkyl groups are exemplified by benzyl, phenylethyl, 3-(4-methoxyphenyl)propyl, bis(4-fluorophenyl)methyl, and the like.

The term “heteroaralkyl” refers to an heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 2-pyrimidinylmethyl-, 3-pyridinylethyl, 3-(4-methoxypyridyl)propyl, and the like.

The term “cyclopropylalkyl” refers to a cyclopropyl group covalently linked to an alkylene group, where cyclopropyl and alkylene are defined herein. “Optionally substituted cyclopropylalkyl” refers to an optionally substituted cyclopropyl group covalently linked to an optionally substituted alkylene group. Such cyclopropylalkyl groups are exemplified by cyclopropylmethyl, cyclopropylethyl, 1-(trifluoromethyl)cyclopropyl)methyl, and the like.

### 17β-HSD13 Biochemical Assay:

17β-HSD13 biochemical assays are generically described in patents WO 2022/020730, WO 2021/003295, and WO 2023/023310. More specifically, recombinant 17β-HSD13 protein was assayed in a buffer containing 200 mM Tris pH 7.5, 0.01% Triton X-100, and 0.02% BSA into a 384-well assay plate. Compounds were incubated with 17β-HSD13 (final 50 nM) and NAD+ (final 10 mM) at room temperature for 1 h prior to substrate addition. The assay reaction was then initiated by addition of β-estradiol (final 20 μM), and the reaction mixture was incubated for 2 hours at room temperature. Product formation was detected with chemiluminescence by adding equal volume of NAD+/NADH Glo reagent (Promega, #G9062) and read on a PHERAstar microplate reader (BMG LABTECH). IC50 values were determined with GraphPad Prism®, where log-transformed concentration values and the inhibition data were fitted to a four-parameter logistic equation. Y=Bottom+(Top−Bottom)/(1+10\hat{ }((Log IC50−X)*HillSlope)). The original, % control, or % inhibition data are represented by Y along with their minimal (Bottom) and maximal (Top) values. The inhibitor concentration is represented by X, IC50 is the concentration at 50% maximal value, and Hill Slope is the slope factor.

### 17β-HSD13 Cell-Based Assay:

HEK293 cells expressing 17β-HSD13 either stably or transiently were incubated with compounds for 60 min at 37° C. in a humidified incubator. After this first incubation step, β-estradiol was added to each well of the microtiter plate and incubated for 3 h at 37° C. in the humidified incubator. After treatment with β-Estradiol, 50 μL of supernatant was transferred to a 96-V plate containing 100 μL acetonitrile and centrifuged at 3900 rpm for 45 min. Finally, 50 μL of supernatant were taken for LC-MS/MS analysis. The chromatography part was run on a Xbridge 30 mm×2.1 mm column (Waters), at 40 degrees Celsius and a flow rate of 0.8 mL/min for 5 min, while the MS was done on a Q Exactive Orbitrap (Fisher Scientific) in Target SIM mode and negative polarity with a spray voltage of 3.5 kV. EC50 values were determined with GraphPad Prism®, where log-transformed concentration values and the inhibition data were fitted to a four-parameter logistic equation. Y=Bottom+(Top−Bottom)/(1+10\hat{ }((Log EC50−X)*Hill Slope)). The original, % control, or % inhibition data are represented by Y along with their minimal (Bottom) and maximal (Top) values. The inhibitor concentration is represented by X, EC50 is the concentration at 50% maximal value, and Hill Slope is the slope factor.

### Biological Activity of Compounds of Formula I:

| | | IC₅₀, nM | EC₅₀, nM |
|---|---|---|---|
| | | Estradiol | Transientª |
| Compound | Structure | NAD+ | Stable^{b} |
| I-1 | 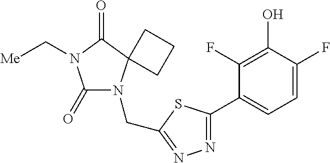 [SMILES: CCN1C(=O)N(Cc2nnc(-c3ccc(F)c(O)c3F)s2)C2(CCC2)C1=O] | 56.7 n = 1 | N/D |
| I-2 | 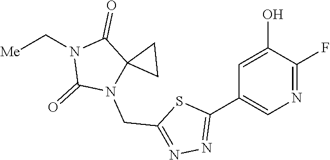 [SMILES: CCN1C(=O)N(Cc2nnc(-c3cnc(F)c(O)c3)s2)C2(CC2)C1=O] | 156 n = 1 | N/D |
| I-3 | 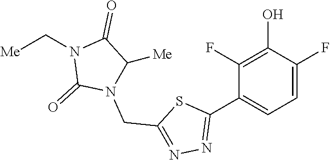 [SMILES: CCN1C(=O)C(C)N(Cc2nnc(-c3ccc(F)c(O)c3F)s2)C1=O] | 86.1 n = 1 | 175ª n = 1 |
| I-4 | 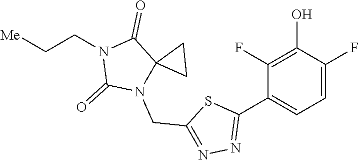 [SMILES: CCCN1C(=O)N(Cc2nnc(-c3ccc(F)c(O)c3F)s2)C2(CC2)C1=O] | 10.6 ± 0.919 n = 2 | 20.6ª n = 1 |
| I-5 | 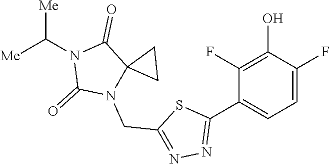 [SMILES: CC(C)N1C(=O)N(Cc2nnc(-c3ccc(F)c(O)c3F)s2)C2(CC2)C1=O] | 8.99 ± 0.322 n = 2 | 42.2ª n = 1 |
| I-6 | 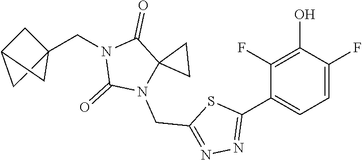 [SMILES: O=C1N(CC23CC(C2)C3)C(=O)C2(CC2)N1Cc1nnc(-c2ccc(F)c(O)c2F)s1] | 22.1 ± 2.18 n = 2 | 165ª n = 1 |
| I-7 | 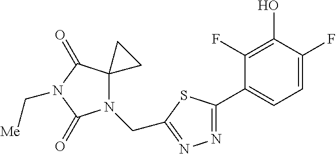 [SMILES: CCN1C(=O)N(Cc2nnc(-c3ccc(F)c(O)c3F)s2)C2(CC2)C1=O] | 16.6 ± 8.74 n = 3 | 18.8ª n = 1 |
| I-8 | 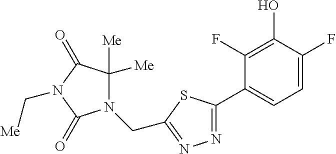 [SMILES: CCN1C(=O)N(Cc2nnc(-c3ccc(F)c(O)c3F)s2)C(C)(C)C1=O] | 90.8 n = 1 | N/D |
| I-9 | 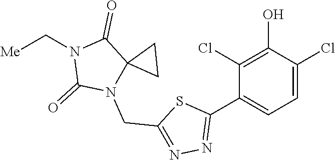 [SMILES: CCN1C(=O)N(Cc2nnc(-c3ccc(Cl)c(O)c3Cl)s2)C2(CC2)C1=O] | 150 n = 1 | 960ª n = 1 |
| I-10 | 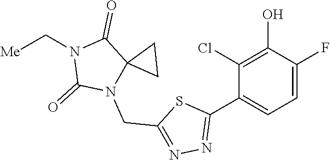 [SMILES: CCN1C(=O)N(Cc2nnc(-c3ccc(F)c(O)c3Cl)s2)C2(CC2)C1=O] | 17.9 ± 0.0566 n = 2 | 118ª n = 1 51.0^{b} n = 1 |
| I-11 | 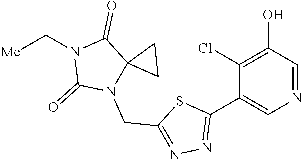 [SMILES: CCN1C(=O)N(Cc2nnc(-c3cncc(O)c3Cl)s2)C2(CC2)C1=O] | 245 n = 1 | N/D |
| I-12 | 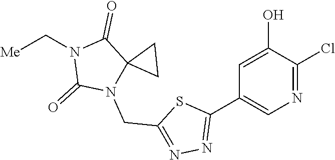 [SMILES: CCN1C(=O)N(Cc2nnc(-c3cnc(Cl)c(O)c3)s2)C2(CC2)C1=O] | 323 n = 1 | N/D |
| I-13 | 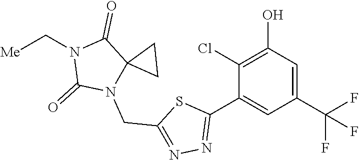 [SMILES: CCN1C(=O)N(Cc2nnc(-c3cc(C(F)(F)F)cc(O)c3Cl)s2)C2(CC2)C1=O] | 13.8 ± 2.2 n = 2 | N/D |
| I-14 | 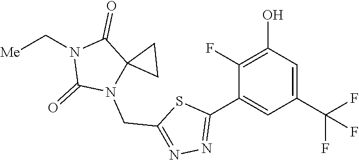 [SMILES: CCN1C(=O)N(Cc2nnc(-c3cc(C(F)(F)F)cc(O)c3F)s2)C2(CC2)C1=O] | 9.6 n = 1 | 125^{b} n = 1 |
| I-15 | 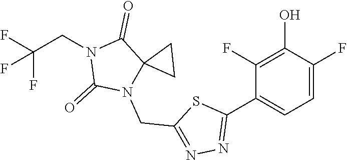 [SMILES: O=C1N(CC(F)(F)F)C(=O)C2(CC2)N1Cc1nnc(-c2ccc(F)c(O)c2F)s1] | 8.07 ± 1.77 n = 4 | 40.9 ± 25^{b} n = 2 |
| I-16 | 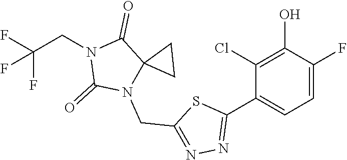 [SMILES: O=C1N(CC(F)(F)F)C(=O)C2(CC2)N1Cc1nnc(-c2ccc(F)c(O)c2Cl)s1] | 12.9 ± 2.43 n = 3 | 107ª n = 1 37.1 ± 9.99^{b} n = 3 |
| I-17 | 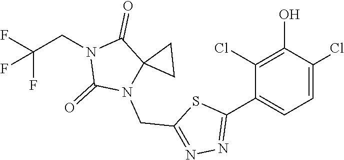 [SMILES: O=C1N(CC(F)(F)F)C(=O)C2(CC2)N1Cc1nnc(-c2ccc(Cl)c(O)c2Cl)s1] | 106 n = 1 | N/D |
| I-18 | 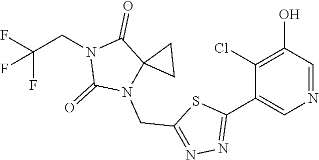 [SMILES: O=C1N(CC(F)(F)F)C(=O)C2(CC2)N1Cc1nnc(-c2cncc(O)c2Cl)s1] | 193 n = 1 | N/D |
| I-19 | 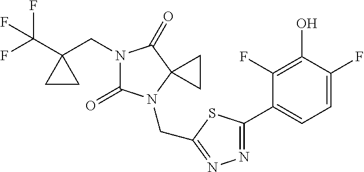 [SMILES: O=C1N(CC2(C(F)(F)F)CC2)C(=O)C2(CC2)N1Cc1nnc(-c2ccc(F)c(O)c2F)s1] | 14.8 ± 2.51 n = 2 | 78.8^{b} n = 1 |
| I-20 | 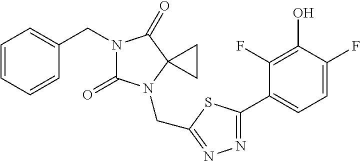 [SMILES: O=C1N(Cc2ccccc2)C(=O)C2(CC2)N1Cc1nnc(-c2ccc(F)c(O)c2F)s1] | 2.28 ± 0.933 n = 2 | 11.6^{b} n = 1 |
| I-21 | 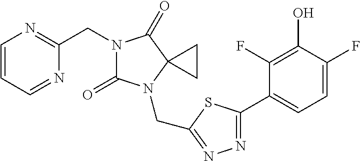 [SMILES: O=C1N(Cc2ncccn2)C(=O)C2(CC2)N1Cc1nnc(-c2ccc(F)c(O)c2F)s1] | 620 n = 1 | N/D |
| I-22 | 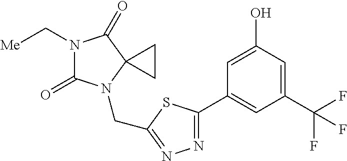 [SMILES: CCN1C(=O)N(Cc2nnc(-c3cc(O)cc(C(F)(F)F)c3)s2)C2(CC2)C1=O] | 41.0 ± 3.90 n = 2 | 144^{b} n = 1 |
| I-23 | 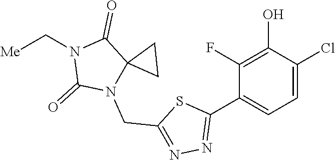 [SMILES: CCN1C(=O)N(Cc2nnc(-c3ccc(Cl)c(O)c3F)s2)C2(CC2)C1=O] | 78.8 n = 1 | N/D |
| I-24 | 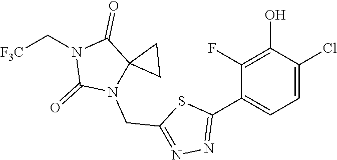 [SMILES: O=C1N(CC(F)(F)F)C(=O)C2(CC2)N1Cc1nnc(-c2ccc(Cl)c(O)c2F)s1] | 71.4 n = 1 | N/D |
| I-25 | 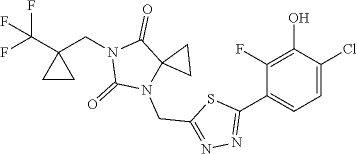 [SMILES: O=C1N(CC2(C(F)(F)F)CC2)C(=O)C2(CC2)N1Cc1nnc(-c2ccc(Cl)c(O)c2F)s1] | 146 n = 1 | N/D |
| I-26 | 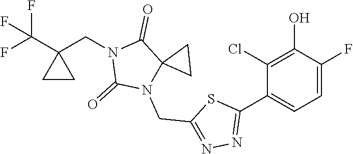 [SMILES: O=C1N(CC2(C(F)(F)F)CC2)C(=O)C2(CC2)N1Cc1nnc(-c2ccc(F)c(O)c2Cl)s1] | 18.7 ± 2.67 n = 2 | 107^{b} n = 1 |
| I-27 | 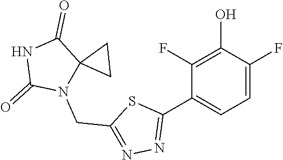 [SMILES: O=C1NC(=O)C2(CC2)N1Cc1nnc(-c2ccc(F)c(O)c2F)s1] | 355 n = 1 | N/D |
| I-28 | 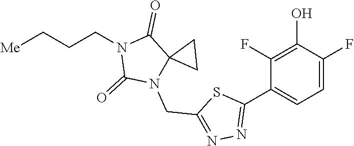 [SMILES: CCCCN1C(=O)N(Cc2nnc(-c3ccc(F)c(O)c3F)s2)C2(CC2)C1=O] | 8.11 n = 1 | N/D |
| I-29 | 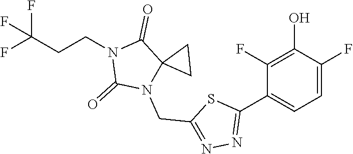 [SMILES: O=C1N(CCC(F)(F)F)C(=O)C2(CC2)N1Cc1nnc(-c2ccc(F)c(O)c2F)s1] | 18.6 n = 1 | N/D |
| I-30 | 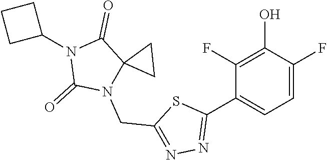 [SMILES: O=C1N(C2CCC2)C(=O)C2(CC2)N1Cc1nnc(-c2ccc(F)c(O)c2F)s1] | 7.08 n = 1 | 35.9^{b} |
| I-31 | 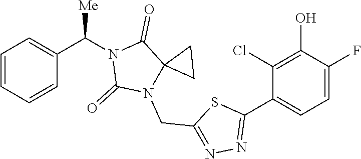 [SMILES: C[C@H](c1ccccc1)N1C(=O)N(Cc2nnc(-c3ccc(F)c(O)c3Cl)s2)C2(CC2)C1=O] | 2.68 n = 1 | 10.8^{b} |
| I-32 | 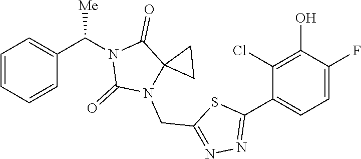 [SMILES: C[C@@H](c1ccccc1)N1C(=O)N(Cc2nnc(-c3ccc(F)c(O)c3Cl)s2)C2(CC2)C1=O] | 3.72 n = 1 | 14.7^{b} |
| I-33 | 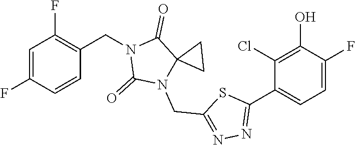 [SMILES: O=C1N(Cc2ccc(F)cc2F)C(=O)C2(CC2)N1Cc1nnc(-c2ccc(F)c(O)c2Cl)s1] | 8.83 n = 1 | N/D |
| I-34 | 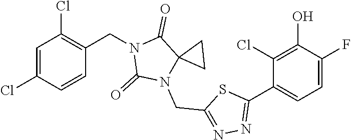 [SMILES: O=C1N(Cc2ccc(Cl)cc2Cl)C(=O)C2(CC2)N1Cc1nnc(-c2ccc(F)c(O)c2Cl)s1] | 4.60 n = 1 | 119^{b} n = 1 |
| I-35 | 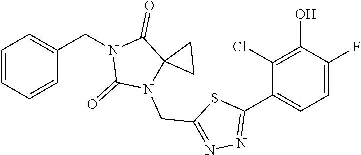 [SMILES: O=C1N(Cc2ccccc2)C(=O)C2(CC2)N1Cc1nnc(-c2ccc(F)c(O)c2Cl)s1] | 5.33 n = 1 | 40.9^{b} n = 1 |
| I-36 | 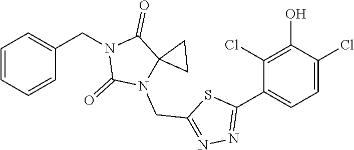 [SMILES: O=C1N(Cc2ccccc2)C(=O)C2(CC2)N1Cc1nnc(-c2ccc(Cl)c(O)c2Cl)s1] | 38.5 n = 1 | N/D |
| I-37 | 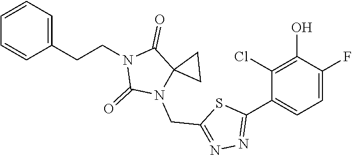 [SMILES: O=C1N(CCc2ccccc2)C(=O)C2(CC2)N1Cc1nnc(-c2ccc(F)c(O)c2Cl)s1] | 32.7 n = 1 | N/D |
| I-38 | 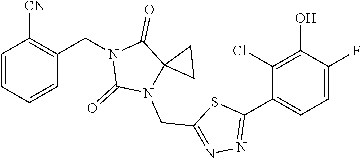 [SMILES: N#Cc1ccccc1CN1C(=O)N(Cc2nnc(-c3ccc(F)c(O)c3Cl)s2)C2(CC2)C1=O] | 39.4 n = 1 | N/D |
| I-39 | 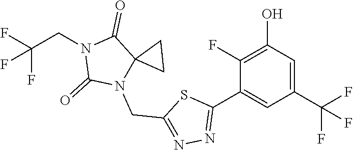 [SMILES: O=C1N(CC(F)(F)F)C(=O)C2(CC2)N1Cc1nnc(-c2cc(C(F)(F)F)cc(O)c2F)s1] | 8.45 n = 1 | N/D |
| I-40 | 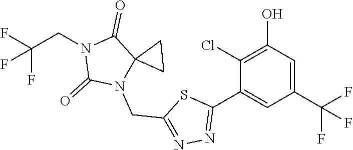 [SMILES: O=C1N(CC(F)(F)F)C(=O)C2(CC2)N1Cc1nnc(-c2cc(C(F)(F)F)cc(O)c2Cl)s1] | 9.80 n = 1 | 66.4^{b} n = 1 |
| I-41 | 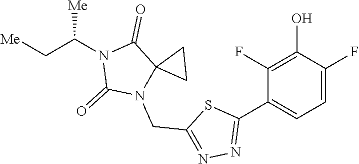 [SMILES: CC[C@H](C)N1C(=O)N(Cc2nnc(-c3ccc(F)c(O)c3F)s2)C2(CC2)C1=O] | 4.15 n = 1 | 18.9^{b} |
| I-42 | 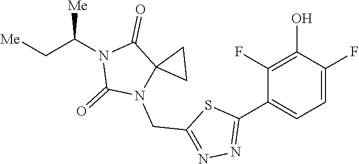 [SMILES: CC[C@@H](C)N1C(=O)N(Cc2nnc(-c3ccc(F)c(O)c3F)s2)C2(CC2)C1=O] | 4.08 n = 1 | 15.7^{b} |
| I-43 | 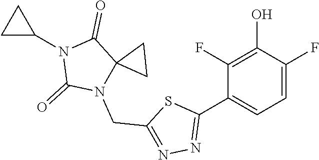 [SMILES: O=C1N(C2CC2)C(=O)C2(CC2)N1Cc1nnc(-c2ccc(F)c(O)c2F)s1] | 18.5 n = 1 | N/D |
| I-44 | 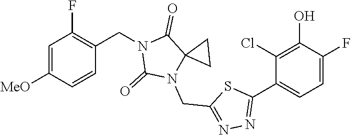 [SMILES: COc1ccc(CN2C(=O)N(Cc3nnc(-c4ccc(F)c(O)c4Cl)s3)C3(CC3)C2=O)c(F)c1] | 7.38 n = 1 | N/D |
| I-45 | 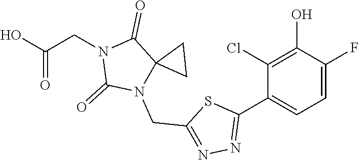 [SMILES: O=C(O)CN1C(=O)N(Cc2nnc(-c3ccc(F)c(O)c3Cl)s2)C2(CC2)C1=O] | 1030 n = 1 | N/D |
| I-46 | 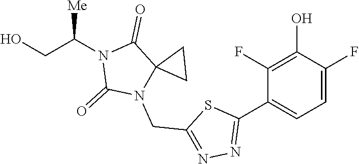 [SMILES: C[C@H](CO)N1C(=O)N(Cc2nnc(-c3ccc(F)c(O)c3F)s2)C2(CC2)C1=O] | 66 n = 1 | N/D |
| I-47 | 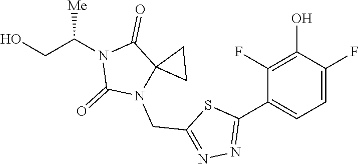 [SMILES: C[C@@H](CO)N1C(=O)N(Cc2nnc(-c3ccc(F)c(O)c3F)s2)C2(CC2)C1=O] | 53 n = 1 | N/D |
| I-48 | 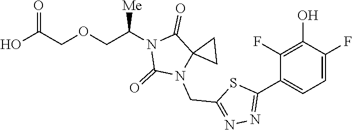 [SMILES: C[C@H](COCC(=O)O)N1C(=O)N(Cc2nnc(-c3ccc(F)c(O)c3F)s2)C2(CC2)C1=O] | 1060 n = 1 | N/D |
| I-49 | 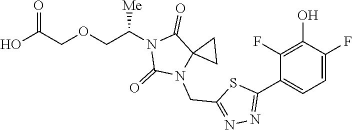 [SMILES: C[C@@H](COCC(=O)O)N1C(=O)N(Cc2nnc(-c3ccc(F)c(O)c3F)s2)C2(CC2)C1=O] | 426 n = 1 | N/D |
| I-50 | 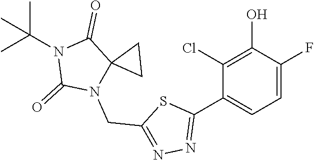 [SMILES: CC(C)(C)N1C(=O)N(Cc2nnc(-c3ccc(F)c(O)c3Cl)s2)C2(CC2)C1=O] | 4.6 n = 1 | 35.1 ± 6.03^{b} n = 2 |
| I-52 | 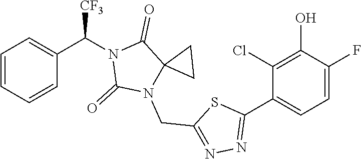 [SMILES: O=C1N([C@@H](c2ccccc2)C(F)(F)F)C(=O)C2(CC2)N1Cc1nnc(-c2ccc(F)c(O)c2Cl)s1] | 1.089 ± 0.23 n = 3 | 6.41 ± 1.23^{b} n = 2 |
| I-53 | 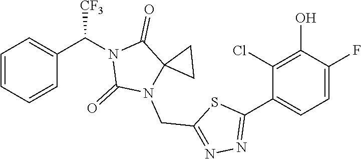 [SMILES: O=C1N([C@H](c2ccccc2)C(F)(F)F)C(=O)C2(CC2)N1Cc1nnc(-c2ccc(F)c(O)c2Cl)s1] | 5.6 n = 1 | 26.3^{b} n = 1 |
| I-54 | 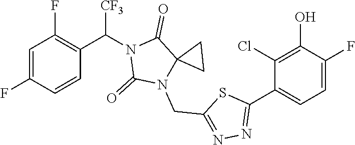 [SMILES: O=C1N(C(c2ccc(F)cc2F)C(F)(F)F)C(=O)C2(CC2)N1Cc1nnc(-c2ccc(F)c(O)c2Cl)s1] | 1.53 n = 1 | 10.7^{b} n = 1 |
| I-54A Enantiomer 1 | 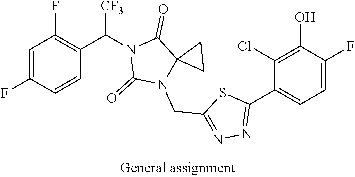 [SMILES: O=C1N(C(c2ccc(F)cc2F)C(F)(F)F)C(=O)C2(CC2)N1Cc1nnc(-c2ccc(F)c(O)c2Cl)s1] | 0.857 ± 0.153 n = 2 | 5.63 ± 0.961^{b} n = 2 |
| I-54B Enantiomer 2 | 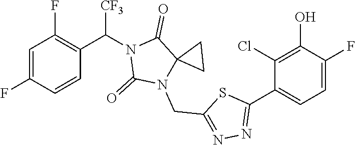 [SMILES: O=C1N(C(c2ccc(F)cc2F)C(F)(F)F)C(=O)C2(CC2)N1Cc1nnc(-c2ccc(F)c(O)c2Cl)s1] | 8.43 n = 1 | ND |
| I-55 | 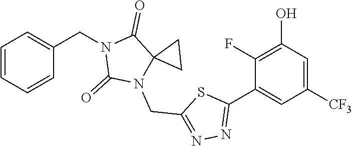 [SMILES: O=C1N(Cc2ccccc2)C(=O)C2(CC2)N1Cc1nnc(-c2cc(C(F)(F)F)cc(O)c2F)s1] | 2.14 n = 1 | 29.2^{b} n = 1 |
| I-56 | 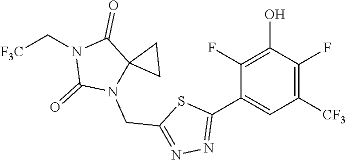 [SMILES: O=C1N(CC(F)(F)F)C(=O)C2(CC2)N1Cc1nnc(-c2cc(C(F)(F)F)c(F)c(O)c2F)s1] | 12.5 n = 1 | ND |
| I-57 | 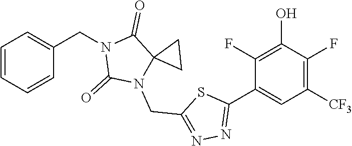 [SMILES: O=C1N(Cc2ccccc2)C(=O)C2(CC2)N1Cc1nnc(-c2cc(C(F)(F)F)c(F)c(O)c2F)s1] | 2.46 n = 1 | 52.7^{b} n = 1 |
| I-58 | 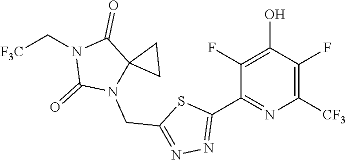 [SMILES: O=C1N(CC(F)(F)F)C(=O)C2(CC2)N1Cc1nnc(-c2nc(C(F)(F)F)c(F)c(O)c2F)s1] | 293 n = 1 | ND |
| I-59 | 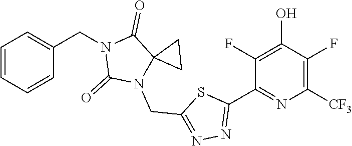 [SMILES: O=C1N(Cc2ccccc2)C(=O)C2(CC2)N1Cc1nnc(-c2nc(C(F)(F)F)c(F)c(O)c2F)s1] | 161 n = 1 | ND |
| I-60 | 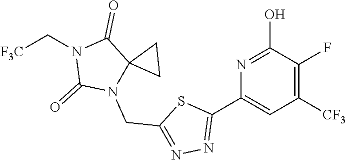 [SMILES: O=C1N(CC(F)(F)F)C(=O)C2(CC2)N1Cc1nnc(-c2cc(C(F)(F)F)c(F)c(O)n2)s1] | 2960 n = 1 | ND |
| I-61 | 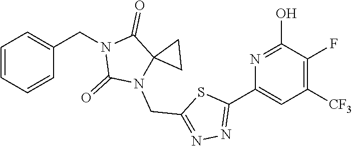 [SMILES: O=C1N(Cc2ccccc2)C(=O)C2(CC2)N1Cc1nnc(-c2cc(C(F)(F)F)c(F)c(O)n2)s1] | 505 n = 1 | ND |
| I-62 | 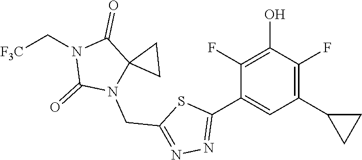 [SMILES: O=C1N(CC(F)(F)F)C(=O)C2(CC2)N1Cc1nnc(-c2cc(C3CC3)c(F)c(O)c2F)s1] | 84.9 n = 1 | ND |
| I-63 | 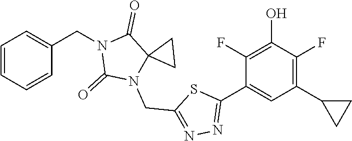 [SMILES: O=C1N(Cc2ccccc2)C(=O)C2(CC2)N1Cc1nnc(-c2cc(C3CC3)c(F)c(O)c2F)s1] | 18.0 n = 1 | ND |
| I-64 | 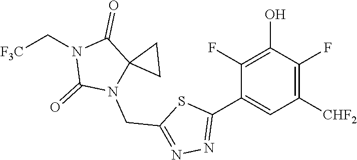 [SMILES: O=C1N(CC(F)(F)F)C(=O)C2(CC2)N1Cc1nnc(-c2cc(C(F)F)c(F)c(O)c2F)s1] | 22.5 n = 1 | ND |
| I-65 | 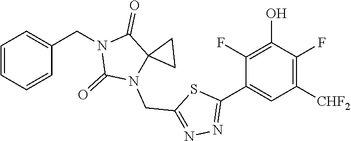 [SMILES: O=C1N(Cc2ccccc2)C(=O)C2(CC2)N1Cc1nnc(-c2cc(C(F)F)c(F)c(O)c2F)s1] | 5.78 n = 1 | 19.2^{b} n = 1 |
| I-68 | 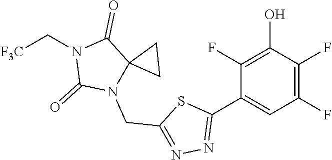 [SMILES: O=C1N(CC(F)(F)F)C(=O)C2(CC2)N1Cc1nnc(-c2cc(F)c(F)c(O)c2F)s1] | 8.58 n = 1 | 35.0^{b} n = 1 |
| I-69 | 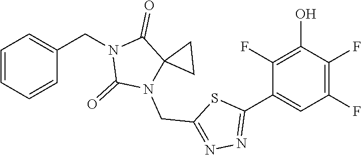 [SMILES: O=C1N(Cc2ccccc2)C(=O)C2(CC2)N1Cc1nnc(-c2cc(F)c(F)c(O)c2F)s1] | 3.23 n = 1 | 21.5^{b} n = 1 |
| I-73 | 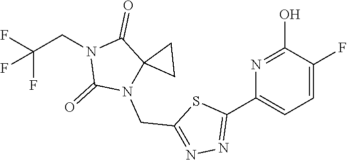 [SMILES: O=C1N(CC(F)(F)F)C(=O)C2(CC2)N1Cc1nnc(-c2ccc(F)c(O)n2)s1] | 1390 n = 1 | ND |
| I-74 | 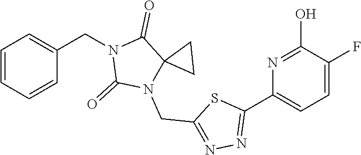 [SMILES: O=C1N(Cc2ccccc2)C(=O)C2(CC2)N1Cc1nnc(-c2ccc(F)c(O)n2)s1] | 147 n = 1 | ND |
| I-75 | 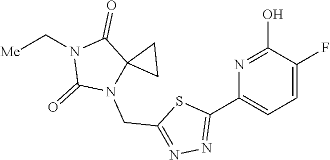 [SMILES: CCN1C(=O)N(Cc2nnc(-c3ccc(F)c(O)n3)s2)C2(CC2)C1=O] | 1360 n = 1 | ND |
| I-76 | 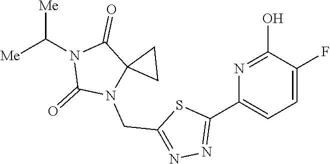 [SMILES: CC(C)N1C(=O)N(Cc2nnc(-c3ccc(F)c(O)n3)s2)C2(CC2)C1=O] | 1390 n = 1 | ND |
| I-77 | 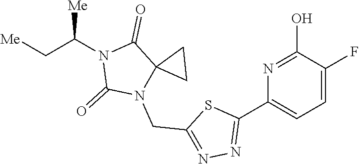 [SMILES: CC[C@@H](C)N1C(=O)N(Cc2nnc(-c3ccc(F)c(O)n3)s2)C2(CC2)C1=O] | 1060 n = 1 | ND |
| I-78 | 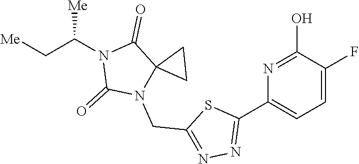 [SMILES: CC[C@H](C)N1C(=O)N(Cc2nnc(-c3ccc(F)c(O)n3)s2)C2(CC2)C1=O] | 934 n = 1 | ND |
| I-79 | 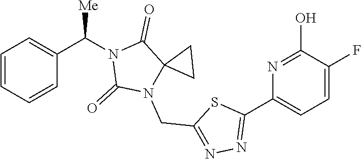 [SMILES: C[C@H](c1ccccc1)N1C(=O)N(Cc2nnc(-c3ccc(F)c(O)n3)s2)C2(CC2)C1=O] | 122 n = 1 | ND |
| I-80 | 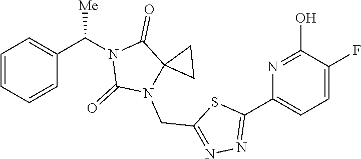 [SMILES: C[C@@H](c1ccccc1)N1C(=O)N(Cc2nnc(-c3ccc(F)c(O)n3)s2)C2(CC2)C1=O] | 76.8 n = 1 | ND |
| I-81 | 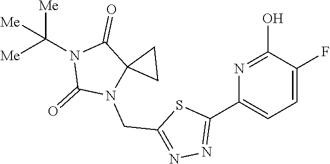 [SMILES: CC(C)(C)N1C(=O)N(Cc2nnc(-c3ccc(F)c(O)n3)s2)C2(CC2)C1=O] | 602 n = 1 | ND |
| I-82 | 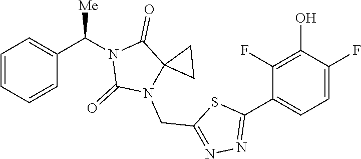 [SMILES: C[C@H](c1ccccc1)N1C(=O)N(Cc2nnc(-c3ccc(F)c(O)c3F)s2)C2(CC2)C1=O] | 1.76 ± 0.26 n = 2 | 8.42^{b} n = 1 |
| I-83 | 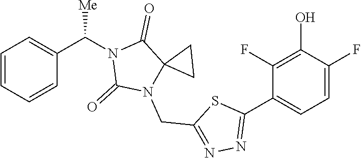 [SMILES: C[C@@H](c1ccccc1)N1C(=O)N(Cc2nnc(-c3ccc(F)c(O)c3F)s2)C2(CC2)C1=O] | 2.00 ± 0.588 n =2 | 10.4^{b} n = 1 |
| I-84 | 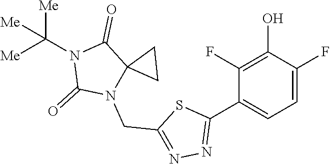 [SMILES: CC(C)(C)N1C(=O)N(Cc2nnc(-c3ccc(F)c(O)c3F)s2)C2(CC2)C1=O] | 4.07 n = 1 | ND |
| I-85 | 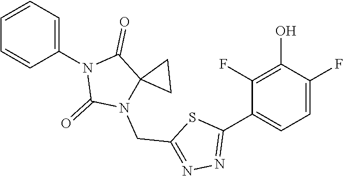 [SMILES: O=C1N(c2ccccc2)C(=O)C2(CC2)N1Cc1nnc(-c2ccc(F)c(O)c2F)s1] | 13.2 n = 1 | ND |
| I-86 | 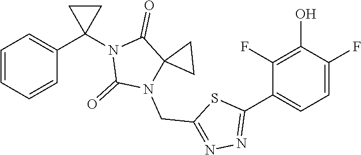 [SMILES: O=C1N(Cc2nnc(-c3ccc(F)c(O)c3F)s2)C2(CC2)C(=O)N1C1(c2ccccc2)CC1] | 2.50 n = 1 | 4.74^{b} n = 1 |
| I-87 | 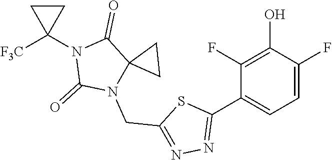 [SMILES: O=C1N(Cc2nnc(-c3ccc(F)c(O)c3F)s2)C2(CC2)C(=O)N1C1(C(F)(F)F)CC1] | 11.3 n = 1 | ND |
| I-88 | 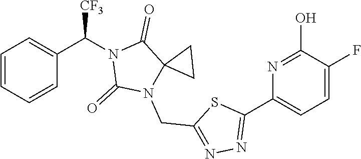 [SMILES: O=C1N([C@@H](c2ccccc2)C(F)(F)F)C(=O)C2(CC2)N1Cc1nnc(-c2ccc(F)c(O)n2)s1] | 29.1 n = 1 | ND |
| I-89 | 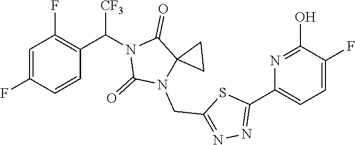 [SMILES: O=C1N(C(c2ccc(F)cc2F)C(F)(F)F)C(=O)C2(CC2)N1Cc1nnc(-c2ccc(F)c(O)n2)s1] | 15.6 n = 1 | ND |
| I-90A | 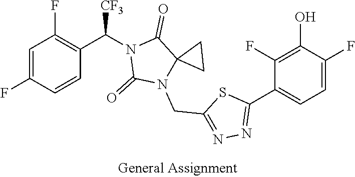 [SMILES: O=C1N([C@@H](c2ccc(F)cc2F)C(F)(F)F)C(=O)C2(CC2)N1Cc1nnc(-c2ccc(F)c(O)c2F)s1] | 1.01 ± 0.125 n = 2 | 8.38^{b} n = 1 |
| I-90B | 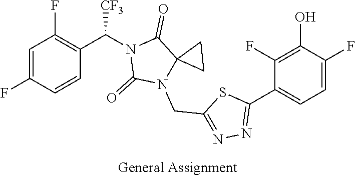 [SMILES: O=C1N([C@H](c2ccc(F)cc2F)C(F)(F)F)C(=O)C2(CC2)N1Cc1nnc(-c2ccc(F)c(O)c2F)s1] | 6.53 n = 1 | ND |
| I-91 | 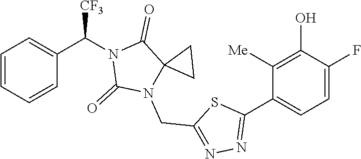 [SMILES: Cc1c(-c2nnc(CN3C(=O)N([C@@H](c4ccccc4)C(F)(F)F)C(=O)C34CC4)s2)ccc(F)c1O] | 4.00 n = 1 | 108^{b} n = 1 |
| I-92 | 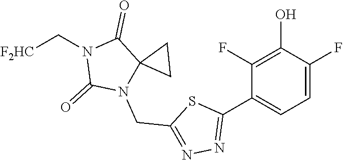 [SMILES: O=C1N(CC(F)F)C(=O)C2(CC2)N1Cc1nnc(-c2ccc(F)c(O)c2F)s1] | 14.3 n = 1 | ND |
| I-93 | 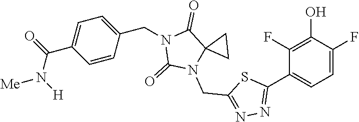 [SMILES: CNC(=O)c1ccc(CN2C(=O)N(Cc3nnc(-c4ccc(F)c(O)c4F)s3)C3(CC3)C2=O)cc1] | 75.4 n = 1 | ND |
| I-94 | 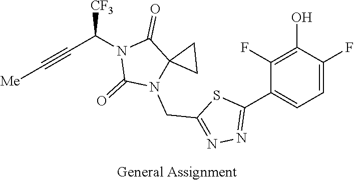 [SMILES: CC#C[C@H](N1C(=O)N(Cc2nnc(-c3ccc(F)c(O)c3F)s2)C2(CC2)C1=O)C(F)(F)F] | 6.77 n = 1 | ND |
| I-95 | 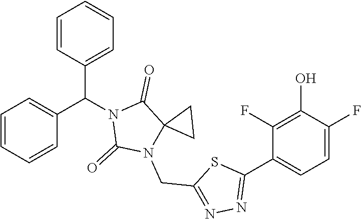 [SMILES: O=C1N(C(c2ccccc2)c2ccccc2)C(=O)C2(CC2)N1Cc1nnc(-c2ccc(F)c(O)c2F)s1] | 62.0 n = 1 | ND |
| I-96 | 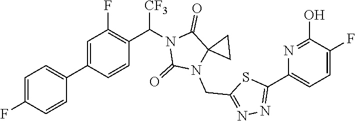 [SMILES: O=C1N(C(c2ccc(-c3ccc(F)cc3)cc2F)C(F)(F)F)C(=O)C2(CC2)N1Cc1nnc(-c2ccc(F)c(O)n2)s1] | 3.11 n = 1 | 121^{b} n = 1 |
| I-97 | 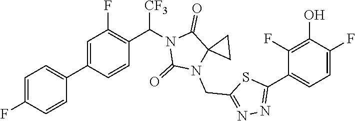 [SMILES: O=C1N(C(c2ccc(-c3ccc(F)cc3)cc2F)C(F)(F)F)C(=O)C2(CC2)N1Cc1nnc(-c2ccc(F)c(O)c2F)s1] | 0.79 n = 1 | 4.66^{b} n = 1 |
| I-99A | 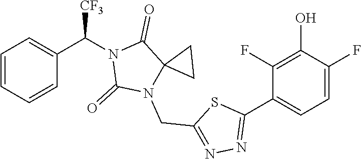 [SMILES: O=C1N([C@@H](c2ccccc2)C(F)(F)F)C(=O)C2(CC2)N1Cc1nnc(-c2ccc(F)c(O)c2F)s1] | 1.17 ± 0.185 n = 4 | 4.95 ± 0.0122 ^{b}n = 2 |
| I-103 | 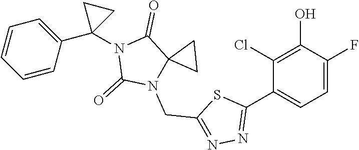 [SMILES: O=C1N(Cc2nnc(-c3ccc(F)c(O)c3Cl)s2)C2(CC2)C(=O)N1C1(c2ccccc2)CC1] | 4.39 n = 1 | ND |
| I-104 | 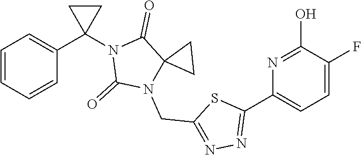 [SMILES: O=C1N(Cc2nnc(-c3ccc(F)c(O)n3)s2)C2(CC2)C(=O)N1C1(c2ccccc2)CC1] | 176 n = 1 | ND |

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A compound, a pharmaceutically acceptable salt thereof, or a pharmaceutically acceptable prodrug thereof, of Formula I: 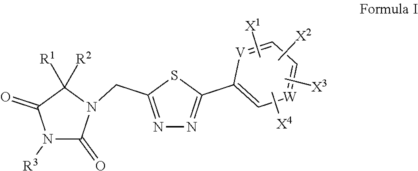
wherein X¹, X², X³, and X⁴ are each independently selected from H, F, Cl, Br, optionally substituted C₁-C₆ alkyl, C₁-C₄ haloalkyl, optionally substituted C₃-C₆ cycloalkyl, optionally substituted alkoxy, and hydroxy;
V is CH or N;
W is CH or N;
with a proviso that V and W are not simultaneously N and when either one or both of V and W are CH they can be independently substituted by X¹, X², X³, or X⁴ thereby becoming CX¹, CX², CX³, or CX⁴;
wherein R¹ and R² are independently selected from hydrogen or optionally substituted C₁-C₈ linear or branched alkyl, with the proviso that R¹ and R² may be connected to one another to form a ring of 3 to 6 carbons; and wherein R³ is selected from hydrogen, optionally substituted C₁-C₈ linear or branched alkyl, optionally substituted C₁-C₈ cycloalkyl, optionally substituted phenyl, optionally substituted aralkyl, optionally substituted heteroaralkyl, optionally substituted cyclopropylalkyl, or optionally substituted C₁-C₈ alkynyl.

2. The compound, pharmaceutically acceptable salt thereof, or pharmaceutically acceptable prodrug thereof, of claim 1, wherein R³ is selected from hydrogen, optionally substituted C₁—C₈ linear or branched alkyl, optionally substituted C₁-C₈ cycloalkyl, optionally substituted phenyl, optionally substituted benzyl, optionally substituted 2-phenethyl, optionally substituted cyclopropylalkyl, optionally substituted C₁-C₈ alkynyl.

3. The compound, pharmaceutically acceptable salt thereof, or pharmaceutically acceptable prodrug thereof, of claim 1, wherein the compound is the compound of Formula Ib: 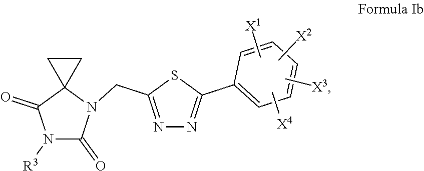
wherein X¹, X², X³, X⁴ and R³ are defined in the same way as in Formula I.

4. The compound, pharmaceutically acceptable salt thereof, or pharmaceutically acceptable prodrug thereof, of claim 1, wherein the compound is of Formula Ic: 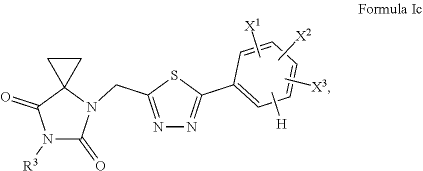
wherein X¹, X², X³, and R³ are defined in the same way as in Formula I.

5. The compound, pharmaceutically acceptable salt thereof, or pharmaceutically acceptable prodrug thereof, of claim 1, wherein the compound is of Formula Id: 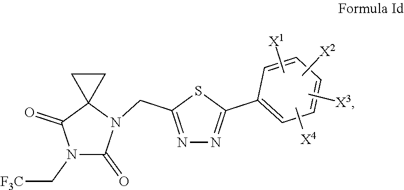
wherein X¹, X², X³, and X⁴ are defined in the same way as in Formula I.

6. The compound, pharmaceutically acceptable salt thereof, or pharmaceutically acceptable prodrug thereof, of claim 1, wherein compound is of Formula Ie: 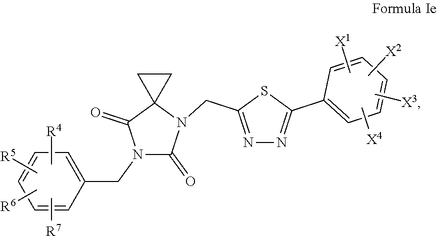
wherein X¹, X², X³, and X⁴ are defined in the same way as in Formula I; and wherein R⁴, R⁵, R⁶, and R⁷ are independently selected from halo, CN, amino, hydroxy, —C₁₋₃ alkyl, C₁-C₄ haloalkyl, optionally substituted phenyl, —OR*, —NR*₂, —SR*, —SO₂R*, —COOR*, or —CONR*₂, where each R* is independently H, C₁₋₄ alkyl, C₁-C₄ haloalkyl, or C₁-C₄ haloalkyl, C₃₋₈ cycloalkyl with the proviso that two R* on —CONR*₂ may form a ring of C₃-C₈ carbons.

7. The compound, pharmaceutically acceptable salt thereof, or pharmaceutically acceptable prodrug thereof, of claim 1, wherein the compound is of Formula If: 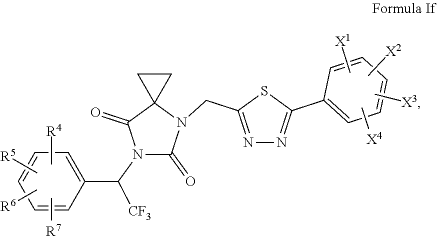
wherein X¹, X², X³, and X⁴ are defined in the same way as in Formula I; and wherein R⁴, R⁵, R⁶, and R⁷ are independently selected from halo, CN, amino, hydroxy, —C₁₋₃ alkyl, C₁-C₄ haloalkyl, optionally substituted phenyl, —OR*, —NR*₂, —SR*, —SO₂R*, —COOR*, or —CONR*₂, where each R* is independently H, C₁₋₄ alkyl, C₁-C₄ haloalkyl or C₃₋₈ cycloalkyl with the proviso that two R* on —CONR*₂ may form a ring of C₃-C₈ carbons.

8. The compound, pharmaceutically acceptable salt thereof, or pharmaceutically acceptable prodrug thereof, of claim 1, wherein the compound is of Formula Ig: 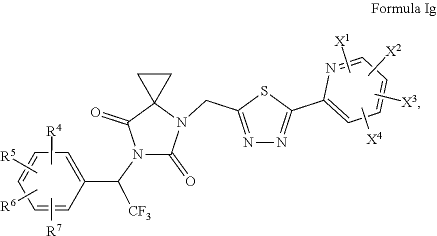
wherein X¹, X², X³, and X⁴ are defined in the same way as in Formula I; and wherein R⁴, R⁵, R⁶, and R⁷ are independently selected from halo, CN, amino, hydroxy, —C₁₋₃ alkyl, C₁-C₄ haloalkyl, optionally substituted phenyl, optionally substituted cyclopropyl, —OR*, —NR*₂, —SR*, —SO₂R*, —COOR*, or —CONR*₂, where each R* is independently H, C₁₋₄ alkyl, C₁-C₄ haloalkyl, or C₃₋₈ cycloalkyl with the proviso that two R* on —CONR*₂ may form a ring of C₃-C₈ carbons.

9. The compound, pharmaceutically acceptable salt thereof, or pharmaceutically acceptable prodrug thereof, of claim 1, wherein the compound is selected from the group consisting of:
5-((5-(2,4-difluoro-3-hydroxyphenyl)-1,3,4-thiadiazol-2-yl)methyl)-7-ethyl-5,7-diazaspiro[3.4]octane-6,8-dione;
6-ethyl-4-((5-(6-fluoro-5-hydroxypyridin-3-yl)-1,3,4-thiadiazol-2-yl)methyl)-4,6-diazaspiro[2.4]heptane-5,7-dione;
1-((5-(2,4-difluoro-3-hydroxyphenyl)-1,3,4-thiadiazol-2-yl)methyl)-3-ethyl-5-methylimidazolidine-2,4-dione;
4-((5-(2,4-difluoro-3-hydroxyphenyl)-1,3,4-thiadiazol-2-yl)methyl)-6-propyl-4,6-diazaspiro[2.4]heptane-5,7-dione;
4-((5-(2,4-difluoro-3-hydroxyphenyl)-1,3,4-thiadiazol-2-yl)methyl)-6-isopropyl-4,6-diazaspiro[2.4]heptane-5,7-dione;
6-(bicyclo[1.1.1]pentan-1-ylmethyl)-4-((5-(2,4-difluoro-3-hydroxyphenyl)-1,3,4-thiadiazol-2-yl)methyl)-4,6-diazaspiro[2.4]heptane-5,7-dione;
4-((5-(2,4-difluoro-3-hydroxyphenyl)-1,3,4-thiadiazol-2-yl)methyl)-6-ethyl-4,6-diazaspiro[2.4]heptane-5,7-dione;
1-((5-(2,4-difluoro-3-hydroxyphenyl)-1,3,4-thiadiazol-2-yl)methyl)-3-ethyl-5,5-dimethylimidazolidine-2,4-dione;
4-((5-(2,4-dichloro-3-hydroxyphenyl)-1,3,4-thiadiazol-2-yl)methyl)-6-ethyl-4,6-diazaspiro[2.4]heptane-5,7-dione;
4-((5-(2-chloro-4-fluoro-3-hydroxyphenyl)-1,3,4-thiadiazol-2-yl)methyl)-6-ethyl-4,6-diazaspiro[2.4]heptane-5,7-dione;
4-((5-(4-chloro-5-hydroxypyridin-3-yl)-1,3,4-thiadiazol-2-yl)methyl)-6-ethyl-4,6-diazaspiro[2.4]heptane-5,7-dione;
4-((5-(6-chloro-5-hydroxypyridin-3-yl)-1,3,4-thiadiazol-2-yl)methyl)-6-ethyl-4,6-diazaspiro[2.4]heptane-5,7-dione;
4-((5-(2-chloro-3-hydroxy-5-(trifluoromethyl)phenyl)-1,3,4-thiadiazol-2-yl)methyl)-6-ethyl-4,6-diazaspiro[2.4]heptane-5,7-dione;
6-ethyl-4-((5-(2-fluoro-3-hydroxy-5-(trifluoromethyl)phenyl)-1,3,4-thiadiazol-2-yl)methyl)-4,6-diazaspiro[2.4]heptane-5,7-dione;
4-((5-(2,4-difluoro-3-hydroxyphenyl)-1,3,4-thiadiazol-2-yl)methyl)-6-(2,2,2-trifluoroethyl)-4,6-diazaspiro[2.4]heptane-5,7-dione;
4-((5-(2-chloro-4-fluoro-3-hydroxyphenyl)-1,3,4-thiadiazol-2-yl)methyl)-6-(2,2,2-trifluoroethyl)-4,6-diazaspiro[2.4]heptane-5,7-dione;
4-((5-(2,4-dichloro-3-hydroxyphenyl)-1,3,4-thiadiazol-2-yl)methyl)-6-(2,2,2-trifluoroethyl)-4,6-diazaspiro[2.4]heptane-5,7-dione;
4-((5-(4-chloro-5-hydroxypyridin-3-yl)-1,3,4-thiadiazol-2-yl)methyl)-6-(2,2,2-trifluoroethyl)-4,6-diazaspiro[2.4]heptane-5,7-dione;
4-((5-(2,4-difluoro-3-hydroxyphenyl)-1,3,4-thiadiazol-2-yl)methyl)-6-((1-(trifluoromethyl)cyclopropyl)methyl)-4,6-diazaspiro[2.4]heptane-5,7-dione;
6-benzyl-4-((5-(2,4-difluoro-3-hydroxyphenyl)-1,3,4-thiadiazol-2-yl)methyl)-4,6-diazaspiro[2.4]heptane-5,7-dione;
4-((5-(2,4-difluoro-3-hydroxyphenyl)-1,3,4-thiadiazol-2-yl)methyl)-6-(pyrimidin-2-ylmethyl)-4,6-diazaspiro[2.4]heptane-5,7-dione;
6-ethyl-4-((5-(3-hydroxy-5-(trifluoromethyl)phenyl)-1,3,4-thiadiazol-2-yl)methyl)-4,6-diazaspiro[2.4]heptane-5,7-dione;
4-((5-(4-chloro-2-fluoro-3-hydroxyphenyl)-1,3,4-thiadiazol-2-yl)methyl)-6-ethyl-4,6-diazaspiro[2.4]heptane-5,7-dione;
4-((5-(4-chloro-2-fluoro-3-hydroxyphenyl)-1,3,4-thiadiazol-2-yl)methyl)-6-(2,2,2-trifluoroethyl)-4,6-diazaspiro[2.4]heptane-5,7-dione;
4-((5-(4-chloro-2-fluoro-3-hydroxyphenyl)-1,3,4-thiadiazol-2-yl)methyl)-6-((1-(trifluoromethyl)cyclopropyl)methyl)-4,6-diazaspiro[2.4]heptane-5,7-dione;
4-((5-(2-chloro-4-fluoro-3-hydroxyphenyl)-1,3,4-thiadiazol-2-yl)methyl)-6-((1-(trifluoromethyl)cyclopropyl)methyl)-4,6-diazaspiro[2.4]heptane-5,7-dione;
4-((5-(2,4-difluoro-3-hydroxyphenyl)-1,3,4-thiadiazol-2-yl)methyl)-4,6-diazaspiro[2.4]heptane-5,7-dione;
6-butyl-4-((5-(2,4-difluoro-3-hydroxyphenyl)-1,3,4-thiadiazol-2-yl)methyl)-4,6-diazaspiro[2.4]heptane-5,7-dione;
4-((5-(2,4-difluoro-3-hydroxyphenyl)-1,3,4-thiadiazol-2-yl)methyl)-6-(3,3,3-trifluoropropyl)-4,6-diazaspiro[2.4]heptane-5,7-dione;
6-cyclobutyl-4-((5-(2,4-difluoro-3-hydroxyphenyl)-1,3,4-thiadiazol-2-yl)methyl)-4,6-diazaspiro[2.4]heptane-5,7-dione;
(R)-4-((5-(2-chloro-4-fluoro-3-hydroxyphenyl)-1,3,4-thiadiazol-2-yl)methyl)-6-(1-phenylethyl)-4,6-diazaspiro[2.4]heptane-5,7-dione;
(S)-4-((5-(2-chloro-4-fluoro-3-hydroxyphenyl)-1,3,4-thiadiazol-2-yl)methyl)-6-(1-phenylethyl)-4,6-diazaspiro[2.4]heptane-5,7-dione;
4-((5-(2-chloro-4-fluoro-3-hydroxyphenyl)-1,3,4-thiadiazol-2-yl)methyl)-6-(2,4-difluorobenzyl)-4,6-diazaspiro[2.4]heptane-5,7-dione;
4-((5-(2-chloro-4-fluoro-3-hydroxyphenyl)-1,3,4-thiadiazol-2-yl)methyl)-6-(2,4-dichlorobenzyl)-4,6-diazaspiro[2.4]heptane-5,7-dione;
6-benzyl-4-((5-(2-chloro-4-fluoro-3-hydroxyphenyl)-1,3,4-thiadiazol-2-yl)methyl)-4,6-diazaspiro[2.4]heptane-5,7-dione;
6-benzyl-4-((5-(2,4-dichloro-3-hydroxyphenyl)-1,3,4-thiadiazol-2-yl)methyl)-4,6-diazaspiro[2.4]heptane-5,7-dione;
4-((5-(2-chloro-4-fluoro-3-hydroxyphenyl)-1,3,4-thiadiazol-2-yl)methyl)-6-phenethyl-4,6-diazaspiro[2.4]heptane-5,7-dione;
2-((4-((5-(2-chloro-4-fluoro-3-hydroxyphenyl)-1,3,4-thiadiazol-2-yl)methyl)-5,7-dioxo-4,6-diazaspiro[2.4]heptan-6-yl)methyl)benzonitrile;
4-((5-(2-fluoro-3-hydroxy-5-(trifluoromethyl)phenyl)-1,3,4-thiadiazol-2-yl)methyl)-6-(2,2,2-trifluoroethyl)-4,6-diazaspiro[2.4]heptane-5,7-dione;
4-((5-(2-chloro-3-hydroxy-5-(trifluoromethyl)phenyl)-1,3,4-thiadiazol-2-yl)methyl)-6-(2,2,2-trifluoroethyl)-4,6-diazaspiro[2.4]heptane-5,7-dione;
(S)-6-(sec-butyl)-4-((5-(2,4-difluoro-3-hydroxyphenyl)-1,3,4-thiadiazol-2-yl)methyl)-4,6-diazaspiro[2.4]heptane-5,7-dione;
(R)-6-(sec-butyl)-4-((5-(2,4-difluoro-3-hydroxyphenyl)-1,3,4-thiadiazol-2-yl)methyl)-4,6-diazaspiro[2.4]heptane-5,7-dione;
6-cyclopropyl-4-((5-(2,4-difluoro-3-hydroxyphenyl)-1,3,4-thiadiazol-2-yl)methyl)-4,6-diazaspiro[2.4]heptane-5,7-dione;
4-((5-(2-chloro-4-fluoro-3-hydroxyphenyl)-1,3,4-thiadiazol-2-yl)methyl)-6-(2-fluoro-4-methoxybenzyl)-4,6-diazaspiro[2.4]heptane-5,7-dione;
2-(4-((5-(2-chloro-4-fluoro-3-hydroxyphenyl)-1,3,4-thiadiazol-2-yl)methyl)-5,7-dioxo-4,6-diazaspiro[2.4]heptan-6-yl)acetic acid;
(R)-4-((5-(2,4-difluoro-3-hydroxyphenyl)-1,3,4-thiadiazol-2-yl)methyl)-6-(1-hydroxypropan-2-yl)-4,6-diazaspiro[2.4]heptane-5,7-dione;
(S)-4-((5-(2,4-difluoro-3-hydroxyphenyl)-1,3,4-thiadiazol-2-yl)methyl)-6-(1-hydroxypropan-2-yl)-4,6-diazaspiro[2.4]heptane-5,7-dione;
(R)-2-(2-(4-((5-(2,4-difluoro-3-hydroxyphenyl)-1,3,4-thiadiazol-2-yl)methyl)-5,7-dioxo-4,6-diazaspiro[2.4]heptan-6-yl)propoxy)acetic acid;
(S)-2-(2-(4-((5-(2,4-difluoro-3-hydroxyphenyl)-1,3,4-thiadiazol-2-yl)methyl)-5,7-dioxo-4,6-diazaspiro[2.4]heptan-6-yl)propoxy)acetic acid;
6-(tert-butyl)-4-((5-(2-chloro-4-fluoro-3-hydroxyphenyl)-1,3,4-thiadiazol-2-yl)methyl)-4,6-diazaspiro[2.4]heptane-5,7-dione;
(S)-4-((5-(2-chloro-4-fluoro-3-hydroxyphenyl)-1,3,4-thiadiazol-2-yl)methyl)-6-(2,2,2-trifluoro-1-phenylethyl)-4,6-diazaspiro[2.4]heptane-5,7-dione;
(R)-4-((5-(2-chloro-4-fluoro-3-hydroxyphenyl)-1,3,4-thiadiazol-2-yl)methyl)-6-(2,2,2-trifluoro-1-phenylethyl)-4,6-diazaspiro[2.4]heptane-5,7-dione;
4-((5-(2-chloro-4-fluoro-3-hydroxyphenyl)-1,3,4-thiadiazol-2-yl)methyl)-6-(1-(2,4-difluorophenyl)-2,2,2-trifluoroethyl)-4,6-diazaspiro[2.4]heptane-5,7-dione;
6-benzyl-4-((5-(2-fluoro-3-hydroxy-5-(trifluoromethyl)phenyl)-1,3,4-thiadiazol-2-yl)methyl)-4,6-diazaspiro[2.4]heptane-5,7-dione;
4-((5-(2,4-difluoro-3-hydroxy-5-(trifluoromethyl)phenyl)-1,3,4-thiadiazol-2-yl)methyl)-6-(2,2,2-trifluoroethyl)-4,6-diazaspiro[2.4]heptane-5,7-dione;
6-benzyl-4-((5-(2,4-difluoro-3-hydroxy-5-(trifluoromethyl)phenyl)-1,3,4-thiadiazol-2-yl)methyl)-4,6-diazaspiro[2.4]heptane-5,7-dione;
4-((5-(3,5-difluoro-4-hydroxy-6-(trifluoromethyl)pyridin-2-yl)-1,3,4-thiadiazol-2-yl)methyl)-6-(2,2,2-trifluoroethyl)-4,6-diazaspiro[2.4]heptane-5,7-dione;
6-benzyl-4-((5-(3,5-difluoro-4-hydroxy-6-(trifluoromethyl)pyridin-2-yl)-1,3,4-thiadiazol-2-yl)methyl)-4,6-diazaspiro[2.4]heptane-5,7-dione;
4-((5-(5-cyclopropyl-2,4-difluoro-3-hydroxyphenyl)-1,3,4-thiadiazol-2-yl)methyl)-6-(2,2,2-trifluoroethyl)-4,6-diazaspiro[2.4]heptane-5,7-dione;
6-benzyl-4-((5-(5-cyclopropyl-2,4-difluoro-3-hydroxyphenyl)-1,3,4-thiadiazol-2-yl)methyl)-4,6-diazaspiro[2.4]heptane-5,7-dione;
4-((5-(5-(difluoromethyl)-2,4-difluoro-3-hydroxyphenyl)-1,3,4-thiadiazol-2-yl)methyl)-6-(2,2,2-trifluoroethyl)-4,6-diazaspiro[2.4]heptane-5,7-dione;
6-benzyl-4-((5-(5-(difluoromethyl)-2,4-difluoro-3-hydroxyphenyl)-1,3,4-thiadiazol-2-yl)methyl)-4,6-diazaspiro[2.4]heptane-5,7-dione;
4-((5-(2,4,5-trifluoro-3-hydroxyphenyl)-1,3,4-thiadiazol-2-yl)methyl)-6-(2,2,2-trifluoroethyl)-4,6-diazaspiro[2.4]heptane-5,7-dione;
6-benzyl-4-((5-(2,4,5-trifluoro-3-hydroxyphenyl)-1,3,4-thiadiazol-2-yl)methyl)-4,6-diazaspiro[2.4]heptane-5,7-dione;
4-((5-(5-fluoro-6-hydroxypyridin-2-yl)-1,3,4-thiadiazol-2-yl)methyl)-6-(2,2,2-trifluoroethyl)-4,6-diazaspiro[2.4]heptane-5,7-dione;
6-benzyl-4-((5-(5-fluoro-6-hydroxypyridin-2-yl)-1,3,4-thiadiazol-2-yl)methyl)-4,6-diazaspiro[2.4]heptane-5,7-dione;
6-ethyl-4-((5-(5-fluoro-6-hydroxypyridin-2-yl)-1,3,4-thiadiazol-2-yl)methyl)-4,6-diazaspiro[2.4]heptane-5,7-dione;
4-((5-(5-fluoro-6-hydroxypyridin-2-yl)-1,3,4-thiadiazol-2-yl)methyl)-6-isopropyl-4,6-diazaspiro[2.4]heptane-5,7-dione;
(R)-6-(sec-butyl)-4-((5-(5-fluoro-6-hydroxypyridin-2-yl)-1,3,4-thiadiazol-2-yl)methyl)-4,6-diazaspiro[2.4]heptane-5,7-dione;
(S)-6-(sec-butyl)-4-((5-(5-fluoro-6-hydroxypyridin-2-yl)-1,3,4-thiadiazol-2-yl)methyl)-4,6-diazaspiro[2.4]heptane-5,7-dione;
(R)-4-((5-(5-fluoro-6-hydroxypyridin-2-yl)-1,3,4-thiadiazol-2-yl)methyl)-6-(1-phenylethyl)-4,6-diazaspiro[2.4]heptane-5,7-dione;
(S)-4-((5-(5-fluoro-6-hydroxypyridin-2-yl)-1,3,4-thiadiazol-2-yl)methyl)-6-(1-phenylethyl)-4,6-diazaspiro[2.4]heptane-5,7-dione;
6-(tert-butyl)-4-((5-(5-fluoro-6-hydroxypyridin-2-yl)-1,3,4-thiadiazol-2-yl)methyl)-4,6-diazaspiro[2.4]heptane-5,7-dione;
(R)-4-((5-(2,4-difluoro-3-hydroxyphenyl)-1,3,4-thiadiazol-2-yl)methyl)-6-(1-phenylethyl)-4,6-diazaspiro[2.4]heptane-5,7-dione;
(S)-4-((5-(2,4-difluoro-3-hydroxyphenyl)-1,3,4-thiadiazol-2-yl)methyl)-6-(1-phenylethyl)-4,6-diazaspiro[2.4]heptane-5,7-dione;
6-(tert-butyl)-4-((5-(2,4-difluoro-3-hydroxyphenyl)-1,3,4-thiadiazol-2-yl)methyl)-4,6-diazaspiro[2.4]heptane-5,7-dione;
4-((5-(2,4-difluoro-3-hydroxyphenyl)-1,3,4-thiadiazol-2-yl)methyl)-6-phenyl-4,6-diazaspiro[2.4]heptane-5,7-dione;
4-((5-(2,4-difluoro-3-hydroxyphenyl)-1,3,4-thiadiazol-2-yl)methyl)-6-(1-phenylcyclopropyl)-4,6-diazaspiro[2.4]heptane-5,7-dione;
4-((5-(2,4-difluoro-3-hydroxyphenyl)-1,3,4-thiadiazol-2-yl)methyl)-6-(1-(trifluoromethyl)cyclopropyl)-4,6-diazaspiro[2.4]heptane-5,7-dione;
(S)-4-((5-(5-fluoro-6-hydroxypyridin-2-yl)-1,3,4-thiadiazol-2-yl)methyl)-6-(2,2,2-trifluoro-1-phenylethyl)-4,6-diazaspiro[2.4]heptane-5,7-dione;
6-(1-(2,4-difluorophenyl)-2,2,2-trifluoroethyl)-4-((5-(5-fluoro-6-hydroxypyridin-2-yl)-1,3,4-thiadiazol-2-yl)methyl)-4,6-diazaspiro[2.4]heptane-5,7-dione;
(R)-4-((5-(2,4-difluoro-3-hydroxyphenyl)-1,3,4-thiadiazol-2-yl)methyl)-6-(1-(2,4-difluorophenyl)-2,2,2-trifluoroethyl)-4,6-diazaspiro[2.4]heptane-5,7-dione;
(S)-4-((5-(2,4-difluoro-3-hydroxyphenyl)-1,3,4-thiadiazol-2-yl)methyl)-6-(1-(2,4-difluorophenyl)-2,2,2-trifluoroethyl)-4,6-diazaspiro[2.4]heptane-5,7-dione;
(S)-4-((5-(4-fluoro-3-hydroxy-2-methylphenyl)-1,3,4-thiadiazol-2-yl)methyl)-6-(2,2,2-trifluoro-1-phenylethyl)-4,6-diazaspiro[2.4]heptane-5,7-dione;
4-((5-(2,4-difluoro-3-hydroxyphenyl)-1,3,4-thiadiazol-2-yl)methyl)-6-(2,2-difluoroethyl)-4,6-diazaspiro[2.4]heptane-5,7-dione;
4-((4-((5-(2,4-difluoro-3-hydroxyphenyl)-1,3,4-thiadiazol-2-yl)methyl)-5,7-dioxo-4,6-diazaspiro[2.4]heptan-6-yl)methyl)-N-methylbenzamide;
4-((5-(2,4-difluoro-3-hydroxyphenyl)-1,3,4-thiadiazol-2-yl)methyl)-6-(1,1,1-trifluoropent-3-yn-2-yl)-4,6-diazaspiro[2.4]heptane-5,7-dione;
6-benzhydryl-4-((5-(2,4-difluoro-3-hydroxyphenyl)-1,3,4-thiadiazol-2-yl)methyl)-4,6-diazaspiro[2.4]heptane-5,7-dione;
6-(1-(3,4′-difluoro-[1,1′-biphenyl]-4-yl)-2,2,2-trifluoroethyl)-4-((5-(5-fluoro-6-hydroxypyridin-2-yl)-1,3,4-thiadiazol-2-yl)methyl)-4,6-diazaspiro[2.4]heptane-5,7-dione;
4-((5-(2,4-difluoro-3-hydroxyphenyl)-1,3,4-thiadiazol-2-yl)methyl)-6-(1-(3,4′-difluoro-[1,1′-biphenyl]-4-yl)-2,2,2-trifluoroethyl)-4,6-diazaspiro[2.4]heptane-5,7-dione;
(S)-4-((5-(2,4-difluoro-3-hydroxyphenyl)-1,3,4-thiadiazol-2-yl)methyl)-6-(2,2,2-trifluoro-1-phenylethyl)-4,6-diazaspiro[2.4]heptane-5,7-dione;
4-((5-(2-chloro-4-fluoro-3-hydroxyphenyl)-1,3,4-thiadiazol-2-yl)methyl)-6-(1-phenylcyclopropyl)-4,6-diazaspiro[2.4]heptane-5,7-dione; and
4-((5-(5-fluoro-6-hydroxypyridin-2-yl)-1,3,4-thiadiazol-2-yl)methyl)-6-(1-phenylcyclopropyl)-4,6-diazaspiro[2.4]heptane-5,7-dione.

10. A pharmaceutical composition comprising a compound, a pharmaceutically acceptable salt thereof, or a pharmaceutically acceptable prodrug thereof, of any of claims 1 to 9, and a pharmaceutically acceptable carrier.

11. A method for preventing or treating a 17β-HSD13 mediated disease or condition in a subject in need thereof, comprising administering to the subject a therapeutically effective amount of a compound, a pharmaceutically acceptable salt thereof, or a pharmaceutically acceptable prodrug thereof, of any of claims 1 to 9.

12. The method of claim 11, wherein the 17β-HSD13 mediated disease or condition is selected from the group consisting of nonalcoholic fatty liver disease (NAFLD), nonalcoholic steatohepatitis (NASH), alcoholic fatty liver (alcoholic steatosis), alcoholic hepatitis, liver cirrhosis, liver fibrosis, and hepatocellular carcinoma (HCC).

13. A compound, a pharmaceutically acceptable salt thereof, or a pharmaceutically acceptable prodrug thereof, as claimed in any one of claims 1 to 9, for use in treatment of a liver disease.

14. A compound, or a pharmaceutically acceptable salt thereof, or a pharmaceutically acceptable prodrug thereof, as claimed in any one of claims 1 to 9, for use in therapy.

15. A compound, a pharmaceutically acceptable salt thereof, or a pharmaceutically acceptable prodrug thereof, as claimed in any one of claims 1 to 9, for use in the treatment of NASH.

16. A method of treating liver disease in a patient comprising administering to the patient a compound, a pharmaceutically acceptable salt thereof, or a pharmaceutically acceptable prodrug thereof, as claimed in any one of the claims 1 to 9.

17. A method of reducing the development of liver cirrhosis, cirrhotic decompensation, progression to model of end-stage liver disease (MELD), liver transplant, liver-related death, or hepatocellular carcinoma by administering to a human a compound, a pharmaceutically acceptable salt of, or a pharmaceutically acceptable prodrug thereof, of any of claims 1-9.

18. A pharmaceutical combination composition comprising:
a first compound, said first compound being a compound of any of claims 1-9 or a pharmaceutically acceptable salt of said compound;
a second compound, said second compound being an anti-diabetic agent; a non-alcoholic steatohepatitis treatment agent, or a non-alcoholic fatty liver disease treatment agent; and
a pharmaceutically acceptable carrier.

19. The pharmaceutical combination composition as recited in claim 18 wherein said non-alcoholic steatohepatitis treatment agent or non-alcoholic fatty liver disease treatment agent is an ACC inhibitor, a FASN inhibitor, a KHK inhibitor, a DGAT-2 inhibitor, an FXR agonist, metformin, incretin analogs, or an incretin receptor modulator.

20. The pharmaceutical combination composition as recited in claim 18 wherein said anti-diabetic agent is an SGLT-2 inhibitor, metformin, incretin analogs, an incretin receptor modulator, a DPP-4 inhibitor, or a PPAR agonist.
